(19) United States
(12) Patent Application Publication
(10) Pub. No.: US 2023/0065495 A1
(43) Pub. Date: Mar. 2, 2023

(54) MULTIPARTITE AND CIRCULARLY PERMUTED BETA-BARREL POLYPEPTIDES AND METHODS FOR THEIR USE

(71) Applicant: University of Washington, Seattle, WA (US)

(72) Inventors: Jason C. KLIMA, Seattle, WA (US); David BAKER, Seattle, WA (US)

(21) Appl. No.: 17/760,165

(22) PCT Filed: Feb. 5, 2021
(86) PCT No.: PCT/US2021/016712

§ 371 Date: Aug. 4, 2022

Related U.S. Application Data
(60) Provisional application No. 62/971,490, filed on Feb. 7, 2020.
(60) Provisional application No. 63/116,875, filed on Nov. 22, 2020.

(51) Int. Cl. C07K 14/00; C12N 15/64

(52) U.S. Cl. CPC C07K 14/001; C12N 15/64

(57) ABSTRACT

Disclosed herein are β-barrel polypeptides including self-complementing multipartite β-barrel polypeptides and circularly permuted β-barrel polypeptides and methods for their design and use in mediating real-time monitoring of polypeptide-polypeptide association and dissociation events.

### CROSS REFERENCE

This application claims priority to U.S. Provisional Patent Application Serial Nos. 62/971,490 filed Feb. 7, 2020 and 63/116,875 filed Nov. 22, 2020, incorporated by reference herein in their entirety.

### SEQUENCE LISTING STATEMENT

A computer readable form of the Sequence Listing is filed with this application by electronic submission and is incorporated into this application by reference in its entirety. The Sequence Listing is contained in the file created on Jan. 28, 2021, having the file name “19-2449-PCT_SequenceListing_ST25.txt” and is 479 kb in size.

### BACKGROUND

β-barrels with antiparallel β-strands are excellent polypeptide scaffolds for ligand binding, as the base of the β-barrel can accommodate a hydrophobic core to provide overall stability, and the top of the β-barrel can provide a recessed cavity for ligand binding (often flanked by loops which can contribute further ligand binding affinity and selectivity). However, β-sheet topologies are notoriously difficult to design from scratch, much less multipartite or circularly permuted β-sheet topologies.

### SUMMARY

In one aspect, the disclosure provides non-naturally occurring, self-complementing multipartite β-barrel proteins, comprising at least a first polypeptide component and a second polypeptide component, wherein the at least first polypeptide component and the second polypeptide component are not covalently linked, wherein in total the at least first polypeptide component and the second polypeptide component comprise domains X1-X2-X3-X4-X5-X6-X7-X8-X9-X10-X11-X12-X13-X14-X15-X16-X17-X18-X19, wherein:

X1 comprises a capping domain;

X2 comprises a beta strand,

wherein a contiguous C-terminal portion of X1 and N-terminal portion of X2 comprise the amino acid sequence Z1-P-G-Z2-W, where Z1 and Z2 are any amino acid;

X3 comprises a beta turn;

X4 comprises a beta strand that includes an internal G residue and a P at its C-terminus;

X5 comprises a single polar amino acid;

X6 comprises a beta turn;

X7 comprises a beta strand including an internal G residue;

X8 comprises a beta turn;

X9 comprises a beta strand including an internal P residue and 2 internal G residues;

X10 comprises a single polar amino acid;

X11 comprises a beta turn;

X12 comprises a beta strand;

X13 comprises a beta turn;

X14 comprises a beta strand with an internal G residue;

X15 comprises a single polar amino acid;

X16 comprises a beta turn;

X17 comprises a beta strand;

X18 comprises a beta turn; and

X19 comprises a beta strand;

wherein (a) each beta strand is fully present within one polypeptide component of the at least first polypeptide component and the second polypeptide component, (b) none of the at least first polypeptide component and the second polypeptide component include each of X2, X4, X7, X9, X12, X14, X17, and X19; and (c) one of domains X3, X6, X8, X11, X13, X16, and X18 may be partially or wholly absent in each of the first polypeptide and the second polypeptide.

In a second aspect, the disclosure provides polypeptides comprising a first polypeptide component or a second polypeptide component of any embodiment or combination of embodiments of the first aspect of the disclosure, including but not limited to polypeptides comprising an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOs:1-308, wherein residues in parentheses are optional, and wherein the optional residues may be present or absent.

In a third aspect, the disclosure provides β-barrel polypeptides, comprising domains X1-X2-X3-X4-X5-X6-X7-X8-X9-X10-X11-X12-X13-X14-X15-X16-X17-X18-X19, wherein:

X1 comprises a capping domain;

X2 comprises a beta strand,

wherein a contiguous C-terminal portion of X1 and N-terminal portion of X2 comprise the amino acid sequence Z1-P-G-Z2-W, where Z1 and Z2 are any amino acid;

X3 comprises a beta turn;

X4 comprises a beta strand that includes an internal G residue and a P at its C-terminus;

X5 comprises a single polar amino acid;

X6 comprises a beta turn;

X7 comprises a beta strand including an internal G residue;

X8 comprises a beta turn;

X9 comprises a beta strand including an internal P residue and 2 internal G residues;

X10 comprises a single polar amino acid;

X11 comprises a beta turn;

X12 comprises a beta strand;

X13 comprises a beta turn;

X14 comprises a beta strand with an internal G residue;

X15 comprises a single polar amino acid;

X16 comprises a beta turn;

X17 comprises a beta strand;

X18 comprises a beta turn; and

X19 comprises a beta strand;

wherein the last residue of the X19 domain is N-terminal to and connected to the first residue of X1 domain via an amino acid linker;

wherein 1, 2, or 3 contiguous domains X1, X2, X3, X4, X5, X6, X7, X8, X9, X10, X11, X12, X13, X14, X15, X16, X17, X18, and X19 may be partially or wholly absent. In one embodiment, 0 or 1 domain is wholly absent.

In one non-limiting embodiment, the polypeptides of the third aspect may comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOs: 309-532.

In a fourth aspect, the disclosure provides β-barrel polypeptides comprising an amino acid sequence at least 80%, 85%, 90%, 91%, 92%, 93%, 94%, 95%, 96%, 97%, 98%, 99%, or 100% identical to the amino acid sequence selected from the group consisting of SEQ ID Nos:533-534.

In other aspects, the disclosure provides nucleic acids encoding the polypeptides and polypeptide components of the disclosure, expression vectors comprising the nucleic acids operatively linked to a control sequence, host cells comprising the polypeptides, polypeptide components, nucleic acids and/or expression vectors, and pharmaceutical compositions, comprising the self-complementing multipartite β-barrel protein, the polypeptide, the nucleic acid, the expression vector, the recombinant cell, and/or the 0-barrel polypeptide of any of the aspects and embodiments herein; and a pharmaceutically acceptable carrier.

In a further aspect, the disclosure provides methods for using the self-complementing multipartite β-barrel protein, the polypeptide, the nucleic acid, the expression vector, the recombinant cell, and/or the β-barrel polypeptide of any of aspects and embodiments herein, for uses including, but not limited to, pH sensing, ion-sensing/detection (including but not limited to Ca′, La′, Tb′, and other ion sensing/detection/quantification), temporal sensing, voltage sensing, mechanical sensing, thermal sensing, super-resolution microscopy, localization microscopy, fluorescence microscopy, fluorescence lifetime imaging, fluorimetry, and detection and quantification of other small-molecules, ions, peptides, nucleic acids, organic substrates, or inorganic substrates by insertion of their respective binding peptides into the loops, beta turns, or beta strands of any of the polypeptides of any of the claims herein, or by covalent fusion or non-covalent linkage of their respective binding peptides to any of the polypeptides of any of the aspects and embodiments herein.

### DESCRIPTION OF THE FIGURES

FIG. 1(a-g). Design and biophysical characterization of bipartite split mFAP2a variants. (a) Computational model of mFAP2a (top row) used to design self-complementing split mFAPs (bottom row). Separate polypeptide chains (lightly shaded and darkly shaded cartoons, respectively) and the chromophore DFHBI-1T (spheres) are shown. Split mFAP fragment combinations are annotated above split mFAP models (bottom row), showing (left to right): β-strand 1 with β-strands 2-8 (m1+m28); β-strands 1-2 with β-strands 3-8 (m12+m38); β-strands 1-3 with β-strands 4-8 (m13+m48); β-strands 1˜4 with β-strands 5-8 (m14+m58); β-strands 1-5 with β-strands 6-8 (m15+m68); β-strands 1-6 with β-strands 7-8 (m16+m78); and β-strands 1-7 with β-strand 8 (m17+m8). (b) Self-complementation of maltose binding protein (MBP)-tagged split mFAPs incubated at the annotated equimolar concentrations in 50.004 DFHBI-1T showing the average (n=3) fluorescence intensity. Error bars represent the standard deviation of the mean of 3 technical replicates. (c) Normalized fluorescence excitation (dotted lines) and emission (solid lines) spectra (n=1) of assembled MBP-tagged split mFAP fragments. (d-g) Titrations of MBP-tagged split mFAP fragments into their complementary MBP-tagged split mFAP fragments showing normalized fluorescence intensity in 25.0 μM DFHBI-1T (points). (d) MBP-tagged m12 was fixed at 21.9 μM final concentration as MBP-tagged m38 was titrated (n=1). (e) MBP-tagged m14 was fixed at 20.3 μM final concentration as MBP-tagged m58 was titrated (n=1). (f) MBP-tagged m16 was fixed at 16.8 μM final concentration as MBP-tagged m78 was titrated (n=1). (g) MBP-tagged m17 was fixed at 14.1 μM final concentration as MBP-tagged m8 was titrated (n=1). The annotated thermodynamic dissociation constants (K_{d} values) are at least the highest concentration of titrant measured.

FIG. 2(a-f). Assembly and disassembly of bipartite split mFAP fragments m14 and m58. (a-d) Assembly of split mFAP fragments. (a) Association model in which BCLXL is fused to m58 (BCLXL_m58), aBCLXL is fused to m14 (m14_aBCLXL), and fluorescence of DFHBI-1T (spheres) is activated upon association (arrow) of BCLXL_m58 and m14_aBCLXL. (b) Normalized fluorescence intensity (points) of BCLXL_m58 titration into a constant m14_aBCLXL concentration in excess DFHBI-1T after reaching equilibrium, showing the fit to a bimolecular association model (line) using non-linear least squares fitting. (c) Split mFAP competitor pre-incubation model in which fluorescence of DFHBI-1T (spheres) is activated upon competition (arrow) of m14_aBCLXL with unfused aBCLXL for the BCLXL binding cleft of BCLXL_m58 (the reaction evolves analogously for BFL1-aBFL1 and BCL2-aBCL2 cognate binding partners). (d) Temporal evolution of fluorescence fold-change in excess DFHBI-1T upon (n=1) addition of equimolar m14_aBFL1 or buffer to pre-incubated equimolar BFL1_m58 and aBFL1, addition of equimolar m14_aBCL2 or buffer to pre-incubated equimolar BCL2 m58 and aBCL2, and addition of equimolar m14_aBCLXL or buffer to pre-incubated equimolar BCLXL_m58 and aBCLXL, showing fits to a monophasic exponential function (lines) using non-linear least squares fitting. (e,f) Disassembly of split mFAP fragments. (e) Pre-assembled split mFAP competition model in which BCL2 is fused to m58 (BCL2_m58) aBFL1 is fused to m14 (m14_aBFL1), and fluorescence of DFHBI-1T (spheres) is activated before unfused aBCL2 competes with m14_aBFL1 for the BCL2 binding cleft of BCL2_m58 (arrow), resulting in fluorescence deactivation. (f) Temporal evolution of fluorescence fold-change in excess DFHBI-1T of pre-incubated equimolar BCL2_m58 and m14_aBFL1 at 2.00 μM final concentrations with unfused aBCL2 titrated in at (n=1) 0 μM, 4.00 μM, and 10.0 μM final concentrations, showing fits to a monophasic exponential function (lines) using non-linear least squares fitting.

FIG. 3(a-b). Photophysical characterization of split mFAP2a fragments m14 and m58 fused to BCL2 family proteins. (a) Normalized fluorescence excitation (dotted lines) and emission (solid lines) spectra (n=1) after equilibrium was reached in FIG. 2d, in which BCLXL_m58 was pre-incubated with aBCLXL in excess DFHBI-1T before addition of m14_aBCLXL or buffer. The reaction evolved analogously for BFL1-aBFL1 and BCL2-aBCL2 cognate binding partners. (b) Normalized fluorescence excitation (dotted lines) and emission (solid lines) spectra (n=1) after equilibrium was reached in FIG. 2f, in which BCL2_m58 was pre-assembled with m14_aBFL1 at 2.00 μM final concentrations in excess DFHBI-1T before addition of unfused aBCL2 at 0 μM, 4.00 μM, and 10.0 μM final concentrations.

FIG. 4(a-d). Computational design and photophysical characterization of circularly permuted mFAP (cpmFAP) variants. (a) Superimposed and overlaid computational models of de novo designed circularly permuted mFAP2a variants from (b), showing circularly permuted β-barrel protein backbones (cartoons) bound to the chromophore DFHBI-1T (spheres) and de novo designed linkers covalently fusing together the N- and C-termini of mFAP2a (cartoons). (b) Average fluorescence intensity of 50.0 μM cpmFAPs versus mFAP2a in 500 nM DFHBI-1T. (c) Computational model of the cpmFAP cp35-34_mFAP2a 12, the brightest cpmFAP variant in this study (b,d), showing the protein backbone (cartoon) with N-terminus and C-terminus bound to the chromophore DFHBI-1T (spheres). (d) Average fluorescence intensity of 40.0 μM cpmFAP variants versus mFAP2a in 50.0 nM DFHBI-1T. (b,d) Error bars represent the standard deviation of the mean of 3 technical replicates.

FIG. 5(a-g). Size-exclusion chromatography (SEC) and SEC with multi-angle light scattering (MALS) of mFAP10 and circularly permuted mFAP (cpmFAP) variants. (a-f) SEC traces of protein samples run on a Superdex™ 75 Increase 10/300 GL column measuring absorbance at 280 nm (n=1), showing representative traces for 6×His-tagged (a) mFAP10, (b) cp89-88_mFAP2a_06, (c) cp106-105_mFAP2a_12_t, (d) cp63-62_mFAP2a_08_t, (e) cp35-34_mFAP2a_10, and (f) the brightest cpmFAP tested, cp35-34_mFAP2a_12. (g) SEC-MALS analysis (n=1) revealed a monomer peak for 6×His-tagged cp35-34_mFAP2a_12 in which the measured molecular mass (1.684·10⁴±8.338% Da) corroborated the expected monomeric molecular mass (1.691·10⁴ Da), showing light scattering (LS) signal, ultraviolet absorbance (UV) signal, and differential refractive index (dRI) signal.

FIG. 6(a-e). Characterization of brighter and chromophore-specific mFAPs. (a) Computational model of de novo designed β-barrel variant mFAP2b showing protein backbone (cartoon) and bound DFHBI chromophore (sticks). (b,c) Chemical structures of DFHBI and DFHBI-1T, respectively. (d,e) In vitro titration of (d) DFHBI or (e) DFHBI-1T with mFAP2, mFAP2b, mFAP2a, and mFAP10 proteins. Error bars represent the standard deviation of the mean of 8 technical replicates. Normalized means were fit to a single binding site isotherm function using non-linear least squares fitting to obtain K_{d} values (Table 4), and the fits scaled to the maximum mean relative fluorescence unit (RFU) values (lines).

FIG. 7. Engineering of mFAP9 and mFAP10 from mFAP2a. Average (n=3) fluorescence intensity (points) from the deprotonated (phenolate) forms of DFHBI (bars) and DFHBI-1T (hatched bars) labeled 680-fold below protein concentration of equimolar mFAP2a, mFAP9 and mFAP10.

FIG. 8(a-f). Photophysical characterization of mFAP10, mFAP2a and mFAP2b in complex with DFHBI or DFHBI-1T chromophores, and chromophores only. (a,c,e) Absorbance spectra (n=1) of saturated protein-chromophore complexes or chromophores only. (b,d,f) Normalized fluorescence excitation (dotted lines) and emission (solid lines) spectra (n=1) of saturated protein-chromophore complexes. (a,b,c,d) The chromophores are at 1.00 μM final concentration. (e,f) The final concentrations of DFHBI are 836 nM and the final concentrations of DFHBI-1T are 919 nM. (a-f) In conditions containing protein and chromophore, the total protein concentration is in excess to the total chromophore concentration, and the percent of the chromophore bound in complex with protein is reported in Table 4.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure and appendices can be used in combination, unless the context clearly dictates otherwise.

Unless the context clearly requires otherwise, throughout the description, appendix, and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides non-naturally occurring, self-complementing multipartite β-barrel proteins, comprising at least a first polypeptide component and a second polypeptide component, wherein the at least first polypeptide component and the second polypeptide component are not covalently linked, wherein in total the at least first polypeptide component and the second polypeptide component comprise domains X1-X2-X3-X4-X5-X6-X7-X8-X9-X10-X11-X12-X13-X14-X15-X16-X17-X18-X19, wherein:

X1 comprises a capping domain;

X2 comprises a beta strand,

wherein a contiguous C-terminal portion of X1 and N-terminal portion of X2 comprise the amino acid sequence Z1-P-G-Z2-W, where Z1 and Z2 are any amino acid;

X3 comprises a beta turn;

X4 comprises a beta strand that includes an internal G residue and a P at its C-terminus;

X5 comprises a single polar amino acid;

X6 comprises a beta turn;

X7 comprises a beta strand including an internal G residue;

X8 comprises a beta turn;

X9 comprises a beta strand including an internal P residue and 2 internal G residues;

X10 comprises a single polar amino acid;

X11 comprises a beta turn;

X12 comprises a beta strand;

X13 comprises a beta turn;

X14 comprises a beta strand with an internal G residue;

X15 comprises a single polar amino acid;

X16 comprises a beta turn;

X17 comprises a beta strand;

X18 comprises a beta turn; and

X19 comprises a beta strand;

wherein (a) each beta strand is fully present within one polypeptide component of the at least first polypeptide component and the second polypeptide component, (b) none of the at least first polypeptide component and the second polypeptide component include each of X2, X4, X7, X9, X12, X14, X17, and X19; and (c) one of domains X3, X6, X8, X11, X13, X16, and X18 may be partially or wholly absent in each of the first polypeptide and the second polypeptide.

As disclosed herein, the inventors have produced self-complementing multipartite β-barrel polypeptides (“split mFAPs”, where each polypeptide component is non-covalently linked) capable of mediating real-time monitoring of polypeptide-polypeptide association and dissociation events through self-complementation, into a reporter complex capable of activating the fluorescence of exogenous fluorogenic compounds such as DFHBI (3,5-difluoro-4-hydroxybenzylidene imidazolinone)^{1,2,3,4}, DFHBI-1T [(Z)-4-(3,5-difluoro-4-hydroxybenzylidene)-2-methyl-1-(2,2,2-trifluoroethyl)-1H-imidazol-5(4H)-one]⁵, and DFHO (3,5-difluoro-4-hydroxybenzylidene imidazolinone-2-oxime), with different degrees of specificity and affinity. Such multipartite β-barrel polypeptides and other β-barrel polypeptides disclosed herein may be used as versatile polypeptide scaffolds in the engineering of novel oligomeric polypeptide assemblies and novel fluorescent biosensors for the detection of analytes of interest in real-time using fluorescence microscopy and fluorimetry techniques. Exemplary starting β-barrel polypeptides (i.e.: non-split β-barrel polypeptides, also known as canonical mFAPs) can be found, for example in WO2019/195525 published Oct. 10, 2019, incorporated by reference herein in its entirety.

The split mFAPs comprise at least a first polypeptide component and a second polypeptide component in which β-strands are preserved while split points in the β-barrel polypeptides are taken only in the beta turns. In other words, each beta strand (X2, X4, X7, X9, X12, X14, X17, ands X19) is fully present within one polypeptide component of the at least first polypeptide component and the second polypeptide component, while the β-barrel polypeptide is split into separate components in beta turns (X3, X6, X8, X11, X13, X16, or X18). By way of non-limiting example, in various embodiment of a bipartite β-barrel protein, the first polypeptide component and the second polypeptide component may comprise as follows:

| | First polypeptide | Second polypeptide |
|---|---|---|
| Example | component comprises | component comprises |
| 1: Split at X3 | X1-X2-(X3) | (X3)-X4-X5-X6-X7-X8-X9- |
| beta turn | | X10-X11-X12-X13-X14- |
| | | X15-X16-X17-X18-X19 |
| 2: Split at X6 | X1-X2-X3-X4-X5-(X6) | (X6)-X7-X8-X9-X10-X11- |
| beta turn | | X12-X13-X14-X15-X16- |
| | | X17-X18-X19 |
| 3: Split at X8 | X1-X2-X3-X4-X5- | (X8)-X9-X10-X11-X12-X13- |
| beta turn | X6-X7-(X8) | X14-X15-X16-X17-X18-X19 |
| 4: Split at X11 | X1-X2-X3-X4-X5-X6-X7- | (X11)-X12-X13-X14-X15- |
| beta turn | X8-X9-X10-(X11) | X16-X17-X18-X19 |
| 5: Split at X13 | X1-X2-X3-X4-X5-X6-X7- | (X13)-X14-X15-X16-X17- |
| beta turn | X8-X9-X10-X11-X12-(X13) | X18-X19 |
| 6: Split at X16 | X1-X2-X3-X4-X5-X6-X7- | (X16)-X17-X18-X19 |
| beta turn | X8-X9-X10-X11-X12-X13- | |
| | X14-X15-(X16) | |
| 7: Split at X18 | X1-X2-X3-X4-X5-X6-X7-X8- | (X18)-X19 |
| beta turn | X9-X10-X11-X12-X13-X14- | |
| | X15-X16-X17-(X18) | |

In each embodiment, the point at which the original non-split β-barrel polypeptide is split (i.e. the “split point”) can be present in the first polypeptide component, the second polypeptide component, or neither of the polypeptide components after splitting. In the case of neither, this is due to the elimination of the beta turn at which the split point is made, such that the original beta turn is transformed into residues on each component comprising polypeptide fragments acquiring loop, beta-strand, or alpha-helical secondary structures. For this reason, the split point is noted in parentheses, to note that it is optional in each of the first and second polypeptide component, but is not required to be present in one or the other polypeptide component.

In various embodiments, the at least a first polypeptide component and a second polypeptide component may comprise 2, 3, 4, 5, 6, 7, or 8 polypeptide components. As will be understood by those of skill in the art based on the teachings herein, there exists one split point for bipartite β-barrel polypeptides, two split points for tripartite β-barrel polypeptides, three split points for tetrapartite β-barrel polypeptides, four split points for pentapartite β-barrel polypeptides, five split points for hexapartite β-barrel polypeptides, six split points for heptapartite β-barrel polypeptides, and seven split points for octapartite β-barrel polypeptides. In one non-limiting embodiment, two examples of a tripartite β-barrel polypeptide may be as follows:

| | First polypeptide | Second polypeptide | Third polypeptide |
|---|---|---|---|
| Example | component comprises | component comprises | component comprises |
| 1: Split at X3 and | X1-X2-(X3) | (X3)-X4- | (X6)-X7-X8-X9-X10- |
| X6 beta turns | | X5-(X6) | X11-X12-X13-X14- |
| | | | X15-X16-X17-X18- |
| | | | X19 |
| 2 Split at X3 and | X1-X2-(X3) | (X3)-X4-X5- | (X8)-X9-X10-X11- |
| X8 beta turns | | X6-X7-(X8) | X12-X13-X14-X15- |
| | | | X16-X17-X18-X19 |

In other embodiments, redundant (and hence identical) beta-strands are allowed on the different polypeptide components that comprise the fluorescently active complex, so long as all 8 unique beta-strands (i.e.: X2, X4, X7, X9, X12, X14, X17, ands X19) participate in the fluorescently active complex, regardless of the number of polypeptide components participating in the fluorescently active complex, i.e. between 2 and 8 different polypeptide components for multipartite beta-barrels. Thus, for example, in another non-limiting embodiment, three examples of a tripartite β-barrel polypeptide may be as follows:

| | First polypeptide | Second polypeptide | Third polypeptide |
|---|---|---|---|
| Example | component comprises | component comprises | component comprises |
| 1: Split at X3 and | X1-X2-(X3) | (X3)-X4-X5-X6-X7-X8- | (X6)-X7-X8-X9-X10- |
| X6 beta turns | | X9-X10-X11-X12-X13- | X11-X12-X13-X14- |
| | | X14-X15-X16-X17-X18- | X15-X16-X17-X18- |
| | | X19 | X19 |
| 2: Split at X3 and | X1-X2-(X3) | (X3)-X4-X5-X6-X7-X8- | (X8)-X9-X10-X11- |
| X8 beta turns | | X9-X10-X11-X12-X13- | X12-X13-X14-X15- |
| | | X14-X15-X16-X17-X18- | X16-X17-X18-X19 |
| | | X19 | |
| 3: Split at X3, X8 | X1-X2-(X3) | (X3)-X4-X5-X6-X7-X8- | (X8)-X9-X10-X11- |
| and X13 beta | | X9-X10-X11-X12-(X13) | X12-X13-X14-X15- |
| turns | | | X16-X17-X18-X19 |

Based on the teachings herein, those of skill in the art will understand the various other tripartite and multipartite embodiments exemplified above.

The at least first polypeptide component and the at least second polypeptide component are not covalently linked (for example, not both present in a single fusion protein), but spontaneously assemble in solution. The molar fraction of polypeptide components participating in a fluorescently active assembled complex to individual polypeptide components not participating in an assembled complex depends on the unique thermodynamic dissociation constants of the polypeptide components for one another, the unique thermodynamic dissociation constants of the individual polypeptide components for the chromophore, and the concentrations of polypeptide components and chromophore in solution.

As used herein, a “capping domain” is any sequence of amino acids that appropriately position the Z1-P-G-Z2-W domain noted above (also referred to herein as the “tryptophan corner”). As such, the capping domain may be of any suitable length and amino acid composition. In one non-limiting embodiment, the capping domain may comprise an alpha-helical domain. Exemplary capping domains are provided in the specific polypeptide sequences disclosed herein.

In one embodiment, Z1 is a hydrophobic amino acid and Z2 is a polar amino acid. In another embodiment, Z1 is selected from the group consisting of L, A, and F, or Z1 is L. In a further embodiment, Z2 is selected from the group consisting of T, K, N, and D, or Z2 is T. In one embodiment, X1 comprises the amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO: 535) or RAAQLLP (SEQ ID NO: 536), wherein the highlighted residue is invariant.

As used herein, each “beta strand” may be any suitable series of amino acids that include alternating hydrophobic and polar amino acid residues (in whole or in part). In some embodiments, each beta strand independently is between 8-12, 8-11, 8-10, 8-9, 9-12, 9-11, 9-10, 10-12, 10-11, 8, 9, 10, 11, or 12 amino acid residues in length when not including a functional domain, as discussed below.

As used herein, each “beta turn” may be any suitable sequence that can serve to transition between two beta strands in the polypeptide. In various embodiments, each beta turn may independently be 3-5, 4-5, 3, 4, or 5 amino acids in length when not including a functional domain, as discussed below. In other embodiments, one or more beta turn may include a proline residue.

In various embodiments (which may be combined) based on the various designs disclosed herein:
Z2 is selected from the group consisting of T, K, N, and D
the X1 capping domain comprises an alpha helix;
X1 comprises the amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO: 535) or RAAQLLP (SEQ ID NO: 536), wherein the highlighted residue is invariant.
X2 comprises an amino acid sequence at least 50%, 55%, 60%, 65%, 70%, 75%, 80%, 85%, 90%, 95%, or 100% identical to the amino acid sequence G (T/K/N/D) WQZT(M/F)TN (SEQ ID NO: 537) wherein Z is any amino acid, or GTWQ(V/L/A/I) T(M/F)TN (SEQ ID NO: 538), wherein the highlighted residues are invariant;
X3 comprises the amino acid sequence (E/S)DG or EDG;
X4 comprises an amino acid sequence at least 50%, 55%, 60%, 65%, 70%, 75%, 80%, 85%, 90%, 95%, or 100% identical to the amino acid sequence QTSQGQMHFQP (SEQ ID NO: 539), wherein the highlighted residues are invariant;
X5 comprises a single polar amino acid selected from the group consisting of R, T, Q, N, K, E, D, S, or wherein X5 is R;
X6 comprises the amino acid sequence (T/S)PZ3, where Z3 is polar amino acid or Tyr; or X6 is SPY;
X7 comprises an amino acid sequence at least 50%, 55%, 60%, 65%, 70%, 75%, 80%, 85%, 90%, 95%, or 100% identical to the amino acid sequence T(L/A/M)D(IN)(K/V)(A/S) GT(I/M) (SEQ ID NO:540) or TMDIVAQGTI (SEQ ID NO:541), wherein the highlighted residues are invariant;
X8 comprises the amino acid sequence (S/A)DG or SDG;
X9 comprises an amino acid sequence at least 50%, 55%, 60%, 65%, 70%, 75%, 80%, 85%, 90%, 95%, or 100% identical to the amino acid sequence RPI(Q/S/T/V)G(Y/K)GK(L/V/A)T(V/C/A) (SEQ ID NO: 542) or RPIVGYGKATV (SEQ ID NO: 543), wherein the highlighted residues are invariant;
X10 is selected from the group consisting of R, T, Q, N, K, E, D, or S; or X10 is K;
X11 comprises the amino acid sequence (S/T)(P/C)(polar or Y), or X 11 is TPD;
X12 comprises an amino acid sequence at least 50%, 55%, 60%, 65%, 70%, 75%, 80%, 85%, 90%, 95%, or 100% identical to the amino acid sequence T(M/L/V)(D/H/Q/N)(V/A/L/I)(D/N/H/Q)(I/L/V) T(Y/W) (SEQ ID NO:544) or TLDIDITY (SEQ ID NO:545);
X13 comprises the amino acid sequence (S/E)DG, or wherein X13 comprises an amino acid sequence at least 60%, 80%, or 100% identical to PSLGN (SEQ ID NO: 546);
X14 comprises an amino acid sequence at least 50%, 55%, 60%, 65%, 70%, 75%, 80%, 85%, 90%, 95%, or 100% identical to the amino acid sequence (K/M/I/L)(Q/K)(V/A/G)QGQ(V/I)T(M/L/Y) (SEQ ID NO:547) or IKAQGQITM (SEQ ID NO:548), wherein the highlighted residues are invariant;
X15 is selected from the group consisting of R, T, Q, N, K, E, D, or S, or X15 is D;
X16 comprises the amino acid sequence (S/T)P(D/T/Y), or X16 comprises the amino acid sequence SPT;
X17 comprises an amino acid sequence at least 50%, 55%, 60%, 65%, 70%, 75%, 80%, 85%, 90%, 95%, or 100% identical to the amino acid sequence Q(F/A)(K/T/H)(F/W)(D/N)(V/A/S/G)(T/Q/H/E) (T/FN/Y) (SEQ ID NO:549) or QFKFDATT (SEQ ID NO:550);
X19 comprises an amino acid sequence at least 50%, 55%, 60%, 65%, 70%, 75%, 80%, 85%, 90%, 95%, or 100% identical to the amino acid sequence [(S/K/N/H)](K/R/I/N)(V/L)TGT(L/I/M)QRQE (SEQ ID NO:551) or RLTGTLQRQE (SEQ ID NO:552), wherein residues in brackets are optional; and/or
X18 comprises the amino acid sequence selected from the group consisting of (S/E/N/A/Q)DG, SDG, K(G/Q/K/T)(A/D/E/N)(G/D/N)(N/G/D/Y/S) (SEQ ID NO:553), KG(A/D/E)(G/D/N)(N/G/D/Y) (SEQ ID NO:554), KGENDFHG (SEQ ID NO:555), KGADGWHG (SEQ ID NO:556), and KGAGNFTG (SEQ ID NO:557).

In another embodiment, the first polypeptide component and/or the second polypeptide component comprise an amino acid sequence at least 80%, 85%, 90%, 91%, 92%, 93%, 94%, 95%, 96%, 97%, 98%, 99%, or 100% identical to the amino acid sequence of a polypeptide in Table 1 (SEQ ID NOS:1-308), wherein residues in parentheses are optional. In one embodiment, the optional residues are present. In other embodiments, 1, 2, 3, 4, 5, 6, 7, 8, 9, or all optional residues at the N terminus and/or the C-terminus of any one of SEQ ID NOS: 1-308 may independently be absent. As will be understood by those of skill in the art, if less than all optional residues are absent, those residues at the termini of the optional region would be absent. By way of non-limiting example:
If one residue of the N-terminal optional region of SEQ ID NO:67 was absent, the N-terminal “S” residue would be absent;
If two residues of the C-terminal optional region of SEQ ID NO:67 was absent, the C-terminal “HG” residues would be absent

(SEQ ID NO: 67)
(SR)AAQLLPGTWQATETNEDGQTSQGQWHFQPRSPYTMDIVAQGTISD
GRPIVGYGKATVKTPDTLDIDITYPSLGNIKAQGQITMDSPTQFKWDA
(TTKGENDFHG)

TABLE 1
Amino acid sequences of self-complementing multipartite β-barrel
polypeptide fragments. The design naming convention used was: “m” (shorthand for mFAP)
+ the β-barrel polypeptide segment numbered by β-strands (e.g. “14” harbors β-strands 1-4,
“8” only harbors β-strand 8, etc.) + an optional “.” followed by a sequence variant number
(e.g. “.1”). Due to the redundancy of each of the 8 unique β-strands amongst many of the
multipartite β-barrel polypeptide fragments (Table 1) such as by way of a non-limiting
example of β-strand 8 (e.g. m8), β-barrel polypeptide β-strands 1-7 (e.g. m17) may assemble
together with β-strands 2-8 (e.g. m28), β-strands 3-8 (e.g. m38), β-strands 4-8 (e.g. m48), β-
strands 5-8 (e.g. m58), β-strands 6-8 (e.g. m68), or β-strand 7-8 (e.g. m78) to form an active
reporter complex. As long as all eight unique β-strands are structurally associated forming the
fluorescently active multipartite β-barrel polypeptide complex, then any combination of β-
barrel polypeptide fragments may be used to monitor association and dissociation events of
homooligomeric and heterooligomeric polypeptide complexes of interest. In various non-
limiting embodiments of bipartite split mFAPs by naming convention: any m1 + any m28;
any m12 + any m38; etc.; but also any m17 + any m28; any m16 + any m28; etc; since all 8
β-strands are present in the active complex. In relation to the naming convention, so long as
the β-barrel polypeptide segments (numbered by β-strands) of the components in question
cover all 8 β-strands, then the polypeptide components are capable of assembling into a
fluorescently active complex.

| Design | |
|---|---|
| Name | Sequence |
| m1 | (SR)AAQLLPGTWQATFTN(E) (SEQ ID NO: 1) |
| | SRAAQLLPGTWQATTTNE (SEQ ID NO: 2) |
| m1.1 | (SR)AAQLLPGTWQVTMTN(E) (SEQ ID NO: 3) |
| | SRAAQLLPGTWQVTMTNE (SEQ ID NO: 4) |
| m12 | (SR)AAQLLPGTWQATFTNEDGQTSQGQWHFQPRS(P) (SEQ ID NO: 5) |
| | SRAAQLLPGTWQATTTNEDGQTSQGQWHFQPRSP (SEQ ID NO: 6) |
| m12.1 | (SR)AAQLLPGTWQVTMTNEDGQTSQGQWHFQPRS(P) (SEQ ID NO: 7) |
| | SRAAQLLPGTWQVTMTNEDGQTSQGQWHFQPRSP (SEQ ID NO: 8) |
| m12.2 | (SR)AAQLLPGTWQATFTNEDGQTSQGQFHFQPRS(P) (SEQ ID NO: 9) |
| | SRAAQLLPGTWQATFTNEDGQTSQGQFHFQPRSP (SEQ ID NO: 10) |
| m12.3 | (SR)AAQLLPGTWQATFTNEDGQTSQGQIHFQPRS(P) (SEQ ID NO: 11) |
| | SRAAQLLPGTWQATFTNEDGQTSQGQIHFQPRSP (SEQ ID NO: 12) |
| m12.4 | (SR)AAQLLPGTWQVTMTNEDGQTSQGQMHFQPRS(P) (SEQ ID NO: 13) |
| | SRAAQLLPGTWQVTMTNEDGQTSQGQMHFQPRSP (SEQ ID NO: 14) |
| m13 | (SR)AAQLLPGTWQATFTNEDGQTSQGQWHFQPRSPYTMDIVAQGTI(S) (SEQ ID NO: 15) |
| | SRAAQLLPGTWQATTTNEDGQTSQGQWHFQPRSPYTMDIVAQGTIS (SEQ ID NO: 16) |
| m13.1 | (SR)AAQLLPGTWQVTMTNEDGQTSQGQWHFQPRSPYTMDIVAQGTI(S) (SEQ ID NO: 17) |
| | SRAAQLLPGTWQVTMTNEDGQTSQGQWHFQPRSPYTMDIVAQGTIS (SEQ ID NO: 18) |
| m13.2 | (SR)AAQLLPGTWQATFTNEDGQTSQGQFHFQPRSPYTMDIVAQGTI(S) (SEQ ID NO: 19) |
| | SRAAQLLPGTWQATFTNEDGQTSQGQFHFQPRSPYTMDIVAQGTIS (SEQ ID NO: 20) |
| m13.3 | (SR)AAQLLPGTWQATFTNEDGQTSQGQIHFQPRSPYTMDIVAQGTI(S) (SEQ ID NO: 21) |
| | SRAAQLLPGTWQATTTNEDGQTSQGQIHFQPRSPYTMDIVAQGTIS (SEQ ID NO: 22) |
| m13.4 | (SR)AAQLLPGTWQATFTNEDGQTSQGQIHFQPRSPYTMDIVSQGTI(S) (SEQ ID NO: 23) |
| | SRAAQLLPGTWQATFTNEDGQTSQGQIHFQPRSPYTMDIVSQGTIS (SEQ ID NO: 24) |
| m13.5 | (SR)AAQLLPGTWQVTMTNEDGQTSQGQMHFQPRSPYTMDIVAQGTI (S) (SEQ ID NO: 25) |
| | SRAAQLLPGTWQVTMTNEDGQTSQGQMHFQPRSPYTMDIVAQGTIS (SEQ ID NO: 26) |
| m14 | (SR)AAQLLPGTWQATFTNEDGQTSQGQWHFQPRSPYTMDIVAQGTISDGRPIVGYGKATV(KTP) |
| | (SEQ ID NO: 27) |
| | SRAAQLLPGTWQAT7TNEDGQTSQGQWHFQPRSPYTMDIVAQGTISDGRPIVGYGKATVKTP (SEQ |
| | ID NO: 28) |
| m14.1 | (SR)AAQLLPGTWQVTMTNEDGQTSQGQWHFQPRSPYTMDIVAQGTISDGRPIVGYGKATV(KTP) |
| | (SEQ ID NO: 29) |
| | SRAAQLLPGTWQVTMTNEDGQTSQGQWHFQPRSPYTMDIVAQGTISDGRPIVGYGKATVKTP (SEQ |
| | ID NO: 30) |
| m14.2 | (SR)AAQLLPGTWQATFTNEDGQTSQGQFHFQPRSPYTMDIVAQGTISDGRPIVGYGKATV(KTP) |
| | (SEQ ID NO: 31) |
| | SRAAQLLPGTWQATFTNEDGQTSQGQFHFQPRSPYTMDIVAQGTISDGRPIVGYGKATVKTP (SEQ |
| | ID NO: 32) |
| m14.3 | (SR)AAQLLPGTWQATFTNEDGQTSQGQIHFQPRSPYTMDIVAQGTISDGRPIVGYGKATV(KTP) |
| | (SEQ ID NO: 33) |
| | SRAAQLLPGTWQATFTNEDGQTSQGQIHFQPRSPYTMDIVAQGTISDGRPIVGYGKATVKTP (SEQ |
| | ID NO: 34) |
| m14.4 | (SR)AAQLLPGTWQATFTNEDGQTSQGQIHFQPRSPYTMDIVSQGTISDGRPIVGYGKATV(KTP) |
| | (SEQ ID NO: 35) |
| | SRAAQLLPGTWQATFTNEDGQTSQGQIHFQPRSPYTMDIVSQGTISDGRPIVGYGKATVKTP (SEQ |
| | ID NO: 36) |
| m14.5 | (SR)AAQLLPGTWQVTMTNEDGQTSQGQMHFQPRSPYTMDIVAQGTISDGRPIVGYGKATV(KTP) |
| | (SEQ ID NO: 37) |
| | SRAAQLLPGTWQVTMTNEDGQTSQGQMHFQPRSPYTMDIVAQGTISDGRPIVGYGKATVKTP (SEQ |
| | ID NO: 38) |
| m15 | (SR)AAQLLPGTWQATFTNEDGQTSQGQWHFQPRSPYTMDIVAQGTISDGRPIVGYGKATVKTPDTLDI |
| | DITYP(S) (SEQ ID NO: 39) |
| | SRAAQLLPGTWQATTTNEDGQTSQGQWHFQPRSPYTMDIVAQGTISDGRPIVGYGKATVKTPDTLDIDI |
| | TYPS (SEQ ID NO: 40) |
| m15.1 | (SR)AAQLLPGTWQVTMTNEDGQTSQGQWHFQPRSPYTMDIVAQGTISDGRPIVGYGKATVKTPDTLDI |
| | DITYP(S) (SEQ ID NO: 41) |
| | SRAAQLLPGTWQVTMTNEDGQTSQGQWHFQPRSPYTMDIVAQGTISDGRPIVGYGKATVKTPDTLDIDI |
| | TYPS (SEQ ID NO: 42) |
| m15.2 | (SR)AAQLLPGTWQATFTNEDGQTSQGQWHFQPRSPYTMDIVAQGTISDGRPIVGYGKATVKTPDTLDI |
| | DITWP(S) (SEQ ID NO: 43) |
| | SRAAQLLPGTWQATFTNEDGQTSQGQWHFQPRSPYTMDIVAQGTISDGRPIVGYGKATVKTPDTLDIDI |
| | TWPS (SEQ ID NO: 44) |
| m15.3 | (SR)AAQLLPGTWQATFTNEDGQTSQGQFHFQPRSPYTMDIVAQGTISDGRPIVGYGKATVKTPDTLDI |
| | DITYP(S) (SEQ ID NO: 45) |
| | SRAAQLLPGTWQATTTNEDGQTSQGQFHFQPRSPYTMDIVAQGTISDGRPIVGYGKATVKTPDTLDIDI |
| | TYPS (SEQ ID NO: 46) |
| m15.4 | (SR)AAQLLPGTWQATFTNEDGQTSQGQIHFQPRSPYTMDIVAQGTISDGRPIVGYGKATVKTPDTLDI |
| | DITYP(S) (SEQ ID NO: 47) |
| | SRAAQLLPGTWQATTTNEDGQTSQGQIHFQPRSPYTMDIVAQGTISDGRPIVGYGKATVKTPDTLDIDI |
| | TYPS (SEQ ID NO: 48) |
| m15.5 | (SR)AAQLLPGTWQATFTNEDGQTSQGQIHFQPRSPYTMDIVSQGTISDGRPIVGYGKATVKTPDTLDI |
| | DITYP(S) (SEQ ID NO: 49) |
| | SRAAQLLPGTWQATFTNEDGQTSQGQIHFQPRSPYTMDIVSQGTISDGRPIVGYGKATVKTPDTLDIDI |
| | TYPS (SEQ ID NO: 50) |
| m15.6 | (SR)AAQLLPGTWQVTMTNEDGQTSQGQMHFQPRSPYTMDIVAQGTISDGRPIVGYGKATVKTPDTLDI |
| | DITYP(S) (SEQ ID NO: 51) |
| | SRAAQLLPGTWQVTMTNEDGQTSQGQMHFQPRSPYTMDIVAQGTISDGRPIVGYGKATVKTPDTLDIDI |
| | TYPS (SEQ ID NO: 52) |
| m16 | (SR)AAQLLPGTWQATFTNEDGQTSQGQWHFQPRSPYTMDIVAQGTISDGRPIVGYGKATVKTPDTLDI |
| | DITYPSLGNIKAQGQITMDS(P) (SEQ ID NO: 53) |
| | SRAAQLLPGTWQATTTNEDGQTSQGQWHFQPRSPYTMDIVAQGTISDGRPIVGYGKATVKTPDTLDIDI |
| | TYPSLGNIKAQGQITMDSP (SEQ ID NO: 54) |
| m16.1 | (SR)AAQLLPGTWQVTMTNEDGQTSQGQWHFQPRSPYTMDIVAQGTISDGRPIVGYGKATVKTPDTLDI |
| | DITYPSLGNIKAQGQITMDS(P) (SEQ ID NO: 55) |
| | SRAAQLLPGTWQVTMTNEDGQTSQGQWHFQPRSPYTMDIVAQGTISDGRPIVGYGKATVKTPDTLDIDI |
| | TYPSLGNIKAQGQITMDSP (SEQ ID NO: 56) |
| m16.2 | (SR)AAQLLPGTWQATFTNEDGQTSQGQWHFQPRSPYTMDIVAQGTISDGRPIVGYGKATVKTPDTLDI |
| | DITWPSLGNIKGQGQITMDS(P) (SEQ ID NO: 57) |
| | SRAAQLLPGTWQATFTNEDGQTSQGQWHFQPRSPYTMDIVAQGTISDGRPIVGYGKATVKTPDTLDIDI |
| | TWPSLGNIKGQGQITMDSP (SEQ ID NO: 58) |
| m16.3 | (SR)AAQLLPGTWQATFTNEDGQTSQGQFHFQPRSPYTMDIVAQGTISDGRPIVGYGKATVKTPDTLDI |
| | DITYPSLGNIKAQGQITMDS(P) (SEQ ID NO: 59) |
| | SRAAQLLPGTWQATFTNEDGQTSQGQFHFQPRSPYTMDIVAQGTISDGRPIVGYGKATVKTPDTLDIDI |
| | TYPSLGNIKAQGQITMDSP (SEQ ID NO: 60) |
| m16.4 | (SR)AAQLLPGTWQATFTNEDGQTSQGQIHFQPRSPYTMDIVAQGTISDGRPIVGYGKATVKTPDTLDI |
| | DITYPSLGNIKAQGQITMDS(P) (SEQ ID NO: 61) |
| | SRAAQLLPGTWQATTTNEDGQTSQGQIHFQPRSPYTMDIVAQGTISDGRPIVGYGKATVKTPDTLDIDI |
| | TYPSLGNIKAQGQITMDSP (SEQ ID NO: 62) |
| m16.5 | (SR)AAQLLPGTWQATFTNEDGQTSQGQIHFQPRSPYTMDIVSQGTISDGRPIVGYGKATVKTPDTLDI |
| | DITYPSLGNIKFQGQITMDS(P) (SEQ ID NO: 63) |
| | SRAAQLLPGTWQATFTNEDGQTSQGQIHFQPRSPYTMDIVSQGTISDGRPIVGYGKATVKTPDTLDIDI |
| | TYPSLGNIKFQGQITMDSP (SEQ ID NO: 64) |
| m16.6 | (SR)AAQLLPGTWQVTMTNEDGQTSQGQMHFQPRSPYTMDIVAQGTISDGRPIVGYGKATVKTPDTLDI |
| | DITYPSLGNIKAQGQITMDS(P) (SEQ ID NO: 65) |
| | SRAAQLLPGTWQVTMTNEDGQTSQGQMHFQPRSPYTMDIVAQGTISDGRPIVGYGKATVKTPDTLDIDI |
| | TYPSLGNIKAQGQITMDSP (SEQ ID NO: 66) |
| m17 | (SR)AAQLLPGTWQATFTNEDGQTSQGQWHFQPRSPYTMDIVAQGTISDGRPIVGYGKATVKTPDTLDI |
| | DITYPSLGNIKAQGQITMDSPTQFKWDA(TTKGENDFHG) (SEQ ID NO: 67) |
| | SRAAQLLPGTWQAT7TNEDGQTSQGQWHFQPRSPYTMDIVAQGTISDGRPIVGYGKATVKTPDTLDIDI |
| | TYPSLGNIKAQGQITMDSPTQFKWDATTKGENDFHG (SEQ ID NO: 68) |
| m17.1 | (SR)AAQLLPGTWQVTMTNEDGQTSQGQWHFQPRSPYTMDIVAQGTISDGRPIVGYGKATVKTPDTLDI |
| | DITYPSLGNIKAQGQITMDSPTQFKWDA(TTKGENDFHG) (SEQ ID NO: 69) |
| | SRAAQLLPGTWQVTMTNEDGQTSQGQWHFQPRSPYTMDIVAQGTISDGRPIVGYGKATVKTPDTLDIDI |
| | TYPSLGNIKAQGQITMDSPTQFKWDATTKGENDFHG (SEQ ID NO: 70) |
| m17.2 | (SR)AAQLLPGTWQATFTNEDGQTSQGQWHFQPRSPYTMDIVAQGTISDGRPIVGYGKATVKTPDTLDI |
| | DITWPSLGNIKGQGQITMDSPTQFKWDG(TTKGENDFHG) (SEQ ID NO: 71) |
| | SRAAQLLPGTWQATTTNEDGQTSQGQWHFQPRSPYTMDIVAQGTISDGRPIVGYGKATVKTPDTLDIDI |
| | TWPSLGNIKGQGQITMDSPTQFKWDGTTKGENDFHG (SEQ ID NO: 72) |
| m17.3 | (SR)AAQLLPGTWQATFTNEDGQTSQGQFHFQPRSPYTMDIVAQGTISDGRPIVGYGKATVKTPDTLDI |
| | DITYPSLGNIKAQGQITMDSPTQFKFDA(TTKGENDFHG) (SEQ ID NO: 73) |
| | SRAAQLLPGTWQATTTNEDGQTSQGQFHFQPRSPYTMDIVAQGTISDGRPIVGYGKATVKTPDTLDIDI |
| | TYPSLGNIKAQGQITMDSPTQFKFDATTKGENDFHG (SEQ ID NO: 74) |
| m17.4 | (SR)AAQLLPGTWQATFTNEDGQTSQGQIHFQPRSPYTMDIVAQGTISDGRPIVGYGKATVKTPDTLDI |
| | DITYPSLGNIKAQGQITMDSPTQFKFDA(TTKGENDFHG) (SEQ ID NO: 75) |
| | SRAAQLLPGTWQATTTNEDGQTSQGQIHFQPRSPYTMDIVAQGTISDGRPIVGYGKATVKTPDTLDIDI |
| | TYPSLGNIKAQGQITMDSPTQFKFDATTKGENDFHG (SEQ ID NO: 76) |
| m17.5 | (SR)AAQLLPGTWQATFTNEDGQTSQGQIHFQPRSPYTMDIVSQGTISDGRPIVGYGKATVKTPDTLDI |
| | DITYPSLGNIKFQGQITMDSPTQFKFDA(TTKGENDFHG) (SEQ ID NO: 77) |
| | SRAAQLLPGTWQATTTNEDGQTSQGQIHFQPRSPYTMDIVSQGTISDGRPIVGYGKATVKTPDTLDIDI |
| | TYPSLGNIKFQGQITMDSPTQFKFDATTKGENDFHG (SEQ ID NO: 78) |
| m17.6 | (SR)AAQLLPGTWQATFTNEDGQTSQGQIHFQPRSPYTMDIVAQGTISDGRPIVGYGKATVKTPDTLDI |
| | DITYPSLGNIKAQGQITMDSPTQFKFDA(TTSGSGGFKG) (SEQ ID NO: 79) |
| | SRAAQLLPGTWQATFTNEDGQTSQGQIHFQPRSPYTMDIVAQGTISDGRPIVGYGKATVKTPDTLDIDI |
| | TYPSLGNIKAQGQITWDSPTQFKFDATTSGSGGFKG (SEQ ID NO: 80) |
| m17.7 | (SR)AAQLLPGTWQVTMTNEDGQTSQGQMHFQPRSPYTMDIVAQGTISDGRPIVGYGKATVKTPDTLDI |
| | DITYPSLGNIKAQGQITMDSPTQFKFDA(TTKGENDFHG) (SEQ ID NO: 81) |
| | SRAAQLLPGTWQVTMTNEDGQTSQGQMHFQPRSPYTMDIVAQGTISDGRPIVGYGKATVKTPDTLDIDI |
| | TYPSLGNIKAQGQITMDSPTQFKFDATTKGENDFHG (SEQ ID NO: 82) |
| m2 | (DGQ)TSQGQWHFQPRS(P) (SEQ ID NO: 83) |
| | DGQTSQGQWHFQPRSP (SEQ ID NO: 84) |
| m2.1 | (DGQ)TSQGQFHFQPRS(P) (SEQ ID NO: 85) |
| | DGQTSQGQFHFQPRSP (SEQ ID NO: 86) |
| m2.2 | (DGQ)TSQGQIHFQPRS(P) (SEQ ID NO: 87) |
| | DGQTSQGQIHFQPRSP (SEQ ID NO: 88) |
| m2.3 | (DGQ)TSQGQWHFQPRS(P) (SEQ ID NO: 89) |
| | DGQTSQGQWHFQPRSP (SEQ ID NO: 90) |
| m23 | (DGQ)TSQGQWHFQPRSPYTMDIVAQGTI(S) (SEQ ID NO: 91) |
| | DGQTSQGQWHFQPRSPYTWDIVAQGTIS (SEQ ID NO: 92) |
| m23.1 | (DGQ)TSQGQFHFQPRSPYTMDIVAQGTI(S) (SEQ ID NO: 93) |
| | DGQTSQGQFHFQPRSPYTWDIVAQGTIS (SEQ ID NO: 94) |
| m23.2 | (DGQ)TSQGQIHFQPRSPYTMDIVAQGTI(S) (SEQ ID NO: 95) |
| | DGQTSQGQIHFQPRSPYTMDIVAQGTIS (SEQ ID NO: 96) |
| m23.3 | (DGQ)TSQGQIHFQPRSPYTMDIVSQGTI(S) (SEQ ID NO: 97) |
| | DGQTSQGQIHFQPRSPYTMDIVSQGTIS (SEQ ID NO: 98) |
| m23.4 | (DGQ)TSQGQWHFQPRSPYTMDIVAQGTI(S) (SEQ ID NO: 99) |
| | DGQTSQGQWHFQPRSPYTWDIVAQGTIS (SEQ ID NO: 100) |
| m24 | (DGQ)TSQGQWHFQPRSPYTMDIVAQGTISDGRPIVGYGKATV(KTP) (SEQ ID NO: 101) |
| | DGQTSQGQWHFQPRSPYTWDIVAQGTISDGRPIVGYGKATVKTP (SEQ ID NO: 102) |
| m24.1 | (DGQ)TSQGQFHFQPRSPYTMDIVAQGTISDGRPIVGYGKATV(KTP) (SEQ ID NO: 103) |
| | DGQTSQGQFHFQPRSPYTWDIVAQGTISDGRPIVGYGKATVKTP (SEQ ID NO: 104) |
| m24.2 | (DGQ)TSQGQIHFQPRSPYTMDIVAQGTISDGRPIVGYGKATV(KTP) (SEQ ID NO: 105) |
| | DGQTSQGQIHFQPRSPYTWDIVAQGTISDGRPIVGYGKATVKTP (SEQ ID NO: 106) |
| m24.3 | (DGQ)TSQGQIHFQPRSPYTMDIVSQGTISDGRPIVGYGKATV(KTP) (SEQ ID NO: 107) |
| | DGQTSQGQIHFQPRSPYTWDIVSQGTISDGRPIVGYGKATVKTP (SEQ ID NO: 108) |
| m24.4 | (DGQ)TSQGQWHFQPRSPYTMDIVAQGTISDGRPIVGYGKATV(KTP) (SEQ ID NO: 109) |
| | DGQTSQGQWHFQPRSPYTWDIVAQGTISDGRPIVGYGKATVKTP (SEQ ID NO: 110) |
| m25 | (DGQ)TSQGQWHFQPRSPYTMDIVAQGTISDGRPIVGYGKATVKTPDTLDIDITYP(S) (SEQ ID |
| | NO: 111) |
| | DGQTSQGQWHFQPRSPYTWDIVAQGTISDGRPIVGYGKATVKTPDTLDIDITYPS (SEQ ID |
| | NO: 112) |
| m25.1 | (DGQ)TSQGQWHFQPRSPYTMDIVAQGTISDGRPIVGYGKATVKTPDTLDIDITWP(S) (SEQ ID |
| | NO: 113) |
| | DGQTSQGQWHFQPRSPYTMDIVAQGTISDGRPIVGYGKATVKTPDTLDIDITWPS (SEQ ID |
| | NO: 114) |
| m25.2 | (DGQ)TSQGQFHFQPRSPYTMDIVAQGTISDGRPIVGYGKATVKTPDTLDIDITYP(S) (SEQ ID |
| | NO: 115) |
| | DGQTSQGQFHFQPRSPYTMDIVAQGTISDGRPIVGYGKATVKTPDTLDIDITYPS (SEQ ID |
| | NO: 116) |
| m25.3 | (DGQ)TSQGQIHFQPRSPYTMDIVAQGTISDGRPIVGYGKATVKTPDTLDIDITYP(S) (SEQ ID |
| | NO: 117) |
| | DGQTSQGQIHFQPRSPYTMDIVAQGTISDGRPIVGYGKATVKTPDTLDIDITYPS (SEQ ID |
| | NO: 118) |
| m25.4 | (DGQ)TSQGQIHFQPRSPYTMDIVSQGTISDGRPIVGYGKATVKTPDTLDIDITYP(S) (SEQ ID |
| | NO: 119) |
| | DGQTSQGQIHFQPRSPYTMDIVSQGTISDGRPIVGYGKATVKTPDTLDIDITYPS (SEQ ID |
| | NO: 120) |
| m25.5 | (DGQ)TSQGQMHFQPRSPYTMDIVAQGTISDGRPIVGYGKATVKTPDTLDIDITYP(S) (SEQ ID |
| | NO: 121) |
| | DGQTSQGQMHFQPRSPYTMDIVAQGTISDGRPIVGYGKATVKTPDTLDIDITYPS (SEQ ID |
| | NO: 122) |
| m26 | (DGQ)TSQGQWHFQPRSPYTMDIVAQGTISDGRPIVGYGKATVKTPDTLDIDITYPSLGNIKAQGQITM |
| | DS(P) (SEQ ID NO: 123) |
| | DGQTSQGQWHFQPRSPYTMDIVAQGTISDGRPIVGYGKATVKTPDTLDIDITYPSLGNIKAQGQITMDS |
| | P (SEQ ID NO: 124) |
| m26.1 | (DGQ)TSQGQWHFQPRSPYTMDIVAQGTISDGRPIVGYGKATVKTPDTLDIDITWPSLGNIKGQGQITM |
| | DS(P) (SEQ ID NO: 125) |
| | DGQTSQGQWHFQPRSPYTMDIVAQGTISDGRPIVGYGKATVKTPDTLDIDITWPSLGNIKGQGQITMDS |
| | P (SEQ ID NO: 126) |
| m26.2 | (DGQ)TSQGQFHFQPRSPYTMDIVAQGTISDGRPIVGYGKATVKTPDTLDIDITYPSLGNIKAQGQITM |
| | DS(P) (SEQ ID NO: 127) |
| | DGQTSQGQFHFQPRSPYTMDIVAQGTISDGRPIVGYGKATVKTPDTLDIDITYPSLGNIKAQGQITMDS |
| | P (SEQ ID NO: 128) |
| m26.3 | (DGQ)TSQGQIHFQPRSPYTMDIVAQGTISDGRPIVGYGKATVKTPDTLDIDITYPSLGNIKAQGQITM |
| | DS(P) (SEQ ID NO: 129) |
| | DGQTSQGQIHFQPRSPYTMDIVAQGTISDGRPIVGYGKATVKTPDTLDIDITYPSLGNIKAQGQITMDS |
| | P (SEQ ID NO: 130) |
| m26.4 | (DGQ)TSQGQIHFQPRSPYTMDIVSQGTISDGRPIVGYGKATVKTPDTLDIDITYPSLGNIKFQGQITM |
| | DS(P) (SEQ ID NO: 131) |
| | DGQTSQGQIHFQPRSPYTMDIVSQGTISDGRPIVGYGKATVKTPDTLDIDITYPSLGNIKFQGQITMDS |
| | P (SEQ ID NO: 132) |
| m26.5 | (DGQ)TSQGQMHFQPRSPYTMDIVAQGTISDGRPIVGYGKATVKTPDTLDIDITYPSLGNIKAQGQITM |
| | DS(P) (SEQ ID NO: 133) |
| | DGQTSQGQMHFQPRSPYTMDIVAQGTISDGRPIVGYGKATVKTPDTLDIDITYPSLGNIKAQGQITMDS |
| | P (SEQ ID NO: 134) |
| m27 | (DGQ)TSQGQWHFQPRSPYTMDIVAQGTISDGRPIVGYGKATVKTPDTLDIDITYPSLGNIKAQGQITM |
| | DSPTQFKWDA(TTKGENDFHG) (SEQ ID NO: 135) |
| | DGQTSQGQWHFQPRSPYTMDIVAQGTISDGRPIVGYGKATVKTPDTLDIDITYPSLGNIKAQGQITMDS |
| | PTQFKWDATTKGENDFHG (SEQ ID NO: 136) |
| m27.1 | (DGQ)TSQGQWHFQPRSPYTMDIVAQGTISDGRPIVGYGKATVKTPDTLDIDITWPSLGNIKGQGQITM |
| | DSPTQFKWDG(TTKGENDFHG) (SEQ ID NO: 137) |
| | DGQTSQGQWHFQPRSPYTMDIVAQGTISDGRPIVGYGKATVKTPDTLDIDITWPSLGNIKGQGQITMDS |
| | PTQFKWDGTTKGENDFHG (SEQ ID NO: 138) |
| m27.2 | (DGQ)TSQGQFHFQPRSPYTMDIVAQGTISDGRPIVGYGKATVKTPDTLDIDITYPSLGNIKAQGQITM |
| | DSPTQFKFDA(TTKGENDFHG) (SEQ ID NO: 139) |
| | DGQTSQGQFHFQPRSPYTMDIVAQGTISDGRPIVGYGKATVKTPDTLDIDITYPSLGNIKAQGQITMDS |
| | PTQFKFDATTKGENDFHG (SEQ ID NO: 140) |
| m27.3 | (DGQ)TSQGQIHFQPRSPYTMDIVAQGTISDGRPIVGYGKATVKTPDTLDIDITYPSLGNIKAQGQITM |
| | DSPTQFKFDA(TTKGENDFHG) (SEQ ID NO: 141) |
| | DGQTSQGQIHFQPRSPYTMDIVAQGTISDGRPIVGYGKATVKTPDTLDIDITYPSLGNIKAQGQITMDS |
| | PTQFKFDATTKGENDFHG (SEQ ID NO: 142) |
| m27.4 | (DGQ)TSQGQIHFQPRSPYTMDIVSQGTISDGRPIVGYGKATVKTPDTLDIDITYPSLGNIKFQGQITM |
| | DSPTQFKFDA(TTKGENDFHG) (SEQ ID NO: 143) |
| | DGQTSQGQIHFQPRSPYTMDIVSQGTISDGRPIVGYGKATVKTPDTLDIDITYPSLGNIKFQGQITMDS |
| | PTQFKFDATTKGENDFHG (SEQ ID NO: 144) |
| m27.5 | (DGQ)TSQGQIHFQPRSPYTMDIVAQGTISDGRPIVGYGKATVKTPDTLDIDITYPSLGNIKAQGQITM |
| | DSPTQFKFDA(TTSGSGGFKG) (SEQ ID NO: 145) |
| | DGQTSQGQIHFQPRSPYTMDIVAQGTISDGRPIVGYGKATVKTPDTLDIDITYPSLGNIKAQGQITMDS |
| | PTQFKFDATTSGSGGFKG (SEQ ID NO: 146) |
| m27.6 | (DGQ)TSQGQMHFQPRSPYTMDIVAQGTISDGRPIVGYGKATVKTPDTLDIDITYPSLGNIKAQGQITM |
| | DSPTQFKFDA(TTKGENDFHG) (SEQ ID NO: 147) |
| | DGQTSQGQMHFQPRSPYTMDIVAQGTISDGRPIVGYGKATVKTPDTLDIDITYPSLGNIKAQGQITMDS |
| | PTQFKFDATTKGENDFHG (SEQ ID NO: 148) |
| m28 | (DGQ)TSQGQWHFQPRSPYTMDIVAQGTISDGRPIVGYGKATVKTPDTLDIDITYPSLGNIKAQGQITM |
| | DSPTQFKWDATTKGENDFHGRLTGTLQR(QE) (SEQ ID NO: 14 9) |
| | DGQTSQGQWHFQPRSPYTMDIVAQGTISDGRPIVGYGKATVKTPDTLDIDITYPSLGNIKAQGQITMDS |
| | PTQFKWDATTKGENDFHGRLTGTLQRQE (SEQ ID NO: 150) |
| m28.1 | (DGQ)TSQGQWHFQPRSPYTMDIVAQGTISDGRPIVGYGKATVKTPDTLDIDITWPSLGNIKGQGQITM |
| | DSPTQFKWDGTTKGENDFHGRLTGTLQR(QE) (SEQ ID NO: 151) |
| | DGQTSQGQWHFQPRSPYTMDIVAQGTISDGRPIVGYGKATVKTPDTLDIDITWPSLGNIKGQGQITMDS |
| | PTQFKWDGTTKGENDFHGRLTGTLQRQE (SEQ ID NO: 152) |
| m28.2 | (DGQ)TSQGQFHFQPRSPYTMDIVAQGTISDGRPIVGYGKATVKTPDTLDIDITYPSLGNIKAQGQITM |
| | DSPTQFKFDATTKGENDFHGRLTGTLQR(QE) (SEQ ID NO: 153) |
| | DGQTSQGQFHFQPRSPYTMDIVAQGTISDGRPIVGYGKATVKTPDTLDIDITYPSLGNIKAQGQITMDS |
| | PTQFKFDATTKGENDFHGRLTGTLQRQE (SEQ ID NO: 154) |
| m28.3 | (DGQ)TSQGQIHFQPRSPYTMDIVAQGTISDGRPIVGYGKATVKTPDTLDIDITYPSLGNIKAQGQITM |
| | DSPTQFKFDATTKGENDFHGRLTGTLQR(QE) (SEQ ID NO: 155) |
| | DGQTSQGQIHFQPRSPYTMDIVAQGTISDGRPIVGYGKATVKTPDTLDIDITYPSLGNIKAQGQITMDS |
| | PTQFKFDATTKGENDFHGRLTGTLQRQE (SEQ ID NO: 156) |
| m28.4 | (DGQ)TSQGQIHFQPRSPYTMDIVSQGTISDGRPIVGYGKATVKTPDTLDIDITYPSLGNIKFQGQITM |
| | DSPTQFKFDATTKGENDFHGRLTGTLQR(QE) (SEQ ID NO: 157) |
| | DGQTSQGQIHFQPRSPYTMDIVSQGTISDGRPIVGYGKATVKTPDTLDIDITYPSLGNIKFQGQITMDS |
| | PTQFKFDATTKGENDFHGRLTGTLQRQE (SEQ ID NO: 158) |
| m28.5 | (DGQ)TSQGQIHFQPRSPYTMDIVAQGTISDGRPIVGYGKATVKTPDTLDIDITYPSLGNIKAQGQITM |
| | DSPTQFKFDATTSGSGGFKGRLTGTLQR(QE) (SEQ ID NO: 159) |
| | DGQTSQGQIHFQPRSPYTMDIVAQGTISDGRPIVGYGKATVKTPDTLDIDITYPSLGNIKAQGQITMDS |
| | PTQFKFDATTSGSGGFKGRLTGTLQRQE (SEQ ID NO: 160) |
| m28.6 | (DGQ)TSQGQMHFQPRSPYTMDIVAQGTISDGRPIVGYGKATVKTPDTLDIDITYPSLGNIKAQGQITM |
| | DSPTQFKFDATTKGENDFHGRLTGTLQR(QE) (SEQ ID NO: 161) |
| | DGQTSQGQMHFQPRSPYTMDIVAQGTISDGRPIVGYGKATVKTPDTLDIDITYPSLGNIKAQGQITMDS |
| | PTQFKFDATTKGENDFHGRLTGTLQRQE (SEQ ID NO: 162) |
| m3 | (Y)TMDIVAQGTI(S) (SEQ ID NO: 163) |
| | YTMDIVAQGTIS (SEQ ID NO: 164) |
| m3.1 | (Y)TMDIVSQGTI(S) (SEQ ID NO: 165) |
| | YTMDIVSQGTIS (SEQ ID NO: 166) |
| m34 | (Y)TMDIVAQGTISDGRPIVGYGKATV(KTP) (SEQ ID NO: 167) |
| | YTMDIVAQGTISDGRPIVGYGKATVKTP (SEQ ID NO: 168) |
| m34.1 | (Y)TMDIVSQGTISDGRPIVGYGKATV(KTP) (SEQ ID NO: 169) |
| | YTMDIVSQGTISDGRPIVGYGKATVKTP (SEQ ID NO: 170) |
| m35 | (Y)TMDIVAQGTISDGRPIVGYGKATVKTPDTLDIDITYP(S) (SEQ ID NO: 171) |
| | YTMDIVAQGTISDGRPIVGYGKATVKTPDTLDIDITYPS (SEQ ID NO: 172) |
| m35.1 | (Y)TMDIVAQGTISDGRPIVGYGKATVKTPDTLDIDITWP(S) (SEQ ID NO: 173) |
| | YTMDIVAQGTISDGRPIVGYGKATVKTPDTLDIDITWPS (SEQ ID NO: 174) |
| m35.2 | (Y)TMDIVSQGTISDGRPIVGYGKATVKTPDTLDIDITYP(S) (SEQ ID NO: 175) |
| | YTMDIVSQGTISDGRPIVGYGKATVKTPDTLDIDITYPS (SEQ ID NO: 176) |
| m36 | (Y)TMDIVAQGTISDGRPIVGYGKATVKTPDTLDIDITYPSLGNIKAQGQITMDS(P) (SEQ ID |
| | NO: 177) |
| | YTMDIVAQGTISDGRPIVGYGKATVKTPDTLDIDITYPSLGNIKAQGQITMDSP (SEQ ID |
| | NO: 178) |
| m36.1 | (Y)TMDIVAQGTISDGRPIVGYGKATVKTPDTLDIDITWPSLGNIKGQGQITMDS(P) (SEQ ID |
| | NO: 179) |
| | YTMDIVAQGTISDGRPIVGYGKATVKTPDTLDIDITWPSLGNIKGQGQITMDSP (SEQ ID |
| | NO: 180) |
| m36.2 | (Y)TMDIVSQGTISDGRPIVGYGKATVKTPDTLDIDITYPSLGNIKFQGQITMDS(P) (SEQ ID |
| | NO: 181) |
| | YTMDIVSQGTISDGRPIVGYGKATVKTPDTLDIDITYPSLGNIKFQGQITMDSP (SEQ ID |
| | NO: 182) |
| m37 | (Y)TMDIVAQGTISDGRPIVGYGKATVKTPDTLDIDITYPSLGNIKAQGQITMDSPTQFKWDA |
| | (TTKGENDFHG) (SEQ ID NO: 183) |
| | YTMDIVAQGTISDGRPIVGYGKATVKTPDTLDIDITYPSLGNIKAQGQITMDSPTQFKWDATTKGENDF |
| | HG (SEQ ID NO: 184) |
| m37.1 | (Y) TMDIVAQGTISDGRPIVGYGKATVKTPDTLDIDITWPSLGNIKGQGQITMDSPTQFKWDG |
| | (TTKGENDFHG) (SEQ ID NO: 185) |
| | YTMDIVAQGTISDGRPIVGYGKATVKTPDTLDIDITWPSLGNIKGQGQITMDSPTQFKWDGTTKGENDF |
| | HG (SEQ ID NO: 186) |
| m37.2 | (Y)TMDIVAQGTISDGRPIVGYGKATVKTPDTLDIDITYPSLGNIKAQGQITMDSPTQFKFDA |
| | (TTKGENDFHG) (SEQ ID NO: 187) |
| | YTMDIVAQGTISDGRPIVGYGKATVKTPDTLDIDITYPSLGNIKAQGQITMDSPTQFKFDATTKGENDF |
| | HG (SEQ ID NO: 188) |
| m37.3 | (Y)TMDIVSQGTISDGRPIVGYGKATVKTPDTLDIDITYPSLGNIKFQGQITMDSPTQFKFDA |
| | (TTKGENDFHG) (SEQ ID NO: 189) |
| | YTMDIVSQGTISDGRPIVGYGKATVKTPDTLDIDITYPSLGNIKFQGQITMDSPTQFKFDATTKGENDF |
| | HG (SEQ ID NO: 190) |
| m37.4 | (Y)TMDIVAQGTISDGRPIVGYGKATVKTPDTLDIDITYPSLGNIKAQGQITMDSPTQFKFDA |
| | (TTSGSGGFKG) (SEQ ID NO: 191) |
| | YTMDIVAQGTISDGRPIVGYGKATVKTPDTLDIDITYPSLGNIKAQGQITMDSPTQFKFDATTSGSGGF |
| | KG (SEQ ID NO: 192) |
| m3 8 | (Y)TMDIVAQGTISDGRPIVGYGKATVKTPDTLDIDITYPSLGNIKAQGQITMDSPTQFKWDATTKGEN |
| | DFHGRLTGTLQR(QE) (SEQ ID NO: 193) |
| | YTMDIVAQGTISDGRPIVGYGKATVKTPDTLDIDITYPSLGNIKAQGQITMDSPTQFKWDATTKGENDF |
| | HGRLTGTLQRQE (SEQ ID NO: 194) |
| m38.1 | (Y)TMDIVAQGTISDGRPIVGYGKATVKTPDTLDIDITWPSLGNIKGQGQITMDSPTQFKWDGTTKGEN |
| | DFHGRLTGTLQR(QE) (SEQ ID NO: 195) |
| | YTMDIVAQGTISDGRPIVGYGKATVKTPDTLDIDITWPSLGNIKGQGQITMDSPTQFKWDGTTKGENDF |
| | HGRLTGTLQRQE (SEQ ID NO: 196) |
| m38.2 | (Y)TMDIVAQGTISDGRPIVGYGKATVKTPDTLDIDITYPSLGNIKAQGQITMDSPTQFKFDATTKGEN |
| | DFHGRLTGTLQR(QE) (SEQ ID NO: 197) |
| | YTMDIVAQGTISDGRPIVGYGKATVKTPDTLDIDITYPSLGNIKAQGQITMDSPTQFKFDATTKGENDF |
| | HGRLTGTLQRQE (SEQ ID NO: 198) |
| m38.3 | (Y)TMDIVSQGTISDGRPIVGYGKATVKTPDTLDIDITYPSLGNIKFQGQITMDSPTQFKFDATTKGEN |
| | DFHGRLTGTLQR(QE) (SEQ ID NO: 199) |
| | YTMDIVSQGTISDGRPIVGYGKATVKTPDTLDIDITYPSLGNIKFQGQITMDSPTQFKFDATTKGENDF |
| | HGRLTGTLQRQE (SEQ ID NO: 200) |
| m38.4 | (Y)TMDIVAQGTISDGRPIVGYGKATVKTPDTLDIDITYPSLGNIKAQGQITMDSPTQFKFDATTSGSG |
| | GFKGRLTGTLQR(QE) (SEQ ID NO: 201) |
| | YTMDIVAQGTISDGRPIVGYGKATVKTPDTLDIDITYPSLGNIKAQGQITMDSPTQFKFDATTSGSGGF |
| | KGRLTGTLQRQE (SEQ ID NO: 202) |
| m4 | (DGRP)IVGYGKATV(KTP) (SEQ ID NO: 203) |
| | DGRPIVGYGKATVKTP (SEQ ID NO: 204) |
| m45 | (DGRP)IVGYGKATVKTPDTLDIDITYP(S) (SEQ ID NO: 205) |
| | DGRPIVGYGKATVKTPDTLDIDITYPS (SEQ ID NO: 206) |
| m45.1 | (DGRP)IVGYGKATVKTPDTLDIDITWP(S) (SEQ ID NO: 207) |
| | DGRPIVGYGKATVKTPDTLDIDITWPS (SEQ ID NO: 208) |
| m46 | (DGRP)IVGYGKATVKTPDTLDIDITYPSLGNIKAQGQITMDS(P) (SEQ ID NO: 209) |
| | DGRPIVGYGKATVKTPDTLDIDITYPSLGNIKAQGQITMDSP (SEQ ID NO: 210) |
| m46.1 | (DGRP)IVGYGKATVKTPDTLDIDITWPSLGNIKGQGQITMDS(P) (SEQ ID NO: 211) |
| | DGRPIVGYGKATVKTPDTLDIDITWPSLGNIKGQGQITMDSP (SEQ ID NO: 212) |
| m46.2 | (DGRP)IVGYGKATVKTPDTLDIDITYPSLGNIKFQGQITMDS (P) (SEQ ID NO: 213) |
| | DGRPIVGYGKATVKTPDTLDIDITYPSLGNIKFQGQITMDSP (SEQ ID NO: 214) |
| m47 | (DGRP)IVGYGKATVKTPDTLDIDITYPSLGNIKAQGQITMDSPTQFKWDA(TTKGENDFHG) (SEQ |
| | ID NO: 215) |
| | DGRPIVGYGKATVKTPDTLDIDITYPSLGNIKAQGQITMDSPTQFKWDATTKGENDFHG (SEQ ID |
| | NO: 216) |
| m47.1 | (DGRP)IVGYGKATVKTPDTLDIDITWPSLGNIKGQGQITMDSPTQFKWDG(TTKGENDFHG) (SEQ |
| | ID NO: 217) |
| | DGRPIVGYGKATVKTPDTLDIDITWPSLGNIKGQGQITMDSPTQFKWDGTTKGENDFHG (SEQ ID |
| | NO: 218) |
| m47.2 | (DGRP)IVGYGKATVKTPDTLDIDITYPSLGNIKAQGQITMDSPTQFKFDA(TTKGENDFHG) (SEQ |
| | ID NO: 219) |
| | DGRPIVGYGKATVKTPDTLDIDITYPSLGNIKAQGQITMDSPTQFKFDATTKGENDFHG (SEQ ID |
| | NO: 220) |
| m47.3 | (DGRP)IVGYGKATVKTPDTLDIDITYPSLGNIKFQGQITMDSPTQFKFDA(TTKGENDFHG) (SEQ |
| | ID NO: 221) |
| | DGRPIVGYGKATVKTPDTLDIDITYPSLGNIKFQGQITMDSPTQFKFDATTKGENDFHG (SEQ ID |
| | NO: 222) |
| m47.4 | (DGRP)IVGYGKATVKTPDTLDIDITYPSLGNIKAQGQITMDSPTQFKFDA(TTSGSGGFKG) (SEQ |
| | ID NO: 223) |
| | DGRPIVGYGKATVKTPDTLDIDITYPSLGNIKAQGQITMDSPTQFKFDATTSGSGGFKG (SEQ ID |
| | NO: 224) |
| m48 | (DGRP)IVGYGKATVKTPDTLDIDITYPSLGNIKAQGQITMDSPTQFKWDATTKGENDFHGRLTGTLQR |
| | (QE) (SEQ ID NO: 225) |
| | DGRPIVGYGKATVKTPDTLDIDITYPSLGNIKAQGQITMDSPTQFKWDATTKGENDFHGRLTGTLQRQE |
| | (SEQ ID NO: 226) |
| m48.1 | (DGRP)IVGYGKATVKTPDTLDIDITWPSLGNIKGQGQITMDSPTQFKWDGTTKGENDFHGRLTGTLQR |
| | (QE) (SEQ ID NO: 227) |
| | DGRPIVGYGKATVKTPDTLDIDITWPSLGNIKGQGQITMDSPTQFKWDGTTKGENDFHGRLTGTLQRQE |
| | (SEQ ID NO: 228) |
| m48.2 | (DGRP)IVGYGKATVKTPDTLDIDITYPSLGNIKAQGQITMDSPTQFKFDATTKGENDFHGRLTGTLQR |
| | (QE) (SEQ ID NO: 229) |
| | DGRPIVGYGKATVKTPDTLDIDITYPSLGNIKAQGQITMDSPTQFKFDATTKGENDFHGRLTGTLQRQE |
| | (SEQ ID NO: 230) |
| m48.3 | (DGRP)IVGYGKATVKTPDTLDIDITYPSLGNIKFQGQITMDSPTQFKFDATTKGENDFHGRLTGTLQR |
| | (QE) (SEQ ID NO: 231) |
| | DGRPIVGYGKATVKTPDTLDIDITYPSLGNIKFQGQITMDSPTQFKFDATTKGENDFHGRLTGTLQRQE |
| | (SEQ ID NO: 232) |
| m48.4 | (DGRP)IVGYGKATVKTPDTLDIDITYPSLGNIKAQGQITMDSPTQFKFDATTSGSGGFKGRLTGTLQR |
| | (QE) (SEQ ID NO: 233) |
| | DGRPIVGYGKATVKTPDTLDIDITYPSLGNIKAQGQITMDSPTQFKFDATTSGSGGFKGRLTGTLQRQE |
| | (SEQ ID NO: 234) |
| m5 | (D)TLDIDITYP(S) (SEQ ID NO: 235) |
| | DTLDIDITYPS (SEQ ID NO: 236) |
| m5.1 | (D)TLDIDITWP(S) (SEQ ID NO: 237) |
| | DTLDIDITWPS (SEQ ID NO: 238) |
| m56 | (D)TLDIDITYPSLGNIKAQGQITMDS(P) (SEQ ID NO: 239) |
| | DTLDIDITYPSLGNIKAQGQITMDSP (SEQ ID NO: 240) |
| m56.1 | (D)TLDIDITWPSLGNIKGQGQITMDS(P) (SEQ ID NO: 241) |
| | DTLDIDITWPSLGNIKGQGQITMDSP (SEQ ID NO: 242) |
| m56.2 | (D)TLDIDITYPSLGNIKFQGQITMDS(P) (SEQ ID NO: 243) |
| | DTLDIDITYPSLGNIKFQGQITMDSP (SEQ ID NO: 244) |
| m57 | (D)TLDIDITYPSLGNIKAQGQITMDSPTQFKWDA(TTKGENDFHG) (SEQ ID NO: 245) |
| | DTLDIDITYPSLGNIKAQGQITMDSPTQFKWDATTKGENDFHG (SEQ ID NO: 246) |
| m57.1 | (D)TLDIDITWPSLGNIKGQGQITMDSPTQFKWDG(TTKGENDFHG) (SEQ ID NO: 247) |
| | DTLDIDITWPSLGNIKGQGQITMDSPTQFKWDGTTKGENDFHG (SEQ ID NO: 248) |
| m57.2 | (D)TLDIDITYPSLGNIKAQGQITMDSPTQFKFDA(TTKGENDFHG) (SEQ ID NO: 249) |
| | DTLDIDITYPSLGNIKAQGQITMDSPTQFKFDATTKGENDFHG (SEQ ID NO: 250) |
| m57.3 | (D)TLDIDITYPSLGNIKFQGQITMDSPTQFKFDA(TTKGENDFHG) (SEQ ID NO: 251) |
| | DTLDIDITYPSLGNIKFQGQITMDSPTQFKFDATTKGENDFHG (SEQ ID NO: 252) |
| m57.4 | (D)TLDIDITYPSLGNIKAQGQITMDSPTQFKFDA(TTSGSGGFKG) (SEQ ID NO: 253) |
| | DTLDIDITYPSLGNIKAQGQITMDSPTQFKFDATTSGSGGFKG (SEQ ID NO: 254) |
| m58 | (D)TLDIDITYPSLGNIKAQGQITMDSPTQFKWDATTKGENDFHGRLTGTLQR(QE) (SEQ ID |
| | NO: 255) |
| | DTLDIDITYPSLGNIKAQGQITMDSPTQFKWDATTKGENDFHGRLTGTLQRQE (SEQ ID NO: 256) |
| m58.1 | (D) TLDIDITWPSLGNIKGQGQITMDSPTQFKWDGTTKGENDFHGRLTGTLQR(QE) (SEQ ID |
| | NO: 257) |
| | DTLDIDITWPSLGNIKGQGQITMDSPTQFKWDGTTKGENDFHGRLTGTEQRQE (SEQ ID NO: 258) |
| m58.2 | (D) TLDIDITYPSLGNIKAQGQITMDSPTQFKFDATTKGENDFHGRLTGTLQR(QE) (SEQ ID |
| | NO: 259) |
| | DTLDIDITYPSLGNIKAQGQITMDSPTQFKFDATTKGENDFHGRLTGTGQRQE (SEQ ID NO: 260) |
| m58.3 | (D) TLDIDITYPSLGNIKFQGQITMDSPTQFKFDATTKGENDFHGRLTGTLQR(QE) (SEQ ID |
| | NO: 261) |
| | DTLDIDITYPSLGNIKFQGQITMDSPTQFKFDATTKGENDFHGRLTGTLQRQE (SEQ ID NO: 262) |
| m58.4 | (D)TLDIDITYPSLGNIKAQGQITMDSPTQFKFDATTSGSGGFKGRLTGTLQR(QE) (SEQ ID |
| | NO: 263) |
| | DTLDIDITYPSLGNIKAQGQITMDSPTQFKFDATTSGSGGFKGRLTGTLQRQE (SEQ ID NO: 264) |
| m6 | (LGNI)KAQGQITMDS(P) (SEQ ID NO: 265) |
| | LGNIKAQGQITMDS2 (SEQ ID NO: 266) |
| m6.1 | (LGNI)KGQGQITMDS(P) (SEQ ID NO: 267) |
| | LGNTKGQGQITMDS2 (SEQ ID NO: 268) |
| m6.2 | (LGNI)KFQGQITMDS(P) (SEQ ID NO: 269) |
| | LGNIKFQGQITMDSP (SEQ ID NO: 270) |
| m67 | (LGNI)KAQGQITMDSPTQFKWDA(TTKGENDFHG) (SEQ ID NO: 271) |
| | LGNIKAQGQITMDSPTQFKWDATTKGENDFHG (SEQ ID NO: 272) |
| m67.1 | (LGNI)KGQGQITMDSPTQFKWDG(TTKGENDFHG) (SEQ ID NO: 273) |
| | LGNIKGQGQITMDSPTQFKWDGTTKGENDFHG (SEQ ID NO: 274) |
| m67.2 | (LGNI)KAQGQITMDSPTQFKFDA(TTKGENDFHG) (SEQ ID NO: 275) |
| | LGNIKAQGQITMDSPTQFKFDATTKGENDFHG (SEQ ID NO: 276) |
| m67.3 | (LGNI)KFQGQITMDSPTQFKFDA(TTKGENDFHG) (SEQ ID NO: 277) |
| | LGNIKFQGQITMDSPTQFKFDATTKGENDFHG (SEQ ID NO: 278) |
| m67.4 | (LGNI)KAQGQITMDSPTQFKFDA(TTSGSGGFKG) (SEQ ID NO: 279) |
| | LGNIKAQGQITMDSPTQFKFDATTSGSGGFKG (SEQ ID NO: 280) |
| m68 | (LGNI)KAQGQITMDSPTQFKWDATTKGENDFHGRLTGTLQR(QE) (SEQ ID NO: 281) |
| | LGNIKAQGQITMDSPTQFKWDATTKGENDFHGRLTGTLQRQE (SEQ ID NO: 282) |
| m68.1 | (LGNI)KGQGQITMDSPTQFKWDGTTKGENDFHGRLTGTLQR(QE) (SEQ ID NO: 283) |
| | LGNIKGQGQITMDSPTQFKWDGTTKGENDFHGRLTGTLQRQE (SEQ ID NO: 284) |
| m68.2 | (LGNI)KAQGQITMDSPTQFKFDATTKGENDFHGRLTGTLQR(QE) (SEQ ID NO: 285) |
| | LGNIKAQGQITMDSPTQFKFDATTKGENDFHGRLTGTLQRQE (SEQ ID NO: 286) |
| m68.3 | (LGNI)KFQGQITMDSPTQFKFDATTKGENDFHGRLTGTLQR(QE) (SEQ ID NO: 287) |
| | LGNTKFQGQITMDS2TQFKFDATTKGENDFHGRLTGTLORQE (SEQ ID NO: 288) |
| m68.4 | (LGNI)KAQGQITMDSPTQFKFDATTSGSGGFKGRLTGTLQR(QE) (SEQ ID NO: 289) |
| | LGNIKAQGQITMDSPTQFKFDATTSGSGGFKGRLTGTLQRQE (SEQ ID NO: 290) |
| m7 | (T)QFKWDA(TTKGENDFHG) (SEQ ID NO: 291) |
| | TQFKWDATTKGENDFHG(SEQ ID NO: 292) |
| m7.1 | (T)QFKWDG(TTKGENDFHG) (SEQ ID NO: 293) |
| | TQFKWDGTTKGENDZHG (SEQ ID NO: 294) |
| m7.2 | (T)QFKFDA(TTKGENDFHG) (SEQ ID NO: 295) |
| | TQFKFDATTKGENDFHG (SEQ ID NO: 296) |
| m7.3 | (T)QFKFDA(TTSGSGGFKG) (SEQ ID NO: 297) |
| | TQFKFDATTSGSGGFKG (SEQ ID NO: 298) |
| m78 | (T)QFKWDATTKGENDFHGRLTGTLQR(QE) (SEQ ID NO: 299) |
| | TQFKWDATTKGENDFHGRLTGTLQRQE (SEQ ID NO: 300) |
| m78.1 | (T)QFKWDGTTKGENDFHGRLTGTLQR(QE) (SEQ ID NO: 301) |
| | TQFKWDGTTKGENDZHGRLTGTLQRQE (SEQ ID NO: 302) |
| m78.2 | (T)QFKFDATTKGENDFHGRLTGTLQR(QE) (SEQ ID NO: 303) |
| | TQFKFDATTKGENDFHGRLTGTLQRQE (SEQ ID NO: 304) |
| m78.3 | (T)QFKFDATTSGSGGFKGRLTGTLQR(QE) (SEQ ID NO: 305) |
| | TQFKFDATTSGSGGFKGRLTGTLQRQE (SEQ ID NO: 306) |
| m8 | (R)LTGTLQR(QE) (SEQ ID NO: 307) |
| | RLTGTLQRQE (SEQ ID NO: 308) |

As described herein, the self-complementing multipartite β-barrel proteins and the β-barrel polypeptides of the disclosure are excellent scaffolds for ligand binding. Thus, in another embodiment the multipartite β-barrel proteins, β-barrel polypeptides, and polypeptides of any embodiment of the disclosure may further comprise one or more functional domains. As used herein, a “functional domain” is any polypeptide or post-translational modification that has an activity that adds functionality to the polypeptides of the disclosure. In non-limiting embodiments, such functional domains may comprise one or more polypeptide antigens, polypeptide therapeutics, ion-binding polypeptides (including but not limited to calcium-binding polypeptides), small-molecule binding polypeptides, inorganic or organic substrate-binding polypeptides, pH-sensitive polypeptides, voltage-sensitive polypeptides, mechanically-sensitive polypeptides, thermally-responsive polypeptides, nucleic acid-binding polypeptides, luminescent or fluorescent polypeptides, fluorescence quenching polypeptides, detectable markers including but not limited to covalent linking or non-covalent interaction of fluorescent molecules, luminescent or fluorescent or fluorescence quenching proteins or functional portions thereof, etc. The one or more functional domains may be fused at any appropriate regions within the multipartite β-barrel proteins, β-barrel polypeptides, and polypeptides of the disclosure. In various embodiments, the one or more functional domains may be fused to one or more of the beta turn domains (i.e.: X3, X6, X8, X11, X13, X16, and/or X18). In one specific embodiment, X18 comprises a functional domain. In various other embodiments, the capping domain and/or X19 may comprise a functional domain. In one specific embodiment, the functional domain comprises a detectable moiety including but not limited to a fluorescent protein or other chromophore; and a detector polypeptide including but not limited to a pH-responsive polypeptide, an ion-binding polypeptide, a small molecule-binding polypeptide, a polypeptide-binding polypeptide, or a nucleic acid-binding polypeptide.

In a second aspect, the disclosure provides a polypeptide comprising a first polypeptide component or a second polypeptide component of any embodiment of the first aspect of the disclosure. The individual polypeptides are useful, for example, in generating the multipartite β-barrel proteins of the first aspect of the disclosure. In one specific embodiment, the polypeptide comprises an amino acid sequence at least 80%, 85%, 90%, 91%, 92%, 93%, 94%, 95%, 96%, 97%, 98%, 99%, or 100% identical to the amino acid sequence of a polypeptide in Table 1 (SEQ ID NOS:1-308), wherein residues in parentheses are optional. In one embodiment, the optional residues are present. In other embodiments, 1, 2, 3, 4, 5, 6, 7, 8, 9, or all optional residues at the N terminus and/or the C-terminus of any one of SEQ ID NOS: 1-308 may independently be absent. As will be understood by those of skill in the art, if less than all optional residues are absent, those residues at the termini of the optional region would be absent. In another embodiment, the polypeptides of any embodiment of the disclosure may further comprise one or more functional domains, as described above in the first aspect of the disclosure.

In a third aspect, the disclosure provides β-barrel polypeptides, comprising domains X1-X2-X3-X4-X5-X6-X7-X8-X9-X10-X11-X12-X13-X14-X15-X16-X17-X18-X19, wherein:

X1 comprises a capping domain;

X2 comprises a beta strand,

wherein a contiguous C-terminal portion of X1 and N-terminal portion of X2 comprise the amino acid sequence Z1-P-G-Z2-W, where Z1 and Z2 are any amino acid;

X3 comprises a beta turn;

X4 comprises a beta strand that includes an internal G residue and a P at its C-terminus;

X5 comprises a single polar amino acid;

X6 comprises a beta turn;

X7 comprises a beta strand including an internal G residue;

X8 comprises a beta turn;

X9 comprises a beta strand including an internal P residue and 2 internal G residues;

X10 comprises a single polar amino acid;

X11 comprises a beta turn;

X12 comprises a beta strand;

X13 comprises a beta turn;

X14 comprises a beta strand with an internal G residue;

X15 comprises a single polar amino acid;

X16 comprises a beta turn;

X17 comprises a beta strand;

X18 comprises a beta turn; and

X19 comprises a beta strand;

wherein the last residue of the X19 domain is N-terminal to and connected to the first residue of X1 domain via an amino acid linker:

wherein 1, 2, or 3 contiguous domains X1, X2, X3, X4, X5, X6, X7, X8, X9, X10, X11, X12, X13, X14, X15, X16, X17, X18, and X19 may be partially or wholly absent. In one embodiment, 0 or 1 domain is wholly absent.

This third aspect of the disclosure provides circularly permuted β-barrel polypeptides having a changed order of domains X1, X2, X3, X4, X5, X6, X7, X8, X9, X10, X11, X12, X13, X14, X15, X16, X17, X18, and X19, and therefore a changed order of amino acids in their protein sequences, while retaining the β-barrel structure.

In one embodiment, a single split point results in removing G residues (i.e.: cleavage of a covalent bond between adjacent amino acid residues). In this embodiment, the starting β-barrel polypeptide may be split within any one domain or between any two adjacent domains. By way of non-limiting example, in various embodiment of a β-barrel protein of this aspect, the β-barrel polypeptides may comprise as follows:

| Example | β-barrel polypeptide |
|---|---|
| 1: Split at or | (X3)-X4-X5-X6-X7-X8-X9-X10-X11-X12- |
| within X3 beta turn | X13-X14-X15-X16-X17-X18-X19-linker-X1-X2-(X3) |
| 2: Split at or | (X4)-X5-X6-X7-X8-X9-X10-X11-X12-X13- |
| within X4 beta | X14-X15-X16-X17-X18-X19-linker-X1-X2-X3-(X4) |
| strand | |
| 3: Split at or | (X8)-X9-X10-X11-X12-X13-X14-X15-X16-X17- |
| within X8 beta turn | X18-X19-linker-X1-X2-X3-X4-X5-X6-X7-(X8) |
| 4: Split at or within | (X12)-X13-X14-X15-X16-X17-X18-X19-linker- |
| X12 beta strand | X1-X2-X3-X4-X5-X6-X7-X8-X9-X10-X11-(X12) |

Alternatively, the split point may comprise two split points such that 1 or more amino acid residue between the two split points are removed. In this embodiment, the two split points are made between non-adjacent residues, non-contiguously in the sequence, e.g. the split point is made between residues 94 and 97 while removing 95 and 96. This embodiment provides the possibility of, by way of a non-limiting example, making a split point and removing 1 or more residues in a beta-turn, such that the protein still folds and functions as when removing no residues at the split point. In the embodiment of the split point comprising two split points such that 1 or more amino acid residues between the two split points are removed, the two split points are: in the same domain; in adjacent domains; or in non-adjacent domains surrounding one other domain. As a result, up to three domains may be modified relative to the starting β-barrel polypeptide. By way of a non-limiting example, two split points in which the first split point is within X8 and the second split point is within X9 may yield the following circularly permuted β-barrel polypeptides:
(X9)-X10-X11-X12-X13-X14-X15-X16-X17-X18-X19-linker-X1-X2-X3-X4-X5-X6-X7-(X8): possible partial elimination of one or both of X8 and X9 (denoted by parentheses).
   By way of a further non-limiting example, two split points in which the first split point is within X7 and the second split point is within X9 may yield the following circularly permuted β-barrel polypeptides:
(X9)-X10-X11-X12-X13-X14-X15-X16-X17-X18-X19-linker-X1-X2-X3-X4-X5-X6-(X7): complete elimination of X8; partial elimination of X7 and/or X9. In this embodiment (full elimination of one domain), the fully eliminated domain cannot be a beta-strand domain (e.g. it can be of beta-turns, loops, alpha-helices, etc.), while beta-strands can still be partially eliminated (denoted by parentheses). In this way, a whole beta-turn comprising the residues of domain X8 could be eliminated if the split point is taken wholly at the C-terminus of the preceding beta-strand X7 and wholly at the N-terminus of the following beta-strand X9, giving: (X9)-X10-X11-X12-X13-X14-X15-X16-X17-X18-X19-linker-X1-X2-X3-X4-X5-X6-(X7).

In each embodiment, the point at which the original non-split β-barrel polypeptide is split (i.e. the “split point”) can be within one or two of the domains X1, X2, X3, X4, X5, X6, X7, X8, X9, X10, X11, X12, X13, X14, X15, X16, X17, X18, and X19, and can be present wholly at the N-termini of any of the domains, wholly at the C-termini of any of the domains, partially at the N-terminus and partially at the C-terminus of any of the domains, and at any of the amino acid residues in any of the domains. The two residues between which the split point is made need not be contiguous in amino acid sequence, and the one, two, or three domains within or between which the split point is made may be wholly or partially absent after making the split point. In the case of the domain being absent, in a non-limiting example in which the domain is a beta-turn, this is due to the elimination of the beta-turn secondary structural element (i.e. that domain) at which the split point is made, such that the original beta-turn is transformed into residues on each component comprising polypeptide fragments acquiring loop, beta-strand, or alpha-helical secondary structures. In the case of the domains being absent, in another non-limiting example in which the split point is made between any two domains of which the first domain is a beta-turn and the second domain is a beta-strand, the domains may be wholly or partially absent after making the split point due to the elimination of the beta-turn and beta-strand secondary structural elements (i.e. the first domain and the second domain) in which the split point is made, such that the original beta-turn and original beta-strand are transformed into residues on each component comprising polypeptide fragments acquiring loop, beta-strand, or alpha-helical secondary structures. For this reason, the split point is noted in parentheses, to note that the domain(s) are optional.

Those of skill in the art will readily understand the various other permutations possible based on the teachings herein.

In various embodiments that may be combined:
One domain is fully absent, wherein the fully absent domain is selected from domains X3, X5, X6, X8, X10, X11, X13, X15, X16, and X18;
Z1 is a hydrophobic amino acid and Z2 is a polar amino acid;
Z1 is selected from the group consisting of L, A, and F;
Z2 is selected from the group consisting of T, K, N, and D;
the X1 capping domain comprises an alpha helix;
X1 comprises the amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO:535) or RAAQLLP (SEQ ID NO:536), wherein the highlighted residue is invariant;
X2 comprises the amino acid sequence at least 50%, 55%, 60%, 65%, 70%, 75%, 80%, 85%, 90%, 95%, or 100% identical to the amino acid sequence G (T/K/N/D) WQZT(M/F)TN (SEQ ID NO:537) wherein Z is any amino acid, or GTWQ(V/L/A/I) T(M/F)TN (SEQ ID NO:538), wherein the highlighted residues are invariant;
X3 comprises the amino acid sequence (E/S)DG or EDG;
X4 comprises an amino acid sequence at least 50%, 55%, 60%, 65%, 70%, 75%, 80%, 85%, 90%, 95%, or 100% identical to the amino acid sequence QTSQGQMHFQP (SEQ ID NO:539), wherein the highlighted residues are invariant;
X5 comprises a single polar amino acid selected from the group consisting of R. T, Q. N, K, E, D, S, or wherein X5 is R;
X6 comprises the amino acid sequence (T/S)PZ3, where Z3 is polar amino acid or Tyr; or X6 is SPY;
X7 comprises an amino acid sequence at least 50%, 55%, 60%, 65%, 70%, 75%, 80%, 85%, 90%, 95%, or 100% identical to the amino acid sequence T(L/A/M)D(I/V)(K/V)(A/S) GT(I/M) (SEQ ID NO:540) or TMDIVAQGTI (SEQ ID NO:541), wherein the highlighted residues are invariant;
X8 comprises the amino acid sequence (S/A)DG or SDG;
X9 comprises an amino acid sequence at least 50%, 55%, 60%, 65%, 70%, 75%, 80%, 85%, 90%, 95%, or 100% identical to the amino acid sequence RPI(Q/S/T/V)G(Y/K)GK(L/V/A)T(V/C/A) (SEQ ID NO:542) or RPIVGYGKATV (SEQ ID NO:543), wherein the highlighted residues are invariant;
X10 is selected from the group consisting of R, T, Q, N, K, E, D, or S; or X10 is K;
X11 comprises the amino acid sequence (S/T)(P/C)(polar or Y), or X 11 is TPD;
X12 comprises an amino acid sequence at least 50%, 55%, 60%, 65%, 70%, 75%, 80%, 85%, 90%, 95%, or 100% identical to the amino acid sequence T(M/LN)(D/H/Q/N)(V/A/L/I)(D/N/H/Q)(I/LN)T(Y/W) (SEQ ID NO:544) or TLDIDITY (SEQ ID NO:545);
X13 comprises the amino acid sequence (S/E)DG, or X13 comprises an amino acid sequence at least 60%, 80%, or 100% identical to PSLGN (SEQ ID NO:546);
X14 comprises an amino acid sequence at least 50%, 55%, 60%, 65%, 70%, 75%, 80%, 85%, 90%, 95%, or 100% identical to the amino acid sequence (K/M/I/L)(Q/K)(V/A/G)QGQ(V/I)T(M/L/Y) (SEQ ID NO:547) or IKAQGQITM (SEQ ID NO:548), wherein the highlighted residues are invariant;
X15 is selected from the group consisting of R, T, Q, N, K, E, D, or S, or X15 is D;
X16 comprises the amino acid sequence (S/T)P(D/T/Y), or X16 comprises the amino acid sequence SPT;
X17 comprises an amino acid sequence at least 50%, 55%, 60%, 65%, 70%, 75%, 80%, 85%, 90%, 95%, or 100% identical to the amino acid sequence Q(F/A)(K/T/H)(F/W)(D/N)(V/A/S/G)(T/Q/H/E) (T/F/V/Y) (SEQ ID NO:549) or QFKFDATT (SEQ ID NO:550);
X19 comprises an amino acid sequence at least 50%, 55%, 60%, 65%, 70%, 75%, 80%, 85%, 90%, 95%, or 100% identical to the amino acid sequence [(S/K/N/H)](K/R/I/N)(V/L)TGT(L/I/M)QRQE (SEQ ID NO:551) or RLTGTLQRQE (SEQ ID NO:552), wherein residues in brackets are optional; and/or
X18 comprises the amino acid sequence selected from the group consisting of (S/E/N/A/Q)DG, SDG, K(G/Q/K/T)(A/D/E/N)(G/D/N)(N/G/D/Y/S) (SEQ ID NO:553), KG(A/D/E)(G/D/N)(N/G/D/Y) (SEQ ID NO:554), KGENDFHG (SEQ ID NO:555), KGADGWHG (SEQ ID NO:556), and KGAGNFTG (SEQ ID NO:557).

Any amino acid linker suitable for an intended use may be used in the polypeptides of this third aspect of the disclosure. For example, any structured or unstructured polypeptide linker can be used to create circularly permuted mFAPs (even whole structured domains of other proteins that act as polypeptide linkers), so long as the polypeptide linker fuses the C-terminus of X19 to the N-terminus of X1. In one non-limiting embodiment, the amino acid linker is at least 5-6 amino acids in length. In other non-limiting embodiments, the amino acid linker of claim 36 or 37, wherein the linker comprises a sequence selected from the group consisting of:

(SEQ ID NO: 558)
LPGGGGGDGTR
(SEQ ID NO: 559)
TPNAEEYLKELEERKRKGMQPLNE
(SEQ ID NO: 560)
TPKAGDEEYAKRLEEEARKKGGTI
(SEQ ID NO: 561)
TEPTGGGGGGGVT
(SEQ ID NO: 562)
LPTAEEWYKRWEKELRKRGTSWEQTL
(SEQ ID NO: 563)
EPRSEEIVKKAQHTWKGGSL
(SEQ ID NO: 564)
LPTAEEAQKEVKKKGLTGSN
(SEQ ID NO: 565)
LPGTEEWAKRIQEELKKKGYGTTK
(SEQ ID NO: 566)
TEEAKKILKEIQKKHKDEVQTDR
(SEQ ID NO: 567)
LPSAEEADEELKRQGVRGTL
(SEQ ID NO: 568)
TSDGGHGPDN

In another embodiment, the polypeptide of this third aspect comprises the first polypeptide component and the second polypeptide component of any embodiment or combination of embodiments of the first aspect of the disclosure, wherein the X19 domain is N-terminal to and connected directly to the X1 domain via an amino acid linker. In another embodiment, the β-barrel polypeptide comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOs:309-532, as shown below in Table 2.

TABLE 2
Amino acid sequences of circularly permuted β-barrel polypeptides.
The design naming convention used was: cp (shorthand for “circularly permuted”) +
the canonical mFAP residue number downstream (i.e. the C-terminal side) of the
split point + a dash (“_”) + the canonical mFAP residue number upstream (i.e. the
N-terminal side) of the split point + an underscore (“_”) + the canonical mFAP
design undergoing circular permutation (e.g. “mFAP2a”) + an underscore (“_”) + a de
novo designed linker sequence variant number (e.g. “08”) + an optional “_t”
designating that the two N-terminal and two C-terminal residues of the circularly
permuted mFAP maintain their respective residue types in the canonical mFAP (i.e.
the absence of “_t” designates that the two N-terminal and two C-terminal
residues of the circularly permuted mFAP were re-designed compared to their
respective residue types in the canonical mFAP).

| Design Name | Sequence |
|---|---|
| cp35-34_mFAP2a_08 | QEMDIVAQGTISDGRPIVGYGKATVKTPDTLDIDITYPSLGNIKAQGQIT |
| | MDSPTQFKWDATTKGENDFHGRLTGTEQRLPGTEEWAKRIQEEEKKKGYG |
| | TTKAAQLLPGTWQATFTNEDGQTSQGQWHFQPRDG (SEQ ID |
| | NO: 309) |
| cp35-34_mFAP2b_08 | QEMDIVAQGTISDGRPIVGYGKATVKTPDTLDIDITYPSLGNIKAQGQIT |
| | MDSPTQFKWDATTKGENDFHGRLTGTLQRLPGTEEWAKRIQEEEKKKGYG |
| | TTKAAQLLPGTWAVTMTNEDGQTSQGQWHFQPRDG (SEQ ID |
| | NO: 310) |
| cp35-34_mFAP3_08 | QEMDIVAQGTISDGRPIVGYGKATVKTPDTLDIDITWPSLGNIKGQGQIT |
| | MDSPTQFKWDGTTKGENDFHGRLTGTLQRLPGTEEWAKRIQEEEKKKGYG |
| | TTKAAQLLPGTWQATFTNEDGQTSQGQWHFQPRDG (SEQ ID |
| | NO: 311) |
| cp35-34_mFAP9_08 | QEMDIVAQGTISDGRPIVGYGKATVKTPDTLDIDITYPSLGNIKAQGQIT |
| | MDSPTQFKFDATTKGENDFHGRLTGTLQRLPGTEEWAKRIQEELKKKGYG |
| | TTKAAQLLPGTWQATFTNEDGQTSQGQFHFQPRDG (SEQ ID |
| | NO: 312) |
| cp35-34_mFAP10_08 | QEMDIVAQGTISDGRPIVGYGKATVKTPDTLDIDITYPSLGNIKAQGQIT |
| | MDSPTQFKFDATTKGENDFHGRLTGTLQRLPGTEEWAKRIQEELKKKGYG |
| | TTKAAQLLPGTWQATFTNEDGQTSQGQIHFQPRDG (SEQ ID |
| | NO: 313) |
| cp35-34_mFAP11_08 | QEMDIVSQGTISDGRPIVGYGKATVKTPDTLDIDITYPSLGNIKFQGQIT |
| | MDSPTQFKFDATTKGENDFHGRLTGTLQRLPGTEEWAKRIQEELKKKGYG |
| | TTKAAQLLPGTWQATFTNEDGQTSQGQIHFQPRDG (SEQ ID |
| | NO: 314) |
| cp35-34_mFAP12_08 | QEMDIVAQGTISDGRPIVGYGKATVKTPDTLDIDITYPSLGNIKAQGQIT |
| | MDSPTQFKFDATTSGSGGFKGRLTGTLQRLPGTEEWAKRIQEELKKKGYG |
| | TTKAAQLLPGTWQATFTNEDGQTSQGQIHFQPRDG (SEQ ID |
| | NO: 315) |
| cp35-34_mFAP_pH_08 | QEMDIVAQGTISDGRPIVGYGKATVKTPDTLDIDITYPSLGNIKAQGQIT |
| | MDSPTQFKFDATTKGENDFHGRLTGTLQRLPGTEEWAKRIQEELKKKGYG |
| | TTKAAQLLPGTWAVTMTNEDGQTSQGQMHFQPRDG (SEQ ID |
| | NO: 316) |
| cp35-34_mFAP2a_09 | QEMDIVAQGTISDGRPIVGYGKATVKTPDTLDIDITYPSLGNIKAQGQIT |
| | MDSPTQFKWDATTKGENDFHGRLTGTLQRTEEAKKILKEIQKKHKDEVQT |
| | DRAAQLLPGTWQATFTNEDGQTSQGQWHFQPRDG (SEQ ID NO: 317) |
| cp35-34_mFAP2b_09 | QEMDIVAQGTISDGRPIVGYGKATVKTPDTLDIDITYPSLGNIKAQGQIT |
| | MDSPTQFKWDATTKGENDFHGRLTGTLQRTEEAKKILKEIQKKHKDEVQT |
| | DRAAQLLPGTWAVTMTNEDGQTSQGQWHFQPRDG (SEQ ID NO: 318) |
| cp35-34_mFAP3_09 | QEMDIVAQGTISDGRPIVGYGKATVKTPDTLDIDITWPSLGNIKGQGQIT |
| | MDSPTQFKWDGTTKGENDFHGRLTGTLQRTEEAKKILKEIQKKHKDEVQT |
| | DRAAQLLPGTWQATFTNEDGQTSQGQWHFQPRDG (SEQ ID NO: 319) |
| cp35-34_mFAP9_09 | QEMDIVAQGTISDGRPIVGYGKATVKTPDTLDIDITYPSLGNIKAQGQIT |
| | MDSPTQFKFDATTKGENDFHGRLTGTLQRTEEAKKILKEIQKKHKDEVQT |
| | DRAAQLLPGTWQATFTNEDGQTSQGQFHFQPRDG (SEQ ID NO: 320) |
| cp35-34_mFAP10_09 | QEMDIVAQGTISDGRPIVGYGKATVKTPDTLDIDITYPSLGNIKAQGQIT |
| | MDSPTQFKFDATTKGENDFHGRLTGTEQRTEEAKKILKEIQKKHKDEVQT |
| | DRAAQLLPGTWQATFTNEDGQTSQGQIHFQPRDG (SEQ ID NO: 321) |
| cp35-34_mFAP11_09 | QEMDIVSQGTISDGRPIVGYGKATVKTPDTLDIDITYPSLGNIKFQGQIT |
| | MDSPTQFKFDATTKGENDFHGRLTGTLQRTEEAKKILKEIQKKHKDEVQT |
| | DRAAQLLPGTWQATFTNEDGQTSQGQIHFQPRDG (SEQ ID NO: 322) |
| cp35-34_mFAP12_09 | QEMDIVAQGTISDGRPIVGYGKATVKTPDTLDIDITYPSLGNIKAQGQIT |
| | MDSPTQFKFDATTSGSGGFKGRLTGTLQRTEEAKKILKEIQKKHKDEVQT |
| | DRAAQLLPGTWQATFTNEDGQTSQGQIHFQPRDG (SEQ ID NO: 323) |
| cp35-34_mFAP_pH_09 | QEMDIVAQGTISDGRPIVGYGKATVKTPDTLDIDITYPSLGNIKAQGQIT |
| | MDSPTQFKFDATTKGENDFHGRLTGTLQRTEEAKKILKEIQKKHKDEVQT |
| | DRAAQLLPGTWAVTMTNEDGQTSQGQMHFQPRDG (SEQ ID NO: 324) |
| cp35-34_mFAP2a_10 | QEMDIVAQGTISDGRPIVGYGKATVKTPDTLDIDITYPSLGNIKAQGQIT |
| | MDSPTQFKWDATTKGENDFHGRLTGTLQRLPSAEEADEELKRQGVRGTLA |
| | AQLLPGTWQATFTNEDGQTSQGQWHFQPRDG (SEQ ID NO: 325) |
| cp35-34_mFAP2b_10 | QEMDIVAQGTISDGRPIVGYGKATVKTPDTLDIDITYPSLGNIKAQGQIT |
| | MDSPTQFKWDATTKGENDFHGRLTGTLQRLPSAEEADEELKRQGVRGTLA |
| | AQLLPGTWAVTMTNEDGQTSQGQWHFQPRDG (SEQ ID NO: 326) |
| cp35-34_mFAP3_10 | QEMDIVAQGTISDGRPIVGYGKATVKTPDTLDIDITWPSLGNIKGQGQIT |
| | MDSPTQFKWDGTTKGENDFHGRLTGTLQRLPSAEEADEELKRQGVRGTLA |
| | AQLLPGTWQATFTNEDGQTSQGQWHFQPRDG (SEQ ID NO: 327) |
| cp35-34_mFAP9_10 | QEMDIVAQGTISDGRPIVGYGKATVKTPDTLDIDITYPSLGNIKAQGQIT |
| | MDSPTQFKFDATTKGENDFHGRLTGTLQRLPSAEEADEELKRQGVRGTLA |
| | AQLLPGTWQATFTNEDGQTSQGQFHFQPRDG (SEQ ID NO: 328) |
| cp35-34_mFAP10_10 | QEMDIVAQGTISDGRPIVGYGKATVKTPDTLDIDITYPSLGNIKAQGQIT |
| | MDSPTQFKFDATTKGENDFHGRLTGTLQRLPSAEEADEELKRQGVRGTLA |
| | AQLLPGTWQATFTNEDGQTSQGQIHFQPRDG (SEQ ID NO: 329) |
| cp35-34_mFAP11_10 | QEMDIVSQGTISDGRPIVGYGKATVKTPDTLDIDITYPSLGNIKFQGQIT |
| | MDSPTQFKFDATTKGENDFHGRLTGTLQRLPSAEEADEELKRQGVRGTLA |
| | AQLLPGTWQATFTNEDGQTSQGQIHFQPRDG (SEQ ID NO: 330) |
| cp35-34_mFAP12_10 | QEMDIVAQGTISDGRPIVGYGKATVKTPDTLDIDITYPSLGNIKAQGQIT |
| | MDSPTQFKFDATTSGSGGFKGRLTGTLQRLPSAEEADEELKRQGVRGTLA |
| | AQLLPGTWQATFTNEDGQTSQGQIHFQPRDG (SEQ ID NO: 331) |
| cp35-34_mFAP_pH_10 | QEMDIVAQGTISDGRPIVGYGKATVKTPDTLDIDITYPSLGNIKAQGQIT |
| | MDSPTQFKFDATTKGENDFHGRLTGTLQRLPSAEEADEELKRQGVRGTLA |
| | AQLLPGTWAVTMTNEDGQTSQGQMHFQPRDG (SEQ ID NO: 332) |
| cp35-34_mFAP2a_11 | QEMDIVAQGTISDGRPIVGYGKATVKTPDTLDIDITYPSLGNIKAQGQIT |
| | MDSPTQFKWDATTKGENDFHGRLTGTLQRTSDGGHGPDNAAQLLPGTWQA |
| | TFTNEDGQTSQGQWHFQPRDG (SEQ ID NO: 333) |
| cp35-34_mFAP2b_11 | QEMDIVAQGTISDGRPIVGYGKATVKTPDTLDIDITYPSLGNIKAQGQIT |
| | MDSPTQFKWDATTKGENDFHGRLTGTLQRTSDGGHGPDNAAQLLPGTWAV |
| | TMTNEDGQTSQGQWHFQPRDG (SEQ ID NO: 334) |
| cp35-34_mFAP3_11 | QEMDIVAQGTISDGRPIVGYGKATVKTPDTLDIDITWPSLGNIKGQGQIT |
| | MDSPTQFKWDGTTKGENDFHGRLTGTLQRTSDGGHGPDNAAQLLPGTWQA |
| | TFTNEDGQTSQGQWHFQPRDG (SEQ ID NO: 335) |
| cp35-34_mFAP9_11 | QEMDIVAQGTISDGRPIVGYGKATVKTPDTLDIDITYPSLGNIKAQGQIT |
| | MDSPTQFKFDATTKGENDFHGRLTGTLQRTSDGGHGPDNAAQLLPGTWQA |
| | TFTNEDGQTSQGQFHFQPRDG (SEQ ID NO: 336) |
| cp35-34_mFAP10_11 | QEMDIVAQGTISDGRPIVGYGKATVKTPDTLDIDITYPSLGNIKAQGQIT |
| | MDSPTQFKFDATTKGENDFHGRLTGTLQRTSDGGHGPDNAAQL1PGTWQA |
| | TFTNEDGQTSQGQIHFQPRDG (SEQ ID NO: 337) |
| cp35-34_mFAP11_11 | QEMDIVSQGTISDGRPIVGYGKATVKTPDTLDIDITYPSLGNIKFQGQIT |
| | MDSPTQFKFDATTKGENDFHGRLTGTLQRTSDGGHGPDNAAQLLPGTWQA |
| | TFTNEDGQTSQGQIHFQPRDG (SEQ ID NO: 338) |
| cp35-34_mFAP12_11 | QEMDIVAQGTISDGRPIVGYGKATVKTPDTLDIDITYPSLGNIKAQGQIT |
| | MDSPTQFKFDATTSGSGGFKGRLTGTLQRTSDGGHGPDNAAQLLPGTWQA |
| | TFTNEDGQTSQGQIHFQPRDG (SEQ ID NO: 339) |
| cp35-34_mFAP_pH_11 | QEMDIVAQGTISDGRPIVGYGKATVKTPDTLDIDITYPSLGNIKAQGQIT |
| | MDSPTQFKFDATTKGENDFHGRLTGTLQRTSDGGHGPDNAAQLLPGTWAV |
| | TMTNEDGQTSQGQMHFQPRDG (SEQ ID NO: 340) |
| cp35-34_mFAPZa_12 | QEMDIVAQGTISDGRPIVGYGKATVKTPDTLDIDITYPSLGNIKAQGQIT |
| | MDSPTQFKWDATTKGENDFHGRLTGTLQRTEEAKEATEEARRRGITTQAA |
| | QLLPGTWQATFTNEDGQTSQGQWHFQPRDG (SEQ ID NO: 341) |
| Cp35-34_mFAP2b_12 | QEMDIVAQGTISDGRPIVGYGKATVKTPDTLDIDITYPSLGNIKAQGQIT |
| | MDSPTQFKWDATTKGENDFHGRLTGTLQRTEEAKEATEEARRRGITTQAA |
| | QLLPGTWAVTMTNEDGQTSQGQWHFQPRDG (SEQ ID NO: 342) |
| cp35-34_mFAP3_12 | QEMDIVAQGTISDGRPIVGYGKATVKTPDTLDIDITWPSLGNIKGQGQIT |
| | MDSPTQFKWDGTTKGENDFHGRLTGTLQRTEEAKEATEEARRRGITTQAA |
| | QLLPGTWQATFTNEDGQTSQGQWHFQPRDG (SEQ ID NO: 343) |
| cp35-34_mFAP9_12 | QEMDIVAQGTISDGRPIVGYGKATVKTPDTLDIDITYPSLGNIKAQGQIT |
| | MDSPTQFKFDATTKGENDFHGRLTGTLQRTEEAKEATEEARRRGITTQAA |
| | QLLPGTWQATFTNEDGQTSQGQFHFQPRDG (SEQ ID NO: 34 4) |
| cp35-34_mFAP10_12 | QEMDIVAQGTISDGRPIVGYGKATVKTPDTLDIDITYPSLGNIKAQGQIT |
| | MDSPTQFKFDATTKGENDFHGRLTGTLQRTEEAKEATEEARRRGITTQAA |
| | QLLPGTWQATFTNEDGQTSQGQIHFQPRDG (SEQ ID NO: 34 5) |
| cp35-34_mFAP11_12 | QEMDIVSQGTISDGRPIVGYGKATVKTPDTLDIDITYPSLGNIKFQGQIT |
| | MDSPTQFKFDATTKGENDFHGRLTGTLQRTEEAKEATEEARRRGITTQAA |
| | QLLPGTWQATFTNEDGQTSQGQIHFQPRDG (SEQ ID NO: 346) |
| cp35-34_mFAP12_12 | QEMDIVAQGTISDGRPIVGYGKATVKTPDTLDIDITYPSLGNIKAQGQIT |
| | MDSPTQFKFDATTSGSGGFKGRLTGTLQRTEEAKEATEEARRRGITTQAA |
| | QLLPGTWQATFTNEDGQTSQGQIHFQPRDG (SEQ ID NO: 347) |
| cp35-34_mFAP_pH_12 | QEMDIVAQGTISDGRPIVGYGKATVKTPDTLDIDITYPSLGNIKAQGQIT |
| | MDSPTQFKFDATTKGENDFHGRLTGTLQRTEEAKEATEEARRRGITTQAA |
| | QLLPGTWAVTMTNEDGQTSQGQMHFQPRDG (SEQ ID NO: 348) |
| cp63-62_mFAPZa_07 | ATLDIDITYPSLGNIKAQGQITMDSPTQFKWDATTKGENDFHGRLTGTLQ |
| | RLPTAEEAQKEVKKKGLTGSNAAQLLPGTWQATFTNEDGQTSQGQWHFQP |
| | RSPYTMDIVAQGTISDGRPIVGYGKATVKDE (SEQ ID NO: 349) |
| cp63-62_mFAP2b_07 | ATLDIDITYPSLGNIKAQGQITMDSPTQFKWDATTKGENDFHGRLTGTLQ |
| | RLPTAEEAQKEVKKKGLTGSNAAQLLPGTWAVTMTNEDGQTSQGQWHFQP |
| | RSPYTMDIVAQGTISDGRPIVGYGKATVKDE (SEQ ID NO: 350) |
| cp63-62_mFAP3_07 | ATLDIDITWPSLGNIKGQGQITMDSPTQFKWDGTTKGENDFHGRLTGTLQ |
| | RLPTAEEAQKEVKKKGLTGSNAAQLLPGTWQATFTNEDGQTSQGQWHFQP |
| | RSPYTMDIVAQGTISDGRPIVGYGKATVKDE (SEQ ID NO: 351) |
| cp63-62_mFAP9_07 | ATLDIDITYPSLGNIKAQGQITMDSPTQFKFDATTKGENDFHGRLTGTLQ |
| | RLPTAEEAQKEVKKKGLTGSNAAQLLPGTWQATFTNEDGQTSQGQFHFQP |
| | RSPYTMDIVAQGTISDGRPIVGYGKATVKDE (SEQ ID NO: 352) |
| cp63-62_mFAP10_07 | ATLDIDITYPSLGNIKAQGQITMDSPTQFKFDATTKGENDFHGRLTGTLQ |
| | RLPTAEEAQKEVKKKGLTGSNAAQLLPGTWQATFTNEDGQTSQGQIHFQP |
| | RSPYTMDIVAQGTISDGRPIVGYGKATVKDE (SEQ ID NO: 353) |
| cp63-62_mFAP11_07 | ATLDIDITYPSLGNIKFQGQITMDSPTQFKFDATTKGENDFHGRLTGTLQ |
| | RLPTAEEAQKEVKKKGLTGSNAAQLLPGTWQATFTNEDGQTSQGQIHFQP |
| | RSPYTMDIVSQGTISDGRPIVGYGKATVKDE (SEQ ID NO: 354) |
| cp63-62_mFAP12_07 | ATLDIDITYPSLGNIKAQGQITMDSPTQFKFDATTSGSGGFKGRLTGTLQ |
| | RLPTAEEAQKEVKKKGLTGSNAAQLLPGTWQATFTNEDGQTSQGQIHFQP |
| | RSPYTMDIVAQGTISDGRPIVGYGKATVKDE (SEQ ID NO: 355) |
| cp63-62_mFAP_pH_07 | ATLDIDITYPSLGNIKAQGQITMDSPTQFKFDATTKGENDFHGRLTGTLQ |
| | RLPTAEEAQKEVKKKGLTGSNAAQLLPGTWAVTMTNEDGQTSQGQMHFQP |
| | RSPYTMDIVAQGTISDGRPIVGYGKATVKDE (SEQ ID NO: 356) |
| cp89-88_mFAPZa_05 | KQFKWDATTKGENDFHGRLTGTLQRLPTAEEWYKRWEKELRKRGTSWEQT |
| | LAAQLLPGTWQATFTNEDGQTSQGQWHFQPRSPYTMDIVAQGTISDGRPI |
| | VGYGKATVKTPDTLDIDITYPSLGNIKAQGQITMDEP (SEQ ID |
| | NO: 357) |
| cp89-88_mFAP2b_05 | KQFKWDATTKGENDFHGRLTGTLQRLPTAEEWYKRWEKELRKRGTSWEQT |
| | LAAQLLPGTWAVTMTNEDGQTSQGQWHFQPRSPYTMDIVAQGTISDGRPI |
| | VGYGKATVKTPDTLDIDITYPSLGNIKAQGQITMDEP (SEQ ID |
| | NO: 358) |
| cp89-88_mFAP3_05 | KQFKWDGTTKGENDFHGRLTGTLQRLPTAEEWYKRWEKELRKRGTSWEQT |
| | LAAQLLPGTWQATFTNEDGQTSQGQWHFQPRSPYTMDIVAQGTISDGRPI |
| | VGYGKATVKTPDTLDIDITWPSLGNIKGQGQITMDEP (SEQ ID |
| | NO: 359) |
| cp89-88_mFAP9_05 | KQFKFDATTKGENDFHGRLTGTLQRLPTAEEWYKRWEKELRKRGTSWEQT |
| | LAAQLLPGTWQATFTNEDGQTSQGQFHFQPRSPYTMDIVAQGTISDGRPI |
| | VGYGKATVKTPDTLDIDITYPSLGNIKAQGQITMDEP (SEQ ID |
| | NO: 360) |
| cp89-88_mFAP10_05 | KQFKFDATTKGENDFHGRLTGTLQRL?TAEEWYKRWEKELRKRGTSWEQT |
| | LAAQLLPGTWQATFTNEDGQTSQGQIHFQPRSPYTMDIVAQGTISDGRPI |
| | VGYGKATVKTPDTLDIDITYPSLGNIKAQGQITMDEP (SEQ ID |
| | NO: 361) |
| cp89-88_mFAP11_05 | KQFKFDATTKGENDFHGRLTGTLQRLPTAEEWYKRWEKELRKRGTSWEQT |
| | LAAQLLPGTWQATFTNEDGQTSQGQIHFQPRSPYTMDIVSQGTISDGRPI |
| | VGYGKATVKTPDTLDIDITYPSLGNIKFQGQITMDEP (SEQ ID |
| | NO: 362) |
| cp89-88_mFAP12_05 | KQFKFDATTSGSGGFKGRLTGTLQRLPTAEEWYKRWEKELRKRGTSWEQT |
| | LAAQLLPGTWQATFTNEDGQTSQGQIHFQPRSPYTMDIVAQGTISDGRPI |
| | VGYGKATVKTPDTLDIDITYPSLGNIKAQGQITMDEP (SEQ ID |
| | NO: 363) |
| cp89-88_mFAP_pH_05 | KQFKFDATTKGENDFHGRLTGTLQRLPTAEEWYKRWEKELRKRGTSWEQT |
| | LAAQLLPGTWAVTMTNEDGQTSQGQMHFQPRSPYTMDIVAQGTISDGRPI |
| | VGYGKATVKTPDTLDIDITYPSLGNIKAQGQITMDEP (SEQ ID |
| | NO: 364) |
| cp89-88_mFAP2a_06 | KQFKWDATTKGENDFHGRLTGTLQREPRSEEIVKKAQHTWKGGSLAAQLL |
| | PGTWQATFTNEDGQTSQGQWHFQPRSPYTMDIVAQGTISDGRPIVGYGKA |
| | TVKTPDTLDIDITYPSLGNIKAQGQITMDEP (SEQ ID NO: 365) |
| cp89-88_mFAP2b_06 | KQFKWDATTKGENDFHGRLTGTLQREPRSEEIVKKAQHTWKGGSLAAQLL |
| | PGTWAVTMTNEDGQTSQGQWHFQPRSPYTMDIVAQGTISDGRPIVGYGKA |
| | TVKTPDTLDIDITYPSLGNIKAQGQITMDEP (SEQ ID NO: 366) |
| cp89-88_mFAP3_06 | KQFKWDGTTKGENDFHGRLTGTLQREPRSEEIVKKAQHTWKGGSLAAQLL |
| | PGTWQATFTNEDGQTSQGQWHFQPRSPYTMDIVAQGTISDGRPIVGYGKA |
| | TVKTPDTLDIDITWPSLGNIKGQGQITMDEP (SEQ ID NO: 367) |
| cp89-88_mFAP9_06 | KQFKFDATTKGENDFHGRLTGTLQREPRSEEIVKKAQHTWKGGS1AAQLL |
| | PGTWQATFTNEDGQTSQGQFHFQPRSPYTMDIVAQGTISDGRPIVGYGKA |
| | TVKTPDTLDIDITYPSLGNIKAQGQITMDEP (SEQ ID NO: 368) |
| cp89-88_mFAP10_06 | KQFKFDATTKGENDFHGRLTGTLQREPRSEEIVKKAQHTWKGGSLAAQLL |
| | PGTWQATFTNEDGQTSQGQIHFQPRSPYTMDIVAQGTISDGRPIVGYGKA |
| | TVKTPDTLDIDITYPSLGNIKAQGQITMDEP (SEQ ID NO: 369) |
| cp89-88_mFAP11_06 | KQFKFDATTKGENDFHGRLTGTLQREPRSEEIVKKAQHTWKGGS1AAQLL |
| | PGTWQATFTNEDGQTSQGQIHFQPRSPYTMDIVSQGTISDGRPIVGYGKA |
| | TVKTPDTLDIDITYPSLGNIKFQGQITMDEP (SEQ ID NO: 370) |
| cp89-88_mFAP12_06 | KQFKFDATTSGSGGFKGRLTGTLQREPRSEEIVKKAQHTWKGGSLAAQLL |
| | PGTWQATFTNEDGQTSQGQIHFQPRSPYTMDIVAQGTISDGRPIVGYGKA |
| | TVKTPDTLDIDITYPSLGNIKAQGQITMDEP (SEQ ID NO: 371) |
| cp89-88_mFAP_pH_06 | KQFKFDATTKGENDFHGRLTGTLQREPRSEEIVKKAQHTWKGGSIAAQLL |
| | PGTWAVTMTNEDGQTSQGQMHFQPRSPYTMDIVAQGTISDGRPIVGYGKA |
| | TVKTPDTLDIDITYPSLGNIKAQGQITMDEP (SEQ ID NO: 372) |
| cp106-105_mFAP2a_01 | RLTGTLQRLPGGGGGDGTRAAQLLPGTWQATFTNEDGQTSQGQWHFQPRS |
| | PYTMDIVAQGTISDGRPIVGYGKATVKTPDTLDIDITYPSLGNIKAQGQI |
| | TMDSPTQFKWDATTKGENDFPG (SEQ ID NO: 373) |
| cp106-105_mFAP2b_01 | RLTGTLQRLPGGGGGDGTRAAQLLPGTWAVTMTNEDGQTSQGQWHFQPRS |
| | PYTMDIVAQGTISDGRPIVGYGKATVKTPDTLDIDITYPSLGNIKAQGQI |
| | TMDSPTQFKWDATTKGENDFPG (SEQ ID NO: 374) |
| cp106-105_mFAP3_01 | RLTGTLQRLPGGGGGDGTRAAQLLPGTWQATFTNEDGQTSQGQWHFQPRS |
| | PYTMDIVAQGTISDGRPIVGYGKATVKTPDTLDIDITWPSLGNIKGQGQI |
| | TMDSPTQFKWDGTTKGENDFPG (SEQ ID NO: 375) |
| cp106-105_mFAP9_01 | RLTGTLQRLPGGGGGDGTRAAQLLPGTWQATFTNEDGQTSQGQFHFQPRS |
| | PYTMDIVAQGTISDGRPIVGYGKATVKTPDTLDIDITYPSLGNIKAQGQI |
| | TMDSPTQFKFDATTKGENDFPG (SEQ ID NO: 376) |
| cp106-105_mFAP10_01 | RLTGTLQRLPGGGGGDGTRAAQLLPGTWQATFTNEDGQTSQGQIHFQPRS |
| | PYTMDIVAQGTISDGRPIVGYGKATVKTPDTLDIDITYPSLGNIKAQGQI |
| | TMDSPTQFKFDATTKGENDFPG (SEQ ID NO: 377) |
| cp106-105_mFAP11_01 | RLTGTLQRLPGGGGGDGTRAAQLLPGTWQATFTNEDGQTSQGQIHFQPRS |
| | PYTMDIVSQGTISDGRPIVGYGKATVKTPDTLDIDITYPSLGNIKFQGQI |
| | TMDSPTQFKFDATTKGENDFPG (SEQ ID NO: 37 8) |
| cp106-105_mFAP12_01 | RLTGTLQRLPGGGGGDGTRAAQLLPGTWQATFTNEDGQTSQGQIHFQPRS |
| | PYTMDIVAQGTISDGRPIVGYGKATVKTPDTLDIDITYPSLGNIKAQGQI |
| | TMDSPTQFKFDATTSGSGGFPG (SEQ ID NO: 379) |
| cp106-105_mFAP_pH_01 | RLTGTLQRLPGGGGGDGTRAAQLLPGTWAVTMTNEDGQTSQGQMHFQPRS |
| | PYTMDIVAQGTISDGRPIVGYGKATVKTPDTLDIDITYPSLGNIKAQGQI |
| | TMDSPTQFKFDATTKGENDFPG (SEQ ID NO: 380) |
| cp106-105_mFAP2a_02 | RLTGTLQRTPNAEEYLKELEERKRKGMQPLNEAAQLLPGTWQATFTNEDG |
| | QTSQGQWHFQPRSPYTMDIVAQGTISDGRPIVGYGKATVKTPDTLDIDIT |
| | YPSLGNIKAQGQITMDSPTQFKWDATTKGENDFPG (SEQ ID |
| | NO: 381) |
| cp106-105_mFAP2b_02 | RLTGTLQRTPNAEEYLKELEERKRKGMQPLNEAAQLLPGTWAVTMTNEDG |
| | QTSQGQWHFQPRSPYTMDIVAQGTISDGRPIVGYGKATVKTPDTLDIDIT |
| | YPSLGNIKAQGQITMDSPTQFKWDATTKGENDFPG (SEQ ID |
| | NO: 382) |
| cp106-105_mFAP3_02 | RLTGTLQRTPNAEEYLKELEERKRKGMQPLNEAAQLLPGTWQATFTNEDG |
| | QTSQGQWHFQPRSPYTMDIVAQGTISDGRPIVGYGKATVKTPDTLDIDIT |
| | WPSLGNIKGQGQITMDSPTQFKWDGTTKGENDFPG (SEQ ID |
| | NO: 383) |
| cp106-105_mFAP9_02 | RLTGTLQRTPNAEEYLKELEERKRKGMQPLNEAAQLLPGTWQATFTNEDG |
| | QTSQGQFHFQPRSPYTMDIVAQGTISDGRPIVGYGKATVKTPDTLDIDIT |
| | YPSLGNIKAQGQITMDSPTQFKFDATTKGENDFPG (SEQ ID |
| | NO: 384) |
| cp106-105_mFAP10_02 | RLTGTLQRTPNAEEYLKELEERKRKGMQPLNEAAQLLPGTWQATFTNEDG |
| | QTSQGQIHFQPRSPYTMDIVAQGTISDGRPIVGYGKATVKTPDTLDIDIT |
| | YPSLGNIKAQGQITMDSPTQFKFDATTKGENDFPG (SEQ ID |
| | NO: 385) |
| cp106-105_mFAP11_02 | RLTGTLQRTPNAEEYLKELEERKRKGMQPLNEAAQLLPGTWQATFTNEDG |
| | QTSQGQIHFQPRSPYTMDIVSQGTISDGRPIVGYGKATVKTPDTLDIDIT |
| | YPSLGNIKFQGQITMDSPTQFKFDATTKGENDFPG (SEQ ID |
| | NO: 386) |
| cp106-105_mFAP12_02 | RLTGTLQRTPNAEEYLKELEERKRKGMQPLNEAAQLLPGTWQATFTNEDG |
| | QTSQGQIHFQPRSPYTMDIVAQGTISDGRPIVGYGKATVKTPDTLDIDIT |
| | YPSLGNIKAQGQITMDSPTQFKFDATTSGSGGFPG (SEQ ID |
| | NO: 387) |
| cp106-105_mFAP_pH_02 | RLTGTLQRTPNAEEYLKELEERKRKGMQPLNEAAQLLPGTWAVTMTNEDG |
| | QTSQGQMHFQPRSPYTMDIVAQGTISDGRPIVGYGKATVKTPDTLDIDIT |
| | YPSLGNIKAQGQITMDSPTQFKFDATTKGENDFPG (SEQ ID |
| | NO: 388) |
| cp106-105_mFAP2a_03 | RLTGTLQRTPKAGDEEYAKRLEEEARKKGGTIAAQLLPGTWQATFTNEDG |
| | QTSQGQWHFQPRSPYTMDIVAQGTISDGRPIVGYGKATVKTPDTLDIDIT |
| | YPSLGNIKAQGQITMDSPTQFKWDATTKGENDFPG (SEQ ID |
| | NO: 389) |
| cp106-105_mFAP2b_03 | RLTGTLQRTPKAGDEEYAKRLEEEARKKGGTIAAQLLPGTWAVTMTNEDG |
| | QTSQGQWHFQPRSPYTMDIVAQGTISDGRPIVGYGKATVKTPDTLDIDIT |
| | YPSLGNIKAQGQITMDSPTQFKWDATTKGENDFPG (SEQ ID |
| | NO: 390) |
| cp106-105_mFAP3_03 | RLTGTLQRTPKAGDEEYAKRLEEEARKKGGTIAAQLLPGTWQATFTNEDG |
| | QTSQGQWHFQPRSPYTMDIVAQGTISDGRPIVGYGKATVKTPDTLDIDIT |
| | WPSLGNIKGQGQITMDSPTQFKWDGTTKGENDFPG (SEQ ID |
| | NO: 391) |
| cp106-105_mFAP9_03 | RLTGTLQRTPKAGDEEYAKRLEEEARKKGGTIAAQLLPGTWQATFTNEDG |
| | QTSQGQFHFQPRSPYTMDIVAQGTISDGRPIVGYGKATVKTPDTLDIDIT |
| | YPSLGNIKAQGQITMDSPTQFKFDATTKGENDFPG (SEQ ID |
| | NO: 392) |
| cp106-105_mFAP10_03 | RLTGTLQRTPKAGDEEYAKRLEEEARKKGGTIAAQLLPGTWQATFTNEDG |
| | QTSQGQIHFQPRSPYTMDIVAQGTISDGRPIVGYGKATVKTPDTLDIDIT |
| | YPSLGNIKAQGQITMDSPTQFKFDATTKGENDFPG (SEQ ID |
| | NO: 393) |
| Cp106-105_mFAP11_03 | RLTGTLQRTPKAGDEEYAKRLEEEARKKGGTIAAQLLPGTWQATFTNEDG |
| | QTSQGQIHFQPRSPYTMDIVSQGTISDGRPIVGYGKATVKTPDTLDIDIT |
| | YPSLGNIKFQGQITMDSPTQFKFDATTKGENDFPG (SEQ ID |
| | NO: 394) |
| cp106-105_mFAP12_03 | RLTGTLQRTPKAGDEEYAKRLEEEARKKGGTIAAQLLPGTWQATFTNEDG |
| | QTSQGQIHFQPRSPYTMDIVAQGTISDGRPIVGYGKATVKTPDTLDIDIT |
| | YPSLGNIKAQGQITMDSPTQFKFDATTSGSGGFPG (SEQ ID |
| | NO: 395) |
| cp106-105_mFAP_pH_03 | RLTGTLQRTPKAGDEEYAKRLEEEARKKGGTIAAQLLPGTWAVTMTNEDG |
| | QTSQGQMHFQPRSPYTMDIVAQGTISDGRPIVGYGKATVKTPDTLDIDIT |
| | YPSLGNIKAQGQITMDSPTQFKFDATTKGENDFPG (SEQ ID |
| | NO: 396) |
| cp106-105_mFAPZa_04 | RLTGTLQRTEPTGGGGGGGVTAAQLLPGTWQATFTNEDGQTSQGQWHFQP |
| | RSPYTMDIVAQGTISDGRPIVGYGKATVKTPDTLDIDITYPSLGNIKAQG |
| | QITMDSPTQFKWDATTKGENDFPG (SEQ ID NO: 397) |
| cp106-105_mFAP2b_04 | RLTGTLQRTEPTGGGGGGGVTAAQLLPGTWAVTMTNEDGQTSQGQWHFQP |
| | RSPYTMDIVAQGTISDGRPIVGYGKATVKTPDTLDIDITYPSLGNIKAQG |
| | QITMDSPTQFKWDATTKGENDFPG (SEQ ID NO: 398) |
| cp106-105_mFAP3_04 | RLTGTLQRTEPTGGGGGGGVTAAQLLPGTWQATFTNEDGQTSQGQWHFQP |
| | RSPYTMDIVAQGTISDGRPIVGYGKATVKTPDTLDIDITWPSLGNIKGQG |
| | QITMDSPTQFKWDGTTKGENDFPG (SEQ ID NO: 399) |
| cp106-105_mFAP9_04 | RLTGTLQRTEPTGGGGGGGVTAAQLLPGTWQATFTNEDGQTSQGQFHFQP |
| | RSPYTMDIVAQGTISDGRPIVGYGKATVKTPDTLDIDITYPSLGNIKAQG |
| | QITMDSPTQFKFDATTKGENDFPG (SEQ ID NO: 400) |
| cp106-105_mFAP10_04 | RLTGTLQRTEPTGGGGGGGVTAAQLLPGTWQATFTNEDGQTSQGQIHFQP |
| | RSPYTMDIVAQGTISDGRPIVGYGKATVKTPDTLDIDITYPSLGNIKAQG |
| | QITMDSPTQFKFDATTKGENDFPG (SEQ ID NO: 401) |
| cp106-105_mFAP11_04 | RLTGTLQRTEPTGGGGGGGVTAAQLLPGTWQATFTNEDGQTSQGQIHFQP |
| | RSPYTMDIVSQGTISDGRPIVGYGKATVKTPDTLDIDITYPSLGNIKFQG |
| | QITMDSPTQFKFDATTKGENDFPG (SEQ ID NO: 402) |
| cp106-105_mFAP12_04 | RLTGTLQRTEPTGGGGGGGVTAAQLLPGTWQATFTNEDGQTSQGQIHFQP |
| | RSPYTMDIVAQGTISDGRPIVGYGKATVKTPDTLDIDITYPSLGNIKAQG |
| | QITMDSPTQFKFDATTSGSGGFPG (SEQ ID NO: 403) |
| cp106-105_mFAP_pH_04 | RLTGTLQRTEPTGGGGGGGVTAAQLLPGTWAVTMTNEDGQTSQGQMHFQP |
| | RSPYTMDIVAQGTISDGRPIVGYGKATVKTPDTLDIDITYPSLGNIKAQG |
| | QITMDSPTQFKFDATTKGENDFPG (SEQ ID NO: 404) |
| cp35-34_mFAP2a_08_t | YTMDIVAQGTISDGRPIVGYGKATVKTPDTLDIDITYPSLGNIKAQGQIT |
| | MDSPTQFKWDATTKGENDFHGRLTGTLQRLPGTEEWAKRIQEELKKKGYG |
| | TTKAAQLLPGTWQATFTNEDGQTSQGQWHFQPRSP (SEQ ID |
| | NO: 405) |
| cp35-34_mFAP2b_08_t | YTMDIVAQGTISDGRPIVGYGKATVKTPDTLDIDITYPSLGNIKAQGQIT |
| | MDSPTQFKWDATTKGENDFHGRLTGTLQRLPGTEEWAKRIQEELKKKGYG |
| | TTKAAQLLPGTWAVTMTNEDGQTSQGQWHFQPRSP (SEQ ID |
| | NO: 406) |
| cp35-34_mFAP3_08_t | YTMDIVAQGTISDGRPIVGYGKATVKTPDTLDIDITWPSLGNIKGQGQIT |
| | MDSPTQFKWDGTTKGENDFHGRLTGTLQRLPGTEEWAKRIQEELKKKGYG |
| | TTKAAQLLPGTWQATFTNEDGQTSQGQWHFQPRSP (SEQ ID |
| | NO: 407) |
| cp35-34_mFAP9_08_t | YTMDIVAQGTISDGRPIVGYGKATVKTPDTLDIDITYPSLGNIKAQGQIT |
| | MDSPTQFKFDATTKGENDFHGRLTGTLQRLPGTEEWAKRIQEELKKKGYG |
| | TTKAAQLLPGTWQATFTNEDGQTSQGQFHFQPRSP (SEQ ID |
| | NO: 408) |
| cp35-34_mFAP10_08_t | YTMDIVAQGTISDGRPIVGYGKATVKTPDTLDIDITYPSLGNIKAQGQIT |
| | MDSPTQFKFDATTKGENDFHGRLTGTLQRLPGTEEWAKRIQEELKKKGYG |
| | TTKAAQLLPGTWQATFTNEDGQTSQGQIHFQPRSP (SEQ ID |
| | NO: 409) |
| cp35-34_mFAP11_08_t | YTMDIVSQGTISDGRPIVGYGKATVKTPDTLDIDITYPSLGNIKFQGQIT |
| | MDSPTQFKFDATTKGENDFHGRLTGTLQRLPGTEEWAKRIQEELKKKGYG |
| | TTKAAQLLPGTWQATFTNEDGQTSQGQIHFQPRSP (SEQ ID |
| | NO: 410) |
| Cp35-34_mFAP12_08_t | YTMDIVAQGTISDGRPIVGYGKATVKTPDTLDIDITYPSLGNIKAQGQIT |
| | MDSPTQFKFDATTSGSGGFKGRLTGTLQRLPGTEEWAKRIQEESKKKGYG |
| | TTKAAQLLPGTWQATFTNEDGQTSQGQIHFQPRSP (SEQ ID |
| | NO: 411) |
| cp35-34_mFAP_pH_08_t | YTMDIVAQGTISDGRPIVGYGKATVKTPDTLDIDITYPSLGNIKAQGQIT |
| | MDSPTQFKFDATTKGENDFHGRLTGTLQRLPGTEEWAKRIQEEEKKKGYG |
| | TTKAAQLLPGTWAVTMTNEDGQTSQGQMHFQPRSP (SEQ ID |
| | NO: 412) |
| cp35-34_mFAP2a_10_t | YTMDIVAQGTISDGRPIVGYGKATVKTPDTLDIDITYPSLGNIKAQGQIT |
| | MDSPTQFKWDATTKGENDFHGRLTGTLQRLPSAEEADEELKRQGVRGTLA |
| | AQLLPGTWQATFTNEDGQTSQGQWHFQPRSP (SEQ ID NO: 413) |
| cp35-34_mFAP2b_10_t | YTMDIVAQGTISDGRPIVGYGKATVKTPDTLDIDITYPSLGNIKAQGQIT |
| | MDSPTQFKWDATTKGENDFHGRLTGTLQRLPSAEEADEELKRQGVRGTLA |
| | AQLLPGTWAVTMTNEDGQTSQGQWHFQPRSP (SEQ ID NO: 414) |
| cp35-34_mFAP3_10_1t | YTMDIVAQGTISDGRPIVGYGKATVKTPDTLDIDITWPSLGNIKGQGQIT |
| | MDSPTQFKWDGTTKGENDFHGRLTGTLQRLPSAEEADEELKRQGVRGTLA |
| | AQLLPGTWQATFTNEDGQTSQGQWHFQPRSP (SEQ ID NO: 415) |
| cp35-34_mFAP9_10_1t | YTMDIVAQGTISDGRPIVGYGKATVKTPDTLDIDITYPSLGNIKAQGQIT |
| | MDSPTQFKFDATTKGENDFHGRLTGT1QRLPSAEEADEELKRQGVRGTLA |
| | AQLLPGTWQATFTNEDGQTSQGQFHFQPRSP (SEQ ID NO: 416) |
| cp35-34_mFAP10_10_t | YTMDIVAQGTISDGRPIVGYGKATVKTPDTLDIDITYPSLGNIKAQGQIT |
| | MDSPTQFKFDATTKGENDFHGRLTGTLQRLPSAEEADEELKRQGVRGTLA |
| | AQLLPGTWQATFTNEDGQTSQGQIHFQPRSP (SEQ ID NO: 417) |
| cp35-34_mFAP11_10_t | YTMDIVSQGTISDGRPIVGYGKATVKTPDTLDIDITYPSLGNIKFQGQIT |
| | MDSPTQFKFDATTKGENDFHGRLTGTLQRLPSAEEADEELKRQGVRGTLA |
| | AQLLPGTWQATFTNEDGQTSQGQIHFQPRSP (SEQ ID NO: 418) |
| cp35-34_mFAP12_10_t | YTMDIVAQGTISDGRPIVGYGKATVKTPDTLDIDITYPSLGNIKAQGQIT |
| | MDSPTQFKFDATTSGSGGFKGRLTGTLQRLPSAEEADEELKRQGVRGTLA |
| | AQLLPGTWQATFTNEDGQTSQGQIHFQPRSP (SEQ ID NO: 419) |
| cp35-34_mFAP_pH_10_t | YTMDIVAQGTISDGRPIVGYGKATVKTPDTLDIDITYPSLGNIKAQGQIT |
| | MDSPTQFKFDATTKGENDFHGRLTGTLQRLPSAEEADEELKRQGVRGTLA |
| | AQLLPGTWAVTMTNEDGQTSQGQMHFQPRSP (SEQ ID NO: 420) |
| cp35-34_mFAP2a_11_t | YTMDIVAQGTISDGRPIVGYGKATVKTPDTLDIDITYPSLGNIKAQGQIT |
| | MDSPTQFKWDATTKGENDFHGRLTGTLQRTSDGGHGPDNAAQLLPGTWQA |
| | TFTNEDGQTSQGQWHFQPRSP (SEQ ID NO: 421) |
| Cp35-34_mFAP2b_11_t | YTMDIVAQGTISDGRPIVGYGKATVKTPDTLDIDITYPSLGNIKAQGQIT |
| | MDSPTQFKWDATTKGENDFHGRLTGTLQRTSDGGHGPDNAAQLLPGTWAV |
| | TMTNEDGQTSQGQWHFQPRSP (SEQ ID NO: 422) |
| cp35-34_mFAP3_11_t | YTMDIVAQGTISDGRPIVGYGKATVKTPDTLDIDITWPSLGNIKGQGQIT |
| | MDSPTQFKWDGTTKGENDFHGRLTGTLQRTSDGGHGPDNAAQLLPGTWQA |
| | TFTNEDGQTSQGQWHFQPRSP (SEQ ID NO: 423) |
| cp35-34_mFAP9_11_t | YTMDIVAQGTISDGRPIVGYGKATVKTPDTLDIDITYPSLGNIKAQGQIT |
| | MDSPTQFKFDATTKGENDFHGRLTGTLQRTSDGGHGPDNAAQLLPGTWQA |
| | TFTNEDGQTSQGQFHFQPRSP (SEQ ID NO: 424) |
| cp35-34_mFAP10_11_t | YTMDIVAQGTISDGRPIVGYGKATVKTPDTLDIDITYPSLGNIKAQGQIT |
| | MDSPTQFKFDATTKGENDFHGRLTGTLQRTSDGGHGPDNAAQLLPGTWQA |
| | TFTNEDGQTSQGQIHFQPRSP (SEQ ID NO: 425) |
| cp35-34_mFAP11_11_t | YTMDIVSQGTISDGRPIVGYGKATVKTPDTLDIDITYPSLGNIKFQGQIT |
| | MDSPTQFKFDATTKGENDFHGRLTGTLQRTSDGGHGPDNAAQLLPGTWQA |
| | TFTNEDGQTSQGQIHFQPRSP (SEQ ID NO: 426) |
| cp35-34_mFAP12_11_t | YTMDIVAQGTISDGRPIVGYGKATVKTPDTLDIDITYPSLGNIKAQGQIT |
| | MDSPTQFKFDATTSGSGGFKGRLTGTLQRTSDGGHGPDNAAQLLPGTWQA |
| | TFTNEDGQTSQGQIHFQPRSP (SEQ ID NO: 427) |
| cp35-34_mFAP_pH_11_t | YTMDIVAQGTISDGRPIVGYGKATVKTPDTLDIDITYPSLGNIKAQGQIT |
| | MDSPTQFKFDATTKGENDFHGRLTGTLQRTSDGGHGPDNAAQLLPGTWAV |
| | TMTNEDGQTSQGQWHFQPRSP (SEQ ID NO: 428) |
| cp35-34_mFAPZa_12_t | YTMDIVAQGTISDGRPIVGYGKATVKTPDTLDIDITYPSLGNIKAQGQIT |
| | MDSPTQFKWDATTKGENDFHGRLTGTLQRTEEAKEATEEARRRGITTQAA |
| | QLLPGTWQATFTNEDGQTSQGQWHFQPRSP (SEQ ID NO: 42 9) |
| Cp35-34_mFAP2b_12_t | YTMDIVAQGTISDGRPIVGYGKATVKTPDTLDIDITYPSLGNIKAQGQIT |
| | MDSPTQFKWDATTKGENDFHGRLTGTLQRTEEAKEATEEARRRGITTQAA |
| | QLLPGTWAVTMTNEDGQTSQGQWHFQPRSP (SEQ ID NO: 430) |
| cp35-34_mFAP3_12_1 | YTMDIVAQGTISDGRPIVGYGKATVKTPDTLDIDITWPSLGNIKGQGQIT |
| | MDSPTQFKWDGTTKGENDFHGRLTGTLQRTEEAKEATEEARRRGITTQAA |
| | QLLPGTWQATFTNEDGQTSQGQWHFQPRSP (SEQ ID NO: 431) |
| cp35-34_mFAP9_12_t | YTMDIVAQGTISDGRPIVGYGKATVKTPDTLDIDITYPSLGNIKAQGQIT |
| | MDSPTQFKFDATTKGENDFHGRLTGTLQRTEEAKEATEEARRRGITTQAA |
| | QLLPGTWQATFTNEDGQTSQGQFHFQPRSP (SEQ ID NO: 432) |
| cp35-34_mFAP10_12_t | YTMDIVAQGTISDGRPIVGYGKATVKTPDTLDIDITYPSLGNIKAQGQIT |
| | MDSPTQFKFDATTKGENDFHGRLTGTLQRTEEAKEATEEARRRGITTQAA |
| | QLLPGTWQATFTNEDGQTSQGQIHFQPRSP (SEQ ID NO: 433) |
| cp35-34_mFAP11_12_t | YTMDIVSQGTISDGRPIVGYGKATVKTPDTLDIDITYPSLGNIKFQGQIT |
| | MDSPTQFKFDATTKGENDFHGRLTGTLQRTEEAKEATEEARRRGITTQAA |
| | QLLPGTWQATFTNEDGQTSQGQIHFQPRSP (SEQ ID NO: 434) |
| cp35-34_mFAP12_12_t | YTMDIVAQGTISDGRPIVGYGKATVKTPDTLDIDITYPSLGNIKAQGQIT |
| | MDSPTQFKFDATTSGSGGFKGRLTGTLQRTEEAKEATEEARRRGITTQAA |
| | QLLPGTWQATFTNEDGQTSQGQIHFQPRSP (SEQ ID NO: 435) |
| cp35-34_mFAP_pH_12_t | YTMDIVAQGTISDGRPIVGYGKATVKTPDTLDIDITYPSLGNIKAQGQIT |
| | MDSPTQFKFDATTKGENDFHGRLTGTLQRTEEAKEATEEARRRGITTQAA |
| | QLLPGTWAVTMTNEDGQTSQGQMHFQPRSP (SEQ ID NO: 436) |
| cp63-62_mFAPZa_08_t | DTLDIDITYPSLGNIKAQGQITMDSPTQFKWDATTKGENDFHGRLTGTLQ |
| | RLPGTEEWAKRIQEELKKKGYGTTKAAQLLPGTWQATFTNEDGQTSQGQW |
| | HFQPRSPYTMDIVAQGTISDGRPIVGYGKATVKTP (SEQ ID |
| | NO: 437) |
| cp63-62_mFAP2b_08_t | DTLDIDITYPSLGNIKAQGQITMDSPTQFKWDATTKGENDFHGRLTGTLQ |
| | RLPGTEEWAKRIQEELKKKGYGTTKAAQLLPGTWAVTMTNEDGQTSQGQW |
| | HFQPRSPYTMDIVAQGTISDGRPIVGYGKATVKTP (SEQ ID |
| | NO: 438) |
| cp63-62_mFAP3_08_t | DTLDIDITWPSLGNIKGQGQITMDSPTQFKWDGTTKGENDFHGRLTGTLQ |
| | RLPGTEEWAKRIQEELKKKGYGTTKAAQLLPGTWQATFTNEDGQTSQGQW |
| | HFQPRSPYTMDIVAQGTISDGRPIVGYGKATVKTP (SEQ ID |
| | NO: 439) |
| cp63-62_mFAP9_08_t | DTLDIDITYPSLGNIKAQGQITMDSPTQFKFDATTKGENDFHGRLTGTLQ |
| | RLPGTEEWAKRIQEELKKKGYGTTKAAQLLPGTWQATFTNEDGQTSQGQF |
| | HFQPRSPYTMDIVAQGTISDGRPIVGYGKATVKTP (SEQ ID |
| | NO: 440) |
| cp63-62_mFAP10_08_t | DTLDIDITYPSLGNIKAQGQITMDSPTQFKFDATTKGENDFHGRLTGTLQ |
| | RLPGTEEWAKRIQEELKKKGYGTTKAAQLLPGTWQATFTNEDGQTSQGQI |
| | HFQPRSPYTMDIVAQGTISDGRPIVGYGKATVKTP (SEQ ID |
| | NO: 441) |
| cp63-62_mFAP11_08_t | DTLDIDITYPSLGNIKFQGQITMDSPTQFKFDATTKGENDFHGRLTGTLQ |
| | RLPGTEEWAKRIQEELKKKGYGTTKAAQLLPGTWQATFTNEDGQTSQGQI |
| | HFQPRSPYTMDIVSQGTISDGRPIVGYGKATVKTP (SEQ ID |
| | NO: 442) |
| cp63-62_mFAP12_08_t | DTLDIDITYPSLGNIKAQGQITMDSPTQFKFDATTSGSGGFKGRLTGTLQ |
| | RLPGTEEWAKRIQEELKKKGYGTTKAAQLLPGTWQATFTNEDGQTSQGQI |
| | HFQPRSPYTMDIVAQGTISDGRPIVGYGKATVKTP (SEQ ID |
| | NO: 443) |
| cp63-62_mFAP_pH_08_t | DTLDIDITYPSLGNIKAQGQITMDSPTQFKFDATTKGENDFHGRLTGTLQ |
| | RLPGTEEWAKRIQEELKKKGYGTTKAAQLLPGTWAVTMTNEDGQTSQGQM |
| | HFQPRSPYTMDIVAQGTISDGRPIVGYGKATVKTP (SEQ ID |
| | NO: 444) |
| cp63-62_mFAPZa_10_t | DTLDIDITYPSLGNIKAQGQITMDSPTQFKWDATTKGENDFHGRLTGTLQ |
| | RLPSAEEADEELKRQGVRGTLAAQLLPGTWQATFTNEDGQTSQGQWHFQP |
| | RSPYTMDIVAQGTISDGRPIVGYGKATVKTP (SEQ ID NO: 445) |
| cp63-62_mFAP2b_10_t | DTLDIDITYPSLGNIKAQGQITMDSPTQFKWDATTKGENDFHGRLTGTLQ |
| | RLPSAEEADEELKRQGVRGTLAAQLLPGTWAVTMTNEDGQTSQGQWHFQP |
| | RSPYTMDIVAQGTISDGRPIVGYGKATVKTP (SEQ ID NO: 446) |
| Cp63-62_mFAP3_10_t | DTLDIDITWPSLGNIKGQGQITMDSPTQFKWDGTTKGENDFHGRLTGTLQ |
| | RLPSAEEADEELKRQGVRGTLAAQLLPGTWQATFTNEDGQTSQGQWHFQP |
| | RSPYTMDIVAQGTISDGRPIVGYGKATVKTP (SEQ ID NO: 447) |
| cp63-62_mFAP9_10_t | DTLDIDITYPSLGNIKAQGQITMDSPTQFKFDATTKGENDFHGRLTGTLQ |
| | RLPSAEEADEELKRQGVRGTLAAQLLPGTWQATFTNEDGQTSQGQFHFQP |
| | RSPYTMDIVAQGTISDGRPIVGYGKATVKTP (SEQ ID NO: 448) |
| cp63-62_mFAP10_10_t | DTLDIDITYPSLGNIKAQGQITMDSPTQFKFDATTKGENDFHGRLTGTLQ |
| | RLPSAEEADEELKRQGVRGTLAAQLLPGTWQATFTNEDGQTSQGQIHFQP |
| | RSPYTMDIVAQGTISDGRPIVGYGKATVKTP (SEQ ID NO: 449) |
| cp63-62_mFAP11_10_t | DTLDIDITYPSLGNIKFQGQITMDSPTQFKFDATTKGENDFHGRLTGTLQ |
| | RLPSAEEADEELKRQGVRGTLAAQLLPGTWQATFTNEDGQTSQGQIHFQP |
| | RSPYTMDIVSQGTISDGRPIVGYGKATVKTP (SEQ ID NO: 450) |
| cp63-62_mFAP12_10_1 | DTLDIDITYPSLGNIKAQGQITMDSPTQFKFDATTSGSGGFKGRLTGTLQ |
| | RLPSAEEADEELKRQGVRGTLAAQLL?GTWQATFTNEDGQTSQGQIHFQP |
| | RSPYTMDIVAQGTISDGRPIVGYGKATVKTP (SEQ ID NO: 451) |
| cp63-62_mFAP_pH_10_t | DTLDIDITYPSLGNIKAQGQITMDSPTQFKFDATTKGENDFHGRLTGTLQ |
| | RLPSAEEADEELKRQGVRGTLAAQLLPGTWAVTMTNEDGQTSQGQMHFQP |
| | RSPYTMDIVAQGTISDGRPIVGYGKATVKTP (SEQ ID NO: 452) |
| cp63-62_mFAP2a_11_t | DTLDIDITYPSLGNIKAQGQITMDSPTQFKWDATTKGENDFHGRLTGTLQ |
| | RTSDGGHGPDNAAQLLPGTWQATFTNEDGQTSQGQWHFQPRSPYTMDIVA |
| | QGTISDGRPIVGYGKATVKTP (SEQ ID NO: 453) |
| cp63-62_mFAP2b_11_t | DTLDIDITYPSLGNIKAQGQITMDSPTQFKWDATTKGENDFHGRLTGTLQ |
| | RTSDGGHGPDNAAQLLPGTWAVTMTNEDGQTSQGQWHFQPRSPYTMDIVA |
| | QGTISDGRPIVGYGKATVKTP (SEQ ID NO: 454) |
| cp63-62_mFAP3_11_t | DTLDIDITWPSLGNIKGQGQITMDSPTQFKWDGTTKGENDFHGRLTGTLQ |
| | RTSDGGHGPDNAAQLLPGTWQATFTNEDGQTSQGQWHFQPRSPYTMDIVA |
| | QGTISDGRPIVGYGKATVKTP (SEQ ID NO: 455) |
| cp63-62_mFAP9_11_t | DTLDIDITYPSLGNIKAQGQITMDSPTQFKFDATTKGENDFHGRLTGTLQ |
| | RTSDGGHGPDNAAQLLPGTWQATFTNEDGQTSQGQFHFQPRSPYTMDIVA |
| | QGTISDGRPIVGYGKATVKTP (SEQ ID NO: 456) |
| cp63-62_mFAP10_11_t | DTLDIDITYPSLGNIKAQGQITMDSPTQFKFDATTKGENDFHGRLTGTLQ |
| | RTSDGGHGPDNAAQLLPGTWQATFTNEDGQTSQGQIHFQPRSPYTMDIVA |
| | QGTISDGRPIVGYGKATVKTP (SEQ ID NO: 457) |
| cp63-62_mFAP11_11_t | DTLDIDITYPSLGNIKFQGQITMDSPTQFKFDATTKGENDFHGRLTGTLQ |
| | RTSDGGHGPDNAAQLLPGTWQATFTNEDGQTSQGQIHFQPRSPYTMDIVS |
| | QGTISDGRPIVGYGKATVKTP (SEQ ID NO: 458) |
| cp63-62_mFAP12_11_t | DTLDIDITYPSLGNIKAQGQITMDSPTQFKFDATTSGSGGFKGRLTGTLQ |
| | RTSDGGHGPDNAAQLLPGTWQATFTNEDGQTSQGQIHFQPRSPYTMDIVA |
| | QGTISDGRPIVGYGKATVKTP (SEQ ID NO: 459) |
| cp63-62_mFAP_pH_11_t | DTLDIDITYPSLGNIKAQGQITMDSPTQFKFDATTKGENDFHGRLTGTLQ |
| | RTSDGGHGPDNAAQLLPGTWAVTMTNEDGQTSQGQMHFQPRSPYTMDIVA |
| | QGTISDGRPIVGYGKATVKTP (SEQ ID NO: 460) |
| cp63-62_mFAP2a_12_t | DTLDIDITYPSLGNIKAQGQITMDSPTQFKWDATTKGENDFHGRLTGTLQ |
| | RTEEAKEATEEARRRGITTQAAQLLPGTWQATFTNEDGQTSQGQWHFQPR |
| | SPYTMDIVAQGTISDGRPIVGYGKATVKTP (SEQ ID NO: 461) |
| cp63-62_mFAP2b_12_t | DTLDIDITYPSLGNIKAQGQITMDSPTQFKWDATTKGENDFHGRLTGTLQ |
| | RTEEAKEATEEARRRGITTQAAQLLPGTWAVTMTNEDGQTSQGQWHFQPR |
| | SPYTMDIVAQGTISDGRPIVGYGKATVKTP (SEQ ID NO: 462) |
| cp63-62_mFAP3_12_t | DTLDIDITWPSLGNIKGQGQITMDSPTQFKWDGTTKGENDFHGRLTGTLQ |
| | RTEEAKEATEEARRRGITTQAAQLLPGTWQATFTNEDGQTSQGQWHFQPR |
| | SPYTMDIVAQGTISDGRPIVGYGKATVKTP (SEQ ID NO: 463) |
| cp63-62_mFAP9_12_t | DTLDIDITYPSLGNIKAQGQITMDSPTQFKFDATTKGENDFHGRLTGTLQ |
| | RTEEAKEATEEARRRGITTQAAQLLPGTWQATFTNEDGQTSQGQFHFQPR |
| | SPYTMDIVAQGTISDGRPIVGYGKATVKTP (SEQ ID NO: 464) |
| cp63-62_mFAP10_12_t | DTLDIDITYPSLGNIKAQGQITMDSPTQFKFDATTKGENDFHGRLTGTLQ |
| | RTEEAKEATEEARRRGITTQAAQLLPGTWQATFTNEDGQTSQGQIHFQPR |
| | SPYTMDIVAQGTISDGRPIVGYGKATVKTP (SEQ ID NO: 465) |
| cp63-62_mFAP11_12_t | DTLDIDITYPSLGNIKFQGQITMDSPTQFKFDATTKGENDFHGRLTGTLQ |
| | RTEEAKEATEEARRRGITTQAAQLLPGTWQATFTNEDGQTSQGQIHFQPR |
| | SPYTMDIVSQGTISDGRPIVGYGKATVKTP (SEQ ID NO: 466) |
| cp63-62_mFAP12_12_t | DTLDIDITYPSLGNIKAQGQITMDSPTQFKFDATTSGSGGFKGRITGTLQ |
| | RTEEAKEATEEARRRGITTQAAQLLPGTWQATFTNEDGQTSQGQIHFQPR |
| | SPYTMDIVAQGTISDGRPIVGYGKATVKTP (SEQ ID NO: 4 67) |
| cp63-62_mFAP_pH_12_t | DTLDIDITYPSLGNIKAQGQITMDSPTQFKFDATTKGENDFHGRLTGTLQ |
| | RTEEAKEATEEARRRGITTQAAQLLPGTWAVTMTNEDGQTSQGQMHFQPR |
| | SPYTMDIVAQGTISDGRPIVGYGKATVKTP (SEQ ID NO: 468) |
| cp89-88_mFAP2a_08_t | TQFKWDATTKGENDFHGRLTGTLQRLPGTEEWAKRIQEELKKKGYGTTKA |
| | AQLLPGTWQATFTNEDGQTSQGQWHFQPRSPYTMDIVAQGTISDGRPIVG |
| | YGKATVKTPDTLDIDITYPSLGNIKAQGQITMDSP (SEQ ID |
| | NO: 469) |
| cp89-88_mFAP2b_08_t | TQFKWDATTKGENDFHGRLTGTLQRLPGTEEWAKRIQEELKKKGYGTTKA |
| | AQLLPGTWAVTMTNEDGQTSQGQWHFQPRSPYTMDIVAQGTISDGRPIVG |
| | YGKATVKTPDTLDIDITYPSLGNIKAQGQITMDSP (SEQ ID |
| | NO: 470) |
| cp89-88_mFAP3_08_t | TQFKWDGTTKGENDFHGRLTGTLQRLPGTEEWAKRIQEELKKKGYGTTKA |
| | AQLLPGTWQATFTNEDGQTSQGQWHFQPRSPYTMDIVAQGTISDGRPIVG |
| | YGKATVKTPDTLDIDITWPSLGNIKGQGQITMDSP (SEQ ID |
| | NO: 471) |
| cp89-88_mFAP9_08_t | TQFKFDATTKGENDFHGRLTGTLQRLPGTEEWAKRIQEELKKKGYGTTKA |
| | AQLLPGTWQATFTNEDGQTSQGQFHFQPRSPYTMDIVAQGTISDGRPIVG |
| | YGKATVKTPDTLDIDITYPSLGNIKAQGQITMDSP (SEQ ID |
| | NO: 472) |
| cp89-88_mFAP10_08_t | TQFKFDATTKGENDFHGRLTGTLQRLPGTEEWAKRIQEELKKKGYGTTKA |
| | AQLLPGTWQATFTNEDGQTSQGQIHFQPRSPYTMDIVAQGTISDGRPIVG |
| | YGKATVKTPDTLDIDITYPSLGNIKAQGQITMDSP (SEQ ID |
| | NO: 473) |
| cp89-88_mFAP11_08_t | TQFKFDATTKGENDFHGRLTGTLQRLPGTEEWAKRIQEELKKKGYGTTKA |
| | AQLLPGTWQATFTNEDGQTSQGQIHFQPRSPYTMDIVSQGTISDGRPIVG |
| | YGKATVKTPDTLDIDITYPSLGNIKFQGQITMDSP (SEQ ID |
| | NO: 474) |
| cp89-88_mFAP12_08_t | TQFKFDATTSGSGGFKGRLTGTLQRLPGTEEWAKRIQEELKKKGYGTTKA |
| | AQLLPGTWQATFTNEDGQTSQGQIHFQPRSPYTMDIVAQGTISDGRPIVG |
| | YGKATVKTPDTLDIDITYPSLGNIKAQGQITMDSP (SEQ ID |
| | NO: 475) |
| cp89-88_mFAP_pH_08_t | TQFKFDATTKGENDFHGRLTGTLQRLPGTEEWAKRIQEELKKKGYGTTKA |
| | AQLLPGTWAVTMTNEDGQTSQGQMHFQPRSPYTMDIVAQGTISDGRPIVG |
| | YGKATVKTPDTLDIDITYPSLGNIKAQGQITMDSP (SEQ ID |
| | NO: 476) |
| cp89-88_mFAP2a_10_t | TQFKWDATTKGENDFHGRLTGTLQRLPSAEEADEELKRQGVRGT1AAQLL |
| | PGTWQATFTNEDGQTSQGQWHFQPRSPYTMDIVAQGTISDGRPIVGYGKA |
| | TVKTPDTLDIDITYPSLGNIKAQGQITMDSP (SEQ ID NO: 477) |
| cp89-88_mFAP2b_10_t | TQFKWDATTKGENDFHGRLTGTLQRLPSAEEADEELKRQGVRGT1AAQLL |
| | PGTWAVTMTNEDGQTSQGQWHFQPRSPYTMDIVAQGTISDGRPIVGYGKA |
| | TVKTPDTLDIDITYPSLGNIKAQGQITMDSP (SEQ ID NO: 478) |
| cp89-88_mFAP3_10_t | TQFKWDGTTKGENDFHGRLTGTLQRLPSAEEADEELKRQGVRGT1AAQLL |
| | PGTWQATFTNEDGQTSQGQWHFQPRSPYTMDIVAQGTISDGRPIVGYGKA |
| | TVKTPDTLDIDITWPSLGNIKGQGQITMDSP (SEQ ID NO: 479) |
| cp89-88_mFAP9_10_t | TQFKFDATTKGENDFHGRLTGTLQRLPSAEEADEELKRQGVRGT1AAQLL |
| | PGTWQATFTNEDGQTSQGQFHFQPRS2YTMDIVAQGTISDGRPIVGYGKA |
| | TVKTPDTLDIDITYPSLGNIKAQGQITMDSP (SEQ ID NO: 4 80) |
| cp89-88_mFAP10_10_t | TQFKFDATTKGENDFHGRLTGTLQRLPSAEEADEELKRQGVRGT1AAQLL |
| | PGTWQATFTNEDGQTSQGQIHFQPRS2YTMDIVAQGTISDGRPIVGYGKA |
| | TVKTPDTLDIDITYPSLGNIKAQGQITMDSP (SEQ ID NO: 481) |
| cp89-88_mFAP11_10_t | TQFKFDATTKGENDFHGRLTGTLQRLPSAEEADEELKRQGVRGT1AAQLL |
| | PGTWQATFTNEDGQTSQGQIHFQPRS2YTMDIVSQGTISDGRPIVGYGKA |
| | TVKTPDTLDIDITYPSLGNIKFQGQITMDSP (SEQ ID NO: 482) |
| cp89-88_mFAP12_10_t | TQFKFDATTSGSGGFKGRLTGTLQRL2SAEEADEELKRQGVRGTLAAQLL |
| | PGTWQATFTNEDGQTSQGQIHFQPRS2YTMDIVAQGTISDGRPIVGYGKA |
| | TVKTPDTLDIDITYPSLGNIKAQGQITMDSP (SEQ ID NO: 483) |
| Cp89-88_mFAP_pH_10_t | TQFKFDATTKGENDFHGRLTGTLQRLPSAEEADEELKRQGVRGTLAAQLL |
| | PGTWAVTMTNEDGQTSQGQMHFQPRS2YTMDIVAQGTISDGRPIVGYGKA |
| | TVKTPDTLDIDITYPSLGNIKAQGQITMDSP (SEQ ID NO: 484) |
| cp89-88_mFAP2a_11_t | TQFKWDATTKGENDFHGRLTGTLQRTSDGGHGPDNAAQLLPGTWQATFTN |
| | EDGQTSQGQWHFQPRSPYTMDIVAQGTISDGRPIVGYGKATVKTPDTLDI |
| | DITYPSLGNIKAQGQITMDSP (SEQ ID NO: 485) |
| cp89-88_mFAP2b_11_t | TQFKWDATTKGENDFHGRLTGTLQRTSDGGHGPDNAAQLLPGTWAVTMTN |
| | EDGQTSQGQWHFQPRSPYTMDIVAQGTISDGRPIVGYGKATVKTPDTLDI |
| | DITYPSLGNIKAQGQITMDSP (SEQ ID NO: 486) |
| cp89-88_mFAP3_11_t | TQFKWDGTTKGENDFHGRLTGTLQRTSDGGHGPDNAAQLLPGTWQATFTN |
| | EDGQTSQGQWHFQPRSPYTMDIVAQGTISDGRPIVGYGKATVKTPDTLDI |
| | DITWPSLGNIKGQGQITMDSP (SEQ ID NO: 487) |
| cp89-88_mFAP9_11_t | TQFKFDATTKGENDFHGRLTGTLQRTSDGGHGPDNAAQLLPGTWQATFTN |
| | EDGQTSQGQFHFQPRSPYTMDIVAQGTISDGRPIVGYGKATVKTPDTLDI |
| | DITYPSLGNIKAQGQITMDSP (SEQ ID NO: 488) |
| cp89-88_mFAP10_11_t | TQFKFDATTKGENDFHGRLTGTLQRTSDGGHGPDNAAQLLPGTWQATFTN |
| | EDGQTSQGQIHFQPRSPYTMDIVAQGTISDGRPIVGYGKATVKTPDTLDI |
| | DITYPSLGNIKAQGQITMDSP (SEQ ID NO: 489) |
| cp89-88_mFAP11_11_t | TQFKFDATTKGENDFHGRLTGTLQRTSDGGHGPDNAAQLLPGTWQATFTN |
| | EDGQTSQGQIHFQPRSPYTMDIVSQGTISDGRPIVGYGKATVKTPDTLDI |
| | DITYPSLGNIKFQGQITMDSP (SEQ ID NO: 490) |
| cp89-88_mFAP12_11_t | TQFKFDATTSGSGGFKGRLTGTLQRTSDGGHGPDNAAQLLPGTWQATFTN |
| | EDGQTSQGQIHFQPRSPYTMDIVAQGTISDGRPIVGYGKATVKTPDTLDI |
| | DITYPSLGNIKAQGQITMDSP (SEQ ID NO: 491) |
| cp89-88_mFAP_pH_11_t | TQFKFDATTKGENDFHGRLTGTLQRTSDGGHGPDNAAQLLPGTWAVTMTN |
| | EDGQTSQGQMHFQPRSPYTMDIVAQGTISDGRPIVGYGKATVKTPDTLDI |
| | DITYPSLGNIKAQGQITMDSP (SEQ ID NO: 492) |
| cp89-88_mFAP2a_12_t | TQFKWDATTKGENDFHGRLTGTLQRTEEAKEATEEARRRGITTQAAQLLP |
| | GTWQATFTNEDGQTSQGQWHFQPRSPYTMDIVAQGTISDGRPIVGYGKAT |
| | VKTPDTLDIDITYPSLGNIKAQGQITMDSP (SEQ ID NO: 493) |
| cp89-88_mFAP2b_12_t | TQFKWDATTKGENDFHGRLTGTLQRTEEAKEATEEARRRGITTQAAQLLP |
| | GTWAVTMTNEDGQTSQGQWHFQPRSPYTMDIVAQGTISDGRPIVGYGKAT |
| | VKTPDTLDIDITYPSLGNIKAQGQITMDSP (SEQ ID NO: 494) |
| cp89-88_mFAP3_12_t | TQFKWDGTTKGENDFHGRLTGTLQRTEEAKEATEEARRRGITTQAAQLLP |
| | GTWQATFTNEDGQTSQGQWHFQPRSPYTMDIVAQGTISDGRPIVGYGKAT |
| | VKTPDTLDIDITWPSLGNIKGQGQITMDSP (SEQ ID NO: 495) |
| cp89-88_mFAP9_12_t | TQFKFDATTKGENDFHGRLTGTLQRTEEAKEATEEARRRGITTQAAQLLP |
| | GTWQATFTNEDGQTSQGQFHFQPRSPYTMDIVAQGTISDGRPIVGYGKAT |
| | VKTPDTLDIDITYPSLGNIKAQGQITMDSP (SEQ ID NO: 496) |
| cp89-88_mFAP10_12_t | TQFKFDATTKGENDFHGRLTGTLQRTEEAKEATEEARRRGITTQAAQLLP |
| | GTWQATFTNEDGQTSQGQIHFQPRSPYTMDIVAQGTISDGRPIVGYGKAT |
| | VKTPDTLDIDITYPSLGNIKAQGQITMDSP (SEQ ID NO: 497) |
| cp89-88_mFAP11_12_t | TQFKFDATTKGENDFHGRLTGTLQRTEEAKEATEEARRRGITTQAAQLLP |
| | GTWQATFTNEDGQTSQGQIHFQPRSPYTMDIVSQGTISDGRPIVGYGKAT |
| | VKTPDTLDIDITYPSLGNIKFQGQITMDSP (SEQ ID NO: 498) |
| cp89-88_mFAP12_12_t | TQFKFDATTSGSGGFKGRLTGTLQRTEEAKEATEEARRRGITTQAAQLLP |
| | GTWQATFTNEDGQTSQGQIHFQPRSPYTMDIVAQGTISDGRPIVGYGKAT |
| | VKTPDTLDIDITYPSLGNIKAQGQITMDSP (SEQ ID NO: 499) |
| cp89-88_mFAP_pH_12_t | TQFKFDATTKGENDFHGRLTGTLQRTEEAKEATEEARRRGITTQAAQLLP |
| | GTWAVTMTNEDGQTSQGQMHFQPRSPYTMDIVAQGTISDGRPIVGYGKAT |
| | VKTPDTLDIDITYPSLGNIKAQGQITMDSP (SEQ ID NO: 500) |
| cpl06-105_mFAP2a_08_t | RLTGTLQRLPGTEEWAKRIQEELKKKGYGTTKAAQLLPGTWQATFTNEDG |
| | QTSQGQWHFQPRSPYTMDIVAQGTISDGRPIVGYGKATVKTPDTLDIDIT |
| | YPSLGNIKAQGQITMDSPTQFKWDATTKGENDFHG (SEQ ID |
| | NO: 501) |
| cp_106-105_mFAP2b_08_t | RLTGTLQRLPGTEEWAKRIQEELKKKGYGTTKAAQLLPGTWAVTMTNEDG |
| | QTSQGQWHFQPRSPYTMDIVAQGTISDGRPIVGYGKATVKTPDTLDIDIT |
| | YPSLGNIKAQGQITMDSPTQFKWDATTKGENDFHG (SEQ ID |
| | NO: 502) |
| Cp106-105_mFAP3_08_t | RLTGTLQRLPGTEEWAKRIQEELKKKGYGTTKAAQLLPGTWQATFTNEDG |
| | QTSQGQWHFQPRSPYTMDIVAQGTISDGRPIVGYGKATVKTPDTLDIDIT |
| | WPSLGNIKGQGQITMDSPTQFKWDGTTKGENDFHG (SEQ ID |
| | NO: 503) |
| cp106-105_mFAP9_08_t | RLTGTLQRLPGTEEWAKRIQEELKKKGYGTTKAAQLLPGTWQATFTNEDG |
| | QTSQGQFHFQPRSPYTMDIVAQGTISDGRPIVGYGKATVKTPDTLDIDIT |
| | YPSLGNIKAQGQITMDSPTQFKFDATTKGENDFHG (SEQ ID |
| | NO: 504) |
| cp106-105_mFAP10_08_t | RLTGTLQRLPGTEEWAKRIQEELKKKGYGTTKAAQLLPGTWQATFTNEDG |
| | QTSQGQIHFQPRSPYTMDIVAQGTISDGRPIVGYGKATVKTPDTLDIDIT |
| | YPSLGNIKAQGQITMDSPTQFKFDATTKGENDFHG (SEQ ID |
| | NO: 505) |
| cp106-105_mFAP11_08_t | RLTGTLQRLPGTEEWAKRIQEELKKKGYGTTKAAQLLPGTWQATFTNEDG |
| | QTSQGQIHFQPRSPYTMDIVSQGTISDGRPIVGYGKATVKTPDTLDIDIT |
| | YPSLGNIKFQGQITMDSPTQFKFDATTKGENDFHG (SEQ ID |
| | NO: 506) |
| cp106-105_mFAP12_08_t | RLTGTLQRLPGTEEWAKRIQEELKKKGYGTTKAAQLLPGTWQATFTNEDG |
| | QTSQGQIHFQPRSPYTMDIVAQGTISDGRPIVGYGKATVKTPDTLDIDIT |
| | YPSLGNIKAQGQITMDSPTQFKFDATTSGSGGFKG (SEQ ID |
| | NO: 507) |
| cp106-105_mFAP_pH_08_t | RLTGTLQRLPGTEEWAKRIQEELKKKGYGTTKAAQLLPGTWAVTMTNEDG |
| | QTSQGQMHFQPRSPYTMDIVAQGTISDGRPIVGYGKATVKTPDTLDIDIT |
| | YPSLGNIKAQGQITMDSPTQFKFDATTKGENDFHG (SEQ ID |
| | NO: 508) |
| cp106-105_mFAP2a_10_t | RLTGTLQRLPSAEEADEELKRQGVRGTLAAQLLPGTWQATFTNEDGQTSQ |
| | GQWHFQPRSPYTMDIVAQGTISDGRPIVGYGKATVKTPDTLDIDITYPSL |
| | GNIKAQGQITMDSPTQFKWDATTKGENDFHG (SEQ ID NO: 509) |
| cp106-105_mFAP2b_10_t | RLTGTLQRLPSAEEADEELKRQGVRGTLAAQLLPGTWAVTMTNEDGQTSQ |
| | GQWHFQPRSPYTMDIVAQGTISDGRPIVGYGKATVKTPDTLDIDITYPSL |
| | GNIKAQGQITMDSPTQFKWDATTKGENDFHG (SEQ ID NO: 510) |
| cp106-105_mFAP3_10_t | RLTGTLQRLPSAEEADEELKRQGVRGTLAAQLLPGTWQATFTNEDGQTSQ |
| | GQWHFQPRSPYTMDIVAQGTISDGRPIVGYGKATVKTPDTLDIDITWPSL |
| | GNIKGQGQITMDSPTQFKWDGTTKGENDFHG (SEQ ID NO: 511) |
| cp106-105_mFAP9_10_t | RLTGTLQRLPSAEEADEELKRQGVRGTLAAQLLPGTWQATFTNEDGQTSQ |
| | GQFHFQPRSPYTMDIVAQGTISDGRPIVGYGKATVKTPDTLDIDITYPSL |
| | GNIKAQGQITMDSPTQFKFDATTKGENDFHG (SEQ ID NO: 512) |
| cp106-105_mFAP10_10_t | RLTGTLQRLPSAEEADEELKRQGVRGTLAAQLLPGTWQATFTNEDGQTSQ |
| | GQIHFQPRSPYTMDIVAQGTISDGRPIVGYGKATVKTPDTLDIDITYPSL |
| | GNIKAQGQITMDSPTQFKFDATTKGENDFHG (SEQ ID NO: 513) |
| cp106-105_mFAP11_10_t | RLTGTLQRLPSAEEADEELKRQGVRGTLAAQLLPGTWQATFTNEDGQTSQ |
| | GQIHFQPRSPYTMDIVSQGTISDGRPIVGYGKATVKTPDTLDIDITYPSL |
| | GNIKFQGQITMDSPTQFKFDATTKGENDFHG (SEQ ID NO: 514) |
| cp106-105_mFAP12_10_t | RLTGTLQRLPSAEEADEELKRQGVRGTLAAQLLPGTWQATFTNEDGQTSQ |
| | GQIHFQPRSPYTMDIVAQGTISDGRPIVGYGKATVKTPDTLDIDITYPSL |
| | GNIKAQGQITMDSPTQFKFDATTSGSGGFKG (SEQ ID NO: 515) |
| cp106-105_mFAP_pH_10_t | RLTGTLQRLPSAEEADEELKRQGVRGTLAAQLLPGTWAVTMTNEDGQTSQ |
| | GQMHFQPRSPYTMDIVAQGTISDGRPIVGYGKATVKTPDTLDIDITYPSL |
| | GNIKAQGQITMDSPTQFKFDATTKGENDFHG (SEQ ID NO: 516) |
| cp106-105_mFAP2a_11_t | RLTGTLQRTSDGGHGPDNAAQLLPGTWQATFTNEDGQTSQGQWHFQPRSP |
| | YTMDIVAQGTISDGRPIVGYGKATVKTPDTLDIDITYPSLGNIKAQGQIT |
| | MDSPTQFKWDATTKGENDFHG (SEQ ID NO: 517) |
| cp106-105_mFAP2b_11_t | RLTGTLQRTSDGGHGPDNAAQLLPGTWAVTMTNEDGQTSQGQWHFQPRSP |
| | YTMDIVAQGTISDGRPIVGYGKATVKTPDTLDIDITYPSLGNIKAQGQIT |
| | MDSPTQFKWDATTKGENDFHG (SEQ ID NO: 518) |
| cp106-105_mFAP3_11_t | RLTGTLQRTSDGGHGPDNAAQLLPGTWQATFTNEDGQTSQGQWHFQPRSP |
| | YTMDIVAQGTISDGRPIVGYGKATVKTPDTLDIDITWPSLGNIKGQGQIT |
| | MDSPTQFKWDGTTKGENDFHG (SEQ ID NO: 519) |
| cp106-105_mFAP9_11_t | RLTGTLQRTSDGGHGPDNAAQLLPGTWQATFTNEDGQTSQGQFHFQPRSP |
| | YTMDIVAQGTISDGRPIVGYGKATVKTPDTLDIDITYPSLGNIKAQGQIT |
| | MDSPTQFKFDATTKGENDFHG (SEQ ID NO: 52 0) |
| cp106-105_mFAP10_11_t | RLTGTLQRTSDGGHGPDNAAQLLPGTWQATFTNEDGQTSQGQIHFQPRSP |
| | YTMDIVAQGTISDGRPIVGYGKATVKTPDTLDIDITYPSLGNIKAQGQIT |
| | MDSPTQFKFDATTKGENDFHG (SEQ ID NO: 521) |
| cp106-105_mFAP11_11_t | RLTGTLQRTSDGGHGPDNAAQLLPGTWQATFTNEDGQTSQGQIHFQPRSP |
| | YTMDIVSQGTISDGRPIVGYGKATVKTPDTLDIDITYPSLGNIKFQGQIT |
| | MDSPTQFKFDATTKGENDFHG (SEQ ID NO: 522) |
| cp106-105_mFAP12_11_t | RLTGTLQRTSDGGHGPDNAAQLLPGTWQATFTNEDGQTSQGQIHFQPRSP |
| | YTMDIVAQGTISDGRPIVGYGKATVKTPDTLDIDITYPSLGNIKAQGQIT |
| | MDSPTQFKFDATTSGSGGFKG (SEQ ID NO: 523) |
| cp106-105_mFAP_pH_11_t | RLTGTLQRTSDGGHGPDNAAQLLPGTWAVTMTNEDGQTSQGQMHFQPRSP |
| | YTMDIVAQGTISDGRPIVGYGKATVKTPDTLDIDITYPSLGNIKAQGQIT |
| | MDSPTQFKFDATTKGENDFHG (SEQ ID NO: 524) |
| cp106-105_mFAP2a_12_t | RLTGTLQRTEEAKEATEEARRRGITTQAAQLLPGTWQATFTNEDGQTSQG |
| | QWHFQPRSPYTMDIVAQGTISDGRPIVGYGKATVKTPDTLDIDITYPSLG |
| | NIKAQGQITMDSPTQFKWDATTKGENDFHG (SEQ ID NO: 525) |
| cp106-105_mFAP2b_12_t | RLTGTLQRTEEAKEATEEARRRGITTQAAQLLPGTWAVTMTNEDGQTSQG |
| | QWHFQPRSPYTMDIVAQGTISDGRPIVGYGKATVKTPDTLDIDITYPSLG |
| | NIKAQGQITMDSPTQFKWDATTKGENDFHG (SEQ ID NO: 526) |
| cp106-105_mFAP3_12_t | RLTGTLQRTEEAKEATEEARRRGITTQAAQLLPGTWQATFTNEDGQTSQG |
| | QWHFQPRSPYTMDIVAQGTISDGRPIVGYGKATVKTPDTLDIDITWPSLG |
| | NIKGQGQITMDSPTQFKWDGTTKGENDFHG (SEQ ID NO: 527) |
| cp106-105_mFAP9_12_t | RLTGTLQRTEEAKEATEEARRRGITTQAAQLLPGTWQATFTNEDGQTSQG |
| | QFHFQPRSPYTMDIVAQGTISDGRPIVGYGKATVKTPDTLDIDITYPSLG |
| | NIKAQGQITMDSPTQFKFDATTKGENDFHG (SEQ ID NO: 528) |
| cp106-105_mFAP10_12_t | RLTGTLQRTEEAKEATEEARRRGITTQAAQLLPGTWQATFTNEDGQTSQG |
| | QIHFQPRSPYTMDIVAQGTISDGRPIVGYGKATVKTPDTLDIDITYPSLG |
| | NIKAQGQITMDSPTQFKFDATTKGENDFHG (SEQ ID NO: 529) |
| cp106-105_mFAP11_12_t | RLTGTLQRTEEAKEATEEARRRGITTQAAQLLPGTWQATFTNEDGQTSQG |
| | QIHFQPRSPYTMDIVSQGTISDGRPIVGYGKATVKTPDTLDIDITYPSLG |
| | NIKFQGQITMDSPTQFKFDATTKGENDFHG (SEQ ID NO: 530) |
| cp106-105_mFAP12_12_t | RLTGTLQRTEEAKEATEEARRRGITTQAAQLLPGTWQATFTNEDGQTSQG |
| | QIHFQPRSPYTMDIVAQGTISDGRPIVGYGKATVKTPDTLDIDITYPSLG |
| | NIKAQGQITMDSPTQFKFDATTSGSGGFKG (SEQ ID NO: 531) |
| cp106-105_mFAP_pH_12_t | RLTGTLQRTEEAKEATEEARRRGITTQAAQLLPGTWAVTMTNEDGQTSQG |
| | QMHFQPRSPYTMDIVAQGTISDGRPIVGYGKATVKTPDTLDIDITYPSLG |
| | NIKAQGQITMDSPTQFKFDATTKGENDFHG (SEQ ID NO: 532) |

In another embodiment, the β-barrel polypeptides of the disclosure comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOs:533-534, as shown below in Table 3.

TABLE 3
Canonical, single-chain mFAPs.

| | |
|---|---|
| mFAP9 | SRAAQLLPGTWQATFTNEDGQTSQGQTHFQPRSPYTMDIVAQGTISDGRP |
| | IVGYGKATVKTPDTLDIDITYPSLGNIKAQGQITMDSPTQFKFDATTKGE |
| | NDFHGRLTGTLQRQE (SEQ ID NO: 533) |
| mFAP10 | SRAAQLLPGTWQATFTNEDGQTSQGQIHFQPRSPYTMDIVAQGTISDGRP |
| | IVGYGKATVKTPDTLDIDITYPSLGNIKAQGQITMDSPTQFKFDATTKGE |
| | NDFHGRLTGTLQRQE (SEQ ID NO: 534) |

In another embodiment, the polypeptides of this third aspect of the disclosure may further comprise one or more functional domains, as described in detail above for the first aspect.

As used throughout the present application, the term “polypeptide” is used in its broadest sense to refer to a sequence of subunit D- or L-amino acids, including canonical and non-canonical amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another aspect the disclosure provides nucleic acids encoding the polypeptides of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptides, nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the present disclosure provides pharmaceutical compositions, comprising one or more the multipartite β-barrel proteins, 0-barrel polypeptides, polypeptides, nucleic acids, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (1) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The multipartite β-barrel proteins, β-barrel polypeptides, polypeptides, nucleic acids, expression vectors, and/or host cells may be the sole active agent in the pharmaceutical composition, or the composition may further comprise one or more other active agents suitable for an intended use.

In a further aspect, the disclosure provides uses and methods for use of the self-complementing multipartite β-barrel protein, the polypeptide, the nucleic acid, the expression vector, the recombinant cell, and/or the β-barrel polypeptide of any aspect, embodiment, or combinations thereof, for uses including, but not limited to, pH sensing, ion-sensing/detection (including but not limited to Ca²⁺, La³⁺, Tb²⁺, and other ion sensing/detection/quantification), temporal sensing, voltage sensing, mechanical sensing, thermal sensing, super-resolution microscopy, localization microscopy, fluorescence microscopy, fluorescence lifetime imaging, fluorimetry, and detection and quantification of other small-molecules, ions, peptides, nucleic acids, organic substrates, or inorganic substrates by insertion of their respective binding peptides into the loops, beta turns, or beta strands of any of the polypeptides of any of the claims herein, or by covalent fusion or non-covalent linkage of their respective binding peptides to any of the polypeptides of any of the claims herein.

The disclosure further provides methods for designing the multipartite β-barrel proteins or the polypeptides of any aspect, embodiment, or combinations thereof, wherein the methods comprise any of the methods disclosed in the examples that follow.

### EXAMPLES

This innovation describes self-complementing multipartite β-barrel polypeptides (“split mFAPs”) capable of mediating real-time monitoring of polypeptide-polypeptide association and dissociation events through reversible self-complementation into a reporter complex capable of activating the fluorescence of exogenous fluorogenic compounds such as, but not limited to, DFHBI (3,5-difluoro-4-hydroxybenzylidene imidazolinone), DFHBI-1T [(Z)-4-(3,5-difluoro-4-hydroxybenzylidene)-2-methyl-1-(2,2,2-trifluoroethyl)-1H-imidazol-5(4H)-one], and DFHO (3,5-difluoro-4-hydroxybenzylidene imidazolinone-2-oxime), with different degrees of specificity and affinity. Multipartite β-barrel polypeptides may be used as versatile polypeptide scaffolds in the engineering of novel oligomeric polypeptide assemblies for the detection of interactions of polypeptides of interest in real-time using fluorescence microscopy and fluorimetry techniques. Additionally, this innovation describes circularly permuted mFAPs (“cpmFAPs”) capable of activating the fluorescence of the exogenous fluorogenic compounds such as, but not limited to, DFHBI-1T. Circularly permuted β-barrel polypeptides may be used as versatile polypeptide scaffolds in the engineering of novel fluorogenic optical biosensors for the detection of analytes of interest in real-time using fluorescence microscopy and fluorimetry techniques.

The fluorescently active structures of canonical, single-chain β-barrel polypeptides (also known as mFAPs) are composed of eight antiparallel β-strands. In the design of multipartite β-barrel polypeptides from an eight β-stranded β-barrel topology such that β-strands are preserved while split points are taken only in the β-hairpin structural motifs, there exists: one split point for bipartite β-barrel polypeptides, two split points for tripartite β-barrel polypeptides, three split points for tetrapartite β-barrel polypeptides, four split points for pentapartite β-barrel polypeptides, five split points for hexapartite β-barrel polypeptides, six split points for heptapartite β-barrel polypeptides, and seven split points for octapartite β-barrel polypeptides. As a prerequisite for high fluorescence reporting activity, exactly one of each of the eight β-strands must participate in the active multipartite β-barrel polypeptide complex, independent of β-strand connectivity and the number of β-strands per polypeptide fragment. Therefore, there is the possibility of extraneous β-strands on multipartite β-barrel polypeptide fragments participating in the active complex. Self-complementing multipartite β-barrel polypeptides allow real-time monitoring of polypeptide-polypeptide association and dissociation events through reversible self-complementation of β-barrel polypeptide fragments into a conformationally active complex capable of binding and activating the fluorescence of exogenous fluorogenic compounds. Herein, we present de novo designed multipartite β-barrel polypeptides (Table 1), also called split mFAPs, and methods for their use.

Circular permutation of fluorescent proteins such as green fluorescent protein (GFP) facilitates the engineering of novel fluorescent optical biosensors capable of real-time detection of analytes of interest using fluorescence microscopy techniques. Circularly permuted fluorescent proteins are ideal polypeptide scaffolds for optical biosensor engineering due to the proximity of the N- and C-termini to the chromophore, which allosterically couples conformational changes in covalently fused analyte-binding polypeptides of interest to conformational changes in the chromophore environment, allowing intensiometric fluorescence measurements of analyte concentrations. Herein, we present de novo designed circularly permuted β-barrel polypeptides (Table 2), also known as circularly permuted mFAPs (cpmFAPs), capable of binding and activating the fluorescence of exogenous fluorogenic compounds, and methods for their use.

Circularly permuted β-barrel polypeptides are designed such that the N- and C-termini of the canonical, single-chain β-barrel polypeptides are covalently fused with a de novo designed structured or unstructured linker, and a single split point is chosen elsewhere in the β-barrel polypeptide to form new N- and C-termini. Because the new N- and C-termini in the circularly permuted mFAPs are adjacent to one another, circularly permuted mFAPs are ideal polypeptide scaffolds for the design of polypeptide-based fluorogenic optical biosensors, in which analyte-binding polypeptide domains covalently fused to the N- and C-termini of circularly permuted mFAPs act as bioreceptor elements and fluorescence activity of the chromophore-bound circularly permuted mFAPs acts as the transducer elements. As such, analyte binding and unbinding events causing conformational changes in the bioreceptor element may be allosterically coupled to conformational changes of the residues coordinating the chromophore in the binding pockets of circularly permuted mFAPs. Analyte binding can thereby modulate the thermodynamic dissociation constants of various exogenous fluorogenic compounds for binding to circularly permuted mFAPs, resulting in modulated fluorescence intensity upon analyte binding due to binding and unbinding of exogenous fluorogenic compounds to the transducer element. Additionally, analyte binding to the bioreceptor element may be allosterically coupled to conformational changes in the transducer element resulting in stabilization or destabilization of the fluorescent conformation of exogenous fluorogenic compounds (e.g., cis-planar conformation of DFHBI-1T) bound to the circularly permuted mFAPs, resulting in modulated fluorescence intensity upon analyte binding. Thus, circularly permuted β-barrel polypeptides may be considered versatile polypeptide scaffolds for the engineering of novel polypeptide-based fluorogenic optical biosensors that detect analytes of interest in real-time using fluorescence microscopy and fluorimetry methodologies.

### Results:

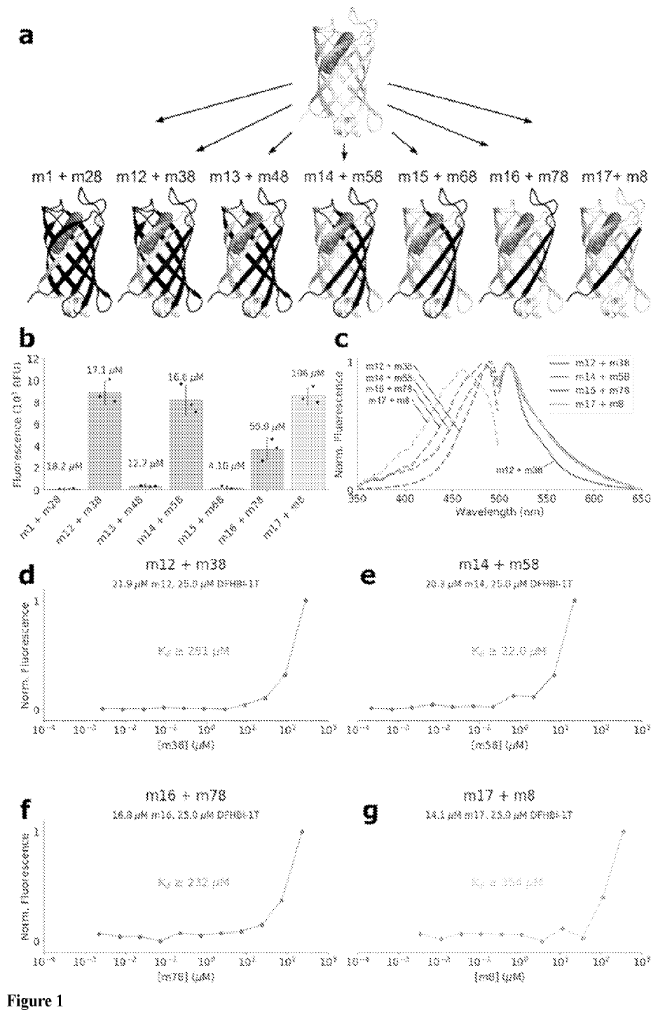

The concept of self-complementing multipartite β-barrel polypeptide fragments to activate reporter activity by fluorescence activation of exogenous fluorogenic compounds is demonstrated using bipartite split mFAPs. Self-complementing bipartite β-barrel polypeptides are designed by creating split points in the β-hairpins and loop7 (i.e. the loop connecting β-strand 7 to β-strand 8) of the mFAP2a scaffold. With a total of eight β-strands in the canonical, single-chain β-barrel polypeptide topology, and by only making split points between β-strands within β-hairpin structural motifs, there exists seven unique, self-complementing bipartite β-barrel polypeptide designs (FIG. 1a). Because the split mFAP fragments would have solvent-exposed hydrophobic patches that could hamper solubility, we initially tagged split mFAP fragments to maltose binding protein (MBP) to improve soluble expression levels. β-barrel self-complementation assays in excess DFHBI-1T showed that β-barrel polypeptide β-strands 1-2 complementing with β-strands 3-8 (i.e. split mFAP fragments m12 and m38, respectively) displayed the highest fluorescence activation above background, with 7.34-fold higher mean fluorescence intensity over mean background fluorescence intensity. After background subtraction, the brightest split mFAP fragment combination, m12 and m38, had 184-fold higher mean fluorescence intensity than the dimmest split mFAP fragment combination, β-strand 1 complementing with β-strands 2-8 (i.e. split mFAP fragments m1 and m28, respectively). Differences in the fluorescence excitation spectra of the fluorescently active β-barrel complexes in excess DFHBI-1T suggest that bipartite split mFAPs stabilize the fluorescently active cis-planar conformation of DFHBI-1T in slightly different chromophore environments (FIG. 1c).

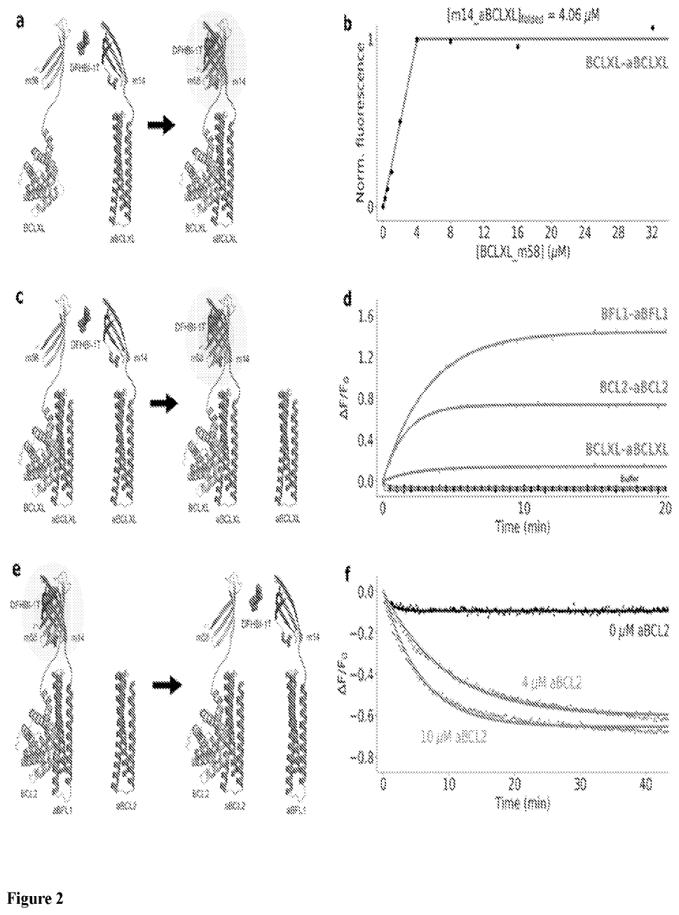

Titrations of MBP-tagged split mFAP fragments into their complementary MBP-tagged split mFAP fragments in excess DFHBI-1T resulted in reconstitution of fluorescence at high protein concentrations, but the signal did not plateau even at the highest concentrations tested. The estimated split mFAP fragment dissociation constants (K_{d} values) are ≥281 μM for m12 and m38, ≥22.0 μM for m14 and m58, ≥232 μM for m16 and m78, and ≥354 μM for m17 and m8 (FIG. 1d,e,f,g). In contrast, when we fused complementary split mFAP fragments to BCL2 family member proteins and high affinity (K_{d}≈1 nM) designed binding partners (FIG. 2a), the fluorescence increased linearly until reaching a plateau at equimolar concentrations of complementary split mFAP fragments (FIG. 2b).

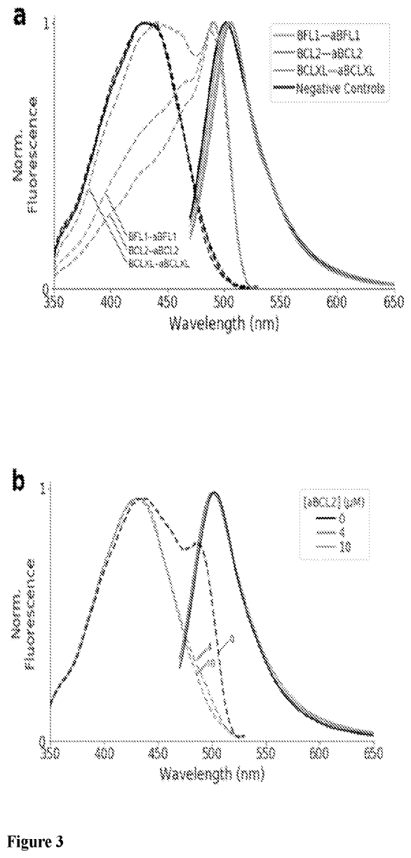

To assess whether split mFAPs could be used for real-time monitoring of protein-protein association, we pre-incubated equimolar BCLXL_m58 with unfused aBCLXL in excess DFHBI-1T to pre-assemble non-fluorescent BCLXL_m58-aBCLXL complex. Upon addition of equimolar m14_aBCLXL (or buffer as a negative control), the fluorescence increased as m14_aBCLXL competed with unfused aBCLXL for the BCLXL binding cleft of BCLXL_m58, resulting in assembly of the m14-m58 complex which activates the fluorescence of DFHBI-1T (FIG. 2c,d). The reaction evolved analogously for BFL1-aBFL1 and BCL2-aBCL2 cognate binding partners. Different peak fluorescence fold-changes observed amongst split mFAP fusions to BCLXL-aBCLXL, BCL2-aBCL2, and BFL1-aBFL1 complexes suggest that the molecular geometry of the heterodimer interaction affects the brightness of the assembled β-barrel complex. Fluorescence excitation spectra revealed a prominent peak in fluorescence excitation wavelength at 488 nm upon combining split mFAP fragments compared to buffer negative controls (FIG. 3a).

To assess whether split mFAPs could be used for real-time monitoring of protein-protein dissociation, we pre-incubated BCL2_m58 with equimolar m14_aBFL1 in excess DFHBI-1T to pre-assemble fluorescent complexes. As the non-cognate BCL2-aBFL1 complex has a dissociation constant (K_{d}) of 320±40 nM⁸, the cognate BCL2-aBCL2 complex has a K_{d} of 0.8±0.5 nM⁸, and aBFL1 and aBCL2 interact with the same binding cleft of BCL2, aBCL2 should outcompete aBFL1 for binding to BCL2 (FIG. 2e). Indeed, titration of aBCL2 into pre-assembled BCL2_m58-m14_aBFL1 complex in excess DFHBI-1T resulted in an aBCL2 concentration-dependent decrease in fluorescence (FIG. 20. Fluorescence excitation spectra showed the disappearance of the fluorescence excitation peak at 488 nm wavelength consistent with chromophore unbinding and deactivation of fluorescence upon split mFAP fragment disassembly (FIG. 3b).

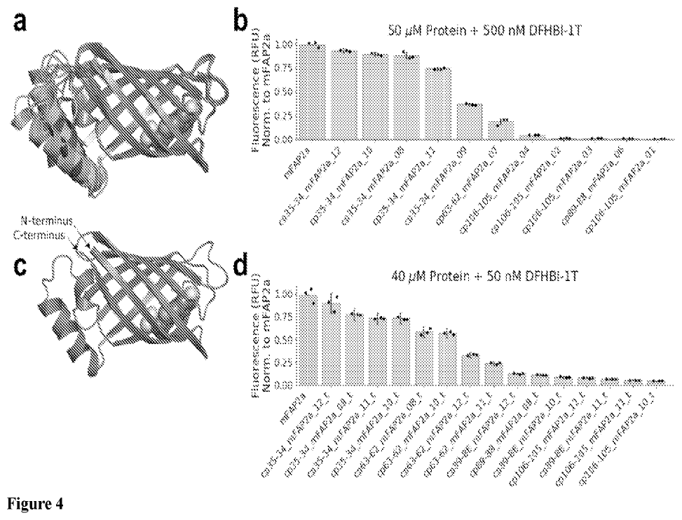

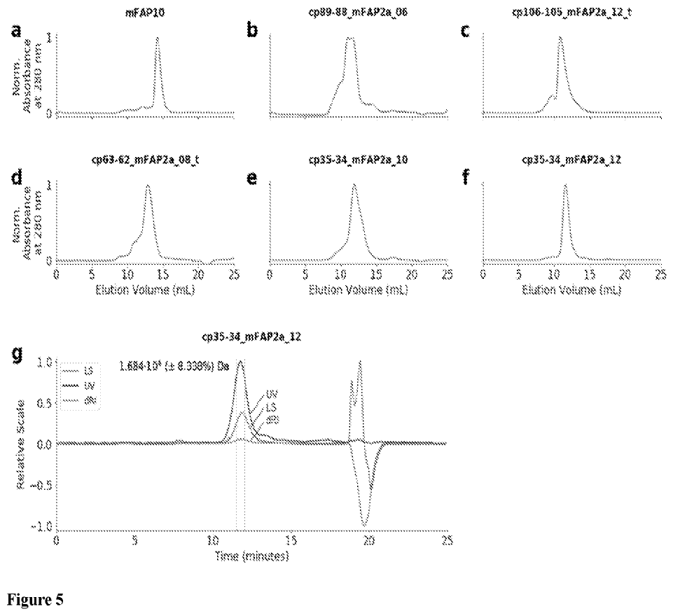

Using the split points from the four brightest self-complementary bipartite β-barrel polypeptides presented in FIG. 1a (i.e. a split point between β-strands 1-2 and β-strands 3-8 corresponding to the canonical, single-chain β-barrel polypeptide residues 34 and 35; a split point between β-strands 1˜4 and β-strands 5-8 corresponding to canonical, single-chain β-barrel polypeptide residues 62 and 63; a split point between β-strands 1-6 and β-strands 7-8 corresponding to canonical, single-chain β-barrel polypeptide residues 88 and 89; and a split point between β-strands 1-7 and β-strand 8 corresponding to canonical, single-chain β-barrel polypeptide residues 105 and 106) as the split points for circularly permuted single-chain mFAPs, structured and unstructured loops were computationally designed with Rosetta™ macromolecular modeling software. Initially, twelve computationally designed circularly permuted mFAP (cpmFAP) sequences were selected out of the thousands of designed sequences for protein expression and fluorescence intensity measurements. One out of the twelve cpmFAP designs did not express well in E. coli. However, in the presence of excess DFHBI-1T the other eleven cpmFAP designs (FIG. 4a) demonstrated variable fluorescence intensity compared with the positive control mFAP2a (i.e. a canonical, single-chain β-barrel polypeptide capable of binding and activating the fluorescence of DFHBI-1T) (FIG. 4b). The brightest cpmFAP tested, cp35-34_mFAP2a_12, has a de novo designed α-helical linker (FIG. 4c) and displayed ˜93% of the fluorescence intensity of mFAP2a at equimolar concentration and excess DFHBI-1T. The four brightest cpmFAP designs were those with split points between the canonical, single-chain β-barrel polypeptide residue numbers 34 and 35 (i.e. cp35-34_mFAP2a_12, cp35-34_mFAP2a_10, cp35-34_mFAP2a_08, and cp35-34_mFAP2a_11), corresponding to the structural equivalent of β-barrel polypeptide β-strands 1-2 (i.e. m12) and β-strands 3-8 (i.e. m38) covalently fused together with de novo designed structured and unstructured linker sequences. Size-exclusion chromatography with multi-angle light scattering showed cp35-34_mFAP2a_12 to be monomeric (FIG. 5g).

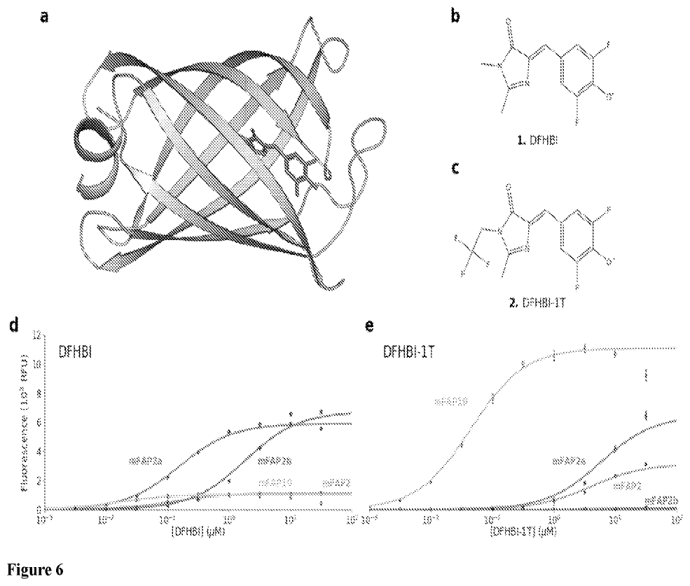

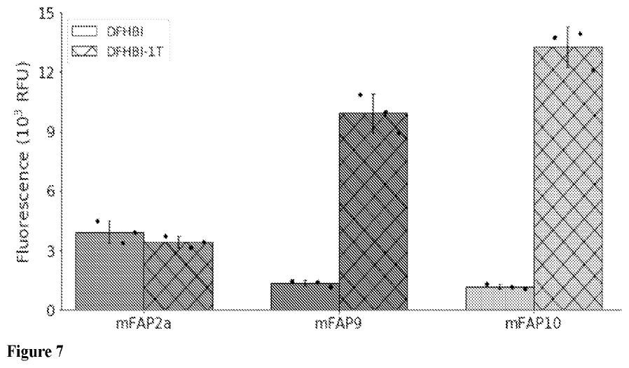

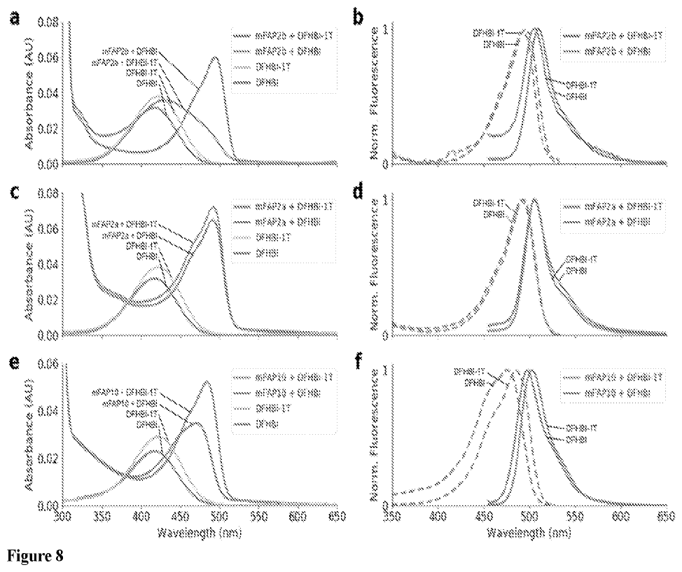

Titrations of mFAP2, mFAP2a, mFAP2b, and mFAP10 (Table 3) with either DFHBI (FIG. 6d) or DFHBI-1T (FIG. 6e) and quantum yield measurements (Table 4; FIG. 8) showed that: mFAP2, mFAP2a, and mFAP10 have ˜2.7-fold, ˜2.5-fold, and ˜12-fold brighter fluorescence with DFHBI-1T than DFHBI, but bind DFHBI with ˜30-fold, ˜39-fold, and ˜2.6-fold higher affinity than DFHBI-1T, respectively; mFAP2b has ˜30-fold brighter fluorescence with DFHBI than DFHBI-1T and binds DFHBI with ˜6.1-fold higher affinity than DFHBI-1T. The mFAP9˜DFHBI complex had ˜1.1-fold the fluorescence intensity of the mFAP10˜DFHBI complex, and the mFAP9˜DFHBI-1T complex had ˜0.75-fold the fluorescence intensity of the mFAP10˜DFHBI-1T complex (FIG. 7; Table 3). The mFAP10˜DFHBI-1T complex is the brightest, with 23.7% absolute quantum yield (under conditions with 99.9% of chromophore bound) and a 17.5-fold increased brightness over the previously reported mFAP2˜DFHBI complex, resulting in a 242-fold fluorescence activation over free DFHBI-1T (Table 4).

TABLE 4
Photophysical properties of mFAPs with DFHBI and DFHBI-1T compared with controls. The % bound values are calculated based on
the reported K_{d} values and final protein and chromophore concentrations used in quantum yield measurements. K_{d} values are obtained
by non-linear least squares fits to the mean fluorescence intensities of the 8 technical replicates per chromophore titration (Figure 6d, e).
K_{d} error estimates are the standard deviation of the mean of the non-linear least squares fits. * λ_{abs} is peak absorbance wavelength, λₑₓ
is peak excitation wavelength, and λₑₘ is peak emission wavelength (Figure 8). ^{†}Extinction coefficients are measured from λ_{abs} estimated
based on 1 data point in this study. ^{‡}Brightness is defined as extinction coefficient multiplied by absolute quantum yield. ^{§}Absolute
quantum yield is the average of 10 scans measured with an integrating sphere; relative quantum yield is reported using Acridine
Yellow G and fluorescein as reference standards. ^{¶}Previously reported value. ^{#}Previously reported value.

| | λ_{abs} | λₑₓ | λₑₘ | Extinction | | Absolute | Relative | Reported | | |
|---|---|---|---|---|---|---|---|---|---|---|
| | (nm) | (nm) | (nm) | Coefficient | Brightness | Quantum | Quantum | Quantum | % | |
| | * | * | * | (M^{-1,} cm⁻¹)^{†} | (M^{-1,} cm⁻¹)^{‡} | Yield^{§} | Yield^{§} | Yield | Bound | K_{d} (μM) |
| EGFP | — | 488^{#} | 507^{#} | 56,000^{#} | 33,600^{#} | — | — | 0.60^{#} | — | — |
| mFAP2a + | 491 | 491 | 505 | 64,900 | 3,890 | 0.060 | 0.063 | — | 99.9 | 0.15 ± 0.011 |
| DFHBI | | | | | | | | | | |
| mFAP2a + | 492 | 493 | 505 | 75,100 | 9,690 | 0.129 | 0.128 | — | 95.8 | 5.8 ± 0.86 |
| DFHBI-1T | | | | | | | | | | |
| mFAP2b + | 495 | 495 | 509 | 60,500 | 5,630 | 0.093 | 0.099 | — | 99.1 | 1.8 ± 0.25 |
| DFHBI | | | | | | | | | | |
| mFAP2b + | 430 | 494 | 505 | 37,800 | 189 | 0.005 | 0.003 | — | 95.1 | 11 ± 3.1 |
| DFHBI-1T | | | | | | | | | | |
| mFAP10 + | 470 | 475 | 497 | 48,900 | 1,290 | 0.026 | 0.029 | — | 100.0 | 0.017 ± 0.0079 |
| DFHBI | | | | | | | | | | |
| mFAP10 + | 484 | 485 | 503 | 67,200 | 15,900 | 0.237 | 0.230 | — | 99.9 | 0.045 ± 0.0065 |
| DFHBI-1T | | | | | (2.1× dimmer | | | | | |
| | | | | | than EGFP) | | | | | |
| DFHBI | 418^{¶} | 423^{¶} | 489^{¶} | 30,100^{¶} | — | 0.001^{#} | — | 0.0007^{¶} | — | — |
| | | | | 31,935^{#} | | | | | | |
| DFHBI-1T | 422^{¶} | 426^{¶} | 495^{¶} | 35,400^{¶} | — | — | — | 0.00098^{¶} | — | — |

### Discussion

Herein, we demonstrate that seven bipartite β-barrel polypeptide designs self-complement (FIG. 1a) and confirm that four of which have different self-complementing affinities (FIG. 1d,e,f,g). The low affinities and therefore weak interaction energies of the reversible self-complementing β-barrel polypeptide fragments for one another is ideal for monitoring association and dissociation events of covalently fused polypeptides of interest because the β-barrel polypeptide fragment interactions do not significantly perturb the binding affinities of the covalently fused polypeptides of interest. Thus, when each β-barrel polypeptide fragment is fused to each subunit of a homooligomeric or heterooligomeric polypeptide complex of interest, when the complex of interest is fully associated the multipartite β-barrel polypeptide fragments can be approximated to be at an infinite local concentration which is higher than the thermodynamic dissociation constant (K_{d}) of the multipartite β-barrel polypeptide fragments on their own without fusion to polypeptides of interest. Therefore, they associate into a fluorescently active complex only when the polypeptides of interest bind and form a complex. When the polypeptides of interest are in the dissociated state and the total concentration of polypeptide subunits is lower than the thermodynamic dissociation constant (K_{d}) of the multipartite β-barrel polypeptide fragments on their own without fusion to polypeptides of interest, then the multipartite β-barrel polypeptide fragments dissociate as well. Under these conditions, multipartite β-barrel polypeptides may be used to detect polypeptide-polypeptide association and dissociation events of transient homooligomeric and heterooligomeric polypeptide complexes of interest because multipartite β-barrel polypeptide fragments only associate into a fluorescently active complex when the covalently fused polypeptides of interest associate.

Each multipartite β-barrel polypeptide fragment has an N-terminus and C-terminus for covalent attachment of structured or unstructured polypeptides of interest, which may either drive or hinder self-complementation of β-barrel polypeptide fragments. For example, to mitigate steric hindrance amongst β-barrel polypeptide fragments, we suggest that β-barrel polypeptide β-strands 1-7 (e.g. m17) assemble together with β-barrel polypeptide β-strand 8 (e.g. m8) alone. However, by way of a non-limiting example, due to the redundancy of β-strand 8 amongst many of the multipartite β-barrel polypeptide fragments (Table 1), β-barrel polypeptide β-strands 1-7 (e.g. m17) may assemble together with β-strands 2-8 (e.g. m28), β-strands 3-8 (e.g. m38), β-strands 4-8 (e.g. m48), β-strands 5-8 (e.g. m58), β-strands 6-8 (e.g. m68), β-strand 7-8 (e.g. m78), or β-strand 8 (e.g. m8) to form a fluorescently active reporter complex. As long as all eight unique β-strands are structurally associated forming the fluorescently active multipartite β-barrel polypeptide complex, then any combination of β-barrel polypeptide fragments may be used to monitor association and dissociation events of homooligomeric and heterooligomeric polypeptide complexes of interest.

The reported split mFAPs are based on the canonical, single-chain β-barrel polypeptide called mFAP2a, but the designed split points can be generalized to other canonical, single-chain β-barrel polypeptides such as, but not limited to, mFAP2b and mFAP10. While mFAP2a has a low affinity for DFHBI-1T (K_{d} of 5.8 μM; Table 4) requiring high final concentrations of DFHBI-1T to approximately saturate the chromophore binding pocket (e.g. 58 μM), mFAP10 has a high affinity for DFHBI-1T (K_{d} of 45 nM; Table 4) allowing lower final concentrations of DFHBI-1T to approximately saturate the chromophore binding pocket (e.g. 450 nM). If the self-complemented multipartite β-barrel polypeptides have similar chromophore affinities as their single-chain counterparts, then high chromophore affinities enable experimentalists to use low concentrations of chromophore to approximately saturate the binding pocket of the assembled multipartite β-barrel polypeptide complex. Saturating the assembled multipartite β-barrel polypeptide complex with chromophore increases the fluorescence intensity signal upon reversible self-complementation, and low total chromophore concentrations reduces background fluorescence noise. Thus, self-complemented multipartite β-barrel polypeptides with high chromophore affinities and labeled at chromophore concentrations low enough to approximately saturate the binding pocket are expected to exhibit high fluorescence signal-to-noise ratios, particularly during live cell imaging when fluorescence background subtraction is not feasible. Additionally, canonical, single-chain β-barrel polypeptides including mFAP9 and mFAP10 have various specificities and affinities for different exogenous fluorogenic compounds such as DFHBI, DFHBI-1T, and DFHO. If the self-complemented multipartite β-barrel polypeptides have similar chromophore specificities and affinities as their single-chain counterparts, then by combining the correct combinations of multipartite β-barrel polypeptide fragments (Table 1) into an active reporter complex, the fluorescence can be tuned by experimentalists by mixing one or more chromophores at various concentrations into the polypeptide system.

Circularly permuted β-barrel polypeptides were de novo designed with novel linkers fusing the N- and C-termini of the canonical, single-chain mFAP, and making a single split point elsewhere in the β-barrel polypeptide to act as the new N- and C-termini. The two new N-terminal residues and two new C-terminal residue types were designed to more optimal amino acid sequences using the Rosetta™ software package (FIG. 4b), or were fixed compared to the canonical, single-chain mFAP sequence (FIG. 4d). Circularly permuted β-barrel polypeptides were demonstrated based on the canonical, single-chain mFAP2a scaffold, but can be generalized to other canonical, single-chain β-barrel polypeptides such as mFAP9 and mFAP10. We demonstrated that circularly permuted β-barrel polypeptides are capable of folding and activating the fluorescence of the exogenous fluorogenic compound DFHBI-1T. It is expected that circularly permuted β-barrel polypeptides are capable of activating the fluorescence of additional fluorogenic compounds such as, but not limited to, DFHBI and DFHO. Circularly permuted β-barrel polypeptides are ideal polypeptide scaffolds for the design of novel fluorogenic optical biosensors that can detect the concentrations of ions, small-molecules, proteins, nucleic acids, organic substrates, and inorganic substrates in real-time using fluorescence microscopy and fluorimetry methodologies.

### Conclusion:

The concept of self-complementing multipartite β-barrel polypeptides capable of monitoring polypeptide-polypeptide association and dissociation events has been experimentally validated herein in vitro using bipartite β-barrel polypeptides. Self-complementing multipartite β-barrel polypeptides allow real-time monitoring of polypeptide-polypeptide association and dissociation events through self-complementation of β-barrel polypeptide fragments into a reporter complex capable of activating the fluorescence of exogenous fluorogenic compounds such as, but not limited to, DFHBI, DFHBI-1T, and DFHO, with different degrees of specificity and affinity. Additionally, we have experimentally validated the concept of circularly permuted mFAPs based on, but not limited to, the mFAP2a scaffold, that are capable of activating the fluorescence of the exogenous fluorogenic compounds such as, but not limited to, DFHBI-1T. Multipartite β-barrel polypeptides and circularly permuted β-barrel polypeptides may be used as versatile polypeptide scaffolds in the engineering of novel oligomeric polypeptide assemblies and novel fluorogenic optical biosensors for the detection of analytes of interest in real-time using fluorescence measurement techniques.

### Methods:

Design of Split mFAPs.

Split mFAPs were designed by manually inspecting the single-chain mFAP2a, mFAP2b and mFAP10 computational design model (Table 1). In designing split mFAP fusions to BCL2 family heterodimers, linker compositions and lengths were chosen by manually inspecting the split mFAP2a computational design models and available crystal structures (Protein Data Bank accession codes 5JSN and 5JSB). Split mFAP2a fragments were fused to maltose binding protein (MBP), BCL2, aBCL2, BFL1, aBFL1, BCLXL, and aBCLXL after cysteine residues unlikely to be participating in disulfide bonds were mutated to serine or alanine residues.

Design of cpmFAPs.

Circularly permuted mFAP2a and mFAP2b were generated from mFAP2a and mFAP2b computational models using Rosetta™ and custom scripts in which N- and C-termini (“split points”) were selected at mFAP loop2 (i.e. the loop connecting β-strand 2 to β-strand 3), loop4 (i.e. the loop connecting β-strand 4 to β-strand 5), loop6 (i.e. the loop connecting β-strand 6 to β-strand 7), and loop7 (i.e. the loop connecting β-strand 7 to β-strand 8) locations, and the two N-terminal and two C-terminal residues of cpmFAP scaffolds were re-designed compared to their respective residue types in mFAP2a. Structured and unstructured linkers covalently fusing the canonical mFAP termini were designed using the Rosetta software package, and 4,000 resulting designs were filtered and sorted on design metrics. The top 12 designs were chosen for experimental testing after 3 circularly permuted mFAP2b variants were mutated to circularly permuted mFAP2a variants using the (V13A, M15F) double point mutation (in canonical mFAP residue numbering) (FIG. 4a,b; Table 2). In the subsequent round of cpmFAP designs, the de novo designed linker sequences from cp35-34_mFAP2a_12, cp35-34_mFAP2a_10, cp35-34_mFAP2a_08, and cp35-34_mFAP2a_11 were each sampled with the four split points described above, and the two N-terminal and two C-terminal residues in the cpmFAP were reverted back to their respective residue types in mFAP2a (FIG. 4d; Table 2).

Split mFAP Titration Assays.

To measure fluorescence intensities in complementation assays (FIG. 1b), fluorescence was measured on a Synergy Neo2 hybrid multi-mode reader (BioTek) in flat bottom, black polystyrene, non-binding surface 96-well half-area microplates (Corning 3686). Each split mFAP fragment covalently fused to maltose binding protein (MBP) was purified by large-scale protein purification in high salt Tev cleavage buffer⁹. In technical triplicate, 12.0 μL of each MBP-tagged split mFAP fragment was mixed to an equimolar concentration supplemented with 1.00 μL of 1.25 mM DFHBI-1T (Lucerna) at 25.0 μL final volumes per well. Fluorescence endpoints were measured using excitation wavelength λₑₓ=478 nm and emission wavelength λₑₘ=520 nm. In technical triplicate, background fluorescence endpoints of wells with identical chromophore concentrations lacking protein (substituted for equivalent volumes of high salt Tev cleavage buffer) were measured, and the mean fluorescence endpoints were subtracted from the mean fluorescence endpoints of samples containing protein (FIG. 1b).

Split mFAP fragment affinities (FIG. 1d,e,f,g) were estimated by preparing MBP-tagged split mFAP fragments by large-scale protein purification in high salt Tev cleavage buffer⁹, with 25.0 μM DFHBI-1T final concentration at 28.0 μL final volumes per well in flat bottom, black polystyrene, non-binding surface 96-well half-area microplates (Corning 3686). 3.00 μL of either 132 μM m12, 122 μM m14, 101 μM m16, or 84.9 μM m17 in high salt Tev cleavage buffer was mixed with 3.00 μL of 150 μM DFHBI-1T in high salt Tev cleavage buffer. For each split mFAP fragment, 12.0 μL of the complementary split mFAP fragment in high salt Tev cleavage buffer (the titrant) was mixed in from eleven serial dilutions (\sqrt{10} dilution factor) starting from 422 μM m38, 33.0 μM m58, 348 μM m78, or 531 μM m8 stock solutions, respectively, including a twelfth condition without titrant. Fluorescence endpoints were measured on a Synergy Neo2 hybrid multi-mode reader (BioTek) using excitation wavelength λₑₓ=468 nm and emission wavelength λₑₘ=530 nm. For each titration, the fluorescence intensity of the condition without titrant was subtracted from the fluorescence intensities of samples containing titrant, then the background subtracted data was normalized from 0 to 1. In collecting fluorescence excitation and emission spectra (FIG. 1c), the conditions with the highest protein concentrations and 25.0 μM DFHBI-1T were used. Excitation spectra were measured using excitation wavelengths in the range λₑₓ=350-498 nm and emission wavelength λₑₘ=530 nm, and emission spectra were measured using excitation wavelength λₑₓ=468 nm and emission wavelengths in the range λₑₘ=500-650 nm. Fluorescence excitation and emission spectra of conditions without the addition of the complementary split mFAP fragment were measured and used for background subtraction at the corresponding wavelengths.

For titrating BCLXL_m58 into m14_aBCLXL (FIG. 2b), m14_aBCLXL and BCLXL_m58 were prepared by large-scale protein purification in high salt Tev cleavage buffer⁹. Fluorescence endpoints were measured on a Synergy Neo2′ hybrid multi-mode reader (BioTek) in flat bottom, black polystyrene, non-binding surface 384-well microplates (Corning 4514) using fluorescence excitation wavelength λₑₓ=468 nm and fluorescence emission wavelength λₑₘ=530 nm. Nine wells each with 3.90 μL of 19.6 μM m14_aBCLXL and 2.20 μL of 114 μM DFHBI-1T were prepared, and 3.90 μL of either high salt Tev cleavage buffer or BCLXL_m58 was aliquoted per well to reach final concentrations of 0 μM, 251 nM, 501 nM, 1.00 μM, 2.01 μM, 4.01 nM, 8.02 μM, 16.0 μM, or 32.1 nM BCLXL_m58, with 25.0 μM DFHBI-1T and 7.64 μM m14_aBCLXL in 10.0 μL final volumes per well. Fluorescence intensities were measured after 2,847 s of double orbital shaking in the dark. Fluorescence from the 0 μM BCLXL_m58 condition was subtracted from each condition, and the background-subtracted fluorescence in relative fluorescence units (RFU), F, was normalized by the formula:

$$Norm . fluorescence = \frac{F - {F}_{min}}{{F}_{max} - {F}_{min}} \qquad ( 1 )$$

where Fₘᵢₙ (RFU) was the minimum fluorescence intensity, and Fₘₐₓ (RFU) was the fit to a constant function using non-linear least squares fitting of the fluorescence intensities of the four highest BCLXL_m58 concentrations. Using a bimolecular association model:

$$BCLXL\_m58 + m14\_aBCLXL {{⇌}^{{k}_{1}}}_{{k}_{2}} BCLXL\_m58 - m 14 \_aBCLXL \qquad ( 2 )$$

it can be shown that:

$$[ BCLXL\_m 58 - m 14 \_aBCLXL ] = 0.5 \cdot ( {[ BCLXL\_m 58 ]}_{total} + {[ m 14 \_aBCLXL ]}_{total} + {K}_{d} ) - 0.5 \cdot \sqrt{\begin{matrix}{( - {[ BCLXL\_m 58 ]}_{total} - {[ m 14 \_aBCLXL ]}_{total} - {K}_{d} )}^{2} - \\ ( 4 \cdot {[ BCLXL\_m 58 ]}_{total} \cdot {[ m 14 \_aBCLXL ]}_{total} )\end{matrix}} \qquad ( 3 )$$

where

$$\frac{{k}_{2}}{{k}_{1}} = {K}_{d} = {K}_{d}^{BCLXL \_ m 58 - m 14 \_ aBCLXL} .$$

The theoretical maximum fluorescent complex concentration, [BCLXL_m58-m14_aBCLXL]ₘₐₓ, is reached at excess [BCLXL_m58]ₜₒₜₐₗ, taken at [BCLXL_m58]_{excess}=10.0 M. Similarly, it can also be shown that:

$${[ BCLXL\_m 58 - m 14 \_aBCLXL ]}_{max} = 0.5 \cdot ( {[ BCLXL\_m 58 ]}_{excess} + {[ m 14 \_aBCLXL ]}_{total} + {K}_{d} ) - 0.5 \cdot \sqrt{\begin{matrix}{( - {[ BCLXL\_m 58 ]}_{excess} - {[ m 14 \_aBCLXL ]}_{total} - {K}_{d} )}^{2} - \\ ( 4 \cdot {[ BCLXL\_m 58 ]}_{excess} \cdot {[ m 14 \_aBCLXL ]}_{total} )\end{matrix}} \qquad ( 4 )$$

As fluorescent complexes only form with the folded fraction of m14_aBCLXL, p_{folded}, under the condition that [m14_aBCLXL]_{folded}=p_{folded}·7.64 μM is the [m14_aBCLXL]ₜₒₜₐₗ, we fit p_{folded} as a free parameter to the normalized fluorescence intensity with the formula:

$$\frac{F - {F}_{min}}{{F}_{max} - {F}_{min}} = \frac{[ BCLXL\_m 58 - m 14 \_aBCLXL ]}{{[ BCLXL\_m 58 - m 14 \_aBCLXL ]}_{max}} \qquad ( 5 )$$

where:

K_{d}^{BCLXL_m58-m14_aBCLXL}=K_{d}^{BCLXL-aBCLXL}·K_{d}^{m14-m58}=1.23·10⁻¹³ M (6)

because the aBCLXL domain of m14_aBCLXL associates with the binding cleft of the BCLXL domain of BCLXL_m58 with the previously reported⁸ BCLXL-aBCLXL thermodynamic dissociation constant of K_{d}^{BCLXL-aBCLXL}=5.59·10⁻⁹ M, and the m14 domain of m14_aBCLXL associates with the m58 domain of BCLXL_m58 with the m14-m58 thermodynamic dissociation constant taken as K_{d}^{m14-m58}=22.0·10⁻⁶ M in 25.0 μM DFHBI-1T (FIG. 1e), under an approximation that the BCLXL_m58-m14_aBCLXL interaction energy comprises only the BCLXL-aBCLXL and m14-m58 interaction energies:

ΔG^{BCLXL_m58-m14_aBCLXL}=ΔG^{BCLXL-aBCLXL}+ΔG^{m14-m58} (7)

where ΔG is the change in Gibbs free energy upon the superscripted protein-protein interaction in 25.0 μM DFHBI-1T. Non-linear least squares fitting yields p_{folded}=0.532±0.0160, and therefore the reported [m14_aBCLXL]_{folded}=4.06 μM (FIG. 2b). The error estimate is the standard deviation of the fit.
Split mFAP Temporal Assays.

In temporally monitoring fluorescence intensities in a protein-fragment complementation assay (FIG. 2d), fluorescence was measured on a Synergy Neo2™ hybrid multi-mode reader (BioTek) in flat bottom, black polystyrene, non-binding surface 96-well microplates (Corning 3650) using excitation wavelength λₑₓ=468 nm and emission wavelength λₑₘ=530 nm. aBCL2, aBFL1, aBCLXL, m14_aBCL2, m14_aBFL1, m14_aBCLXL, BCL2 m58, BFL1_m58, and BCLXL_m58 were prepared by large-scale protein purification in high salt Tev cleavage buffer⁹. Two wells each with 36.0 μL of either aBCL2, aBFL1, or aBCLXL, 36.0 μL of either BCL2_m58, BFL1 m58, or BCLXL_m58, and 12.0 μL of 250 μM DFHBI-1T were prepared with matched cognate binding partners, and samples were mixed by double orbital shaking at room temperature for 30 min in the dark. Subsequently, 36.0 μL of high salt Tev cleavage buffer was aliquoted into the first of the two wells (negative control group), and 36.0 μL of either m14_aBCL2, m14_aBFL1, or m14_aBCLXL was aliquoted into the second of the two wells (experimental group) with matched cognate binding partners, respectively. Fluorescence intensities were measured every 30 s between 5 s double orbital shake steps to mix the samples for 1,200 s. Final sample conditions were: 2.79 μM of aBCL2 and BCL2_m58, and either 0 μM or 2.79 m14_aBCL2; 2.48 μM of aBFL1 and BFL1_m58, and either 0 μM or 2.48 μM m14_aBFL1; 3.88 μM of aBCLXL and BCLXL_m58, and either 0 μM or 3.88 μM m14_aBCLXL; and 25.0 μM DFHBI-1T for all sample conditions in 120 μL final volumes per well. For each condition, fluorescence fold-change was calculated as:

$$\frac{Δ F}{{F}_{0}} = \frac{F - {F}_{0}}{{F}_{0}} \qquad ( 8 )$$

where F (RFU) is the fluorescence intensity per measurement and F₀ (RFU) is the fluorescence intensity of the first measurement, then fluorescence fold-change was fit to a monophasic exponential function using non-linear least squares fitting (FIG. 2d). In collecting fluorescence excitation and emission spectra after reaching equilibrium (FIG. 3a), fluorescence excitation spectra were measured using excitation wavelengths in the range λₑₓ=350-530 nm and emission wavelength λₑₘ=562 nm, and emission spectra were measured using excitation wavelength λₑₓ=438 nm and emission wavelengths in the range λₑₘ=470-650 nm, and the normalized spectra reported without background subtraction.

In temporally monitoring fluorescence intensities (FIG. 21), fluorescence was measured on a Synergy Neo2™ hybrid multi-mode reader (BioTek) in flat bottom, black polystyrene; non-binding surface 96-well half-area microplates (Corning 3686) using excitation wavelength λₑₓ=478 nm and emission wavelength λₑₘ=530 nm. m14_aBFL1, BCL2_m58, and aBCL2 were prepared by large-scale protein purification in high salt Tev cleavage buffer⁹. Three wells of 2.22 μM of m14_aBFL1 with 2.22 μM BCL2_m58 and 27.8 μM DFHBI-1T in high salt Tev cleavage buffer at final volumes of 45.0 μL were prepared and mixed by double orbital shaking at room temperature for 20 min in the dark. Subsequently, 5.00 μL of either 100 μM aBCL2, 40.0 μM aBCL2, or high salt Tev cleavage buffer was aliquoted per well, respectively, and fluorescence intensities measured every 12 s between 5 s double orbital shake steps to mix the samples for 2,604 s. Final sample conditions were 25.0 μM DFHBI-1T, 2.00 μM m14_aBFL1, 2.00 μM BCL2_m58 and either 10.0 04, 4.00 μM, or 0 μM aBCL2 in 50.0 μL final volumes per well. For each condition, fluorescence fold-change was calculated by Eq. (8) where F (RFU) is the fluorescence intensity per measurement and F₀ (RFU) is the fluorescence intensity of the first measurement, then fluorescence fold-change was fit to a monophasic exponential function using non-linear least squares fitting (FIG. 2f). In collecting fluorescence excitation and emission spectra after reaching equilibrium (FIG. 3b), fluorescence excitation spectra were measured using excitation wavelengths in the range λₑₓ=350-530 nm and emission wavelength λₑₘ=570 nm, and fluorescence emission spectra were measured using excitation wavelength λₑₓ=430 nm and emission wavelengths in the range λₑₘ=470-750 nm, and the normalized spectra reported without background subtraction.

cpmFAP Fluorescence Intensity Assays.

To measure the fluorescence intensities of cpmFAPs (FIG. 4b,d), fluorescence endpoints were measured on a Synergy Neo2™ hybrid multi-mode reader (BioTek) in flat bottom, black polystyrene, non-binding surface 96-well microplates (Corning 3650) or half-area microplates (Corning 3686). Fluorescence endpoints were measured in technical triplicate by exciting at λₑₓ=488 nm and measuring fluorescence emission at λₑₘ=510 nm (FIG. 4b), or exciting at λₑₓ=468 nm and measuring fluorescence emission at λₑₘ=530 nm (FIG. 4d). 90.0 μL of 55.6 μM large-scale purified protein in high salt Tev cleavage buffer⁹ was combined with 10.0 μL of 5.00 μM DFHBI-1T in high salt Tev cleavage buffer for final concentrations of 50.0 μM protein and 500 nM DFHBI-1T in 100 μL final volumes (FIG. 4b), or 48.0 μL of 41.7 μM large-scale purified protein in high salt Tev cleavage buffer⁹ was mixed with 2.00 μL of 1.25 μM DFHBI-1T in high salt Tev cleavage buffer for final concentrations of 40.0 μM protein and 50.0 nM DFHBI-1T in 50.04 final volumes (FIG. 4d).

Canonical mFAP Fluorescence Intensity Assays.

In measuring fluorescence intensity at λₑₓ=468 nm and λₑₘ=530 nm of each clone in technical triplicate (FIG. 7), 24.0 μL of 35.4 μM large-scale purified protein⁹ was combined with 1.00 μL of L25 μM DFHBI (Lucerna) or 1.00 μL of 1.25 μM DFHBI-1T (Lucema) (from 2 mM chromophore stock solutions dissolved in 0.5% DMSO and 99.5% high salt Tev cleavage buffer [25.0 mM Tris, 100 mM NaCl, pH 8.00]) for final concentrations of 34.0 μM protein and 50.0 nM chromophore. In triplicate, the fluorescence intensity from each condition was background-subtracted using conditions with equivalent chromophore concentration but protein substituted with high salt Tev cleavage buffer.

### Chromophore Titrations.

Fluorescence endpoints were measured on a Synergy Neo2™ hybrid multi-mode reader (BioTek) in flat bottom, black polystyrene, non-binding surface 96-well microplates (Corning 3650). In measuring chromophore binding affinities (FIG. 6d,e), mFAP2, mFAP2a, and mFAP2b, and mFAP10 were produced by large-scale protein purification and SEC purification⁹. Proteins were aliquoted in eight technical replicates in 200 μL final volumes to 20.0 nM final concentration in ten serial dilutions (\sqrt{10} dilution factor) of DFHBI starting from 31.6 μM DFHBI or 31.6 μM DFHBI-1T final concentrations, including an eleventh condition without chromophore. Fluorescence was excited at λₑₓ=468 nm and fluorescence emission measured at λₑₘ=530 nm. Background fluorescence endpoints of wells with identical chromophore concentrations but purified protein replaced with an identical volume of high salt Tev cleavage buffer were measured, and fluorescence endpoints subtracted from those measured with protein. Background-subtracted data were averaged and the means normalized from 0 to 1 and fit to a single binding site isotherm function using non-linear least squares fitting to obtain a fitted K_{d} value (Table 4), and the fit scaled to the maximum mean value (FIG. 6d,e).

### Size-Exclusion Chromatography.

For FIG. 5a,b,c,d,e,f, large-scale purified proteins were further purified by size-exclusion chromatography as described previously⁹.

Size-Exclusion Chromatography with Multi-Angle Light Scattering.

Protein samples were prepared at 2.0 mg·mL⁻¹ and applied to a Superdex™ 75 10/300 GL column (GE Healthcare) on a LC 1200 Series HPLC machine (Agilent Technologies) for size-based separation, a Heleos™ detector (Wyatt Technologies) for light scattering signals, and a t-Rex detector for differential refractive index detection. Results were analyzed using ASTRA™ 7.2 software for weighted average molecular weight (FIG. 5g).

### Quantum Yield Measurements.

Protein preparation. mFAP2a, mFAP2b and mFAP10 were produced by large-scale protein purification⁹ and dialyzed overnight into DPBS that was adjusted to pH 7.40 using NaOH.

Chromophore preparation. DFHBI (Lucerna) and DFHBI-1T (Lucerna) were dissolved to 20.0 mM in 100% DMSO, and diluted in DPBS (pH 7.40) to measure absorbances on a Jasco V-750 spectrophotometer at peak absorbance wavelengths (417 nm for DFHBI and 422 nm for DFHBI-1T). Following background subtraction of identical buffer without chromophore, Beer's Law was used to calculate the molar chromophore concentrations of the stock solutions using previously reported extinction coefficients⁵.

Preparation of protein-chromophore complexes. For quantum yield measurements, 1.00 μM, 836 nM, or 919 nM chromophore solutions in DPBS (pH 7.40) at 4.00 mL final volumes were prepared for the following eight conditions: DFHBI only, DFHBI-1T only, 43.5 μM 6×His-mFAP10 with DFHBI, 43.5 μM 6×His-mFAP10 with DFHBI-1T, 134 μM 6×His-mFAP2a with DFHBI, 134 μM 6×His-mFAP2a with DFHBI-1T, 206 μM 6×His-mFAP2b with DFHBI, and 206 μM 6×His-mFAP2b with DFHBI-1T.

Extinction coefficients. Absorbance spectra of protein-chromophore complexes were first measured with a Thermo Scientific BioMate™ 3 S UV-vis Spectrophotometer (1 nm interval, 800 nm min⁻¹). The extinction coefficients were then calculated using Beer's Law:

A=ε·b·c (9)

where A is peak absorbance, e is extinction coefficient, b is path length (1 cm), and c is concentration (1.00 μM, 836 nM, or 919 nM).

Relative quantum yield. A Perkin-Elmer LS-B Luminescence Spectrophotometer (10 nm bandwidth, 1 nm interval, 100 nm·min⁻¹) was used. The fluorescence emission spectra of the protein-chromophore complexes (in DPBS, pH 7.40) and reference dye Acridine Yellow G (in methanol) were first obtained, and the quantum yield was then calculated using the equation¹⁰:

$${ϕ}_{c} = {ϕ}_{r} \cdot \frac{1 - {10}^{- {A}_{r} ( {λ}_{ex} )}}{1 - {10}^{- {A}_{c} ( {λ}_{ex} )}} \cdot \frac{\int {F}_{c} ( λ ) \cdot d λ}{\int {F}_{r} ( λ ) \cdot d λ} \cdot \frac{{n}_{c}^{2}}{{n}_{r}^{2}} \qquad ( 10 )$$

where φ is quantum yield, A(λₑₓ) is absorbance at the excitation wavelength λₑₓ (λₑₓ=440 nm). F is fluorescence emission, n is refractive index of the solution (1.3350 for DPBS at pH 7.40 and 1.3284 for methanol), and the subscripts “c” and “r” refer to the protein-chromophore complex measured and the reference dye, respectively. The reference dye Acridine Yellow G (in methanol) has a quantum yield value of 0.57 that was used¹¹.

Absolute quantum yield. An integrating sphere instrument (Hamamatsu C9920-12) (6 nm excitation bandwidth, 1 nm interval) and a high-sensitivity photonic multi-channel analyzer (Hamamatsu C10027-01) were used to measure a light emission spectrum. Absolute quantum yields were measured for solutions of protein-chromophore complexes in DPBS (pH 7.40) in which ≥95% of the total chromophore was occupying the protein binding pocket (Table 4). Protein-chromophore complex samples and control samples were excited at λₑₓ=440 nm and absolute quantum yields were calculated according to the equation:

$${ϕ}_{c} = \frac{{f}_{em}}{{f}_{abs}} \qquad ( 11 )$$

where fₑₘ is the emitted photon flux and f_{abs} is the absorbed photon flux. The absolute quantum yields of the two control samples (Acridine Yellow G and fluorescein) agreed well with literature values^{11,12}. Absolute quantum yield data was analyzed with U6039-05 PLQY measurement software (Table 4; FIG. 8).

### REFERENCES

1. Paige, J. S., Wu, K. Y. & Jaffrey, S. R. RNA Mimics of Green Fluorescent Protein. Science vol. 333 642-646 (2011).
2. Strack, R. L. & Jaffrey, S. R. New approaches for sensing metabolites and proteins in live cells using RNA. Curr. Opin. Chem. Biol. 17, 651-655 (2013).
3. Autour, A., Westhof, E. & Ryckelynck, M. iSpinach: a fluorogenic RNA aptamer optimized for in vitro applications. Nucleic Acids Res. 44, 2491-2500 (2016).
4. Song, W. et al. Imaging RNA polymerase III transcription using a photostable RNA-fluorophore complex. Nat. Chem. Biol. 13, 1187-1194 (2017).
5. Song, W., Strack, R. L., Svensen, N. & Jaffrey, S. R. Plug-and-Play Fluorophores Extend the Spectral Properties of Spinach. Journal of the American Chemical Society vol. 136 1198-1201 (2014).
6. Warner, K. D. et al. A homodimer interface without base pairs in an RNA mimic of red fluorescent protein. Nature Chemical Biology 13(11):1195-1201 (2017).
7. Dou, J. et al. De novo design of a fluorescence-activating β-barrel. Nature 561, 485-491 (2018).
8. Berger, S. et al. Computationally designed high specificity inhibitors delineate the roles of BCL2 family proteins in cancer. Elife 5, (2016).
9. Klima, J. C. et al. Bacterial expression and protein purification of mini-fluorescence-activating proteins. Protocol Exchange (2021).
10. Würth, C., Grabolle, M., Pauli, J., Spieles. M. & Resch-Genger, U. Relative and absolute determination of fluorescence quantum yields of transparent samples. Nature Protocols vol. 8 1535-1550 (2013).
11. Olmsted, J. calorimetric determinations of absolute fluorescence quantum yields. The Journal of Physical Chemistry vol. 83 2581-2584 (1979).
12. Sjöback, R., Nygren, J. & Kubista, M. Absorption and fluorescence properties of fluorescein. Spectrochimica Acta Part A: Molecular and Biomolecular Spectroscopy vol. 51 L7-L21 (1995).

1. A non-naturally occurring, self complementing multipartite β-barrel protein, comprising at least a first polypeptide component and a second polypeptide component, wherein the at least first polypeptide component and the second polypeptide component are not covalently linked, wherein in total the at least first polypeptide component and the second polypeptide component comprise domains X1-X2-X3-X4-X5-X6-X7-X8-X9-X10-X11-X12-X13-X14-X15-X16-X17-X18-X19, wherein:
X1 comprises a capping domain;
X2 comprises a beta strand;
wherein a contiguous C-terminal portion of X1 and N-terminal portion of X2 comprise the amino acid sequence Z1-P-G-Z2-W, where Z1 and Z2 are any amino acid;
X3 comprises a beta turn;
X4 comprises a beta strand that includes an internal G residue and a P at its C-terminus;
X5 comprises a single polar amino acid;
X6 comprises a beta turn;
X7 comprises a beta strand including an internal G residue;
X8 comprises a beta turn;
X9 comprises a beta strand including an internal P residue and 2 internal G residues;
X10 comprises a single polar amino acid;
X11 comprises a beta turn;
X12 comprises a beta strand;
X13 comprises a beta turn;
X14 comprises a beta strand with an internal G residue;
X15 comprises a single polar amino acid;
X16 comprises a beta turn;
X17 comprises a beta strand;
X18 comprises a beta turn; and
X19 comprises a beta strand;
wherein (a) each beta strand is fully present within one polypeptide component of the at least first polypeptide component and the second polypeptide component, (b) none of the at least first polypeptide component and the second polypeptide component include each of X2, X4, X7, X9, X12, X14, X17, and X19; and (c) one of domains X3, X6, X8, X11, X13, X16, and X18 may be partially or wholly absent in each of the first polypeptide and the second polypeptide.

2. (canceled)

3. The self-complementing multipartite β-barrel protein of claim wherein Z1 is a hydrophobic amino acid and Z2 is a polar amino acid.

4. The self-complementing multipartite β-barrel protein of claim 1, wherein Z1 is selected from the group consisting of L, A, and F.

5. The self-complementing multipartite β-barrel protein of claim 1, wherein Z2 is selected from the group consisting of T, K, N, and D.

6. The self-complementing multipartite β-barrel protein of claim 1, wherein the X1 capping domain comprises an alpha helix.

7. The self-complementing multipartite β-barrel protein of claim 1, wherein
(a) X1 comprises an amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO:535) or RAAQLLP (SEQ ID NO:536), wherein the highlighted residue is invariant;
(b) X2 comprises an amino acid sequence at least 50%, 55%, 60%, 65%, 70%, 75%, 80%, 85%, 90%, 95%, or 100% identical to the amino acid sequence G(T/K/N/D) WQZT(M/F)TN (SEQ ID NO:537) wherein Z is any amino acid, or GTWQ(V/L/A/I) T(M/F)TN (SEQ ID NO:538), wherein the highlighted residues are invariant;
(c) X3 comprises the amino acid sequence (E/S)DG or EDG;
(d) X4 comprises an amino acid sequence at least 50%, 55%, 60%, 65%, 70%, 75%, 80%, 85%, 90%, 95%, or 100% identical to the amino acid sequence QTSOGQMHFQP (SEQ ID NO:539, wherein the highlighted residues are invariant;
(e) X5 comprises a single polar amino acid selected from the group consisting of R, T, Q, N, K, E, D, S, or wherein X5 is R;
(f) X6 comprises the amino acid sequence (T/S)PZ3, where Z3 is polar amino acid or Tyr; or wherein X6 is SPY;
(g) X7 comprises an amino acid sequence at least 50%, 55%, 60%, 65%, 70%, 75%, 80%, 85%, 90%, 95%, or 100% identical to the amino acid sequence T(L/A/M)D(I/V)(K/V)(A/S) GT(I/M) (SEQ ID NO:540) or TMDIVAQGTI (SEQ ID NO:541), wherein the highlighted residues are invariant;
(h) X8 comprises the amino acid sequences (S/A)DG or SDG,
(i) X9 comprises an amino acid sequence at least 50%, 55%, 60%, 65%, 70%, 75%, 80%, 85%, 90%, 95%, or 100% identical to the amino acid sequence RPI(Q/S/T/V)G(Y/K)GK(L/V/A)T(V/C/A) (SEQ ID NO:542) or RPIVGYGKATV (SEQ ID NO:543), wherein the highlighted residues are invariant;
(j) X10 is selected from the group consisting of R, T, Q, N, K, E, D, or S; or X10 is K;
(k) X11 comprises the amino acid sequence (S/T)(P/C) (polar or Y), or wherein X11 is TPD;
(l) X12 comprises an amino acid sequence at least 50%, 55%, 60%, 65%, 70%, 75%, 80%, 85%, 90%, 95%, or 100% identical to the amino acid sequence T(M/L/V)(D/H/Q/N)(V/A/L/I)(D/N/H/Q)(I/L/V) T(Y/W) (SEQ ID NO:544) OR TLDIDITY (SEQ ID NO:545);
(m) X13 comprises the amino acid sequence (S/E)DG, or wherein X13 comprises an amino acid sequence at least 60%, 80%, or 100% identical to PSLGN (SEQ ID NO:546);
(n) X14 comprises an amino acid sequence at least 50%, 55%, 60%, 65%, 70%, 75%, (K/M/I/L)(Q/K)(V/A/G)QGQ(V/I)T(M/L/U) (SEQ ID NO:547) or IKAQGQITM (SEQ ID NO:548), wherein the highlighted residues are invariant;
(o) X15 is selected from the group consisting of R, T, Q, N, K, E, D, or S, or wherein X15 is D;
(p) X16 comprises the amino acid sequence (S/T)P(D/T/Y), or wherein X16 comprises the amino acid sequence SPT;
(q) X17 comprises an amino acid sequence at least 50%, 55%, 60%, 65%, 70%, 75%, 80%, 85%, 90%, 95%, or 100% identical to the amino acid sequence Q(F/A)(K/T/H)(F/W)(D/N)(V/A/S/G)(T/Q/H/E) (T/F/V/Y) (SEQ ID NO:549) or QFLFDATT (SEQ ID NO:550);
(r) X19 comprises an amino acid sequence at least 50%, 55%, 60%, 65%, 70%, 75%, 80%, 85%, 90%, 95%, or 100% identical to the amino acid sequence [(S/K/N/H)](K/R/I/N)(V/L)TFT(L/I/M)QRQE (SEQ ID NO:551) or RLTGTLQRQE (SEQ ID NO:552), wherein residues in brackets are optional; and or
(s) X18 comprises the amino acid sequence selected from the group consisting of (S/E/N/A/Q)DG, SDG, K(G/Q/K/T)(A/D/E/N)(G/D/N)(N/G/D/Y/S) (SEQ ID NO:553), KG(A/D/E)(G/D/N)(N/G/D/Y) (SEQ ID NO:554), KGENDFHG (SEQ ID NO:555), KGADGWHG (SEQ ID NO:556), and KGAGNFTG (SEQ ID NO:557).

8.-25. (canceled)

26. The self complementing multipartite β-barrel protein of claim 1, wherein the first polypeptide component and/or the second polypeptide component comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NOS:1-308, wherein residues in parentheses are optional, and wherein the optional residues may be present or absent.

27. (canceled)

28. The self-complementing multipartite β-barrel protein of claim 1, further comprising a functional domain, optionally wherein the functional domain is present within X18.

29. (canceled)

30. The self-complementing multipartite β-barrel protein of claim 28, wherein the functional domain comprises a detectable moiety including but not limited to a fluorescent protein or other chromophore; and a detector polypeptide including but not limited to a pH responsive polypeptide, an ion-binding polypeptide, a nucleic acid binding polypeptide.

31. A polypeptide comprising a first polypeptide component or a second polypeptide component of claim 1.

32. (canceled)

33. A nucleic acid encoding the polypeptide of claim 31.

34. An expression vector comprising the nucleic acid of claim 33 operatively linked to a control sequence.

35. A host cell comprising the expression vector of claim 34.

36. A β-barrel polypeptide, comprising domains X1-X2-X3-X4-X5-X6-X7-X8-X9-X10-X11-X12-X13-X14-X15-X16-X17-X18-X19, wherein:
X1 comprises a capping domain;
X2 comprises a beta strand,
wherein a contiguous C-terminal portion of X1 and N-terminal portion of X2 comprise the amino acid sequence Z1-P-G-Z2-W, where Z1 and Z2 are any amino acid;
X3 comprises a beta turn;
X4 comprises a beta strand that includes an internal G residue and a P at its C terminus;
X5 comprises a single polar amino acid;
X6 comprises a beta turn;
X7 comprises a beta strand including an internal G residue;
X8 comprises a beta turn;
X9 comprises a beta strand including an internal P residue and 2 internal G residues;
X11.0 comprises a single polar amino acid;
X11 comprises a beta turn;
X12 comprises a beta strand;
X13 comprises a beta turn;
X14 comprises a beta strand with an internal G residue;
X15 comprises a single polar amino acid;
X16 comprises a beta turn;
X17 comprises a beta strand;
X18 comprises a beta turn; and
X19 comprises a beta strand;
wherein the last residue of the X19 domain is N-terminal to and connected to the first residue of X1 domain via an amino acid linker;
wherein 1, 2, or 3 contiguous domains X1, X2, X3, X4, X5, X6, X7, X8, X9, X10, X11, X12, X13, X14, X15, X16, X17, X18, and X19 may be partially or wholly absent.

37.-67. (canceled)

68. A nucleic acid encoding the polypeptide of claim 36.

69. An expression vector comprising the nucleic acid of claim 68 operatively linked to a control sequence.

70. A host cell comprising the expression vector of claim 69.

71. A pharmaceutical composition, comprising
(a) the self-complementing multipartite β-barrel protein of claim 1; and
(b) a pharmaceutically acceptable carrier.

72. A method for using the self-complementing multipartite β-barrel protein of claim 1, for uses including, but not limited to, pH sensing, ion-sensing/detection (including but not limited to Ca²⁺, La³⁺, Tb³⁺, and other ion sensing/detection/quantification), temporal sensing, voltage sensing, mechanical sensing, thermal sensing, super-resolution microscopy, localization microscopy, fluorescence microscopy, fluorescence lifetime imaging, fluorimetry, and detection and quantification of other small-molecules, ions, peptides, nucleic acids, organic substrates, or inorganic substrates.


SEQUENCE LISTING (568 sequences)

SEQ ID NO: 1 - protein, 18 aa, Artificial Sequence
  Synthetic peptide
  MISC_FEATURE (1)..(2): Optional residues.
  MISC_FEATURE (18)..(18): Optional residue.
SRAAQLLPGTWQATFTNE

SEQ ID NO: 2 - protein, 18 aa, Artificial Sequence
  Synthetic peptide
SRAAQLLPGTWQATFTNE

SEQ ID NO: 3 - protein, 18 aa, Artificial Sequence
  Synthetic peptide
  MISC_FEATURE (1)..(2): Optional residues.
  MISC_FEATURE (18)..(18): Optional residue.
SRAAQLLPGTWQVTMTNE

SEQ ID NO: 4 - protein, 18 aa, Artificial Sequence
  Synthetic peptide
SRAAQLLPGTWQVTMTNE

SEQ ID NO: 5 - protein, 34 aa, Artificial Sequence
  Synthetic peptide
  MISC_FEATURE (1)..(2): Optional residues.
  MISC_FEATURE (34)..(34): Optional residue.
SRAAQLLPGTWQATFTNEDGQTSQGQWHFQPRSP

SEQ ID NO: 6 - protein, 34 aa, Artificial Sequence; identical to SEQ ID NO: 5
  Synthetic peptide

SEQ ID NO: 7 - protein, 34 aa, Artificial Sequence
  Synthetic peptide
  MISC_FEATURE (1)..(2): Optional residues.
  MISC_FEATURE (34)..(34): Optional residue.
SRAAQLLPGTWQVTMTNEDGQTSQGQWHFQPRSP

SEQ ID NO: 8 - protein, 34 aa, Artificial Sequence; identical to SEQ ID NO: 7
  Synthetic peptide

SEQ ID NO: 9 - protein, 34 aa, Artificial Sequence
  Synthetic peptide
  MISC_FEATURE (1)..(2): Optional residues.
  MISC_FEATURE (34)..(34): Optional residue.
SRAAQLLPGTWQATFTNEDGQTSQGQFHFQPRSP

SEQ ID NO: 10 - protein, 34 aa, Artificial Sequence; identical to SEQ ID NO: 9
  Synthetic peptide

SEQ ID NO: 11 - protein, 34 aa, Artificial Sequence
  Synthetic peptide
  MISC_FEATURE (1)..(2): Optional residues.
  MISC_FEATURE (34)..(34): Optional residue.
SRAAQLLPGTWQATFTNEDGQTSQGQIHFQPRSP

SEQ ID NO: 12 - protein, 34 aa, Artificial Sequence; identical to SEQ ID NO: 11
  Synthetic peptide

SEQ ID NO: 13 - protein, 34 aa, Artificial Sequence
  Synthetic peptide
  MISC_FEATURE (1)..(2): Optional residues.
  MISC_FEATURE (34)..(34): Optional residue.
SRAAQLLPGTWQVTMTNEDGQTSQGQMHFQPRSP

SEQ ID NO: 14 - protein, 34 aa, Artificial Sequence; identical to SEQ ID NO: 13
  Synthetic peptide

SEQ ID NO: 15 - protein, 46 aa, Artificial Sequence
  Synthetic peptide
  MISC_FEATURE (1)..(2): Optional residues.
  MISC_FEATURE (46)..(46): Optional residue.
SRAAQLLPGTWQATFTNEDGQTSQGQWHFQPRSPYTMDIVAQGTIS

SEQ ID NO: 16 - protein, 46 aa, Artificial Sequence; identical to SEQ ID NO: 15
  Synthetic peptide

SEQ ID NO: 17 - protein, 46 aa, Artificial Sequence
  Synthetic peptide
  MISC_FEATURE (1)..(2): Optional residues.
  MISC_FEATURE (46)..(46): Optional residue.
SRAAQLLPGTWQVTMTNEDGQTSQGQWHFQPRSPYTMDIVAQGTIS

SEQ ID NO: 18 - protein, 46 aa, Artificial Sequence; identical to SEQ ID NO: 17
  Synthetic peptide

SEQ ID NO: 19 - protein, 46 aa, Artificial Sequence
  Synthetic peptide
  MISC_FEATURE (1)..(2): Optional residues.
  MISC_FEATURE (46)..(46): Optional residue.
SRAAQLLPGTWQATFTNEDGQTSQGQFHFQPRSPYTMDIVAQGTIS

SEQ ID NO: 20 - protein, 46 aa, Artificial Sequence; identical to SEQ ID NO: 19
  Synthetic peptide

SEQ ID NO: 21 - protein, 46 aa, Artificial Sequence
  Synthetic peptide
  MISC_FEATURE (1)..(2): Optional residues.
  MISC_FEATURE (46)..(46): Optional residue.
SRAAQLLPGTWQATFTNEDGQTSQGQIHFQPRSPYTMDIVAQGTIS

SEQ ID NO: 22 - protein, 46 aa, Artificial Sequence; identical to SEQ ID NO: 21
  Synthetic peptide

SEQ ID NO: 23 - protein, 46 aa, Artificial Sequence
  Synthetic peptide
  MISC_FEATURE (1)..(2): Optional residues.
  MISC_FEATURE (46)..(46): Optional residue.
SRAAQLLPGTWQATFTNEDGQTSQGQIHFQPRSPYTMDIVSQGTIS

SEQ ID NO: 24 - protein, 46 aa, Artificial Sequence; identical to SEQ ID NO: 23
  Synthetic peptide

SEQ ID NO: 25 - protein, 46 aa, Artificial Sequence
  Synthetic peptide
  MISC_FEATURE (1)..(2): Optional residues.
  MISC_FEATURE (46)..(46): Optional residue.
SRAAQLLPGTWQVTMTNEDGQTSQGQMHFQPRSPYTMDIVAQGTIS

SEQ ID NO: 26 - protein, 46 aa, Artificial Sequence; identical to SEQ ID NO: 25
  Synthetic peptide

SEQ ID NO: 27 - protein, 62 aa, Artificial Sequence
  Synthetic peptide
  MISC_FEATURE (1)..(2): Optional residues.
  MISC_FEATURE (60)..(62): Optional residues.
SRAAQLLPGTWQATFTNEDGQTSQGQWHFQPRSPYTMDIVAQGTISDGRPIVGYGKATVKTP

SEQ ID NO: 28 - protein, 62 aa, Artificial Sequence; identical to SEQ ID NO: 27
  Synthetic peptide

SEQ ID NO: 29 - protein, 62 aa, Artificial Sequence
  Synthetic peptide
  MISC_FEATURE (1)..(2): Optional residues.
  MISC_FEATURE (60)..(62): Optional residues.
SRAAQLLPGTWQVTMTNEDGQTSQGQWHFQPRSPYTMDIVAQGTISDGRPIVGYGKATVKTP

SEQ ID NO: 30 - protein, 62 aa, Artificial Sequence; identical to SEQ ID NO: 29
  Synthetic peptide

SEQ ID NO: 31 - protein, 62 aa, Artificial Sequence
  Synthetic peptide
  MISC_FEATURE (1)..(2): Optional residues.
  MISC_FEATURE (60)..(62): Optional residues.
SRAAQLLPGTWQATFTNEDGQTSQGQFHFQPRSPYTMDIVAQGTISDGRPIVGYGKATVKTP

SEQ ID NO: 32 - protein, 62 aa, Artificial Sequence; identical to SEQ ID NO: 31
  Synthetic peptide

SEQ ID NO: 33 - protein, 62 aa, Artificial Sequence
  Synthetic peptide
  MISC_FEATURE (1)..(2): Optional residues.
  MISC_FEATURE (60)..(62): Optional residues.
SRAAQLLPGTWQATFTNEDGQTSQGQIHFQPRSPYTMDIVAQGTISDGRPIVGYGKATVKTP

SEQ ID NO: 34 - protein, 62 aa, Artificial Sequence; identical to SEQ ID NO: 33
  Synthetic peptide

SEQ ID NO: 35 - protein, 62 aa, Artificial Sequence
  Synthetic peptide
  MISC_FEATURE (1)..(2): Optional residues.
  MISC_FEATURE (60)..(62): Optional residues.
SRAAQLLPGTWQATFTNEDGQTSQGQIHFQPRSPYTMDIVSQGTISDGRPIVGYGKATVKTP

SEQ ID NO: 36 - protein, 62 aa, Artificial Sequence; identical to SEQ ID NO: 35
  Synthetic peptide

SEQ ID NO: 37 - protein, 62 aa, Artificial Sequence
  Synthetic peptide
  MISC_FEATURE (1)..(2): Optional residues.
  MISC_FEATURE (60)..(62): Optional residues.
SRAAQLLPGTWQVTMTNEDGQTSQGQMHFQPRSPYTMDIVAQGTISDGRPIVGYGKATVKTP

SEQ ID NO: 38 - protein, 62 aa, Artificial Sequence; identical to SEQ ID NO: 37
  Synthetic peptide

SEQ ID NO: 39 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQATFTNEDGQTSQGQWHFQPRSPYTMDIVAQGTISDGRPIVGYGKATVKTPDTLDIDITYPS

SEQ ID NO: 40 - protein, 73 aa, Artificial Sequence; identical to SEQ ID NO: 39
  Synthetic peptide

SEQ ID NO: 41 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQVTMTNEDGQTSQGQWHFQPRSPYTMDIVAQGTISDGRPIVGYGKATVKTPDTLDIDITYPS

SEQ ID NO: 42 - protein, 73 aa, Artificial Sequence; identical to SEQ ID NO: 41
  Synthetic peptide

SEQ ID NO: 43 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQATFTNEDGQTSQGQWHFQPRSPYTMDIVAQGTISDGRPIVGYGKATVKTPDTLDIDITWPS

SEQ ID NO: 44 - protein, 73 aa, Artificial Sequence; identical to SEQ ID NO: 43
  Synthetic peptide

SEQ ID NO: 45 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQATFTNEDGQTSQGQFHFQPRSPYTMDIVAQGTISDGRPIVGYGKATVKTPDTLDIDITYPS

SEQ ID NO: 46 - protein, 73 aa, Artificial Sequence; identical to SEQ ID NO: 45
  Synthetic peptide

SEQ ID NO: 47 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQATFTNEDGQTSQGQIHFQPRSPYTMDIVAQGTISDGRPIVGYGKATVKTPDTLDIDITYPS

SEQ ID NO: 48 - protein, 73 aa, Artificial Sequence; identical to SEQ ID NO: 47
  Synthetic peptide

SEQ ID NO: 49 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQATFTNEDGQTSQGQIHFQPRSPYTMDIVSQGTISDGRPIVGYGKATVKTPDTLDIDITYPS

SEQ ID NO: 50 - protein, 73 aa, Artificial Sequence; identical to SEQ ID NO: 49
  Synthetic peptide

SEQ ID NO: 51 - protein, 73 aa, Artificial Sequence
  Synthetic peptide
  MISC_FEATURE (1)..(2): Optional residues.
  MISC_FEATURE (73)..(73): Optional residue.
SRAAQLLPGTWQVTMTNEDGQTSQGQMHFQPRSPYTMDIVAQGTISDGRPIVGYGKATVKTPDTLDIDITYPS

SEQ ID NO: 52 - protein, 73 aa, Artificial Sequence; identical to SEQ ID NO: 51
  Synthetic peptide

SEQ ID NO: 53 - protein, 88 aa, Artificial Sequence
  Synthetic peptide
  MISC_FEATURE (1)..(2): Optional residues.
  MISC_FEATURE (88)..(88): Optional residues.
SRAAQLLPGTWQATFTNEDGQTSQGQWHFQPRSPYTMDIVAQGTISDGRPIVGYGKATVKTPDTLDIDITYPSLGNIKAQGQITMDSP

SEQ ID NO: 54 - protein, 88 aa, Artificial Sequence; identical to SEQ ID NO: 53
  Synthetic peptide

SEQ ID NO: 55 - protein, 88 aa, Artificial Sequence
  Synthetic peptide
  MISC_FEATURE (1)..(2): Optional residues.
  MISC_FEATURE (88)..(88): Optional residue.
SRAAQLLPGTWQVTMTNEDGQTSQGQWHFQPRSPYTMDIVAQGTISDGRPIVGYGKATVKTPDTLDIDITYPSLGNIKAQGQITMDSP

SEQ ID NO: 56 - protein, 88 aa, Artificial Sequence; identical to SEQ ID NO: 55
  Synthetic peptide

SEQ ID NO: 57 - protein, 88 aa, Artificial Sequence
  Synthetic peptide
  MISC_FEATURE (1)..(2): Optional residues.
  MISC_FEATURE (88)..(88): Optional residue.
SRAAQLLPGTWQATFTNEDGQTSQGQWHFQPRSPYTMDIVAQGTISDGRPIVGYGKATVKTPDTLDIDITWPSLGNIKGQGQITMDSP

SEQ ID NO: 58 - protein, 88 aa, Artificial Sequence; identical to SEQ ID NO: 57
  Synthetic peptide

SEQ ID NO: 59 - protein, 88 aa, Artificial Sequence
  Synthetic peptide
  MISC_FEATURE (1)..(2): Optional residues.
  MISC_FEATURE (88)..(88): Optional residue.
SRAAQLLPGTWQATFTNEDGQTSQGQFHFQPRSPYTMDIVAQGTISDGRPIVGYGKATVKTPDTLDIDITYPSLGNIKAQGQITMDSP

SEQ ID NO: 60 - protein, 88 aa, Artificial Sequence; identical to SEQ ID NO: 59
  Synthetic peptide

SEQ ID NO: 61 - protein, 88 aa, Artificial Sequence
  Synthetic peptide
  MISC_FEATURE (1)..(2): Optional residues.
  MISC_FEATURE (88)..(88): Optional residue.
SRAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 62 - protein, 88 aa, Artificial Sequence; identical to SEQ ID NO: 61
  Synthetic peptide

SEQ ID NO: 63 - protein, 88 aa, Artificial Sequence
  Synthetic peptide
  MISC_FEATURE (1)..(2): Optional residues.
  MISC_FEATURE (88)..(88): Optional residue.
SRAAQLLPGTWQATFTNEDGQTSQGQIHFQPRSPYTMDIVSQGTISDGRPIVGYGKATVKTPDTLDIDITYPSLGNIKFQGQITMDSP

SEQ ID NO: 64 - protein, 88 aa, Artificial Sequence; identical to SEQ ID NO: 63
  Synthetic peptide

SEQ ID NO: 65 - protein, 88 aa, Artificial Sequence
  Synthetic peptide
  MISC_FEATURE (1)..(2): Optional residues.
  MISC_FEATURE (88)..(88): Optional residue.
SRAAQLLPGTWQVTMTNEDGQTSQGQMHFQPRSPYTMDIVAQGTISDGRPIVGYGKATVKTPDTLDIDITYPSLGNIKAQGQITMDSP

SEQ ID NO: 66 - protein, 88 aa, Artificial Sequence; identical to SEQ ID NO: 65
  Synthetic peptide

SEQ ID NO: 67 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 68 - protein, 105 aa, Artificial Sequence; identical to SEQ ID NO: 67
  Synthetic peptide

SEQ ID NO: 69 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 70 - protein, 105 aa, Artificial Sequence; identical to SEQ ID NO: 69
  Synthetic peptide

SEQ ID NO: 71 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFHG

SEQ ID NO: 72 - protein, 105 aa, Artificial Sequence; identical to SEQ ID NO: 71
  Synthetic peptide

SEQ ID NO: 73 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFHG

SEQ ID NO: 74 - protein, 105 aa, Artificial Sequence; identical to SEQ ID NO: 73
  Synthetic peptide

SEQ ID NO: 75 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFHG

SEQ ID NO: 76 - protein, 105 aa, Artificial Sequence; identical to SEQ ID NO: 75
  Synthetic peptide

SEQ ID NO: 77 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFHG

SEQ ID NO: 78 - protein, 105 aa, Artificial Sequence; identical to SEQ ID NO: 77
  Synthetic peptide

SEQ ID NO: 79 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFKG

SEQ ID NO: 80 - protein, 105 aa, Artificial Sequence; identical to SEQ ID NO: 79
  Synthetic peptide

SEQ ID NO: 81 - protein, 105 aa, Artificial Sequence
  Synthetic peptide
  MISC_FEATURE (1)..(2): Optional residues.
  MISC_FEATURE (96)..(105): Optional residues.
SRAAQLLPGTWQVTMTNEDGQTSQGQMHFQPRSPYTMDIVAQGTISDGRPIVGYGKATVKTPDTLDIDITYPSLGNIKAQGQITMDSPTQFKFDATTKGENDFHG

SEQ ID NO: 82 - protein, 105 aa, Artificial Sequence; identical to SEQ ID NO: 81
  Synthetic peptide

SEQ ID NO: 83 - protein, 16 aa, Artificial Sequence
  Synthetic peptide
  MISC_FEATURE (1)..(3): Optional residues.
  MISC_FEATURE (16)..(16): Optional residue.
DGQTSQGQWHFQPRSP

SEQ ID NO: 84 - protein, 16 aa, Artificial Sequence
  Synthetic peptide
DGQTSQGQWHFQPRSP

SEQ ID NO: 85 - protein, 16 aa, Artificial Sequence
  Synthetic peptide
  MISC_FEATURE (1)..(3): Optional residues.
  MISC_FEATURE (16)..(16): Optional residue.
DGQTSQGQFHFQPRSP

SEQ ID NO: 86 - protein, 16 aa, Artificial Sequence
  Synthetic peptide
DGQTSQGQFHFQPRSP

SEQ ID NO: 87 - protein, 16 aa, Artificial Sequence
  Synthetic peptide
  MISC_FEATURE (1)..(3): Optional residues.
  MISC_FEATURE (16)..(16): Optional residue.
DGQTSQGQIHFQPRSP

SEQ ID NO: 88 - protein, 16 aa, Artificial Sequence
  Synthetic peptide
DGQTSQGQIHFQPRSP

SEQ ID NO: 89 - protein, 16 aa, Artificial Sequence
  Synthetic peptide
  MISC_FEATURE (1)..(3): Optional residues.
  MISC_FEATURE (16)..(16): Optional residue.
DGQTSQGQMHFQPRSP

SEQ ID NO: 90 - protein, 16 aa, Artificial Sequence
  Synthetic peptide
DGQTSQGQMHFQPRSP

SEQ ID NO: 91 - protein, 28 aa, Artificial Sequence
  Synthetic peptide
  MISC_FEATURE (1)..(3): Optional residues.
  MISC_FEATURE (28)..(28): Optional residue.
DGQTSQGQWHFQPRSPYTMDIVAQGTIS

SEQ ID NO: 92 - protein, 28 aa, Artificial Sequence
  Synthetic peptide
DGQTSQGQWHFQPRSPYTMDIVAQGTIS

SEQ ID NO: 93 - protein, 28 aa, Artificial Sequence
  Synthetic peptide
  MISC_FEATURE (1)..(3): Optional residues.
  MISC_FEATURE (28)..(28): Optional residue.
DGQTSQGQFHFQPRSPYTMDIVAQGTIS

SEQ ID NO: 94 - protein, 28 aa, Artificial Sequence
  Synthetic peptide
DGQTSQGQFHFQPRSPYTMDIVAQGTIS

SEQ ID NO: 95 - protein, 28 aa, Artificial Sequence
  Synthetic peptide
  MISC_FEATURE (1)..(3): Optional residues.
  MISC_FEATURE (28)..(28): Optional residue.
DGQTSQGQIHFQPRSPYTMDIVAQGTIS

SEQ ID NO: 96 - protein, 28 aa, Artificial Sequence
  Synthetic peptide
DGQTSQGQIHFQPRSPYTMDIVAQGTIS

SEQ ID NO: 97 - protein, 28 aa, Artificial Sequence
  Synthetic peptide
  MISC_FEATURE (1)..(3): Optional residues.
  MISC_FEATURE (28)..(28): Optional residue.
DGQTSQGQIHFQPRSPYTMDIVSQGTIS

SEQ ID NO: 98 - protein, 28 aa, Artificial Sequence
  Synthetic peptide
DGQTSQGQIHFQPRSPYTMDIVSQGTIS

SEQ ID NO: 99 - protein, 28 aa, Artificial Sequence
  Synthetic peptide
  MISC_FEATURE (1)..(3): Optional residues.
  MISC_FEATURE (28)..(28): Optional residue.
DGQTSQGQMHFQPRSPYTMDIVAQGTIS

SEQ ID NO: 100 - protein, 28 aa, Artificial Sequence
  Synthetic peptide
DGQTSQGQMHFQPRSPYTMDIVAQGTIS

SEQ ID NO: 101 - protein, 44 aa, Artificial Sequence
  Synthetic peptide
  MISC_FEATURE (1)..(3): Optional residues.
  MISC_FEATURE (42)..(44): Optional residues.
DGQTSQGQWHFQPRSPYTMDIVAQGTISDGRPIVGYGKATVKTP

SEQ ID NO: 102 - protein, 44 aa, Artificial Sequence; identical to SEQ ID NO: 101
  Synthetic peptide

SEQ ID NO: 103 - protein, 44 aa, Artificial Sequence
  Synthetic peptide
  MISC_FEATURE (1)..(3): Optional residues.
  MISC_FEATURE (42)..(44): Optional residues.
DGQTSQGQFHFQPRSPYTMDIVAQGTISDGRPIVGYGKATVKTP

SEQ ID NO: 104 - protein, 44 aa, Artificial Sequence; identical to SEQ ID NO: 103
  Synthetic peptide

SEQ ID NO: 105 - protein, 44 aa, Artificial Sequence
  Synthetic peptide
  MISC_FEATURE (1)..(3): Optional residues.
  MISC_FEATURE (42)..(44): Optional residues.
DGQTSQGQIHFQPRSPYTMDIVAQGTISDGRPIVGYGKATVKTP

SEQ ID NO: 106 - protein, 44 aa, Artificial Sequence; identical to SEQ ID NO: 105
  Synthetic peptide

SEQ ID NO: 107 - protein, 44 aa, Artificial Sequence
  Synthetic peptide
  MISC_FEATURE (1)..(3): Optional residues.
  MISC_FEATURE (42)..(44): Optional residues.
DGQTSQGQIHFQPRSPYTMDIVSQGTISDGRPIVGYGKATVKTP

SEQ ID NO: 108 - protein, 44 aa, Artificial Sequence; identical to SEQ ID NO: 107
  Synthetic peptide

SEQ ID NO: 109 - protein, 44 aa, Artificial Sequence
  Synthetic peptide
  MISC_FEATURE (1)..(3): Optional residues.
  MISC_FEATURE (42)..(44): Optional residues.
DGQTSQGQMHFQPRSPYTMDIVAQGTISDGRPIVGYGKATVKTP

SEQ ID NO: 110 - protein, 44 aa, Artificial Sequence; identical to SEQ ID NO: 109
  Synthetic peptide

SEQ ID NO: 111 - protein, 55 aa, Artificial Sequence
  Synthetic peptide
  MISC_FEATURE (1)..(3): Optional residues.
  MISC_FEATURE (55)..(55): Optional residue.
DGQTSQGQWHFQPRSPYTMDIVAQGTISDGRPIVGYGKATVKTPDTLDIDITYPS

SEQ ID NO: 112 - protein, 55 aa, Artificial Sequence; identical to SEQ ID NO: 111
  Synthetic peptide

SEQ ID NO: 113 - protein, 55 aa, Artificial Sequence
  Synthetic peptide
  MISC_FEATURE (1)..(3): Optional residues.
  MISC_FEATURE (55)..(55): Optional residue.
DGQTSQGQWHFQPRSPYTMDIVAQGTISDGRPIVGYGKATVKTPDTLDIDITWPS

SEQ ID NO: 114 - protein, 55 aa, Artificial Sequence; identical to SEQ ID NO: 113
  Synthetic peptide

SEQ ID NO: 115 - protein, 55 aa, Artificial Sequence
  Synthetic peptide
  MISC_FEATURE (1)..(3): Optional residues.
  MISC_FEATURE (55)..(55): Optional residue.
DGQTSQGQFHFQPRSPYTMDIVAQGTISDGRPIVGYGKATVKTPDTLDIDITYPS

SEQ ID NO: 116 - protein, 55 aa, Artificial Sequence; identical to SEQ ID NO: 115
  Synthetic peptide

SEQ ID NO: 117 - protein, 55 aa, Artificial Sequence
  Synthetic peptide
  MISC_FEATURE (1)..(3): Optional residues.
  MISC_FEATURE (55)..(55): Optional residue.
DGQTSQGQIHFQPRSPYTMDIVAQGTISDGRPIVGYGKATVKTPDTLDIDITYPS

SEQ ID NO: 118 - protein, 55 aa, Artificial Sequence; identical to SEQ ID NO: 117
  Synthetic peptide

SEQ ID NO: 119 - protein, 55 aa, Artificial Sequence
  Synthetic peptide
  MISC_FEATURE (1)..(3): Optional residues.
  MISC_FEATURE (55)..(55): Optional residue.
DGQTSQGQIHFQPRSPYTMDIVSQGTISDGRPIVGYGKATVKTPDTLDIDITYPS

SEQ ID NO: 120 - protein, 55 aa, Artificial Sequence; identical to SEQ ID NO: 119
  Synthetic peptide

SEQ ID NO: 121 - protein, 55 aa, Artificial Sequence
  Synthetic peptide
  MISC_FEATURE (1)..(3): Optional residues.
  MISC_FEATURE (55)..(55): Optional residue.
DGQTSQGQMHFQPRSPYTMDIVAQGTISDGRPIVGYGKATVKTPDTLDIDITYPS

SEQ ID NO: 122 - protein, 55 aa, Artificial Sequence; identical to SEQ ID NO: 121
  Synthetic peptide

SEQ ID NO: 123 - protein, 70 aa, Artificial Sequence
  Synthetic peptide
  MISC_FEATURE (1)..(3): Optional residues.
  MISC_FEATURE (70)..(70): Optional residue.
DGQTSQGQWHFQPRSPYTMDIVAQGTISDGRPIVGYGKATVKTPDTLDIDITYPSLGNIKAQGQITMDSP

SEQ ID NO: 124 - protein, 70 aa, Artificial Sequence; identical to SEQ ID NO: 123
  Synthetic peptide

SEQ ID NO: 125 - protein, 70 aa, Artificial Sequence
  Synthetic peptide
  MISC_FEATURE (1)..(3): Optional residues.
  MISC_FEATURE (70)..(70): Optional residue.
DGQTSQGQWHFQPRSPYTMDIVAQGTISDGRPIVGYGKATVKTPDTLDIDITWPSLGNIKGQGQITMDSP

SEQ ID NO: 126 - protein, 70 aa, Artificial Sequence; identical to SEQ ID NO: 125
  Synthetic peptide

SEQ ID NO: 127 - protein, 70 aa, Artificial Sequence
  Synthetic peptide
  MISC_FEATURE (1)..(3): Optional residues.
  MISC_FEATURE (70)..(70): Optional residue.
DGQTSQGQFHFQPRSPYTMDIVAQGTISDGRPIVGYGKATVKTPDTLDIDITYPSLGNIKAQGQITMDSP

SEQ ID NO: 128 - protein, 70 aa, Artificial Sequence; identical to SEQ ID NO: 127
  Synthetic peptide

SEQ ID NO: 129 - protein, 70 aa, Artificial Sequence
  Synthetic peptide
  MISC_FEATURE (1)..(3): Optional residues.
  MISC_FEATURE (70)..(70): Optional residue.
DGQTSQGQIHFQPRSPYTMDIVAQGTISDGRPIVGYGKATVKTPDTLDIDITYPSLGNIKAQGQITMDSP

SEQ ID NO: 130 - protein, 70 aa, Artificial Sequence; identical to SEQ ID NO: 129
  Synthetic peptide

SEQ ID NO: 131 - protein, 70 aa, Artificial Sequence
  Synthetic peptide
  MISC_FEATURE (1)..(3): Optional residues.
  MISC_FEATURE (70)..(70): Optional residue.
DGQTSQGQIHFQPRSPYTMDIVSQGTISDGRPIVGYGKATVKTPDTLDIDITYPSLGNIKFQGQITMDSP

SEQ ID NO: 132 - protein, 70 aa, Artificial Sequence; identical to SEQ ID NO: 131
  Synthetic peptide

SEQ ID NO: 133 - protein, 70 aa, Artificial Sequence
  Synthetic peptide
  MISC_FEATURE (1)..(3): Optional residues.
  MISC_FEATURE (70)..(70): Optional residue.
DGQTSQGQMHFQPRSPYTMDIVAQGTISDGRPIVGYGKATVKTPDTLDIDITYPSLGNIKAQGQITMDSP

SEQ ID NO: 134 - protein, 70 aa, Artificial Sequence; identical to SEQ ID NO: 133
  Synthetic peptide

SEQ ID NO: 135 - protein, 87 aa, Artificial Sequence
  Synthetic peptide
  MISC_FEATURE (1)..(3): Optional residues.
  MISC_FEATURE (78)..(87): Optional residues.
DGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 136 - protein, 87 aa, Artificial Sequence; identical to SEQ ID NO: 135
  Synthetic peptide

SEQ ID NO: 137 - protein, 87 aa, Artificial Sequence
  Synthetic peptide
  MISC_FEATURE (1)..(3): Optional residues.
  MISC_FEATURE (78)..(87): Optional residues.
DGQTSQGQWHFQPRSPYTMDIVAQGTISDGRPIVGYGKATVKTPDTLDIDITWPSLGNIKGQGQITMDSPTQFKWDGTTKGENDFHG

SEQ ID NO: 138 - protein, 87 aa, Artificial Sequence; identical to SEQ ID NO: 137
  Synthetic peptide

SEQ ID NO: 139 - protein, 87 aa, Artificial Sequence
  Synthetic peptide
  MISC_FEATURE (1)..(3): Optional residues.
  MISC_FEATURE (78)..(87): Optional residues.
DGQTSQGQFHFQPRSPYTMDIVAQGTISDGRPIVGYGKATVKTPDTLDIDITYPSLGNIKAQGQITMDSPTQFKFDATTKGENDFHG

SEQ ID NO: 140 - protein, 87 aa, Artificial Sequence; identical to SEQ ID NO: 139
  Synthetic peptide

SEQ ID NO: 141 - protein, 87 aa, Artificial Sequence
  Synthetic peptide
  MISC_FEATURE (1)..(3): Optional residues.
  MISC_FEATURE (78)..(87): Optional residues.
DGQTSQGQIHFQPRSPYTMDIVAQGTISDGRPIVGYGKATVKTPDTLDIDITYPSLGNIKAQGQITMDSPTQFKFDATTKGENDFHG

SEQ ID NO: 142 - protein, 87 aa, Artificial Sequence; identical to SEQ ID NO: 141
  Synthetic peptide

SEQ ID NO: 143 - protein, 87 aa, Artificial Sequence
  Synthetic peptide
  MISC_FEATURE (1)..(3): Optional residues.
  MISC_FEATURE (78)..(87): Optional residues.
DGQTSQGQIHFQPRSPYTMDIVSQGTISDGRPIVGYGKATVKTPDTLDIDITYPSLGNIKFQGQITMDSPTQFKFDATTKGENDFHG

SEQ ID NO: 144 - protein, 87 aa, Artificial Sequence; identical to SEQ ID NO: 143
  Synthetic peptide

SEQ ID NO: 145 - protein, 87 aa, Artificial Sequence
  Synthetic peptide
  MISC_FEATURE (1)..(3): Optional residues.
  MISC_FEATURE (78)..(87): Optional residues.
DGQTSQGQIHFQPRSPYTMDIVAQGTISDGRPIVGYGKATVKTPDTLDIDITYPSLGNIKAQGQITMDSPTQFKFDATTSGSGGFKG

SEQ ID NO: 146 - protein, 87 aa, Artificial Sequence; identical to SEQ ID NO: 145
  Synthetic peptide

SEQ ID NO: 147 - protein, 87 aa, Artificial Sequence
  Synthetic peptide
  MISC_FEATURE (1)..(3): Optional residues.
  MISC_FEATURE (78)..(87): Optional residues.
DGQTSQGQMHFQPRSPYTMDIVAQGTISDGRPIVGYGKATVKTPDTLDIDITYPSLGNIKAQGQITMDSPTQFKFDATTKGENDFHG

SEQ ID NO: 148 - protein, 87 aa, Artificial Sequence; identical to SEQ ID NO: 147
  Synthetic peptide

SEQ ID NO: 149 - protein, 97 aa, Artificial Sequence
  Synthetic peptide
  MISC_FEATURE (1)..(3): Optional residues.
  MISC_FEATURE (96)..(97): Optional residues.
DGQTSQGQWHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 150 - protein, 97 aa, Artificial Sequence; identical to SEQ ID NO: 149
  Synthetic peptide

SEQ ID NO: 151 - protein, 97 aa, Artificial Sequence
  Synthetic peptide
  MISC_FEATURE (1)..(3): Optional residues.
  MISC_FEATURE (96)..(97): Optional residues.
DGQTSQGQWHFQPRSPYTMDIVAQGTISDGRPIVGYGKATVKTPDTLDIDITWPSLGNIKGQGQITMDSPTQFKWDGTTKGENDFHGRLTGTLQRQE

SEQ ID NO: 152 - protein, 97 aa, Artificial Sequence; identical to SEQ ID NO: 151
  Synthetic peptide

SEQ ID NO: 153 - protein, 97 aa, Artificial Sequence
  Synthetic peptide
  MISC_FEATURE (1)..(3): Optional residues.
  MISC_FEATURE (96)..(97): Optional residues.
DGQTSQGQFHFQPRSPYTMDIVAQGTISDGRPIVGYGKATVKTPDTLDIDITYPSLGNIKAQGQITMDSPTQFKFDATTKGENDFHGRLTGTLQRQE

SEQ ID NO: 154 - protein, 97 aa, Artificial Sequence; identical to SEQ ID NO: 153
  Synthetic peptide

SEQ ID NO: 155 - protein, 97 aa, Artificial Sequence
  Synthetic peptide
  MISC_FEATURE (1)..(3): Optional residues.
  MISC_FEATURE (96)..(97): Optional residues.
DGQTSQGQIHFQPRSPYTMDIVAQGTISDGRPIVGYGKATVKTPDTLDIDITYPSLGNIKAQGQITMDSPTQFKFDATTKGENDFHGRLTGTLQRQE

SEQ ID NO: 156 - protein, 97 aa, Artificial Sequence; identical to SEQ ID NO: 155
  Synthetic peptide

SEQ ID NO: 157 - protein, 97 aa, Artificial Sequence
  Synthetic peptide
  MISC_FEATURE (1)..(3): Optional residues.
  MISC_FEATURE (96)..(97): Optional residues.
DGQTSQGQIHFQPRSPYTMDIVSQGTISDGRPIVGYGKATVKTPDTLDIDITYPSLGNIKFQGQITMDSPTQFKFDATTKGENDFHGRLTGTLQRQE

SEQ ID NO: 158 - protein, 97 aa, Artificial Sequence; identical to SEQ ID NO: 157
  Synthetic peptide

SEQ ID NO: 159 - protein, 97 aa, Artificial Sequence
  Synthetic peptide
  MISC_FEATURE (1)..(3): Optional residues.
  MISC_FEATURE (96)..(97): Optional residues.
DGQTSQGQIHFQPRSPYTMDIVAQGTISDGRPIVGYGKATVKTPDTLDIDITYPSLGNIKAQGQITMDSPTQFKFDATTSGSGGFKGRLTGTLQRQE

SEQ ID NO: 160 - protein, 97 aa, Artificial Sequence; identical to SEQ ID NO: 159
  Synthetic peptide

SEQ ID NO: 161 - protein, 97 aa, Artificial Sequence
  Synthetic peptide
  MISC_FEATURE (1)..(3): Optional residues.
  MISC_FEATURE (96)..(97): Optional residues.
DGQTSQGQMHFQPRSPYTMDIVAQGTISDGRPIVGYGKATVKTPDTLDIDITYPSLGNIKAQGQITMDSPTQFKFDATTKGENDFHGRLTGTLQRQE

SEQ ID NO: 162 - protein, 97 aa, Artificial Sequence; identical to SEQ ID NO: 161
  Synthetic peptide

SEQ ID NO: 163 - protein, 12 aa, Artificial Sequence
  Synthetic peptide
  MISC_FEATURE (1)..(1): Optional residue.
  MISC_FEATURE (12)..(12): Optional residue.
YTMDIVAQGTIS

SEQ ID NO: 164 - protein, 12 aa, Artificial Sequence
  Synthetic peptide
YTMDIVAQGTIS

SEQ ID NO: 165 - protein, 12 aa, Artificial Sequence
  Synthetic peptide
  MISC_FEATURE (1)..(1): Optional residue.
  MISC_FEATURE (12)..(12): Optional residue.
YTMDIVSQGTIS

SEQ ID NO: 166 - protein, 12 aa, Artificial Sequence
  Synthetic peptide
YTMDIVSQGTIS

SEQ ID NO: 167 - protein, 28 aa, Artificial Sequence
  Synthetic peptide
  MISC_FEATURE (1)..(1): Optional residue.
  MISC_FEATURE (26)..(28): Optional residues.
YTMDIVAQGTISDGRPIVGYGKATVKTP

SEQ ID NO: 168 - protein, 28 aa, Artificial Sequence
  Synthetic peptide
YTMDIVAQGTISDGRPIVGYGKATVKTP

SEQ ID NO: 169 - protein, 28 aa, Artificial Sequence
  Synthetic peptide
  MISC_FEATURE (1)..(1): Optional residue.
  MISC_FEATURE (26)..(28): Optional residues.
YTMDIVSQGTISDGRPIVGYGKATVKTP

SEQ ID NO: 170 - protein, 28 aa, Artificial Sequence
  Synthetic peptide
YTMDIVSQGTISDGRPIVGYGKATVKTP

SEQ ID NO: 171 - protein, 39 aa, Artificial Sequence
  Synthetic peptide
  MISC_FEATURE (1)..(1): Optional residue.
  MISC_FEATURE (39)..(39): Optional residue.
YTMDIVAQGTISDGRPIVGYGKATVKTPDTLDIDITYPS

SEQ ID NO: 172 - protein, 39 aa, Artificial Sequence; identical to SEQ ID NO: 171
  Synthetic peptide

SEQ ID NO: 173 - protein, 39 aa, Artificial Sequence
  Synthetic peptide
  MISC_FEATURE (1)..(1): Optional residue.
  MISC_FEATURE (39)..(39): Optional residue.
YTMDIVAQGTISDGRPIVGYGKATVKTPDTLDIDITWPS

SEQ ID NO: 174 - protein, 39 aa, Artificial Sequence; identical to SEQ ID NO: 173
  Synthetic peptide

SEQ ID NO: 175 - protein, 39 aa, Artificial Sequence
  Synthetic peptide
  MISC_FEATURE (1)..(1): Optional residue.
  MISC_FEATURE (39)..(39): Optional residue.
YTMDIVSQGTISDGRPIVGYGKATVKTPDTLDIDITYPS

SEQ ID NO: 176 - protein, 39 aa, Artificial Sequence; identical to SEQ ID NO: 175
  Synthetic peptide

SEQ ID NO: 177 - protein, 54 aa, Artificial Sequence
  Synthetic peptide
  MISC_FEATURE (1)..(1): Optional residue.
  MISC_FEATURE (54)..(54): Optional residue.
YTMDIVAQGTISDGRPIVGYGKATVKTPDTLDIDITYPSLGNIKAQGQITMDSP

SEQ ID NO: 178 - protein, 54 aa, Artificial Sequence; identical to SEQ ID NO: 177
  Synthetic peptide

SEQ ID NO: 179 - protein, 54 aa, Artificial Sequence
  Synthetic peptide
  MISC_FEATURE (1)..(1): Optional residue.
  MISC_FEATURE (54)..(54): Optional residue.
YTMDIVAQGTISDGRPIVGYGKATVKTPDTLDIDITWPSLGNIKGQGQITMDSP

SEQ ID NO: 180 - protein, 54 aa, Artificial Sequence; identical to SEQ ID NO: 179
  Synthetic peptide

SEQ ID NO: 181 - protein, 54 aa, Artificial Sequence
  Synthetic peptide
  MISC_FEATURE (1)..(1): Optional residue.
  MISC_FEATURE (54)..(54): Optional residue.
YTMDIVSQGTISDGRPIVGYGKATVKTPDTLDIDITYPSLGNIKFQGQITMDSP

SEQ ID NO: 182 - protein, 54 aa, Artificial Sequence; identical to SEQ ID NO: 181
  Synthetic peptide

SEQ ID NO: 183 - protein, 71 aa, Artificial Sequence
  Synthetic peptide
  MISC_FEATURE (1)..(1): Optional residue.
  MISC_FEATURE (62)..(71): Optional residues.
YTMDIVAQGTISDGRPIVGYGKATVKTPDTLDIDITYPSLGNIKAQGQITMDSPTQFKWDATTKGENDFHG

SEQ ID NO: 184 - protein, 71 aa, Artificial Sequence; identical to SEQ ID NO: 183
  Synthetic peptide

SEQ ID NO: 185 - protein, 71 aa, Artificial Sequence
  Synthetic peptide
  MISC_FEATURE (1)..(1): Optional residue.
  MISC_FEATURE (62)..(71): Optional residues.
YTMDIVAQGTISDGRPIVGYGKATVKTPDTLDIDITWPSLGNIKGQGQITMDSPTQFKWDGTTKGENDFHG

SEQ ID NO: 186 - protein, 71 aa, Artificial Sequence; identical to SEQ ID NO: 185
  Synthetic peptide

SEQ ID NO: 187 - protein, 71 aa, Artificial Sequence
  Synthetic peptide
  MISC_FEATURE (1)..(1): Optional residue.
  MISC_FEATURE (62)..(71): Optional residues.
YTMDIVAQGTISDGRPIVGYGKATVKTPDTLDIDITYPSLGNIKAQGQITMDSPTQFKFDATTKGENDFHG

SEQ ID NO: 188 - protein, 71 aa, Artificial Sequence; identical to SEQ ID NO: 187
  Synthetic peptide

SEQ ID NO: 189 - protein, 71 aa, Artificial Sequence
  Synthetic peptide
  MISC_FEATURE (1)..(1): Optional residue.
  MISC_FEATURE (62)..(71): Optional residues.
YTMDIVSQGTISDGRPIVGYGKATVKTPDTLDIDITYPSLGNIKFQGQITMDSPTQFKFDATTKGENDFHG

SEQ ID NO: 190 - protein, 71 aa, Artificial Sequence; identical to SEQ ID NO: 189
  Synthetic peptide

SEQ ID NO: 191 - protein, 71 aa, Artificial Sequence
  Synthetic peptide
  MISC_FEATURE (1)..(1): Optional residue.
  MISC_FEATURE (62)..(71): Optional residues.
YTMDIVAQGTISDGRPIVGYGKATVKTPDTLDIDITYPSLGNIKAQGQITMDSPTQFKFDATTSGSGGFKG

SEQ ID NO: 192 - protein, 71 aa, Artificial Sequence; identical to SEQ ID NO: 191
  Synthetic peptide

SEQ ID NO: 193 - protein, 81 aa, Artificial Sequence
  Synthetic peptide
  MISC_FEATURE (1)..(1): Optional residue.
  MISC_FEATURE (80)..(81): Optional residues.
YTMDIVAQGTISDGRPIVGYGKATVKTPDTLDIDITYPSLGNIKAQGQITMDSPTQFKWDATTKGENDFHGRLTGTLQRQE

SEQ ID NO: 194 - protein, 81 aa, Artificial Sequence; identical to SEQ ID NO: 193
  Synthetic peptide

SEQ ID NO: 195 - protein, 81 aa, Artificial Sequence
  Synthetic peptide
  MISC_FEATURE (1)..(1): Optional residue.
  MISC_FEATURE (80)..(81): Optional residues.
YTMDIVAQGTISDGRPIVGYGKATVKTPDTLDIDITWPSLGNIKGQGQITMDSPTQFKWDGTTKGENDFHGRLTGTLQRQE

SEQ ID NO: 196 - protein, 81 aa, Artificial Sequence; identical to SEQ ID NO: 195
  Synthetic peptide

SEQ ID NO: 197 - protein, 81 aa, Artificial Sequence
  Synthetic peptide
  MISC_FEATURE (1)..(1): Optional residue.
  MISC_FEATURE (80)..(81): Optional residues.
YTMDIVAQGTISDGRPIVGYGKATVKTPDTLDIDITYPSLGNIKAQGQITMDSPTQFKFDATTKGENDFHGRLTGTLQRQE

SEQ ID NO: 198 - protein, 81 aa, Artificial Sequence; identical to SEQ ID NO: 197
  Synthetic peptide

SEQ ID NO: 199 - protein, 81 aa, Artificial Sequence
  Synthetic peptide
  MISC_FEATURE (1)..(1): Optional residue.
  MISC_FEATURE (80)..(81): Optional residues.
YTMDIVSQGTISDGRPIVGYGKATVKTPDTLDIDITYPSLGNIKFQGQITMDSPTQFKFDATTKGENDFHGRLTGTLQRQE

SEQ ID NO: 200 - protein, 81 aa, Artificial Sequence; identical to SEQ ID NO: 199
  Synthetic peptide

SEQ ID NO: 201 - protein, 81 aa, Artificial Sequence
  Synthetic peptide
  MISC_FEATURE (1)..(1): Optional residue.
  MISC_FEATURE (80)..(81): Optional residues.
YTMDIVAQGTISDGRPIVGYGKATVKTPDTLDIDITYPSLGNIKAQGQITMDSPTQFKFDATTSGSGGFKGRLTGTLQRQE

SEQ ID NO: 202 - protein, 81 aa, Artificial Sequence; identical to SEQ ID NO: 201
  Synthetic peptide

SEQ ID NO: 203 - protein, 16 aa, Artificial Sequence
  Synthetic peptide
  MISC_FEATURE (1)..(4): Optional residues.
  MISC_FEATURE (14)..(16): Optional residues.
DGRPIVGYGKATVKTP

SEQ ID NO: 204 - protein, 16 aa, Artificial Sequence
  Synthetic peptide
DGRPIVGYGKATVKTP

SEQ ID NO: 205 - protein, 27 aa, Artificial Sequence
  Synthetic peptide
  MISC_FEATURE (1)..(4): Optional residues.
  MISC_FEATURE (27)..(27): Optional residue.
DGRPIVGYGKATVKTPDTLDIDITYPS

SEQ ID NO: 206 - protein, 27 aa, Artificial Sequence
  Synthetic peptide
DGRPIVGYGKATVKTPDTLDIDITYPS

SEQ ID NO: 207 - protein, 27 aa, Artificial Sequence
  Synthetic peptide
  MISC_FEATURE (1)..(4): Optional residues.
  MISC_FEATURE (27)..(27): Optional residue.
DGRPIVGYGKATVKTPDTLDIDITWPS

SEQ ID NO: 208 - protein, 27 aa, Artificial Sequence
  Synthetic peptide
DGRPIVGYGKATVKTPDTLDIDITWPS

SEQ ID NO: 209 - protein, 42 aa, Artificial Sequence
  Synthetic peptide
  MISC_FEATURE (1)..(4): Optional residues.
  MISC_FEATURE (42)..(42): Optional residue.
DGRPIVGYGKATVKTPDTLDIDITYPSLGNIKAQGQITMDSP

SEQ ID NO: 210 - protein, 42 aa, Artificial Sequence; identical to SEQ ID NO: 209
  Synthetic peptide

SEQ ID NO: 211 - protein, 42 aa, Artificial Sequence
  Synthetic peptide
  MISC_FEATURE (1)..(4): Optional residues.
  MISC_FEATURE (42)..(42): Optional residue.
DGRPIVGYGKATVKTPDTLDIDITWPSLGNIKGQGQITMDSP

SEQ ID NO: 212 - protein, 42 aa, Artificial Sequence; identical to SEQ ID NO: 211
  Synthetic peptide

SEQ ID NO: 213 - protein, 42 aa, Artificial Sequence
  Synthetic peptide
  MISC_FEATURE (1)..(4): Optional residues.
  MISC_FEATURE (42)..(42): Optional residue.
DGRPIVGYGKATVKTPDTLDIDITYPSLGNIKFQGQITMDSP

SEQ ID NO: 214 - protein, 42 aa, Artificial Sequence; identical to SEQ ID NO: 213
  Synthetic peptide

SEQ ID NO: 215 - protein, 59 aa, Artificial Sequence
  Synthetic peptide
  MISC_FEATURE (1)..(4): Optional residues.
  MISC_FEATURE (50)..(59): Optional residues.
DGRPIVGYGKATVKTPDTLDIDITYPSLGNIKAQGQITMDSPTQFKWDATTKGENDFHG

SEQ ID NO: 216 - protein, 59 aa, Artificial Sequence; identical to SEQ ID NO: 215
  Synthetic peptide

SEQ ID NO: 217 - protein, 59 aa, Artificial Sequence
  Synthetic peptide
  MISC_FEATURE (1)..(4): Optional residues.
  MISC_FEATURE (50)..(59): Optional residues.
DGRPIVGYGKATVKTPDTLDIDITWPSLGNIKGQGQITMDSPTQFKWDGTTKGENDFHG

SEQ ID NO: 218 - protein, 59 aa, Artificial Sequence; identical to SEQ ID NO: 217
  Synthetic peptide

SEQ ID NO: 219 - protein, 59 aa, Artificial Sequence
  Synthetic peptide
  MISC_FEATURE (1)..(4): Optional residues.
  MISC_FEATURE (50)..(59): Optional residues.
DGRPIVGYGKATVKTPDTLDIDITYPSLGNIKAQGQITMDSPTQFKFDATTKGENDFHG

SEQ ID NO: 220 - protein, 59 aa, Artificial Sequence; identical to SEQ ID NO: 219
  Synthetic peptide

SEQ ID NO: 221 - protein, 59 aa, Artificial Sequence
  Synthetic peptide
  MISC_FEATURE (1)..(4): Optional residues.
  MISC_FEATURE (50)..(59): Optional residues.
DGRPIVGYGKATVKTPDTLDIDITYPSLGNIKFQGQITMDSPTQFKFDATTKGENDFHG

SEQ ID NO: 222 - protein, 59 aa, Artificial Sequence; identical to SEQ ID NO: 221
  Synthetic peptide

SEQ ID NO: 223 - protein, 59 aa, Artificial Sequence
  Synthetic peptide
  MISC_FEATURE (1)..(4): Optional residues.
  MISC_FEATURE (50)..(59): Optional residues.
DGRPIVGYGKATVKTPDTLDIDITYPSLGNIKAQGQITMDSPTQFKFDATTSGSGGFKG

SEQ ID NO: 224 - protein, 59 aa, Artificial Sequence; identical to SEQ ID NO: 223
  Synthetic peptide

SEQ ID NO: 225 - protein, 69 aa, Artificial Sequence
  Synthetic peptide
  MISC_FEATURE (1)..(4): Optional residues.
  MISC_FEATURE (68)..(69): Optional residues.
DGRPIVGYGKATVKTPDTLDIDITYPSLGNIKAQGQITMDSPTQFKWDATTKGENDFHGRLTGTLQRQE

SEQ ID NO: 226 - protein, 69 aa, Artificial Sequence; identical to SEQ ID NO: 225
  Synthetic peptide

SEQ ID NO: 227 - protein, 69 aa, Artificial Sequence
  Synthetic peptide
  MISC_FEATURE (1)..(4): Optional residues.
  MISC_FEATURE (68)..(69): Optional residues.
DGRPIVGYGKATVKTPDTLDIDITWPSLGNIKGQGQITMDSPTQFKWDGTTKGENDFHGRLTGTLQRQE

SEQ ID NO: 228 - protein, 69 aa, Artificial Sequence; identical to SEQ ID NO: 227
  Synthetic peptide

SEQ ID NO: 229 - protein, 69 aa, Artificial Sequence
  Synthetic peptide
  MISC_FEATURE (1)..(4): Optional residues.
  MISC_FEATURE (68)..(69): Optional residues.
DGRPIVGYGKATVKTPDTLDIDITYPSLGNIKAQGQITMDSPTQFKFDATTKGENDFHGRLTGTLQRQE

SEQ ID NO: 230 - protein, 69 aa, Artificial Sequence; identical to SEQ ID NO: 229
  Synthetic peptide

SEQ ID NO: 231 - protein, 69 aa, Artificial Sequence
  Synthetic peptide
  MISC_FEATURE (1)..(4): Optional residues.
  MISC_FEATURE (68)..(69): Optional residues.
DGRPIVGYGKATVKTPDTLDIDITYPSLGNIKFQGQITMDSPTQFKFDATTKGENDFHGRLTGTLQRQE

SEQ ID NO: 232 - protein, 69 aa, Artificial Sequence; identical to SEQ ID NO: 231
  Synthetic peptide

SEQ ID NO: 233 - protein, 69 aa, Artificial Sequence
  Synthetic peptide
  MISC_FEATURE (1)..(4): Optional residues.
  MISC_FEATURE (68)..(69): Optional residues.
DGRPIVGYGKATVKTPDTLDIDITYPSLGNIKAQGQITMDSPTQFKFDATTSGSGGFKGRLTGTLQRQE

SEQ ID NO: 234 - protein, 69 aa, Artificial Sequence; identical to SEQ ID NO: 233
  Synthetic peptide

SEQ ID NO: 235 - protein, 11 aa, Artificial Sequence
  Synthetic peptide
  MISC_FEATURE (1)..(1): Optional residue.
  MISC_FEATURE (11)..(11): Optional residue.
DTLDIDITYPS

SEQ ID NO: 236 - protein, 11 aa, Artificial Sequence
  Synthetic peptide
DTLDIDITYPS

SEQ ID NO: 237 - protein, 11 aa, Artificial Sequence
  Synthetic peptide
  MISC_FEATURE (1)..(1): Optional residue.
  MISC_FEATURE (11)..(11): Optional residue.
DTLDIDITWPS

SEQ ID NO: 238 - protein, 11 aa, Artificial Sequence
  Synthetic peptide
DTLDIDITWPS

SEQ ID NO: 239 - protein, 26 aa, Artificial Sequence
  Synthetic peptide
  MISC_FEATURE (1)..(1): Optional residue.
  MISC_FEATURE (26)..(26): Optional residue.
DTLDIDITYPSLGNIKAQGQITMDSP

SEQ ID NO: 240 - protein, 26 aa, Artificial Sequence
  Synthetic peptide
DTLDIDITYPSLGNIKAQGQITMDSP

SEQ ID NO: 241 - protein, 26 aa, Artificial Sequence
  Synthetic peptide
  MISC_FEATURE (1)..(1): Optional residue.
  MISC_FEATURE (26)..(26): Optional residue.
DTLDIDITWPSLGNIKGQGQITMDSP

SEQ ID NO: 242 - protein, 26 aa, Artificial Sequence
  Synthetic peptide
DTLDIDITWPSLGNIKGQGQITMDSP

SEQ ID NO: 243 - protein, 26 aa, Artificial Sequence
  Synthetic peptide
  MISC_FEATURE (1)..(1): Optional residue.
  MISC_FEATURE (26)..(26): Optional residue.
DTLDIDITYPSLGNIKFQGQITMDSP

SEQ ID NO: 244 - protein, 26 aa, Artificial Sequence
  Synthetic peptide
DTLDIDITYPSLGNIKFQGQITMDSP

SEQ ID NO: 245 - protein, 43 aa, Artificial Sequence
  Synthetic peptide
  MISC_FEATURE (1)..(1): Optional residue.
  MISC_FEATURE (34)..(43): Optional residues.
DTLDIDITYPSLGNIKAQGQITMDSPTQFKWDATTKGENDFHG

SEQ ID NO: 246 - protein, 43 aa, Artificial Sequence; identical to SEQ ID NO: 245
  Synthetic peptide

SEQ ID NO: 247 - protein, 43 aa, Artificial Sequence
  Synthetic peptide
  MISC_FEATURE (1)..(1): Optional residue.
  MISC_FEATURE (34)..(43): Optional residues.
DTLDIDITWPSLGNIKGQGQITMDSPTQFKWDGTTKGENDFHG

SEQ ID NO: 248 - protein, 43 aa, Artificial Sequence; identical to SEQ ID NO: 247
  Synthetic peptide

SEQ ID NO: 249 - protein, 43 aa, Artificial Sequence
  Synthetic peptide
  MISC_FEATURE (1)..(1): Optional residue.
  MISC_FEATURE (34)..(43): Optional residues.
DTLDIDITYPSLGNIKAQGQITMDSPTQFKFDATTKGENDFHG

SEQ ID NO: 250 - protein, 43 aa, Artificial Sequence; identical to SEQ ID NO: 249
  Synthetic peptide

SEQ ID NO: 251 - protein, 43 aa, Artificial Sequence
  Synthetic peptide
  MISC_FEATURE (1)..(1): Optional residue.
  MISC_FEATURE (34)..(43): Optional residues.
DTLDIDITYPSLGNIKFQGQITMDSPTQFKFDATTKGENDFHG

SEQ ID NO: 252 - protein, 43 aa, Artificial Sequence; identical to SEQ ID NO: 251
  Synthetic peptide

SEQ ID NO: 253 - protein, 43 aa, Artificial Sequence
  Synthetic peptide
  MISC_FEATURE (1)..(1): Optional residue.
  MISC_FEATURE (34)..(43): Optional residues.
DTLDIDITYPSLGNIKAQGQITMDSPTQFKFDATTSGSGGFKG

SEQ ID NO: 254 - protein, 43 aa, Artificial Sequence; identical to SEQ ID NO: 253
  Synthetic peptide

SEQ ID NO: 255 - protein, 53 aa, Artificial Sequence
  Synthetic peptide
  MISC_FEATURE (1)..(1): Optional residue.
  MISC_FEATURE (52)..(53): Optional residues.
DTLDIDITYPSLGNIKAQGQITMDSPTQFKWDATTKGENDFHGRLTGTLQRQE

SEQ ID NO: 256 - protein, 53 aa, Artificial Sequence; identical to SEQ ID NO: 255
  Synthetic peptide

SEQ ID NO: 257 - protein, 53 aa, Artificial Sequence
  Synthetic peptide
  MISC_FEATURE (1)..(1): Optional residue.
  MISC_FEATURE (52)..(53): Optional residues.
DTLDIDITWPSLGNIKGQGQITMDSPTQFKWDGTTKGENDFHGRLTGTLQRQE

SEQ ID NO: 258 - protein, 53 aa, Artificial Sequence; identical to SEQ ID NO: 257
  Synthetic peptide

SEQ ID NO: 259 - protein, 53 aa, Artificial Sequence
  Synthetic peptide
  MISC_FEATURE (1)..(1): Optional residue.
  MISC_FEATURE (52)..(53): Optional residues.
DTLDIDITYPSLGNIKAQGQITMDSPTQFKFDATTKGENDFHGRLTGTLQRQE

SEQ ID NO: 260 - protein, 53 aa, Artificial Sequence; identical to SEQ ID NO: 259
  Synthetic peptide

SEQ ID NO: 261 - protein, 53 aa, Artificial Sequence
  Synthetic peptide
  MISC_FEATURE (1)..(1): Optional residue.
  MISC_FEATURE (52)..(53): Optional residues.
DTLDIDITYPSLGNIKFQGQITMDSPTQFKFDATTKGENDFHGRLTGTLQRQE

SEQ ID NO: 262 - protein, 53 aa, Artificial Sequence; identical to SEQ ID NO: 261
  Synthetic peptide

SEQ ID NO: 263 - protein, 53 aa, Artificial Sequence
  Synthetic peptide
  MISC_FEATURE (1)..(1): Optional residue.
  MISC_FEATURE (52)..(53): Optional residues.
DTLDIDITYPSLGNIKAQGQITMDSPTQFKFDATTSGSGGFKGRLTGTLQRQE

SEQ ID NO: 264 - protein, 53 aa, Artificial Sequence; identical to SEQ ID NO: 263
  Synthetic peptide

SEQ ID NO: 265 - protein, 15 aa, Artificial Sequence
  Synthetic peptide
  MISC_FEATURE (1)..(4): Optional residues.
  MISC_FEATURE (15)..(15): Optional residue.
LGNIKAQGQITMDSP

SEQ ID NO: 266 - protein, 15 aa, Artificial Sequence
  Synthetic peptide
LGNIKAQGQITMDSP

SEQ ID NO: 267 - protein, 15 aa, Artificial Sequence
  Synthetic peptide
  MISC_FEATURE (1)..(4): Optional residues.
  MISC_FEATURE (15)..(15): Optional residue.
LGNIKGQGQITMDSP

SEQ ID NO: 268 - protein, 15 aa, Artificial Sequence
  Synthetic peptide
LGNIKGQGQITMDSP

SEQ ID NO: 269 - protein, 15 aa, Artificial Sequence
  Synthetic peptide
  MISC_FEATURE (1)..(4): Optional residues.
  MISC_FEATURE (15)..(15): Optional residue.
LGNIKFQGQITMDSP

SEQ ID NO: 270 - protein, 15 aa, Artificial Sequence
  Synthetic peptide
LGNIKFQGQITMDSP

SEQ ID NO: 271 - protein, 32 aa, Artificial Sequence
  Synthetic peptide
  MISC_FEATURE (1)..(4): Optional residues.
  MISC_FEATURE (23)..(32): Optional residues.
LGNIKAQGQITMDSPTQFKWDATTKGENDFHG

SEQ ID NO: 272 - protein, 32 aa, Artificial Sequence; identical to SEQ ID NO: 271
  Synthetic peptide

SEQ ID NO: 273 - protein, 32 aa, Artificial Sequence
  Synthetic peptide
  MISC_FEATURE (1)..(4): Optional residues.
  MISC_FEATURE (23)..(32): Optional residues.
LGNIKGQGQITMDSPTQFKWDGTTKGENDFHG

SEQ ID NO: 274 - protein, 32 aa, Artificial Sequence; identical to SEQ ID NO: 273
  Synthetic peptide

SEQ ID NO: 275 - protein, 32 aa, Artificial Sequence
  Synthetic peptide
  MISC_FEATURE (1)..(4): Optional residues.
  MISC_FEATURE (23)..(32): Optional residues.
LGNIKAQGQITMDSPTQFKFDATTKGENDFHG

SEQ ID NO: 276 - protein, 32 aa, Artificial Sequence; identical to SEQ ID NO: 275
  Synthetic peptide

SEQ ID NO: 277 - protein, 32 aa, Artificial Sequence
  Synthetic peptide
  MISC_FEATURE (1)..(4): Optional residues.
  MISC_FEATURE (23)..(32): Optional residues.
LGNIKFQGQITMDSPTQFKFDATTKGENDFHG

SEQ ID NO: 278 - protein, 32 aa, Artificial Sequence; identical to SEQ ID NO: 277
  Synthetic peptide

SEQ ID NO: 279 - protein, 32 aa, Artificial Sequence
  Synthetic peptide
  MISC_FEATURE (1)..(4): Optional residues.
  MISC_FEATURE (23)..(32): Optional residues.
LGNIKAQGQITMDSPTQFKFDATTSGSGGFKG

SEQ ID NO: 280 - protein, 32 aa, Artificial Sequence; identical to SEQ ID NO: 279
  Synthetic peptide

SEQ ID NO: 281 - protein, 42 aa, Artificial Sequence
  Synthetic peptide
  MISC_FEATURE (1)..(4): Optional residues.
  MISC_FEATURE (41)..(42): Optional residues.
LGNIKAQGQITMDSPTQFKWDATTKGENDFHGRLTGTLQRQE

SEQ ID NO: 282 - protein, 42 aa, Artificial Sequence; identical to SEQ ID NO: 281
  Synthetic peptide

SEQ ID NO: 283 - protein, 42 aa, Artificial Sequence
  Synthetic peptide
  MISC_FEATURE (1)..(4): Optional residues.
  MISC_FEATURE (41)..(42): Optional residues.
LGNIKGQGQITMDSPTQFKWDGTTKGENDFHGRLTGTLQRQE

SEQ ID NO: 284 - protein, 42 aa, Artificial Sequence; identical to SEQ ID NO: 283
  Synthetic peptide

SEQ ID NO: 285 - protein, 42 aa, Artificial Sequence
  Synthetic peptide
  MISC_FEATURE (1)..(4): Optional residues.
  MISC_FEATURE (41)..(42): Optional residues.
LGNIKAQGQITMDSPTQFKFDATTKGENDFHGRLTGTLQRQE

SEQ ID NO: 286 - protein, 42 aa, Artificial Sequence; identical to SEQ ID NO: 285
  Synthetic peptide

SEQ ID NO: 287 - protein, 42 aa, Artificial Sequence
  Synthetic peptide
  MISC_FEATURE (1)..(4): Optional residues.
  MISC_FEATURE (41)..(42): Optional residues.
LGNIKFQGQITMDSPTQFKFDATTKGENDFHGRLTGTLQRQE

SEQ ID NO: 288 - protein, 42 aa, Artificial Sequence; identical to SEQ ID NO: 287
  Synthetic peptide

SEQ ID NO: 289 - protein, 42 aa, Artificial Sequence
  Synthetic peptide
  MISC_FEATURE (1)..(4): Optional residues.
  MISC_FEATURE (41)..(42): Optional residues.
LGNIKAQGQITMDSPTQFKFDATTSGSGGFKGRLTGTLQRQE

SEQ ID NO: 290 - protein, 42 aa, Artificial Sequence; identical to SEQ ID NO: 289
  Synthetic peptide

SEQ ID NO: 291 - protein, 17 aa, Artificial Sequence
  Synthetic peptide
  MISC_FEATURE (1)..(1): Optional residue.
  MISC_FEATURE (8)..(17): Optional residues.
TQFKWDATTKGENDFHG

SEQ ID NO: 292 - protein, 17 aa, Artificial Sequence
  Synthetic peptide
TQFKWDATTKGENDFHG

SEQ ID NO: 293 - protein, 17 aa, Artificial Sequence
  Synthetic peptide
  MISC_FEATURE (1)..(1): Optional residue.
  MISC_FEATURE (8)..(17): Optional residues.
TQFKWDGTTKGENDFHG

SEQ ID NO: 294 - protein, 17 aa, Artificial Sequence
  Synthetic peptide
TQFKWDGTTKGENDFHG

SEQ ID NO: 295 - protein, 17 aa, Artificial Sequence
  Synthetic peptide
  MISC_FEATURE (1)..(1): Optional residue.
  MISC_FEATURE (8)..(17): Optional residues.
TQFKFDATTKGENDFHG

SEQ ID NO: 296 - protein, 17 aa, Artificial Sequence
  Synthetic peptide
TQFKFDATTKGENDFHG

SEQ ID NO: 297 - protein, 17 aa, Artificial Sequence
  Synthetic peptide
  MISC_FEATURE (1)..(1): Optional residue.
  MISC_FEATURE (8)..(17): Optional residues.
TQFKFDATTSGSGGFKG

SEQ ID NO: 298 - protein, 17 aa, Artificial Sequence
  Synthetic peptide
TQFKFDATTSGSGGFKG

SEQ ID NO: 299 - protein, 27 aa, Artificial Sequence
  Synthetic peptide
  MISC_FEATURE (1)..(1): Optional residue.
  MISC_FEATURE (26)..(27): Optional residues.
TQFKWDATTKGENDFHGRLTGTLQRQE

SEQ ID NO: 300 - protein, 27 aa, Artificial Sequence
  Synthetic peptide
TQFKWDATTKGENDFHGRLTGTLQRQE

SEQ ID NO: 301 - protein, 27 aa, Artificial Sequence
  Synthetic peptide
  MISC_FEATURE (1)..(1): Optional residue.
  MISC_FEATURE (26)..(27): Optional residues.
TQFKWDGTTKGENDFHGRLTGTLQRQE

SEQ ID NO: 302 - protein, 27 aa, Artificial Sequence
  Synthetic peptide
TQFKWDGTTKGENDFHGRLTGTLQRQE

SEQ ID NO: 303 - protein, 27 aa, Artificial Sequence
  Synthetic peptide
  MISC_FEATURE (1)..(1): Optional residue.
  MISC_FEATURE (26)..(27): Optional residues.
TQFKFDATTKGENDFHGRLTGTLQRQE

SEQ ID NO: 304 - protein, 27 aa, Artificial Sequence
  Synthetic peptide
TQFKFDATTKGENDFHGRLTGTLQRQE

SEQ ID NO: 305 - protein, 27 aa, Artificial Sequence
  Synthetic peptide
  MISC_FEATURE (1)..(1): Optional residue.
  MISC_FEATURE (26)..(27): Optional residues.
TQFKFDATTSGSGGFKGRLTGTLQRQE

SEQ ID NO: 306 - protein, 27 aa, Artificial Sequence
  Synthetic peptide
TQFKFDATTSGSGGFKGRLTGTLQRQE

SEQ ID NO: 307 - protein, 10 aa, Artificial Sequence
  Synthetic peptide
  MISC_FEATURE (1)..(1): Optional residue.
  MISC_FEATURE (9)..(10): Optional residues.
RLTGTLQRQE

SEQ ID NO: 308 - protein, 10 aa, Artificial Sequence
  Synthetic peptide
RLTGTLQRQE

SEQ ID NO: 309 - protein, 135 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRLPGTEEWAKRIQEELKKKGYGTTKAAQLLPGTWQATFTNEDGQTSQGQWHFQPRDG

SEQ ID NO: 310 - protein, 135 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRLPGTEEWAKRIQEELKKKGYGTTKAAQLLPGTWAVTMTNEDGQTSQGQWHFQPRDG

SEQ ID NO: 311 - protein, 135 aa, Artificial Sequence
  Synthetic peptide
QEMDIVAQGTISDGRPIVGYGKATVKTPDTLDIDITWPSLGNIKGQGQITMDSPTQFKWDGTTKGENDFHGRLTGTLQRLPGTEEWAKRIQEELKKKGYGTTKAAQLLPGTWQATFTNEDGQTSQGQWHFQPRDG

SEQ ID NO: 312 - protein, 135 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRLPGTEEWAKRIQEELKKKGYGTTKAAQLLPGTWQATFTNEDGQTSQGQFHFQPRDG

SEQ ID NO: 313 - protein, 135 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRLPGTEEWAKRIQEELKKKGYGTTKAAQLLPGTWQATFTNEDGQTSQGQIHFQPRDG

SEQ ID NO: 314 - protein, 135 aa, Artificial Sequence
  Synthetic peptide
QEMDIVSQGTISDGRPIVGYGKATVKTPDTLDIDITYPSLGNIKFQGQITMDSPTQFKFDATTKGENDFHGRLTGTLQRLPGTEEWAKRIQEELKKKGYGTTKAAQLLPGTWQATFTNEDGQTSQGQIHFQPRDG

SEQ ID NO: 315 - protein, 135 aa, Artificial Sequence
  Synthetic peptide
QEMDIVAQGTISDGRPIVGYGKATVKTPDTLDIDITYPSLGNIKAQGQITMDSPTQFKFDATTSGSGGFKGRLTGTLQRLPGTEEWAKRIQEELKKKGYGTTKAAQLLPGTWQATFTNEDGQTSQGQIHFQPRDG

SEQ ID NO: 316 - protein, 135 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRLPGTEEWAKRIQEELKKKGYGTTKAAQLLPGTWAVTMTNEDGQTSQGQMHFQPRDG

SEQ ID NO: 317 - protein, 134 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRTEEAKKILKEIQKKHKDEVQTDRAAQLLPGTWQATFTNEDGQTSQGQWHFQPRDG

SEQ ID NO: 318 - protein, 134 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRTEEAKKILKEIQKKHKDEVQTDRAAQLLPGTWAVTMTNEDGQTSQGQWHFQPRDG

SEQ ID NO: 319 - protein, 134 aa, Artificial Sequence
  Synthetic peptide
QEMDIVAQGTISDGRPIVGYGKATVKTPDTLDIDITWPSLGNIKGQGQITMDSPTQFKWDGTTKGENDFHGRLTGTLQRTEEAKKILKEIQKKHKDEVQTDRAAQLLPGTWQATFTNEDGQTSQGQWHFQPRDG

SEQ ID NO: 320 - protein, 134 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTEEAKKILKEIQKKHKDEVQTDRAAQLLPGTWQATFTNEDGQTSQGQFHFQPRDG

SEQ ID NO: 321 - protein, 134 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTEEAKKILKEIQKKHKDEVQTDRAAQLLPGTWQATFTNEDGQTSQGQIHFQPRDG

SEQ ID NO: 322 - protein, 134 aa, Artificial Sequence
  Synthetic peptide
QEMDIVSQGTISDGRPIVGYGKATVKTPDTLDIDITYPSLGNIKFQGQITMDSPTQFKFDATTKGENDFHGRLTGTLQRTEEAKKILKEIQKKHKDEVQTDRAAQLLPGTWQATFTNEDGQTSQGQIHFQPRDG

SEQ ID NO: 323 - protein, 134 aa, Artificial Sequence
  Synthetic peptide
QEMDIVAQGTISDGRPIVGYGKATVKTPDTLDIDITYPSLGNIKAQGQITMDSPTQFKFDATTSGSGGFKGRLTGTLQRTEEAKKILKEIQKKHKDEVQTDRAAQLLPGTWQATFTNEDGQTSQGQIHFQPRDG

SEQ ID NO: 324 - protein, 134 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTEEAKKILKEIQKKHKDEVQTDRAAQLLPGTWAVTMTNEDGQTSQGQMHFQPRDG

SEQ ID NO: 325 - protein, 131 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRLPSAEEADEELKRQGVRGTLAAQLLPGTWQATFTNEDGQTSQGQWHFQPRDG

SEQ ID NO: 326 - protein, 131 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRLPSAEEADEELKRQGVRGTLAAQLLPGTWAVTMTNEDGQTSQGQWHFQPRDG

SEQ ID NO: 327 - protein, 131 aa, Artificial Sequence
  Synthetic peptide
QEMDIVAQGTISDGRPIVGYGKATVKTPDTLDIDITWPSLGNIKGQGQITMDSPTQFKWDGTTKGENDFHGRLTGTLQRLPSAEEADEELKRQGVRGTLAAQLLPGTWQATFTNEDGQTSQGQWHFQPRDG

SEQ ID NO: 328 - protein, 131 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRLPSAEEADEELKRQGVRGTLAAQLLPGTWQATFTNEDGQTSQGQFHFQPRDG

SEQ ID NO: 329 - protein, 131 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRLPSAEEADEELKRQGVRGTLAAQLLPGTWQATFTNEDGQTSQGQIHFQPRDG

SEQ ID NO: 330 - protein, 131 aa, Artificial Sequence
  Synthetic peptide
QEMDIVSQGTISDGRPIVGYGKATVKTPDTLDIDITYPSLGNIKFQGQITMDSPTQFKFDATTKGENDFHGRLTGTLQRLPSAEEADEELKRQGVRGTLAAQLLPGTWQATFTNEDGQTSQGQIHFQPRDG

SEQ ID NO: 331 - protein, 131 aa, Artificial Sequence
  Synthetic peptide
QEMDIVAQGTISDGRPIVGYGKATVKTPDTLDIDITYPSLGNIKAQGQITMDSPTQFKFDATTSGSGGFKGRLTGTLQRLPSAEEADEELKRQGVRGTLAAQLLPGTWQATFTNEDGQTSQGQIHFQPRDG

SEQ ID NO: 332 - protein, 131 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRLPSAEEADEELKRQGVRGTLAAQLLPGTWAVTMTNEDGQTSQGQMHFQPRDG

SEQ ID NO: 333 - protein, 121 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRTSDGGHGPDNAAQLLPGTWQATFTNEDGQTSQGQWHFQPRDG

SEQ ID NO: 334 - protein, 121 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRTSDGGHGPDNAAQLLPGTWAVTMTNEDGQTSQGQWHFQPRDG

SEQ ID NO: 335 - protein, 121 aa, Artificial Sequence
  Synthetic peptide
QEMDIVAQGTISDGRPIVGYGKATVKTPDTLDIDITWPSLGNIKGQGQITMDSPTQFKWDGTTKGENDFHGRLTGTLQRTSDGGHGPDNAAQLLPGTWQATFTNEDGQTSQGQWHFQPRDG

SEQ ID NO: 336 - protein, 121 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTSDGGHGPDNAAQLLPGTWQATFTNEDGQTSQGQFHFQPRDG

SEQ ID NO: 337 - protein, 121 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTSDGGHGPDNAAQLLPGTWQATFTNEDGQTSQGQIHFQPRDG

SEQ ID NO: 338 - protein, 121 aa, Artificial Sequence
  Synthetic peptide
QEMDIVSQGTISDGRPIVGYGKATVKTPDTLDIDITYPSLGNIKFQGQITMDSPTQFKFDATTKGENDFHGRLTGTLQRTSDGGHGPDNAAQLLPGTWQATFTNEDGQTSQGQIHFQPRDG

SEQ ID NO: 339 - protein, 121 aa, Artificial Sequence
  Synthetic peptide
QEMDIVAQGTISDGRPIVGYGKATVKTPDTLDIDITYPSLGNIKAQGQITMDSPTQFKFDATTSGSGGFKGRLTGTLQRTSDGGHGPDNAAQLLPGTWQATFTNEDGQTSQGQIHFQPRDG

SEQ ID NO: 340 - protein, 121 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTSDGGHGPDNAAQLLPGTWAVTMTNEDGQTSQGQMHFQPRDG

SEQ ID NO: 341 - protein, 130 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRTEEAKEATEEARRRGITTQAAQLLPGTWQATFTNEDGQTSQGQWHFQPRDG

SEQ ID NO: 342 - protein, 130 aa, Artificial Sequence
  Synthetic peptide
QEMDIVAQGTISDGRPIVGYGKATVKTPDTLDIDITYPSLGNIKAQGQITMDSPTQFKWDATTKGENDFHGRLTGTLQRTEEAKEATEEARRRGITTQAAQLLPGTWAVTMTNEDGQTSQGQWHFQPRDG

SEQ ID NO: 343 - protein, 130 aa, Artificial Sequence
  Synthetic peptide
QEMDIVAQGTISDGRPIVGYGKATVKTPDTLDIDITWPSLGNIKGQGQITMDSPTQFKWDGTTKGENDFHGRLTGTLQRTEEAKEATEEARRRGITTQAAQLLPGTWQATFTNEDGQTSQGQWHFQPRDG

SEQ ID NO: 344 - protein, 130 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTEEAKEATEEARRRGITTQAAQLLPGTWQATFTNEDGQTSQGQFHFQPRDG

SEQ ID NO: 345 - protein, 130 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTEEAKEATEEARRRGITTQAAQLLPGTWQATFTNEDGQTSQGQIHFQPRDG

SEQ ID NO: 346 - protein, 130 aa, Artificial Sequence
  Synthetic peptide
QEMDIVSQGTISDGRPIVGYGKATVKTPDTLDIDITYPSLGNIKFQGQITMDSPTQFKFDATTKGENDFHGRLTGTLQRTEEAKEATEEARRRGITTQAAQLLPGTWQATFTNEDGQTSQGQIHFQPRDG

SEQ ID NO: 347 - protein, 130 aa, Artificial Sequence
  Synthetic peptide
QEMDIVAQGTISDGRPIVGYGKATVKTPDTLDIDITYPSLGNIKAQGQITMDSPTQFKFDATTSGSGGFKGRLTGTLQRTEEAKEATEEARRRGITTQAAQLLPGTWQATFTNEDGQTSQGQIHFQPRDG

SEQ ID NO: 348 - protein, 130 aa, Artificial Sequence
  Synthetic peptide
QEMDIVAQGTISDGRPIVGYGKATVKTPDTLDIDITYPSLGNIKAQGQITMDSPTQFKFDATTKGENDFHGRLTGTLQRTEEAKEATEEARRRGITTQAAQLLPGTWAVTMTNEDGQTSQGQMHFQPRDG

SEQ ID NO: 349 - protein, 131 aa, Artificial Sequence
  Synthetic peptide
ATLDIDITYPSLGNIKAQGQITMDSPTQFKWDATTKGENDFHGRLTGTLQRLPTAEEAQKEVKKKGLTGSNAAQLLPGTWQATFTNEDGQTSQGQWHFQPRSPYTMDIVAQGTISDGRPIVGYGKATVKDE

SEQ ID NO: 350 - protein, 131 aa, Artificial Sequence
  Synthetic peptide
ATLDIDITYPSLGNIKAQGQITMDSPTQFKWDATTKGENDFHGRLTGTLQRLPTAEEAQKEVKKKGLTGSNAAQLLPGTWAVTMTNEDGQTSQGQWHFQPRSPYTMDIVAQGTISDGRPIVGYGKATVKDE

SEQ ID NO: 351 - protein, 131 aa, Artificial Sequence
  Synthetic peptide
ATLDIDITWPSLGNIKGQGQITMDSPTQFKWDGTTKGENDFHGRLTGTLQRLPTAEEAQKEVKKKGLTGSNAAQLLPGTWQATFTNEDGQTSQGQWHFQPRSPYTMDIVAQGTISDGRPIVGYGKATVKDE

SEQ ID NO: 352 - protein, 131 aa, Artificial Sequence
  Synthetic peptide
ATLDIDITYPSLGNIKAQGQITMDSPTQFKFDATTKGENDFHGRLTGTLQRLPTAEEAQKEVKKKGLTGSNAAQLLPGTWQATFTNEDGQTSQGQFHFQPRSPYTMDIVAQGTISDGRPIVGYGKATVKDE

SEQ ID NO: 353 - protein, 131 aa, Artificial Sequence
  Synthetic peptide
ATLDIDITYPSLGNIKAQGQITMDSPTQFKFDATTKGENDFHGRLTGTLQRLPTAEEAQKEVKKKGLTGSNAAQLLPGTWQATFTNEDGQTSQGQIHFQPRSPYTMDIVAQGTISDGRPIVGYGKATVKDE

SEQ ID NO: 354 - protein, 131 aa, Artificial Sequence
  Synthetic peptide
ATLDIDITYPSLGNIKFQGQITMDSPTQFKFDATTKGENDFHGRLTGTLQRLPTAEEAQKEVKKKGLTGSNAAQLLPGTWQATFTNEDGQTSQGQIHFQPRSPYTMDIVSQGTISDGRPIVGYGKATVKDE

SEQ ID NO: 355 - protein, 131 aa, Artificial Sequence
  Synthetic peptide
ATLDIDITYPSLGNIKAQGQITMDSPTQFKFDATTSGSGGFKGRLTGTLQRLPTAEEAQKEVKKKGLTGSNAAQLLPGTWQATFTNEDGQTSQGQIHFQPRSPYTMDIVAQGTISDGRPIVGYGKATVKDE

SEQ ID NO: 356 - protein, 131 aa, Artificial Sequence
  Synthetic peptide
ATLDIDITYPSLGNIKAQGQITMDSPTQFKFDATTKGENDFHGRLTGTLQRLPTAEEAQKEVKKKGLTGSNAAQLLPGTWAVTMTNEDGQTSQGQMHFQPRSPYTMDIVAQGTISDGRPIVGYGKATVKDE

SEQ ID NO: 357 - protein, 137 aa, Artificial Sequence
  Synthetic peptide
KQFKWDATTKGENDFHGRLTGTLQRLPTAEEWYKRWEKELRKRGTSWEQTLAAQLLPGTWQATFTNEDGQTSQGQWHFQPRSPYTMDIVAQGTISDGRPIVGYGKATVKTPDTLDIDITYPSLGNIKAQGQITMDEP

SEQ ID NO: 358 - protein, 137 aa, Artificial Sequence
  Synthetic peptide
KQFKWDATTKGENDFHGRLTGTLQRLPTAEEWYKRWEKELRKRGTSWEQTLAAQLLPGTWAVTMTNEDGQTSQGQWHFQPRSPYTMDIVAQGTISDGRPIVGYGKATVKTPDTLDIDITYPSLGNIKAQGQITMDEP

SEQ ID NO: 359 - protein, 137 aa, Artificial Sequence
  Synthetic peptide
KQFKWDGTTKGENDFHGRLTGTLQRLPTAEEWYKRWEKELRKRGTSWEQTLAAQLLPGTWQATFTNEDGQTSQGQWHFQPRSPYTMDIVAQGTISDGRPIVGYGKATVKTPDTLDIDITWPSLGNIKGQGQITMDEP

SEQ ID NO: 360 - protein, 137 aa, Artificial Sequence
  Synthetic peptide
KQFKFDATTKGENDFHGRLTGTLQRLPTAEEWYKRWEKELRKRGTSWEQTLAAQLLPGTWQATFTNEDGQTSQGQFHFQPRSPYTMDIVAQGTISDGRPIVGYGKATVKTPDTLDIDITYPSLGNIKAQGQITMDEP

SEQ ID NO: 361 - protein, 137 aa, Artificial Sequence
  Synthetic peptide
KQFKFDATTKGENDFHGRLTGTLQRLPTAEEWYKRWEKELRKRGTSWEQTLAAQLLPGTWQATFTNEDGQTSQGQIHFQPRSPYTMDIVAQGTISDGRPIVGYGKATVKTPDTLDIDITYPSLGNIKAQGQITMDEP

SEQ ID NO: 362 - protein, 137 aa, Artificial Sequence
  Synthetic peptide
KQFKFDATTKGENDFHGRLTGTLQRLPTAEEWYKRWEKELRKRGTSWEQTLAAQLLPGTWQATFTNEDGQTSQGQIHFQPRSPYTMDIVSQGTISDGRPIVGYGKATVKTPDTLDIDITYPSLGNIKFQGQITMDEP

SEQ ID NO: 363 - protein, 137 aa, Artificial Sequence
  Synthetic peptide
KQFKFDATTSGSGGFKGRLTGTLQRLPTAEEWYKRWEKELRKRGTSWEQTLAAQLLPGTWQATFTNEDGQTSQGQIHFQPRSPYTMDIVAQGTISDGRPIVGYGKATVKTPDTLDIDITYPSLGNIKAQGQITMDEP

SEQ ID NO: 364 - protein, 137 aa, Artificial Sequence
  Synthetic peptide
KQFKFDATTKGENDFHGRLTGTLQRLPTAEEWYKRWEKELRKRGTSWEQTLAAQLLPGTWAVTMTNEDGQTSQGQMHFQPRSPYTMDIVAQGTISDGRPIVGYGKATVKTPDTLDIDITYPSLGNIKAQGQITMDEP

SEQ ID NO: 365 - protein, 131 aa, Artificial Sequence
  Synthetic peptide
KQFKWDATTKGENDFHGRLTGTLQREPRSEEIVKKAQHTWKGGSLAAQLLPGTWQATFTNEDGQTSQGQWHFQPRSPYTMDIVAQGTISDGRPIVGYGKATVKTPDTLDIDITYPSLGNIKAQGQITMDEP

SEQ ID NO: 366 - protein, 131 aa, Artificial Sequence
  Synthetic peptide
KQFKWDATTKGENDFHGRLTGTLQREPRSEEIVKKAQHTWKGGSLAAQLLPGTWAVTMTNEDGQTSQGQWHFQPRSPYTMDIVAQGTISDGRPIVGYGKATVKTPDTLDIDITYPSLGNIKAQGQITMDEP

SEQ ID NO: 367 - protein, 131 aa, Artificial Sequence
  Synthetic peptide
KQFKWDGTTKGENDFHGRLTGTLQREPRSEEIVKKAQHTWKGGSLAAQLLPGTWQATFTNEDGQTSQGQWHFQPRSPYTMDIVAQGTISDGRPIVGYGKATVKTPDTLDIDITWPSLGNIKGQGQITMDEP

SEQ ID NO: 368 - protein, 131 aa, Artificial Sequence
  Synthetic peptide
KQFKFDATTKGENDFHGRLTGTLQREPRSEEIVKKAQHTWKGGSLAAQLLPGTWQATFTNEDGQTSQGQFHFQPRSPYTMDIVAQGTISDGRPIVGYGKATVKTPDTLDIDITYPSLGNIKAQGQITMDEP

SEQ ID NO: 369 - protein, 131 aa, Artificial Sequence
  Synthetic peptide
KQFKFDATTKGENDFHGRLTGTLQREPRSEEIVKKAQHTWKGGSLAAQLLPGTWQATFTNEDGQTSQGQIHFQPRSPYTMDIVAQGTISDGRPIVGYGKATVKTPDTLDIDITYPSLGNIKAQGQITMDEP

SEQ ID NO: 370 - protein, 131 aa, Artificial Sequence
  Synthetic peptide
KQFKFDATTKGENDFHGRLTGTLQREPRSEEIVKKAQHTWKGGSLAAQLLPGTWQATFTNEDGQTSQGQIHFQPRSPYTMDIVSQGTISDGRPIVGYGKATVKTPDTLDIDITYPSLGNIKFQGQITMDEP

SEQ ID NO: 371 - protein, 131 aa, Artificial Sequence
  Synthetic peptide
KQFKFDATTSGSGGFKGRLTGTLQREPRSEEIVKKAQHTWKGGSLAAQLLPGTWQATFTNEDGQTSQGQIHFQPRSPYTMDIVAQGTISDGRPIVGYGKATVKTPDTLDIDITYPSLGNIKAQGQITMDEP

SEQ ID NO: 372 - protein, 131 aa, Artificial Sequence
  Synthetic peptide
KQFKFDATTKGENDFHGRLTGTLQREPRSEEIVKKAQHTWKGGSLAAQLLPGTWAVTMTNEDGQTSQGQMHFQPRSPYTMDIVAQGTISDGRPIVGYGKATVKTPDTLDIDITYPSLGNIKAQGQITMDEP

SEQ ID NO: 373 - protein, 122 aa, Artificial Sequence
  Synthetic peptide
RLTGTLQRLPGGGGGDGTRAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 374 - protein, 122 aa, Artificial Sequence
  Synthetic peptide
RLTGTLQRLPGGGGGDGTRAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 375 - protein, 122 aa, Artificial Sequence
  Synthetic peptide
RLTGTLQRLPGGGGGDGTRAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFPG

SEQ ID NO: 376 - protein, 122 aa, Artificial Sequence
  Synthetic peptide
RLTGTLQRLPGGGGGDGTRAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFPG

SEQ ID NO: 377 - protein, 122 aa, Artificial Sequence
  Synthetic peptide
RLTGTLQRLPGGGGGDGTRAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFPG

SEQ ID NO: 378 - protein, 122 aa, Artificial Sequence
  Synthetic peptide
RLTGTLQRLPGGGGGDGTRAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFPG

SEQ ID NO: 379 - protein, 122 aa, Artificial Sequence
  Synthetic peptide
RLTGTLQRLPGGGGGDGTRAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFPG

SEQ ID NO: 380 - protein, 122 aa, Artificial Sequence
  Synthetic peptide
RLTGTLQRLPGGGGGDGTRAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFPG

SEQ ID NO: 381 - protein, 135 aa, Artificial Sequence
  Synthetic peptide
RLTGTLQRTPNAEEYLKELEERKRKGMQPLNEAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 382 - protein, 135 aa, Artificial Sequence
  Synthetic peptide
RLTGTLQRTPNAEEYLKELEERKRKGMQPLNEAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 383 - protein, 135 aa, Artificial Sequence
  Synthetic peptide
RLTGTLQRTPNAEEYLKELEERKRKGMQPLNEAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFPG

SEQ ID NO: 384 - protein, 135 aa, Artificial Sequence
  Synthetic peptide
RLTGTLQRTPNAEEYLKELEERKRKGMQPLNEAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFPG

SEQ ID NO: 385 - protein, 135 aa, Artificial Sequence
  Synthetic peptide
RLTGTLQRTPNAEEYLKELEERKRKGMQPLNEAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFPG

SEQ ID NO: 386 - protein, 135 aa, Artificial Sequence
  Synthetic peptide
RLTGTLQRTPNAEEYLKELEERKRKGMQPLNEAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFPG

SEQ ID NO: 387 - protein, 135 aa, Artificial Sequence
  Synthetic peptide
RLTGTLQRTPNAEEYLKELEERKRKGMQPLNEAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFPG

SEQ ID NO: 388 - protein, 135 aa, Artificial Sequence
  Synthetic peptide
RLTGTLQRTPNAEEYLKELEERKRKGMQPLNEAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFPG

SEQ ID NO: 389 - protein, 135 aa, Artificial Sequence
  Synthetic peptide
RLTGTLQRTPKAGDEEYAKRLEEEARKKGGTIAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 390 - protein, 135 aa, Artificial Sequence
  Synthetic peptide
RLTGTLQRTPKAGDEEYAKRLEEEARKKGGTIAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 391 - protein, 135 aa, Artificial Sequence
  Synthetic peptide
RLTGTLQRTPKAGDEEYAKRLEEEARKKGGTIAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFPG

SEQ ID NO: 392 - protein, 135 aa, Artificial Sequence
  Synthetic peptide
RLTGTLQRTPKAGDEEYAKRLEEEARKKGGTIAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFPG

SEQ ID NO: 393 - protein, 135 aa, Artificial Sequence
  Synthetic peptide
RLTGTLQRTPKAGDEEYAKRLEEEARKKGGTIAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFPG

SEQ ID NO: 394 - protein, 135 aa, Artificial Sequence
  Synthetic peptide
RLTGTLQRTPKAGDEEYAKRLEEEARKKGGTIAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFPG

SEQ ID NO: 395 - protein, 135 aa, Artificial Sequence
  Synthetic peptide
RLTGTLQRTPKAGDEEYAKRLEEEARKKGGTIAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFPG

SEQ ID NO: 396 - protein, 135 aa, Artificial Sequence
  Synthetic peptide
RLTGTLQRTPKAGDEEYAKRLEEEARKKGGTIAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFPG

SEQ ID NO: 397 - protein, 124 aa, Artificial Sequence
  Synthetic peptide
RLTGTLQRTEPTGGGGGGGVTAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 398 - protein, 124 aa, Artificial Sequence
  Synthetic peptide
RLTGTLQRTEPTGGGGGGGVTAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFPG

SEQ ID NO: 399 - protein, 124 aa, Artificial Sequence
  Synthetic peptide
RLTGTLQRTEPTGGGGGGGVTAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFPG

SEQ ID NO: 400 - protein, 124 aa, Artificial Sequence
  Synthetic peptide
RLTGTLQRTEPTGGGGGGGVTAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFPG

SEQ ID NO: 401 - protein, 124 aa, Artificial Sequence
  Synthetic peptide
RLTGTLQRTEPTGGGGGGGVTAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFPG

SEQ ID NO: 402 - protein, 124 aa, Artificial Sequence
  Synthetic peptide
RLTGTLQRTEPTGGGGGGGVTAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFPG

SEQ ID NO: 403 - protein, 124 aa, Artificial Sequence
  Synthetic peptide
RLTGTLQRTEPTGGGGGGGVTAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFPG

SEQ ID NO: 404 - protein, 124 aa, Artificial Sequence
  Synthetic peptide
RLTGTLQRTEPTGGGGGGGVTAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFPG

SEQ ID NO: 405 - protein, 135 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRLPGTEEWAKRIQEELKKKGYGTTKAAQLLPGTWQATFTNEDGQTSQGQWHFQPRSP

SEQ ID NO: 406 - protein, 135 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRLPGTEEWAKRIQEELKKKGYGTTKAAQLLPGTWAVTMTNEDGQTSQGQWHFQPRSP

SEQ ID NO: 407 - protein, 135 aa, Artificial Sequence
  Synthetic peptide
YTMDIVAQGTISDGRPIVGYGKATVKTPDTLDIDITWPSLGNIKGQGQITMDSPTQFKWDGTTKGENDFHGRLTGTLQRLPGTEEWAKRIQEELKKKGYGTTKAAQLLPGTWQATFTNEDGQTSQGQWHFQPRSP

SEQ ID NO: 408 - protein, 135 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRLPGTEEWAKRIQEELKKKGYGTTKAAQLLPGTWQATFTNEDGQTSQGQFHFQPRSP

SEQ ID NO: 409 - protein, 135 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRLPGTEEWAKRIQEELKKKGYGTTKAAQLLPGTWQATFTNEDGQTSQGQIHFQPRSP

SEQ ID NO: 410 - protein, 135 aa, Artificial Sequence
  Synthetic peptide
YTMDIVSQGTISDGRPIVGYGKATVKTPDTLDIDITYPSLGNIKFQGQITMDSPTQFKFDATTKGENDFHGRLTGTLQRLPGTEEWAKRIQEELKKKGYGTTKAAQLLPGTWQATFTNEDGQTSQGQIHFQPRSP

SEQ ID NO: 411 - protein, 135 aa, Artificial Sequence
  Synthetic peptide
YTMDIVAQGTISDGRPIVGYGKATVKTPDTLDIDITYPSLGNIKAQGQITMDSPTQFKFDATTSGSGGFKGRLTGTLQRLPGTEEWAKRIQEELKKKGYGTTKAAQLLPGTWQATFTNEDGQTSQGQIHFQPRSP

SEQ ID NO: 412 - protein, 135 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRLPGTEEWAKRIQEELKKKGYGTTKAAQLLPGTWAVTMTNEDGQTSQGQMHFQPRSP

SEQ ID NO: 413 - protein, 131 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRLPSAEEADEELKRQGVRGTLAAQLLPGTWQATFTNEDGQTSQGQWHFQPRSP

SEQ ID NO: 414 - protein, 131 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRLPSAEEADEELKRQGVRGTLAAQLLPGTWAVTMTNEDGQTSQGQWHFQPRSP

SEQ ID NO: 415 - protein, 131 aa, Artificial Sequence
  Synthetic peptide
YTMDIVAQGTISDGRPIVGYGKATVKTPDTLDIDITWPSLGNIKGQGQITMDSPTQFKWDGTTKGENDFHGRLTGTLQRLPSAEEADEELKRQGVRGTLAAQLLPGTWQATFTNEDGQTSQGQWHFQPRSP

SEQ ID NO: 416 - protein, 131 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRLPSAEEADEELKRQGVRGTLAAQLLPGTWQATFTNEDGQTSQGQFHFQPRSP

SEQ ID NO: 417 - protein, 131 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRLPSAEEADEELKRQGVRGTLAAQLLPGTWQATFTNEDGQTSQGQIHFQPRSP

SEQ ID NO: 418 - protein, 131 aa, Artificial Sequence
  Synthetic peptide
YTMDIVSQGTISDGRPIVGYGKATVKTPDTLDIDITYPSLGNIKFQGQITMDSPTQFKFDATTKGENDFHGRLTGTLQRLPSAEEADEELKRQGVRGTLAAQLLPGTWQATFTNEDGQTSQGQIHFQPRSP

SEQ ID NO: 419 - protein, 131 aa, Artificial Sequence
  Synthetic peptide
YTMDIVAQGTISDGRPIVGYGKATVKTPDTLDIDITYPSLGNIKAQGQITMDSPTQFKFDATTSGSGGFKGRLTGTLQRLPSAEEADEELKRQGVRGTLAAQLLPGTWQATFTNEDGQTSQGQIHFQPRSP

SEQ ID NO: 420 - protein, 131 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRLPSAEEADEELKRQGVRGTLAAQLLPGTWAVTMTNEDGQTSQGQMHFQPRSP

SEQ ID NO: 421 - protein, 121 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRTSDGGHGPDNAAQLLPGTWQATFTNEDGQTSQGQWHFQPRSP

SEQ ID NO: 422 - protein, 121 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRTSDGGHGPDNAAQLLPGTWAVTMTNEDGQTSQGQWHFQPRSP

SEQ ID NO: 423 - protein, 121 aa, Artificial Sequence
  Synthetic peptide
YTMDIVAQGTISDGRPIVGYGKATVKTPDTLDIDITWPSLGNIKGQGQITMDSPTQFKWDGTTKGENDFHGRLTGTLQRTSDGGHGPDNAAQLLPGTWQATFTNEDGQTSQGQWHFQPRSP

SEQ ID NO: 424 - protein, 121 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRTSDGGHGPDNAAQLLPGTWQATFTNEDGQTSQGQFHFQPRSP

SEQ ID NO: 425 - protein, 121 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRTSDGGHGPDNAAQLLPGTWQATFTNEDGQTSQGQIHFQPRSP

SEQ ID NO: 426 - protein, 121 aa, Artificial Sequence
  Synthetic peptide
YTMDIVSQGTISDGRPIVGYGKATVKTPDTLDIDITYPSLGNIKFQGQITMDSPTQFKFDATTKGENDFHGRLTGTLQRTSDGGHGPDNAAQLLPGTWQATFTNEDGQTSQGQIHFQPRSP

SEQ ID NO: 427 - protein, 121 aa, Artificial Sequence
  Synthetic peptide
YTMDIVAQGTISDGRPIVGYGKATVKTPDTLDIDITYPSLGNIKAQGQITMDSPTQFKFDATTSGSGGFKGRLTGTLQRTSDGGHGPDNAAQLLPGTWQATFTNEDGQTSQGQIHFQPRSP

SEQ ID NO: 428 - protein, 121 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRTSDGGHGPDNAAQLLPGTWAVTMTNEDGQTSQGQMHFQPRSP

SEQ ID NO: 429 - protein, 130 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRTEEAKEATEEARRRGITTQAAQLLPGTWQATFTNEDGQTSQGQWHFQPRSP

SEQ ID NO: 430 - protein, 130 aa, Artificial Sequence
  Synthetic peptide
YTMDIVAQGTISDGRPIVGYGKATVKTPDTLDIDITYPSLGNIKAQGQITMDSPTQFKWDATTKGENDFHGRLTGTLQRTEEAKEATEEARRRGITTQAAQLLPGTWAVTMTNEDGQTSQGQWHFQPRSP

SEQ ID NO: 431 - protein, 130 aa, Artificial Sequence
  Synthetic peptide
YTMDIVAQGTISDGRPIVGYGKATVKTPDTLDIDITWPSLGNIKGQGQITMDSPTQFKWDGTTKGENDFHGRLTGTLQRTEEAKEATEEARRRGITTQAAQLLPGTWQATFTNEDGQTSQGQWHFQPRSP

SEQ ID NO: 432 - protein, 130 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRTEEAKEATEEARRRGITTQAAQLLPGTWQATFTNEDGQTSQGQFHFQPRSP

SEQ ID NO: 433 - protein, 130 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRTEEAKEATEEARRRGITTQAAQLLPGTWQATFTNEDGQTSQGQIHFQPRSP

SEQ ID NO: 434 - protein, 130 aa, Artificial Sequence
  Synthetic peptide
YTMDIVSQGTISDGRPIVGYGKATVKTPDTLDIDITYPSLGNIKFQGQITMDSPTQFKFDATTKGENDFHGRLTGTLQRTEEAKEATEEARRRGITTQAAQLLPGTWQATFTNEDGQTSQGQIHFQPRSP

SEQ ID NO: 435 - protein, 130 aa, Artificial Sequence
  Synthetic peptide
YTMDIVAQGTISDGRPIVGYGKATVKTPDTLDIDITYPSLGNIKAQGQITMDSPTQFKFDATTSGSGGFKGRLTGTLQRTEEAKEATEEARRRGITTQAAQLLPGTWQATFTNEDGQTSQGQIHFQPRSP

SEQ ID NO: 436 - protein, 130 aa, Artificial Sequence
  Synthetic peptide
YTMDIVAQGTISDGRPIVGYGKATVKTPDTLDIDITYPSLGNIKAQGQITMDSPTQFKFDATTKGENDFHGRLTGTLQRTEEAKEATEEARRRGITTQAAQLLPGTWAVTMTNEDGQTSQGQMHFQPRSP

SEQ ID NO: 437 - protein, 135 aa, Artificial Sequence
  Synthetic peptide
DTLDIDITYPSLGNIKAQGQITMDSPTQFKWDATTKGENDFHGRLTGTLQRLPGTEEWAKRIQEELKKKGYGTTKAAQLLPGTWQATFTNEDGQTSQGQWHFQPRSPYTMDIVAQGTISDGRPIVGYGKATVKTP

SEQ ID NO: 438 - protein, 135 aa, Artificial Sequence
  Synthetic peptide
DTLDIDITYPSLGNIKAQGQITMDSPTQFKWDATTKGENDFHGRLTGTLQRLPGTEEWAKRIQEELKKKGYGTTKAAQLLPGTWAVTMTNEDGQTSQGQWHFQPRSPYTMDIVAQGTISDGRPIVGYGKATVKTP

SEQ ID NO: 439 - protein, 135 aa, Artificial Sequence
  Synthetic peptide
DTLDIDITWPSLGNIKGQGQITMDSPTQFKWDGTTKGENDFHGRLTGTLQRLPGTEEWAKRIQEELKKKGYGTTKAAQLLPGTWQATFTNEDGQTSQGQWHFQPRSPYTMDIVAQGTISDGRPIVGYGKATVKTP

SEQ ID NO: 440 - protein, 135 aa, Artificial Sequence
  Synthetic peptide
DTLDIDITYPSLGNIKAQGQITMDSPTQFKFDATTKGENDFHGRLTGTLQRLPGTEEWAKRIQEELKKKGYGTTKAAQLLPGTWQATFTNEDGQTSQGQFHFQPRSPYTMDIVAQGTISDGRPIVGYGKATVKTP

SEQ ID NO: 441 - protein, 135 aa, Artificial Sequence
  Synthetic peptide
DTLDIDITYPSLGNIKAQGQITMDSPTQFKFDATTKGENDFHGRLTGTLQRLPGTEEWAKRIQEELKKKGYGTTKAAQLLPGTWQATFTNEDGQTSQGQIHFQPRSPYTMDIVAQGTISDGRPIVGYGKATVKTP

SEQ ID NO: 442 - protein, 135 aa, Artificial Sequence
  Synthetic peptide
DTLDIDITYPSLGNIKFQGQITMDSPTQFKFDATTKGENDFHGRLTGTLQRLPGTEEWAKRIQEELKKKGYGTTKAAQLLPGTWQATFTNEDGQTSQGQIHFQPRSPYTMDIVSQGTISDGRPIVGYGKATVKTP

SEQ ID NO: 443 - protein, 135 aa, Artificial Sequence
  Synthetic peptide
DTLDIDITYPSLGNIKAQGQITMDSPTQFKFDATTSGSGGFKGRLTGTLQRLPGTEEWAKRIQEELKKKGYGTTKAAQLLPGTWQATFTNEDGQTSQGQIHFQPRSPYTMDIVAQGTISDGRPIVGYGKATVKTP

SEQ ID NO: 444 - protein, 135 aa, Artificial Sequence
  Synthetic peptide
DTLDIDITYPSLGNIKAQGQITMDSPTQFKFDATTKGENDFHGRLTGTLQRLPGTEEWAKRIQEELKKKGYGTTKAAQLLPGTWAVTMTNEDGQTSQGQMHFQPRSPYTMDIVAQGTISDGRPIVGYGKATVKTP

SEQ ID NO: 445 - protein, 131 aa, Artificial Sequence
  Synthetic peptide
DTLDIDITYPSLGNIKAQGQITMDSPTQFKWDATTKGENDFHGRLTGTLQRLPSAEEADEELKRQGVRGTLAAQLLPGTWQATFTNEDGQTSQGQWHFQPRSPYTMDIVAQGTISDGRPIVGYGKATVKTP

SEQ ID NO: 446 - protein, 131 aa, Artificial Sequence
  Synthetic peptide
DTLDIDITYPSLGNIKAQGQITMDSPTQFKWDATTKGENDFHGRLTGTLQRLPSAEEADEELKRQGVRGTLAAQLLPGTWAVTMTNEDGQTSQGQWHFQPRSPYTMDIVAQGTISDGRPIVGYGKATVKTP

SEQ ID NO: 447 - protein, 131 aa, Artificial Sequence
  Synthetic peptide
DTLDIDITWPSLGNIKGQGQITMDSPTQFKWDGTTKGENDFHGRLTGTLQRLPSAEEADEELKRQGVRGTLAAQLLPGTWQATFTNEDGQTSQGQWHFQPRSPYTMDIVAQGTISDGRPIVGYGKATVKTP

SEQ ID NO: 448 - protein, 131 aa, Artificial Sequence
  Synthetic peptide
DTLDIDITYPSLGNIKAQGQITMDSPTQFKFDATTKGENDFHGRLTGTLQRLPSAEEADEELKRQGVRGTLAAQLLPGTWQATFTNEDGQTSQGQFHFQPRSPYTMDIVAQGTISDGRPIVGYGKATVKTP

SEQ ID NO: 449 - protein, 131 aa, Artificial Sequence
  Synthetic peptide
DTLDIDITYPSLGNIKAQGQITMDSPTQFKFDATTKGENDFHGRLTGTLQRLPSAEEADEELKRQGVRGTLAAQLLPGTWQATFTNEDGQTSQGQIHFQPRSPYTMDIVAQGTISDGRPIVGYGKATVKTP

SEQ ID NO: 450 - protein, 131 aa, Artificial Sequence
  Synthetic peptide
DTLDIDITYPSLGNIKFQGQITMDSPTQFKFDATTKGENDFHGRLTGTLQRLPSAEEADEELKRQGVRGTLAAQLLPGTWQATFTNEDGQTSQGQIHFQPRSPYTMDIVSQGTISDGRPIVGYGKATVKTP

SEQ ID NO: 451 - protein, 131 aa, Artificial Sequence
  Synthetic peptide
DTLDIDITYPSLGNIKAQGQITMDSPTQFKFDATTSGSGGFKGRLTGTLQRLPSAEEADEELKRQGVRGTLAAQLLPGTWQATFTNEDGQTSQGQIHFQPRSPYTMDIVAQGTISDGRPIVGYGKATVKTP

SEQ ID NO: 452 - protein, 131 aa, Artificial Sequence
  Synthetic peptide
DTLDIDITYPSLGNIKAQGQITMDSPTQFKFDATTKGENDFHGRLTGTLQRLPSAEEADEELKRQGVRGTLAAQLLPGTWAVTMTNEDGQTSQGQMHFQPRSPYTMDIVAQGTISDGRPIVGYGKATVKTP

SEQ ID NO: 453 - protein, 121 aa, Artificial Sequence
  Synthetic peptide
DTLDIDITYPSLGNIKAQGQITMDSPTQFKWDATTKGENDFHGRLTGTLQRTSDGGHGPDNAAQLLPGTWQATFTNEDGQTSQGQWHFQPRSPYTMDIVAQGTISDGRPIVGYGKATVKTP

SEQ ID NO: 454 - protein, 121 aa, Artificial Sequence
  Synthetic peptide
DTLDIDITYPSLGNIKAQGQITMDSPTQFKWDATTKGENDFHGRLTGTLQRTSDGGHGPDNAAQLLPGTWAVTMTNEDGQTSQGQWHFQPRSPYTMDIVAQGTISDGRPIVGYGKATVKTP

SEQ ID NO: 455 - protein, 121 aa, Artificial Sequence
  Synthetic peptide
DTLDIDITWPSLGNIKGQGQITMDSPTQFKWDGTTKGENDFHGRLTGTLQRTSDGGHGPDNAAQLLPGTWQATFTNEDGQTSQGQWHFQPRSPYTMDIVAQGTISDGRPIVGYGKATVKTP

SEQ ID NO: 456 - protein, 121 aa, Artificial Sequence
  Synthetic peptide
DTLDIDITYPSLGNIKAQGQITMDSPTQFKFDATTKGENDFHGRLTGTLQRTSDGGHGPDNAAQLLPGTWQATFTNEDGQTSQGQFHFQPRSPYTMDIVAQGTISDGRPIVGYGKATVKTP

SEQ ID NO: 457 - protein, 121 aa, Artificial Sequence
  Synthetic peptide
DTLDIDITYPSLGNIKAQGQITMDSPTQFKFDATTKGENDFHGRLTGTLQRTSDGGHGPDNAAQLLPGTWQATFTNEDGQTSQGQIHFQPRSPYTMDIVAQGTISDGRPIVGYGKATVKTP

SEQ ID NO: 458 - protein, 121 aa, Artificial Sequence
  Synthetic peptide
DTLDIDITYPSLGNIKFQGQITMDSPTQFKFDATTKGENDFHGRLTGTLQRTSDGGHGPDNAAQLLPGTWQATFTNEDGQTSQGQIHFQPRSPYTMDIVSQGTISDGRPIVGYGKATVKTP

SEQ ID NO: 459 - protein, 121 aa, Artificial Sequence
  Synthetic peptide
DTLDIDITYPSLGNIKAQGQITMDSPTQFKFDATTSGSGGFKGRLTGTLQRTSDGGHGPDNAAQLLPGTWQATFTNEDGQTSQGQIHFQPRSPYTMDIVAQGTISDGRPIVGYGKATVKTP

SEQ ID NO: 460 - protein, 121 aa, Artificial Sequence
  Synthetic peptide
DTLDIDITYPSLGNIKAQGQITMDSPTQFKFDATTKGENDFHGRLTGTLQRTSDGGHGPDNAAQLLPGTWAVTMTNEDGQTSQGQMHFQPRSPYTMDIVAQGTISDGRPIVGYGKATVKTP

SEQ ID NO: 461 - protein, 130 aa, Artificial Sequence
  Synthetic peptide
DTLDIDITYPSLGNIKAQGQITMDSPTQFKWDATTKGENDFHGRLTGTLQRTEEAKEATEEARRRGITTQAAQLLPGTWQATFTNEDGQTSQGQWHFQPRSPYTMDIVAQGTISDGRPIVGYGKATVKTP

SEQ ID NO: 462 - protein, 130 aa, Artificial Sequence
  Synthetic peptide
DTLDIDITYPSLGNIKAQGQITMDSPTQFKWDATTKGENDFHGRLTGTLQRTEEAKEATEEARRRGITTQAAQLLPGTWAVTMTNEDGQTSQGQWHFQPRSPYTMDIVAQGTISDGRPIVGYGKATVKTP

SEQ ID NO: 463 - protein, 130 aa, Artificial Sequence
  Synthetic peptide
DTLDIDITWPSLGNIKGQGQITMDSPTQFKWDGTTKGENDFHGRLTGTLQRTEEAKEATEEARRRGITTQAAQLLPGTWQATFTNEDGQTSQGQWHFQPRSPYTMDIVAQGTISDGRPIVGYGKATVKTP

SEQ ID NO: 464 - protein, 130 aa, Artificial Sequence
  Synthetic peptide
DTLDIDITYPSLGNIKAQGQITMDSPTQFKFDATTKGENDFHGRLTGTLQRTEEAKEATEEARRRGITTQAAQLLPGTWQATFTNEDGQTSQGQFHFQPRSPYTMDIVAQGTISDGRPIVGYGKATVKTP

SEQ ID NO: 465 - protein, 130 aa, Artificial Sequence
  Synthetic peptide
DTLDIDITYPSLGNIKAQGQITMDSPTQFKFDATTKGENDFHGRLTGTLQRTEEAKEATEEARRRGITTQAAQLLPGTWQATFTNEDGQTSQGQIHFQPRSPYTMDIVAQGTISDGRPIVGYGKATVKTP

SEQ ID NO: 466 - protein, 130 aa, Artificial Sequence
  Synthetic peptide
DTLDIDITYPSLGNIKFQGQITMDSPTQFKFDATTKGENDFHGRLTGTLQRTEEAKEATEEARRRGITTQAAQLLPGTWQATFTNEDGQTSQGQIHFQPRSPYTMDIVSQGTISDGRPIVGYGKATVKTP

SEQ ID NO: 467 - protein, 130 aa, Artificial Sequence
  Synthetic peptide
DTLDIDITYPSLGNIKAQGQITMDSPTQFKFDATTSGSGGFKGRLTGTLQRTEEAKEATEEARRRGITTQAAQLLPGTWQATFTNEDGQTSQGQIHFQPRSPYTMDIVAQGTISDGRPIVGYGKATVKTP

SEQ ID NO: 468 - protein, 130 aa, Artificial Sequence
  Synthetic peptide
DTLDIDITYPSLGNIKAQGQITMDSPTQFKFDATTKGENDFHGRLTGTLQRTEEAKEATEEARRRGITTQAAQLLPGTWAVTMTNEDGQTSQGQMHFQPRSPYTMDIVAQGTISDGRPIVGYGKATVKTP

SEQ ID NO: 469 - protein, 135 aa, Artificial Sequence
  Synthetic peptide
TQFKWDATTKGENDFHGRLTGTLQRLPGTEEWAKRIQEELKKKGYGTTKAAQLLPGTWQATFTNEDGQTSQGQWHFQPRSPYTMDIVAQGTISDGRPIVGYGKATVKTPDTLDIDITYPSLGNIKAQGQITMDSP

SEQ ID NO: 470 - protein, 135 aa, Artificial Sequence
  Synthetic peptide
TQFKWDATTKGENDFHGRLTGTLQRLPGTEEWAKRIQEELKKKGYGTTKAAQLLPGTWAVTMTNEDGQTSQGQWHFQPRSPYTMDIVAQGTISDGRPIVGYGKATVKTPDTLDIDITYPSLGNIKAQGQITMDSP

SEQ ID NO: 471 - protein, 135 aa, Artificial Sequence
  Synthetic peptide
TQFKWDGTTKGENDFHGRLTGTLQRLPGTEEWAKRIQEELKKKGYGTTKAAQLLPGTWQATFTNEDGQTSQGQWHFQPRSPYTMDIVAQGTISDGRPIVGYGKATVKTPDTLDIDITWPSLGNIKGQGQITMDSP

SEQ ID NO: 472 - protein, 135 aa, Artificial Sequence
  Synthetic peptide
TQFKFDATTKGENDFHGRLTGTLQRLPGTEEWAKRIQEELKKKGYGTTKAAQLLPGTWQATFTNEDGQTSQGQFHFQPRSPYTMDIVAQGTISDGRPIVGYGKATVKTPDTLDIDITYPSLGNIKAQGQITMDSP

SEQ ID NO: 473 - protein, 135 aa, Artificial Sequence
  Synthetic peptide
TQFKFDATTKGENDFHGRLTGTLQRLPGTEEWAKRIQEELKKKGYGTTKAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 474 - protein, 135 aa, Artificial Sequence
  Synthetic peptide
TQFKFDATTKGENDFHGRLTGTLQRLPGTEEWAKRIQEELKKKGYGTTKAAQLLPGTWQATFTNEDGQTSQGQIHFQPRSPYTMDIVSQGTISDGRPIVGYGKATVKTPDTLDIDITYPSLGNIKFQGQITMDSP

SEQ ID NO: 475 - protein, 135 aa, Artificial Sequence
  Synthetic peptide
TQFKFDATTSGSGGFKGRLTGTLQRLPGTEEWAKRIQEELKKKGYGTTKAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 476 - protein, 135 aa, Artificial Sequence
  Synthetic peptide
TQFKFDATTKGENDFHGRLTGTLQRLPGTEEWAKRIQEELKKKGYGTTKAAQLLPGTWAVTMTNEDGQTSQGQMHFQPRSPYTMDIVAQGTISDGRPIVGYGKATVKTPDTLDIDITYPSLGNIKAQGQITMDSP

SEQ ID NO: 477 - protein, 131 aa, Artificial Sequence
  Synthetic peptide
TQFKWDATTKGENDFHGRLTGTLQRLPSAEEADEELKRQGVRGTLAAQLLPGTWQATFTNEDGQTSQGQWHFQPRSPYTMDIVAQGTISDGRPIVGYGKATVKTPDTLDIDITYPSLGNIKAQGQITMDSP

SEQ ID NO: 478 - protein, 131 aa, Artificial Sequence
  Synthetic peptide
TQFKWDATTKGENDFHGRLTGTLQRLPSAEEADEELKRQGVRGTLAAQLLPGTWAVTMTNEDGQTSQGQWHFQPRSPYTMDIVAQGTISDGRPIVGYGKATVKTPDTLDIDITYPSLGNIKAQGQITMDSP

SEQ ID NO: 479 - protein, 131 aa, Artificial Sequence
  Synthetic peptide
TQFKWDGTTKGENDFHGRLTGTLQRLPSAEEADEELKRQGVRGTLAAQLLPGTWQATFTNEDGQTSQGQWHFQPRSPYTMDIVAQGTISDGRPIVGYGKATVKTPDTLDIDITWPSLGNIKGQGQITMDSP

SEQ ID NO: 480 - protein, 131 aa, Artificial Sequence
  Synthetic peptide
TQFKFDATTKGENDFHGRLTGTLQRLPSAEEADEELKRQGVRGTLAAQLLPGTWQATFTNEDGQTSQGQFHFQPRSPYTMDIVAQGTISDGRPIVGYGKATVKTPDTLDIDITYPSLGNIKAQGQITMDSP

SEQ ID NO: 481 - protein, 131 aa, Artificial Sequence
  Synthetic peptide
TQFKFDATTKGENDFHGRLTGTLQRLPSAEEADEELKRQGVRGTLAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 482 - protein, 131 aa, Artificial Sequence
  Synthetic peptide
TQFKFDATTKGENDFHGRLTGTLQRLPSAEEADEELKRQGVRGTLAAQLLPGTWQATFTNEDGQTSQGQIHFQPRSPYTMDIVSQGTISDGRPIVGYGKATVKTPDTLDIDITYPSLGNIKFQGQITMDSP

SEQ ID NO: 483 - protein, 131 aa, Artificial Sequence
  Synthetic peptide
TQFKFDATTSGSGGFKGRLTGTLQRLPSAEEADEELKRQGVRGTLAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 484 - protein, 131 aa, Artificial Sequence
  Synthetic peptide
TQFKFDATTKGENDFHGRLTGTLQRLPSAEEADEELKRQGVRGTLAAQLLPGTWAVTMTNEDGQTSQGQMHFQPRSPYTMDIVAQGTISDGRPIVGYGKATVKTPDTLDIDITYPSLGNIKAQGQITMDSP

SEQ ID NO: 485 - protein, 121 aa, Artificial Sequence
  Synthetic peptide
TQFKWDATTKGENDFHGRLTGTLQRTSDGGHGPDNAAQLLPGTWQATFTNEDGQTSQGQWHFQPRSPYTMDIVAQGTISDGRPIVGYGKATVKTPDTLDIDITYPSLGNIKAQGQITMDSP

SEQ ID NO: 486 - protein, 121 aa, Artificial Sequence
  Synthetic peptide
TQFKWDATTKGENDFHGRLTGTLQRTSDGGHGPDNAAQLLPGTWAVTMTNEDGQTSQGQWHFQPRSPYTMDIVAQGTISDGRPIVGYGKATVKTPDTLDIDITYPSLGNIKAQGQITMDSP

SEQ ID NO: 487 - protein, 121 aa, Artificial Sequence
  Synthetic peptide
TQFKWDGTTKGENDFHGRLTGTLQRTSDGGHGPDNAAQLLPGTWQATFTNEDGQTSQGQWHFQPRSPYTMDIVAQGTISDGRPIVGYGKATVKTPDTLDIDITWPSLGNIKGQGQITMDSP

SEQ ID NO: 488 - protein, 121 aa, Artificial Sequence
  Synthetic peptide
TQFKFDATTKGENDFHGRLTGTLQRTSDGGHGPDNAAQLLPGTWQATFTNEDGQTSQGQFHFQPRSPYTMDIVAQGTISDGRPIVGYGKATVKTPDTLDIDITYPSLGNIKAQGQITMDSP

SEQ ID NO: 489 - protein, 121 aa, Artificial Sequence
  Synthetic peptide
TQFKFDATTKGENDFHGRLTGTLQRTSDGGHGPDNAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 490 - protein, 121 aa, Artificial Sequence
  Synthetic peptide
TQFKFDATTKGENDFHGRLTGTLQRTSDGGHGPDNAAQLLPGTWQATFTNEDGQTSQGQIHFQPRSPYTMDIVSQGTISDGRPIVGYGKATVKTPDTLDIDITYPSLGNIKFQGQITMDSP

SEQ ID NO: 491 - protein, 121 aa, Artificial Sequence
  Synthetic peptide
TQFKFDATTSGSGGFKGRLTGTLQRTSDGGHGPDNAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 492 - protein, 121 aa, Artificial Sequence
  Synthetic peptide
TQFKFDATTKGENDFHGRLTGTLQRTSDGGHGPDNAAQLLPGTWAVTMTNEDGQTSQGQMHFQPRSPYTMDIVAQGTISDGRPIVGYGKATVKTPDTLDIDITYPSLGNIKAQGQITMDSP

SEQ ID NO: 493 - protein, 130 aa, Artificial Sequence
  Synthetic peptide
TQFKWDATTKGENDFHGRLTGTLQRTEEAKEATEEARRRGITTQAAQLLPGTWQATFTNEDGQTSQGQWHFQPRSPYTMDIVAQGTISDGRPIVGYGKATVKTPDTLDIDITYPSLGNIKAQGQITMDSP

SEQ ID NO: 494 - protein, 130 aa, Artificial Sequence
  Synthetic peptide
TQFKWDATTKGENDFHGRLTGTLQRTEEAKEATEEARRRGITTQAAQLLPGTWAVTMTNEDGQTSQGQWHFQPRSPYTMDIVAQGTISDGRPIVGYGKATVKTPDTLDIDITYPSLGNIKAQGQITMDSP

SEQ ID NO: 495 - protein, 130 aa, Artificial Sequence
  Synthetic peptide
TQFKWDGTTKGENDFHGRLTGTLQRTEEAKEATEEARRRGITTQAAQLLPGTWQATFTNEDGQTSQGQWHFQPRSPYTMDIVAQGTISDGRPIVGYGKATVKTPDTLDIDITWPSLGNIKGQGQITMDSP

SEQ ID NO: 496 - protein, 130 aa, Artificial Sequence
  Synthetic peptide
TQFKFDATTKGENDFHGRLTGTLQRTEEAKEATEEARRRGITTQAAQLLPGTWQATFTNEDGQTSQGQFHFQPRSPYTMDIVAQGTISDGRPIVGYGKATVKTPDTLDIDITYPSLGNIKAQGQITMDSP

SEQ ID NO: 497 - protein, 130 aa, Artificial Sequence
  Synthetic peptide
TQFKFDATTKGENDFHGRLTGTLQRTEEAKEATEEARRRGITTQAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 498 - protein, 130 aa, Artificial Sequence
  Synthetic peptide
TQFKFDATTKGENDFHGRLTGTLQRTEEAKEATEEARRRGITTQAAQLLPGTWQATFTNEDGQTSQGQIHFQPRSPYTMDIVSQGTISDGRPIVGYGKATVKTPDTLDIDITYPSLGNIKFQGQITMDSP

SEQ ID NO: 499 - protein, 130 aa, Artificial Sequence
  Synthetic peptide
TQFKFDATTSGSGGFKGRLTGTLQRTEEAKEATEEARRRGITTQAAQLLPGTWQATFTNEDGQTSQGQIHFQPRSPYTMDIVAQGTISDGRPIVGYGKATVKTPDTLDIDITYPSLGNIKAQGQITMDSP

SEQ ID NO: 500 - protein, 130 aa, Artificial Sequence
  Synthetic peptide
TQFKFDATTKGENDFHGRLTGTLQRTEEAKEATEEARRRGITTQAAQLLPGTWAVTMTNEDGQTSQGQMHFQPRSPYTMDIVAQGTISDGRPIVGYGKATVKTPDTLDIDITYPSLGNIKAQGQITMDSP

SEQ ID NO: 501 - protein, 135 aa, Artificial Sequence
  Synthetic peptide
RLTGTLQRLPGTEEWAKRIQEELKKKGYGTTKAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 502 - protein, 135 aa, Artificial Sequence
  Synthetic peptide
RLTGTLQRLPGTEEWAKRIQEELKKKGYGTTKAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 503 - protein, 135 aa, Artificial Sequence
  Synthetic peptide
RLTGTLQRLPGTEEWAKRIQEELKKKGYGTTKAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFHG

SEQ ID NO: 504 - protein, 135 aa, Artificial Sequence
  Synthetic peptide
RLTGTLQRLPGTEEWAKRIQEELKKKGYGTTKAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFHG

SEQ ID NO: 505 - protein, 135 aa, Artificial Sequence
  Synthetic peptide
RLTGTLQRLPGTEEWAKRIQEELKKKGYGTTKAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFHG

SEQ ID NO: 506 - protein, 135 aa, Artificial Sequence
  Synthetic peptide
RLTGTLQRLPGTEEWAKRIQEELKKKGYGTTKAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFHG

SEQ ID NO: 507 - protein, 135 aa, Artificial Sequence
  Synthetic peptide
RLTGTLQRLPGTEEWAKRIQEELKKKGYGTTKAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFKG

SEQ ID NO: 508 - protein, 135 aa, Artificial Sequence
  Synthetic peptide
RLTGTLQRLPGTEEWAKRIQEELKKKGYGTTKAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFHG

SEQ ID NO: 509 - protein, 131 aa, Artificial Sequence
  Synthetic peptide
RLTGTLQRLPSAEEADEELKRQGVRGTLAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 510 - protein, 131 aa, Artificial Sequence
  Synthetic peptide
RLTGTLQRLPSAEEADEELKRQGVRGTLAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 511 - protein, 131 aa, Artificial Sequence
  Synthetic peptide
RLTGTLQRLPSAEEADEELKRQGVRGTLAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFHG

SEQ ID NO: 512 - protein, 131 aa, Artificial Sequence
  Synthetic peptide
RLTGTLQRLPSAEEADEELKRQGVRGTLAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFHG

SEQ ID NO: 513 - protein, 131 aa, Artificial Sequence
  Synthetic peptide
RLTGTLQRLPSAEEADEELKRQGVRGTLAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFHG

SEQ ID NO: 514 - protein, 131 aa, Artificial Sequence
  Synthetic peptide
RLTGTLQRLPSAEEADEELKRQGVRGTLAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFHG

SEQ ID NO: 515 - protein, 131 aa, Artificial Sequence
  Synthetic peptide
RLTGTLQRLPSAEEADEELKRQGVRGTLAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFKG

SEQ ID NO: 516 - protein, 131 aa, Artificial Sequence
  Synthetic peptide
RLTGTLQRLPSAEEADEELKRQGVRGTLAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFHG

SEQ ID NO: 517 - protein, 121 aa, Artificial Sequence
  Synthetic peptide
RLTGTLQRTSDGGHGPDNAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 518 - protein, 121 aa, Artificial Sequence
  Synthetic peptide
RLTGTLQRTSDGGHGPDNAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 519 - protein, 121 aa, Artificial Sequence
  Synthetic peptide
RLTGTLQRTSDGGHGPDNAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFHG

SEQ ID NO: 520 - protein, 121 aa, Artificial Sequence
  Synthetic peptide
RLTGTLQRTSDGGHGPDNAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFHG

SEQ ID NO: 521 - protein, 121 aa, Artificial Sequence
  Synthetic peptide
RLTGTLQRTSDGGHGPDNAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFHG

SEQ ID NO: 522 - protein, 121 aa, Artificial Sequence
  Synthetic peptide
RLTGTLQRTSDGGHGPDNAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFHG

SEQ ID NO: 523 - protein, 121 aa, Artificial Sequence
  Synthetic peptide
RLTGTLQRTSDGGHGPDNAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFKG

SEQ ID NO: 524 - protein, 121 aa, Artificial Sequence
  Synthetic peptide
RLTGTLQRTSDGGHGPDNAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFHG

SEQ ID NO: 525 - protein, 130 aa, Artificial Sequence
  Synthetic peptide
RLTGTLQRTEEAKEATEEARRRGITTQAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 526 - protein, 130 aa, Artificial Sequence
  Synthetic peptide
RLTGTLQRTEEAKEATEEARRRGITTQAAQLLPGTWAVTMTNEDGQTSQGQWHFQPRSPYTMDIVAQGTISDGRPIVGYGKATVKTPDTLDIDITYPSLGNIKAQGQITMDSPTQFKWDATTKGENDFHG

SEQ ID NO: 527 - protein, 130 aa, Artificial Sequence
  Synthetic peptide
RLTGTLQRTEEAKEATEEARRRGITTQAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFHG

SEQ ID NO: 528 - protein, 130 aa, Artificial Sequence
  Synthetic peptide
RLTGTLQRTEEAKEATEEARRRGITTQAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFHG

SEQ ID NO: 529 - protein, 130 aa, Artificial Sequence
  Synthetic peptide
RLTGTLQRTEEAKEATEEARRRGITTQAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFHG

SEQ ID NO: 530 - protein, 130 aa, Artificial Sequence
  Synthetic peptide
RLTGTLQRTEEAKEATEEARRRGITTQAAQLLPGTWQATFTNEDGQTSQGQIHFQPRSPYTMDIVSQGTISDGRPIVGYGKATVKTPDTLDIDITYPSLGNIKFQGQITMDSPTQFKFDATTKGENDFHG

SEQ ID NO: 531 - protein, 130 aa, Artificial Sequence
  Synthetic peptide
RLTGTLQRTEEAKEATEEARRRGITTQAAQLLPGTWQATFTNEDGQTSQGQIHFQPRSPYTMDIVAQGTISDGRPIVGYGKATVKTPDTLDIDITYPSLGNIKAQGQITMDSPTQFKFDATTSGSGGFKG

SEQ ID NO: 532 - protein, 130 aa, Artificial Sequence
  Synthetic peptide
RLTGTLQRTEEAKEATEEARRRGITTQAAQLLPGTWAVTMTNEDGQTSQGQMHFQPRSPYTMDIVAQGTISDGRPIVGYGKATVKTPDTLDIDITYPSLGNIKAQGQITMDSPTQFKFDATTKGENDFHG

SEQ ID NO: 533 - protein, 115 aa, Artificial Sequence
  Synthetic peptide
SRAAQLLPGTWQATFTNEDGQTSQGQFHFQPRSPYTMDIVAQGTISDGRPIVGYGKATVKTPDTLDIDITYPSLGNIKAQGQITMDSPTQFKFDATTKGENDFHGRLTGTLQRQE

SEQ ID NO: 534 - protein, 115 aa, Artificial Sequence
  Synthetic peptide
SRAAQLLPGTWQATFTNEDGQTSQGQIHFQPRSPYTMDIVAQGTISDGRPIVGYGKATVKTPDTLDIDITYPSLGNIKAQGQITMDSPTQFKFDATTKGENDFHGRLTGTLQRQE

SEQ ID NO: 535 - protein, 7 aa, Artificial Sequence
  Synthetic peptide
  MISC_FEATURE (3)..(3): Xaa is A, I, or Y.
  MISC_FEATURE (4)..(4): Xaa is R, S, Q, or A.
RAXXLLP

SEQ ID NO: 536 - protein, 7 aa, Artificial Sequence
  Synthetic peptide
RAAQLLP

SEQ ID NO: 537 - protein, 9 aa, Artificial Sequence
  Synthetic peptide
  MISC_FEATURE (2)..(2): Xaa is T, K, N, or D.
  MISC_FEATURE (5)..(2): Xaa is any amino acid, or GTWQ(V/L/A/I) T(M/F)TN.
  misc_feature (5)..(5): Xaa can be any naturally occurring amino acid
  MISC_FEATURE (7)..(7): Xaa is M or F.
GXWQXTXTN

SEQ ID NO: 538 - protein, 9 aa, Artificial Sequence
  Synthetic peptide
  MISC_FEATURE (5)..(5): Xaa is V, L, A, or I.
  MISC_FEATURE (7)..(7): Xaa is M or F.
GTWQXTXTN

SEQ ID NO: 539 - protein, 11 aa, Artificial Sequence
  Synthetic peptide
QTSQGQMHFQP

SEQ ID NO: 540 - protein, 9 aa, Artificial Sequence
  Synthetic peptide
  MISC_FEATURE (2)..(2): Xaa is L, A, or M.
  MISC_FEATURE (4)..(4): Xaa is I or V.
  MISC_FEATURE (5)..(5): Xaa is K or V.
  MISC_FEATURE (6)..(6): Xaa is A or S.
  MISC_FEATURE (9)..(9): Xaa is I or M.
TXDXXXGTX

SEQ ID NO: 541 - protein, 10 aa, Artificial Sequence
  Synthetic peptide
TMDIVAQGTI

SEQ ID NO: 542 - protein, 11 aa, Artificial Sequence
  Synthetic peptide
  MISC_FEATURE (4)..(4): Xaa is Q, S, T, or V.
  MISC_FEATURE (6)..(6): Xaa is Y or K.
  MISC_FEATURE (9)..(9): Xaa is L, V, or A.
  MISC_FEATURE (11)..(11): Xaa is V, C, or A.
RPIXGXGKXTX

SEQ ID NO: 543 - protein, 11 aa, Artificial Sequence
  Synthetic peptide
RPIVGYGKATV

SEQ ID NO: 544 - protein, 8 aa, Artificial Sequence
  Synthetic peptide
  MISC_FEATURE (2)..(2): Xaa is M, L, or V.
  MISC_FEATURE (3)..(3): Xaa is D, H, Q, or N.
  MISC_FEATURE (4)..(4): Xaa is V, A, L, or I.
  MISC_FEATURE (5)..(5): Xaa is D, N, H, or Q.
  MISC_FEATURE (6)..(6): Xaa is I, L, or V.
  MISC_FEATURE (8)..(8): Xaa is Y or W.
TXXXXXTX

SEQ ID NO: 545 - protein, 8 aa, Artificial Sequence
  Synthetic peptide
TLDIDITY

SEQ ID NO: 546 - protein, 5 aa, Artificial Sequence
  Synthetic peptide
PSLGN

SEQ ID NO: 547 - protein, 9 aa, Artificial Sequence
  Synthetic peptide
  MISC_FEATURE (1)..(1): Xaa is K, M, I, or L.
  MISC_FEATURE (2)..(2): Xaa is Q or K.
  MISC_FEATURE (3)..(3): Xaa is V, A, or G.
  MISC_FEATURE (7)..(7): Xaa is V or I.
  MISC_FEATURE (9)..(9): Xaa is M, L, or Y.
XXXQGQXTX

SEQ ID NO: 548 - protein, 9 aa, Artificial Sequence
  Synthetic peptide
IKAQGQITM

SEQ ID NO: 549 - protein, 8 aa, Artificial Sequence
  Synthetic peptide
  MISC_FEATURE (2)..(2): Xaa is Q or A.
  MISC_FEATURE (3)..(3): Xaa is K, T, or H.
  MISC_FEATURE (4)..(4): Xaa is F or W.
  MISC_FEATURE (5)..(5): Xaa is D or N.
  MISC_FEATURE (6)..(6): Xaa is V, A, S, or G.
  MISC_FEATURE (7)..(7): Xaa is T, Q, H, or E.
  MISC_FEATURE (8)..(8): Xaa is T, F, V, or Y.
QXXXXXXX

SEQ ID NO: 550 - protein, 8 aa, Artificial Sequence
  Synthetic peptide
QFKFDATT

SEQ ID NO: 551 - protein, 11 aa, Artificial Sequence
  Synthetic peptide
  MISC_FEATURE (1)..(1): Xaa is optional, and when present, is S, K, N or H.
  MISC_FEATURE (2)..(2): Xaa is K, R, I, of N.
  MISC_FEATURE (3)..(3): Xaa is V or L.
  MISC_FEATURE (7)..(7): Xaa is L, I, or M.
XXXTGTXQRQE

SEQ ID NO: 552 - protein, 10 aa, Artificial Sequence
  Synthetic peptide
RLTGTLQRQE

SEQ ID NO: 553 - protein, 5 aa, Artificial Sequence
  Synthetic peptide
  MISC_FEATURE (2)..(2): Xaa is G, Q, K, or T.
  MISC_FEATURE (3)..(3): Xaa is A, D, E, or N.
  MISC_FEATURE (4)..(4): Xaa is G, D, or N.
  MISC_FEATURE (5)..(5): Xaa is N, G, D, Y or S.
KXXXX

SEQ ID NO: 554 - protein, 5 aa, Artificial Sequence
  Synthetic peptide
  MISC_FEATURE (3)..(3): Xaa is A, D, or E.
  MISC_FEATURE (4)..(4): Xaa is G, D, or N.
  MISC_FEATURE (5)..(5): Xaa is N, G, D, or Y.
KGXXX

SEQ ID NO: 555 - protein, 8 aa, Artificial Sequence
  Synthetic peptide
KGENDFHG

SEQ ID NO: 556 - protein, 8 aa, Artificial Sequence
  Synthetic peptide
KGADGWHG

SEQ ID NO: 557 - protein, 8 aa, Artificial Sequence
  Synthetic peptide
KGAGNFTG

SEQ ID NO: 558 - protein, 11 aa, Artificial Sequence
  Synthetic peptide
LPGGGGGDGTR

SEQ ID NO: 559 - protein, 24 aa, Artificial Sequence
  Synthetic peptide
TPNAEEYLKELEERKRKGMQPLNE

SEQ ID NO: 560 - protein, 24 aa, Artificial Sequence
  Synthetic peptide
TPKAGDEEYAKRLEEEARKKGGTI

SEQ ID NO: 561 - protein, 13 aa, Artificial Sequence
  Synthetic peptide
TEPTGGGGGGGVT

SEQ ID NO: 562 - protein, 26 aa, Artificial Sequence
  Synthetic peptide
LPTAEEWYKRWEKELRKRGTSWEQTL

SEQ ID NO: 563 - protein, 20 aa, Artificial Sequence
  Synthetic peptide
EPRSEEIVKKAQHTWKGGSL

SEQ ID NO: 564 - protein, 20 aa, Artificial Sequence
  Synthetic peptide
LPTAEEAQKEVKKKGLTGSN

SEQ ID NO: 565 - protein, 24 aa, Artificial Sequence
  Synthetic peptide
LPGTEEWAKRIQEELKKKGYGTTK

SEQ ID NO: 566 - protein, 23 aa, Artificial Sequence
  Synthetic peptide
TEEAKKILKEIQKKHKDEVQTDR

SEQ ID NO: 567 - protein, 20 aa, Artificial Sequence
  Synthetic peptide
LPSAEEADEELKRQGVRGTL

SEQ ID NO: 568 - protein, 10 aa, Artificial Sequence
  Synthetic peptide
TSDGGHGPDN